SEQUENCE LISTING (88 sequences)

SEQ ID NO: 1 - protein, 765 aa, Escherichia coli
MTNRISRLKTALFANTREISLERALLYTASHRQTEGEPVILRRAKATAYILEHVEISIRDEELIAGNRTVKPRAGIMSPEMDPYWLLKELDQFPTRPQDRFAISEEDKRIYREELFPYWEKRSMKDFINGQMTDEVKAATNTQIFSINQTDKGQGHIIIDYPRLLNHGLGELVAQMQQHCQQQPENHFYQAALLLLEASQKHILRYAELAETMAANCTDAQRREELLTIAEISRHNAQHKPQTFWQACQLFWYMNIILQYESNASSLSLGRFDQYMLPFYQTSLTQGEDAAFLKELLESLWVKCNDIVLLRSTSSARYFAGFPTGYTALLGGLTENGRSAVNVLSFLCLDAYQSVQLPQPNLGVRTNALIDTPFLMKTAETIRFGTGIPQIFNDEVVVPAFLNRGVSLEDARDYSVVGCVELSIPGRTYGLHDIAMFNLLKVMEICLHENEGNAALTYEGLLEQIRAKISHYITLMVEGSNICDIGHRDWAPVPLLSSFISDCLEKGRDITDGGARYNFSGVQGIGIANLSDSLHALKGMVFEQQRLSFDELLSVLKANFATPEGEKVRARLINRFEKYGNDIDEVDNISAELLRHYCKEVEKYQNPRGGYFTPGSYTVSAHVPLGSVVGATPDGRFAGEQLADGGLSPMLGQDAQGPTAVLKSVSKLDNTLLSNGTLLNVKFTPATLEGEAGLRKLADFLRAFTQLKLQHIQFNVVNADTLREAQQRPQDYAGLVVRVAGYSAFFVELSKEIQDDIIRRTAHQL

SEQ ID NO: 2 - protein, 486 aa, Escherichia coli
MTTIVDSNLPVARPSWDHSRLESRIVHLGCGAFHRAHQALYTHHLLESTDSDWGICEVNLMPGNDRVLIENLKKQQLLYTVAEKGAESTELKIIGSMKEALHPEIDGCEGILNAMARPQTAIVSLTVTEKGYCADAASGQLDLNNPLIKHDLENPTAPKSAIGYIVEALRLRREKGLKAFTVMSCDNVRENGHVAKVAVLGLAQARDPQLAAWIEENVTFPCTMVDRIVPAATPETLQEIADQLGVYDPCAIACEPFRQWVIEDNFVNGRPDWDKVGAQFVADVVPFEMMKLRMLNGSHSFLAYLGYLGGYETIADTVTNPAYRKAAFALMMQEQAPTLSMPEGTDLNAYATLLIERFSNPSLRHRTWQIAMDGSQKLPQRLLDPVRLHLQNGGSWRHLALGVAGWMRYTQGVDEQGNAIDVVDPMLAEFQKINAQYQGADRVKALLGLSGIFADDLPQNADFVGAVTAAYQQLCERGARECVAAL

SEQ ID NO: 3 - protein, 655 aa, Metallosphaera sedula
MVEEQTLKTGSQELEEKADYNMRYYAHLMKLSKEKPAEFWGSLAQDLLDWYEPWKETMRQEDPMTRWFIGGKINASYNAVDRHLNGPRKFKAAVIWESELGERKIVTYQDMFYEVNRWANALRSLGVGKGDRVTIYMPLTPEGIAAMLASARIGAIHSVIFAGFGSQAIADRVEDAKAKVVITADAYPRRGKVVELKKTVDEALNSLGERSPVQHVLVYRRMKTDVNMKEGRDVFFDEVGKYRYVEPERMDSNDPLFILYTSGTTGKPKGIMHSTGGYLTGTAVMLLWSYGLSQENDVLFNTSDIGWIVGHSYITYSPLIMGRTVVIYESAPDYPYPDKWAEIIERYRATTFGTSATALRYFMKYGDEYVKNHDLSSIRIIVTNGEVLNYSPWKWGLEVLGGGKVFMSHQWWQTETGAPNLGYLPGIIYMPMKSGPASGFPLPGNFVEVLDENGNPSAPRVRGYLVMRPPFPPNMMMGMWNDNGERLKKTYFSKFGSLYYPGDFAMVDEDGYIWVLGRADETLKIAAHRIGAGEVESAITSHPSVAEAAVIGVPDSVKGEEVHAFVVLKQGYAPSSELAKDIQSHVRKVMGPIVSPQIHFVDKLPKTRSGKVMRRVIKAVMMGSSAGDLTTIEDEASMDEIKKAVEELKKELKTS

SEQ ID NO: 4 - protein, 416 aa, Escherichia coli
MSTPLQGIKVLDFTGVQSGPSCTQMLAWFGADVIKIERPGVGDVTRHQLRDIPDIDALYFTMLNSNKRSIELNTKTAEGKEVMEKLIREADILVENFHPGAIDHMGFTWEHIQEINPRLIFGSIKGFDECSPYVNVKAYENVAQAAGGAASTTGFWDGPPLVSAAALGDSNTGMHLLIGLLAALLHREKTGRGQRVTMSMQDAVLNLCRVKLRDQQRLDKLGYLEEYPQYPNGTFGDAVPRGGNAGGGGQPGWILKCKGWETDPNAYIYFTIQEQNWENTCKAIGKPEWITDPAYSTAHARQPHIFDIFAEIEKYTVTIDKHEAVAYLTQFDIPCAPVLSMKEISLDPSLRQSGSVVEVEQPLRGKYLTVGCPMKFSAFTPDIKAAPLLGEHTAAVLQELGYSDDEIAAMKQNHAI

SEQ ID NO: 5 - protein, 398 aa, Cupriavidus necator
MSASKFAAPDAAPDATASEPAAPRVKKSVALSGVTAGNTALCTVGRTGNDLHYRGYDILDIAETCEFEEIAHLLVHGKLPTKSELAAYKAKLKSLRGLPANVKAALEWVPASAHPMDVMRTGVSVLGTVLPEKEDHNTPGARDIADRLMASLGSMLLYWYHYSHNGRRIEVETDDDSIGGHFLHLLHGEKPSALWERAMHTSLNLYAEHEFNASTFTARVIAGTGSDMYSSISGAIGALRGPKHGGANEVAFEIQKRYDNPDEAQADITRRVGNKEVVIGFGHPVYTTGDPRNQVIKEVAKKLSKDAGSMKMFDIAERLETVMWDIKKMFPNLDWFSAVSYHMMGVPTAMFTPLFVIARTSGWAAHIIEQRIDNKIIRPSANYTGPENLKFVPIGKRK

SEQ ID NO: 6 - protein, 460 aa, Cupriavidus necator
MSQPADTTYPTRQLCDFLADLKLADVPAPVIERTKDLFLDWIASAIAGKDAPAVRKLQEFAAAMGPDQGGAEVLVDRRRTSPYFAALINGASSHVVEQDDVHNGSVLHPAAVVFPAVVAAAQTEGKTGAEVLLASIAGYEAGIRIGEFMGRSHYRVFHTTGTVGTLAAAAAVAKLYGLDAEGINQALGSAGTQAAGLWEFLRDAADSKQLHTAKAAADGLQSAWLARAGFTGAKQILEGAQGMAAGMSSDANPACLTDGLGTRWATAETSFKFFASCRHTHPAADALKALMQREGIGADQVASVTTHVHQGAIDVLGPVVNPTTIHQAKFSMGTVLGLVAVHGHAGLGEFEQHALQDPKVTAFRGKVGMELDAEINAAYPRQWIGRVTVTTTDGRTLAARVDVPKGDPDNTLSRPELEAKALQLGAFRHGASEAEMRAIIQRVWALEQAPNVNDWLPAAG

SEQ ID NO: 7 - protein, 460 aa, Cupriavidus necator; identical to SEQ ID NO: 6

SEQ ID NO: 8 - protein, 302 aa, Cupriavidus necator
MTYSASDLARSAGARFRQALADEHPLQVVGTINANHALLAKRAGYRAIYLSGGGVAAGSLGLPDLGISNLDDVLTDIRRITDVCDTPLLVDVDTGFGASAFNVARTTKSLIKFGAAAMHIEDQVGAKRCGHRPNKEIVTQGEMVDRIRAAVDARTDENFVIMARTDALAVEGLDKAIERAVACVEAGADAIFPEAMTDLAMYRKFVDAVKVPVLANITEFGATPLFTTEELGGAGVSMVLYPLSAFRAMNKAAENVYAAIRRDGTQKNVVDTMQTRAELYESIGYHDFEQKLDALFAQGKGK

SEQ ID NO: 9 - protein, 450 aa, Cupriavidus necator
MFEKILIANRGEIALRIQRACRELGIKTVVVYSEADKEAKYVRLADEAVCIGPAPSPLSYLNMPAIISAAEVTDAQAIHPGYGFLSENADFAERVEKSGFVFIGPTADSIRLMGDKVSAKQAMIKAGVPCVPGSDGALPDDPKEILATARRVGYPVIIKAAGGGGGRGMRVVHTEAALINAVNMTREEAGRAFGNPEVYMEKFLENPRHVEIQILADQHRQAIWLGERDCSMQRRHQKVIEEAPAPHIPRRLIERIGDRCAEACKKMGYRGAGTFEFLYENNEFYFIEMNTRVQVEHPVTEMITGIDIVQEQIRIAFGEKLRLRQKDVQFRGHAIECRVNAEDPFKFTPSPGRITAWHMPGGPGVRVDSHAYDGYFVPPNYDSMIGKIITYGATREQAIARMRIALSEMVVDGIQTNVPLHRELMMDANFVEGGTSIHYLEHRLAERAKA

SEQ ID NO: 10 - protein, 290 aa, Cupriavidus necator
MSWLDKLLPPKIQQTDPSTRKGIPEGLWVKCPSCESTLYRTDVEANLHVCPKCDHHMRISARARLDGLLDAEGRYEIGQEIVPVDALKFKDSKKYPDRIKAAMDDTGETDAMVVMGGAIHTIPVVVSCFEFEFMGGSMGSVVGERFVRGAQAALEQKVPFICFTATGGARMQESLLSLLQMAKTTSMLNQLSAAKLPFISVLTDPTMGGVSASFAFLGDVVIAEPKALIGFAGPRVIEQTVREKLPEGFQRSEFLIQKGAIDMIVDRRKMRAELAQLLALLQKQPADAVA

SEQ ID NO: 11 - protein, 323 aa, Cupriavidus necator
MKTTFLDFEQPIAELEAKIEELRFVQDDSAVDISEEISRLAGKSQQLTKDIYANLTPWQVAQIARHPQRPYTLDYVREIFTDFHELHGDRTFADDLSIIGGLARFNGQACMVIGHQKGRDTKERAMRNFGMPKPEGYRKAKRLMELADKFGLPIFTFVDTPGAFPGIDAEERGQSEAIGHNLYVMAGLKVPLIATIIGEGGSGGALAIAVGDVVQMLQFATYAVISPEGCASILWKTAEKAPEAAEALGLTAHRLKALGLIDKIVSEPLGGAHRDYKGMAAMLKRSLAESLRQFQGMSVKELQARRYERLLAYGKFKETGTQD

SEQ ID NO: 12 - protein, 354 aa, Cupriavidus necator
MPLVSMRQLLDHAAENSYGLPAFNVNNLEQVQAIMQAADEVNAPVIMQASAGARKYAGEHFLRHLIEAAVEAYPHIPVVMHQDHGQSPAICQGAIDLGFSSVMMDGSLREDGKTPAEYDYNIDVTRKVVQLAHAVGVTVEGELGCLGSLETGEAGEEDGIGAVGVLDHSMLLTDPEQAADFVKATQLDALAIAIGTSHGAYKFTRKPTGDILAISRIKEIHARIPNTHLVMHGSSSVPQELLEEIRKFGGDMKETYGVPVEEIQEAIKYGVRKINIDTDIRLAMTGAIRRFFAENPSKFDPREYLKPAREAAKQVCKARYIAFGCEGQAGKIKPIGLTDIAQQYKAGKLSQVVQ

SEQ ID NO: 13 - protein, 256 aa, Cupriavidus necator
MSADANIQVTHEGHVARIVLSRPPHNFVDADIMRRLADTLQALDDNENCRAIVLASGVSAFCAGADFSGADQGAIANDPGGFYAHAMRLYRNRKPIVAAVEGAAIGAGMGLALVADFRVTCAEARFSANFNRLGFHPGFGMSLTLPRLVGEQQAALLFYTGRRITGTDAVEIGLADELVEKAQVNARAMALAQEIAASAPLAVESTRATLREGLADRIAEINQRELAIQHGQFRSEDFREGVAAMAARRAPVFKRR

SEQ ID NO: 14 - protein, 323 aa, Cupriavidus necator
MKAVRVVPGPEGGKVEVQDIPVPVVAAGQVLVRVRAAGLNRGEINQAKELRSGNTITTGVEFAGEIAEVGEGVSGWRKGDRVMGHGRGCQAEYVVADPLALIPVPDGLSWIDAAAFPNVFITAHDALVTNGRLVAGESVLVNGASGGVAMAAIQIASLMGARPVIASSRSAAKLDKISQFGVDVGIDASKDDQVEAVLQATDNKGVDIIIDTVGGPVFEANMKSLAIKGRLVNIARLGSATAQIDLAQLWLKRLQLIGVTFRTRTEQERLECIQACARDMLPFLRAGRVRLPIDRTFAMTDIDAAHAYMKQDQHVGKIVLLVD

SEQ ID NO: 15 - protein, 327 aa, Cupriavidus necator
MAKAPMRVAVTGAAGQIGYSLLFRIANGDMLGKDQPVILQLLDLPQAQQAVKGVVMELEDCAFPLLAGVVITDDPKVAFKDADVALLVGARPRSKGMERKDLLEANAQIFTVQGKALDEVASRNVKVLVVGNPANTNAYIAMKSAPNLPRENFTAMLRLDHNRALSQIAAKTGKPVSSIEKLFVWGNHSPTMYADYRYATVDGKSVKDLINDPVWNNDVFLPTVGKRGAAIIEARGLSSAASAANAAIDHVRDWVLGTNGKVVTMGIPSNGEYGIPADTMFGYPVTTANGKYEIVKGLEIDAYSQEKINITLNELEEEKAGVQHLLG

SEQ ID NO: 16 - protein, 507 aa, Cupriavidus necator
MTVIKQEDLIQSVADSLQYISYYHPMDYITSLGRAYELEQSPAAKDAIAQILTNSRMCAEGKRPICQDTGIVTIFVKVGMGVRWDGATMGLTDMINEGVRRGYTHPDNVLRASIVSPPEGGRKNTKDNTPAVIHYEVVPGNTVDIQVAAKGGGSENKSKFVMLNPSDSIVDWVLKTVPTMGAGWCPPGMLGIGIGGTAEKAMVMAKESLMESIDIQDIIARGPKDWVEELRVELYEKVNALGIGAQGLGGLATVLDVKIMACPTHAASKPVAMIPNCAATRHVHFTLDGSGPAKLEAPDLSQWPKVEWAPNTETSKRVDLNTLTPEEVASWKPGQTLLLNGKMLTGRDAAHKRIADMLAKGEKLPVDFKNRVIYYVGPVDPVRDEAVGPAGPTTATRMDKFTEMMLAETGLISMIGKAERGPVAIEAIKKHKSAYLMAVGGAAYLVAKAIKEAKVVGFEDLGMEAIYEFDVKDMPVTVAVDSEGTSVHKTGPAEWQAKIGKIPVAAL

SEQ ID NO: 17 - protein, 468 aa, Cupriavidus necator
MSAPSTSATRIEKDSLGDVPVPADHLWGAQTERSRQNFRIGNEKMPPALIEAFAILKLCAARANGELGVLKPELAHAIEQAATEVIEGRWPDEFPLSVWQTGSGTQTNMNLNEVIANRAIQLLGGKVGSKTPVHPNDHVNASQSSNDSFPTAMHIAATRAIQQELLPALEQLQQTFARKVEAFADIVKVGRTHLQDAVPLTLGQEFSGYMTQVADAQSRLQQAMLRAMPVAQGGTAVGTGLNAPHGFAAAFARALADYTGLPFEPAPNRYALQASHDALADLSGALSTTASSFLKIARDFMLLGSGPRAGFAELVLPANEPGSSIMPGKVNPTQAEALAMVCCRVIGNHTTVTLANGLGTLELNAYKPVIIYSLLQSVSLLAGAASSFAEHMVEGVEADRERIAELLERSLMPVTALNPHIGYDKAAEIAKLAVKRNLSLREAAIASGHVTEAQFAEWIDLKGMTREV

SEQ ID NO: 18 - protein, 592 aa, Cupriavidus necator
MVAVKTGLPRRRFDVVIVGAGGAGMRASLQLAEAGLNVAVLSKVFPTRSHTVAAQGGIGASLGNMSEDNWHYHFYDTIKGSDWLGDQDAIEFMCREAPKVVYELEHFGMPFDRNPDGTIYQRPFGGHTANYGEKPVQRACAAADRTGHALLHTLYQRNVRAKTHFFVEWMALDLIRDQDGDVLGVTALEMETGEVYILEAKTTLFATGGAGRIYAASTNAFINTGDGLGMAARAGVPLEDMEFWQFHPTGVAGAGVLITEGVRGEGGILRNKDGERFMERYAPTLKDLAPRDFVSRSMDQEIKEGRGCGPNGDYVLLDLTHVGAETIMKRLPSIREIGMKFANVDAIKEPIPVVPTIHYQMGGIPTNYHGQVVVPKNGNPNEVLNGFYAIGECSCVSVHGANRLGTNSLLDLVVFGRAAGNHIIAQNLKQKEHKPLPADAADLALSRLAKLQSSSSGEYTQDVANDIRKNMQSHAGVFRTQKLMDEGVERILEVSQRADNIHLKDKSKVFNTALVEALEVANLVEVAKATMISAAARKESRGAHAHSDFPNRDDQNWLKHTLFYSEGNRLDYKPVKMEPLTVESVPPKARTF

SEQ ID NO: 19 - protein, 136 aa, Cupriavidus necator
MAEAVKQARPEYRNIGIAQIARYRLPWAGKVSILHRVSGALMFLLLPFVLYLFEQSITSELSFAKFSALLSGGFVKLVLLVLIWGYLHHFCAGIRFLLLDVHVGVSKPASAKSAIGVLVVSLLLTLIFGLKLFGLF

SEQ ID NO: 20 - protein, 233 aa, Cupriavidus necator
MKRIFEVYRYDPDKDAAPRMQTYEVELDGHERMLLDALVKLKKLDETISFRRSCREGVCGSDAMNINGKNGLACLTNMRELPDRIVLRPLPGLPVVRDLIVDMTNFFKQYNSIKPFLINDEPPPEKERLQSPEERDELDGLYECILCASCSTSCPSFWWNPDKFVGPAGLLQAYRFIADSRDQATNERLDNLNDPYRLFRCHSIMNCVDVCPKGLNPTKAIGKIKELMVRRAV

SEQ ID NO: 21 - protein, 660 aa, Cupriavidus necator [sequence omitted]

SEQ ID NO: 22 - protein, 314 aa, Cupriavidus necator
MDMKASGRHKVAIIGSGNIGTDLMIKVMRHGRSLEMGAMVGIDAASDGLARAARLGVPTTAEGIDGLVGMPGFGDIRIAFDATSAGAHAYHNRVLQQHGVRVIDLTPASIGPYVVPVVNLDQHLDAPNINMVTCGGQATIPMVAAVSRVANVHYAEIVASISSRSAGPGTRANIDEFTETTSQALCSVGGAARGKAIIVLNPAEPPLMMRDTVFTLSEDGDEAEIAASVEAMAQAVQGYVPGYRLKQRVQFERIAASAPLNIPGLGPMSGLKTSIFLEVEGAAHYLPAYAGNLDIMTSAALACAERLAEARLSA

SEQ ID NO: 23 - protein, 1012 aa, Cupriavidus necator
MTQHAARPHGRRTAPAAKDQSPALGAGQGDANGASTTESKRPATRSGTKRSPKPKLSIVSSNGTAIAPTARRTADKDVPLREDIRFLGRLLGECLREQEGDAAFEVVETIRQTAVRFRRENDRAAGAELDRLLKRLSRDQTNQVVRAFSYFSHLANIAEDQHHNRRRRVHALAGSPPQAGSLAHALEAIDAAGVTGKQLRKFLDEALIVPVLTAHPTEVQRKSILDAEREIARLLAERDLPMTAREREHNTAQLRAKVTTLWQTRMLRDSRLTVADEIENALSYYRTCFLRGIPQLMSELEEDIAAVFPATRKRKGTPGAQPAPLAPFLQMGSWIGGDRDGNPNVTAETLEHAASQQGQMIIDWYLDEVHALGAELSMSTLMVDASPELLALAERSPDHSEHRADEPYRRALIGIYARLAATSKALTGHAVPRRPVAPAEPYDSAEAFAADVQVVVDSLRANHGQALANGRIDALARAIGVFGFHLASVDMRQVSDVHEAVIAELFAAAGIAPDYAALPEARKLELLLAELRQPRLLTLPWHEYSEQTRKELAIFAAARELRARYGKRIARNYIISHTETLSDLVEVMLLQKESGMLQGTLGSKTDPARMELMVIPLFETIEDLRNAAGIMQSLLDLPGFDSVIAHHGVEQEVMLGYSDSNKDGGFLTSTWELYKAELALVQLFEQRQVKLRLFHGRGGTVGRGGGPTYQAILSQPPGTVNGQIRLTEQGEIINSKFANAEIGRRNLETVVAATLEASLLPQQNAPRELDTFEAVMQQLSDRAFTAYRDLVYETPGFKDYFFATTPITEIADLNLGSRPASRKLMDKKNRRIEDLRAIPWGFSWGQCRLLLPGWYGFGSAVKSLLDTAPDDKARKLAVTTLRRMVKTWPFFSTLLSNMDMVLAKTDLAVASRYAQLCDDAALRRTVFNRISKEWHLTCEMLTLVTGHQERLADNPLLARSIKNRFAYLDPLNHLQVELLKRFRSGKDGDDIRVRRGIHLTINGVAAGLRNTG

SEQ ID NO: 24 - protein, 256 aa, Cupriavidus necator
MKAELLLPLLGLQALLGAGTYFQTVTGFGLGMIVMGVTSGLGLAPVATVAAVVSLVTLANSACALPGKLQHIDWRAVAAAAIGILPSVVVGVLVLEYLSSSAATLLQLLLGAVILYGGLSAALKPAPLAQRSGDGTFFVSGVFGGLLSGMFGVSGPPLIFQFYRQPMKPVEIRCALILVFTVTSTVRTLFSAWQGQLDAAVCVQAAIAVPVVVIATLLGRRFPPPFSPATTRRVAFGVLIGIGASLMLPAISVWVL

SEQ ID NO: 25 - protein, 658 aa, Cupriavidus necator
MADAIRVLAMDAVQQANSGHPGAPMGMADIAVALWGRHLKHNPSNPQWADRDRFVLSNGHGSMLIYALLHLTGYDLPVEELKNFRQLHSKTAGHPEYGITPGVETTTGPLGQGITNAVGMALAERLLGEEFNRPGFDIVNHHTYVFLGDGCLMEGISHEASSLAGTLKLNKLIALWDDNGISIDGDVVHWFNDDTPKRFEAYGWNVIRGIDGHDVTAVDMAISQAKASDKPTLICCRTKIGKGAPNKEGGHDVHGAPLGGAEVLATREALGWAHAPFEVPAAVYDTWDAKANGQALERAWNALFDAYAERHPYEAGEFRRRMKGELPGSFDAAVEAFIAKCEEKAETIATRKASQNTIEAFGPVLPEFLGGSADLTGSNLTNWSGSQAVRVDAWGNHINYGVREFGMSAIMNGITLHGGYIPYGATFLTFSDYSRNALRMAALMKIRSLFVFTHDSIGLGEDGPTHQSIEHVASLRLIPNMDVWRTADTTETAVAWAESIRRENGPSCLIFSRQNLPFQRRDETTRANIARGGYVLRDSVNPKTKRPDAVILATGSEVGLAVGAADALATEGVHVRVVSVPATTVFDKQDTAYKASVLPAGVPRVAVEAGVTDFWWKYQVQAVVGIDTFGESAPAGVLFKHFGFTVDNVVRTVKDTLI

SEQ ID NO: 26 - protein, 318 aa, Cupriavidus necator
MNQLEQLRQFTTVVADTGDFQLMKQYTPQDATTNPSLILKAVQKPEYRHLLEQAVRDHHGNGGVDAVMDAVLIAFGCEILAIVPGRVSTEVDARLSFDTEASVAKARHLIQLYEQRGIARERVLIKIASTWEGIRAAEILQRDGIRCNMTLLFSLVQAVACAEAGAQLISPFVGRIFDWYKKQAGEQWDAAANGGDNDPGVRSVRQIYDYYKKFGYPTEVMGASFRSTAQILSLAGCDLLTISPELLEQLAAGQGPVTHKLSVDQAQAANIARIAADEASFRWQLNEDAMATEKLAEGIRLFAADAVKLEKLIGGIAK

SEQ ID NO: 27 - protein, 241 aa, Cupriavidus necator
MHATETNTGHGSLRAIRLAPSILSADFARLGEEVCAIEAGGADLVHFDVMDNHYVPNLTIGPLVCEAIRPLVSIPIDVHLMVEPVDALIPLFAKAGANIISFHPEASRHVDRTIGLIRDHGCKAGLVLNPATPLGWLDHTLDKLDLVLLMSVNPGFGGQAFIPGVLDKVRQARARIDRQVDAGGRPVWLEIDGGVKADNIAGIARAGADTFVAGSAVFGAPDADGGYWSILRRLREAATIT

SEQ ID NO: 28 - protein, 228 aa, Cupriavidus necator
MTQDELKALVAQAAADYVKQEVPEGAVLGVGTGSTANLFIDAVAAFKERFAGAVSSSEASTRRLQQHGFKVLDLNEVDEIPVYVDGADEIDASGAMIKGGGGALTREKIVASVAKRFVCIADGSKLVQTMGTFPLPVEVIPMARAAVARKLQALGGQPRLRMTKEGGIYKTDNGNVILDVVGLKIEDPRGLEQAVNQVPGVVTVGLFALRGADVLLLGTGDGVQRTDY

SEQ ID NO: 29 - DNA, 1671 bp, Clostridium cylindrosporum DSM 605 CLCY_4c
ttatgataatcctatgatttctccgctttcaagtatatccataccattagctgctggaacctttggaagtcctggcattgtcatgatatttccagtaagtgctacgataaatcctgcacctgctgatactcttacttctcttacagtgattctgaatcctgttggtgctccaagtagtgttggatcatctgagaatgagtattgagtcttagccatacagattggcatcttgtctagaccaagtttttcaagttcagcgatttgcttcttacaatctgaagtgaagtcaactccgtctgctctgtatatagtagtagcgatcttttcgatcttttctggaatgcttaggtttacatcgtatagtggtgcaaagtttgcttcattcttttcacagatttcaacaaccttcttagccatttcaactccaccttctccaccgttagcccaaacgtctgaaagaacaacgtctgcacccttagcgttacaagcatcttctacagccttaagttcagcttctgtatctagtgggaacttgttgatagcaacaactgctggtactccgaacttagcaacgttttcaatgtgtctttcaaggttagctacacccttcttaacagcttcaacgttttcagcagctagttcagtcttaggaactccaccgttcatcttaagagctctaactgtagcaacgataactacacagtttggcttaagaccagcgaatcttgacttaatatcaaagaacttttcagctccaaggtcagcaccgaatccaccttcagttactacgtaatcagcaagcttaagagcaaccttagtagcaagaactgagttacatccatgtgcgatgttcgcgaatggtccaccgtggatgaatgcaggtgtgttttctagtgtttgaacaaggtttggcttaacagcatccttaagaagaagtgccatagctcctgtagcttctagttgaccagctgtaactgcgcttccgtccttagcataagctacgatcatctttgaaagtctttccttaaggtctgacattgatgttgcaagacaaagaattgccattatttctgaagcaactgtgatatcgaattgtgcttgtcttacgaatccatcaccatttccaccaagaccgattataacgtttcttagtgatctatcgttcatgtcgataactctcttccaaacaatcttcttaggattgatgtttagagcgtttccttggtgtagatggttgtcaagtagtgctgctagtaggttgtgagctgaagttatagcatggaagtcacctgtaaagtgtaggttgatatcgtccataggtacaacttgtgcgtatccaccacctgctgctcctcccttaactccgaagcaaggtcctagtgatggctctctaagagctgtgattgttctcttaccaattttgttaagacccattgaaagaccaacgtttgtagttgtctttccttctccagctggagttgggttgatagctgttacaagaacaagcttagcatcttccttatctttttgtctttctagaacgtctagagaaaccttagccttatactttccgtatagttctagttcatcatcttgaattccaaggtagttagccacttcagtgattggcttcatttgagcttcttgagctatttgaacgtctgttttcat

SEQ ID NO: 30 - DNA, 975 bp, Methylobacterium extorquens AM1
atgagcttcaccctgatccagcaggccaccccgcgcctgcaccgctcggaactcgcggttcccggctccaacccgaccttcatggagaagtcggccgcctcgaaggccgacgtgatcttcctcgacctcgaggacgcggttgcgcccgacgacaaggagcaggcccgcaagaacatcatccaggccctcaacgacctggattggggcaacaagaccatgatgatccgcatcaacggtctcgacacccactacatgtaccgcgacgtggtggacatcgtggaggcctgcccgcgcctcgacatgatcctgatccccaaggtcggcgtgccggccgacgtctacgccatcgacgtgctgacgacgcagatcgagcaggccaagaagcgcgagaagaagatcggcttcgaggtgctgatcgagaccgcgctcggcatggccaatgtcgaggcgatcgcgacctcgtctaagcgccttgaggcgatgtccttcggtgtcgccgactacgccgcttccacccgcgcccgctccaccgtgatcggcggcgtcaacgccgattacagcgtgctcaccgacaaggacgaggccggcaaccgccagacccactggcaggatccgtggctgttcgcccagaaccgcatgctggtcgcctgccgcgcctacggcctgcgcccgatcgacggtcccttcggcgacttctccgatccggacggctacacctcggccgctcgccgctgcgccgcgctcggcttcgagggcaagtgggcgatccacccctcgcagatcgatctcgccaacgaggtcttcaccccctccgaggccgaggtcaccaaggcccgccgcatcctggaagccatggaagaggccgccaaggccggccgcggcgccgtctcgctcgacggccgtctcatcgacatcgcctcgatccgcatggccgaggcgctgatccagaaggccgacgcgatgggcggaaagtaa

SEQ ID NO: 31 - DNA, 1173 bp, Methylobacterium extorquens AM1
atggacgttcacgagtaccaagccaaggagctgctcgcgagcttcggggtcgccgtcccgaagggcgccgtggctttcagcccggatcaagcggtctatgcggcgaccgagctcggcggctcgttctgggcggtgaaggctcagatccatgccggcgcgcgcggcaaggcgggcgggatcaagctttgccgcacctacaatgaagtgcgcgacgccgcccgcgacctgctgggaaaacgcctcgtgacgctccagaccggccccgagggcaagccggtgcagcgcgtctacgtcgagaccgccgacccgttcgagcgtgaactctatctcggctacgtgctcgatcggaaggccgagcgcgtccgtgtcatcgcctcccagcgcggcggcatggatatcgaggagatcgccgccaaggagcccgaggcgctgatccaggtcgtggtcgagccggcggtgggcctgcagcagttccaggcccgcgagatcgcgttccagctcggcctcaacatcaagcaggtctcggccgcggtgaagaccatcatgaacgcctaccgggcgttccgcgactgcgacggcaccatgctggagatcaacccgctcgtcgtcaccaaggacgaccgggttctggcactcgacgccaagatgtccttcgacgacaacgccctgttccgccgccgcaacatcgcggacatgcacgatccatcgcagggcgatccccgcgaggcccaggctgccgagcacaatctcagctatatcggcctcgagggcgaaattggctgcatcgtcaacggcgcgggtctggccatggcgaccatggacatgatcaagcacgcgggcggcgagccggcaaacttcctggatgtgggcggcggtgccagcccggaccgcgtcgccacggccttccgcctcgttctgtcggaccgcaacgtgaaggcgatcctcgtcaacatcttcgccggcatcaaccgctgcgactgggtcgcggagggcgtggtcaaggccgcgcgcgaggtgaagatcgacgtgccgctcatcgtgcggctcgccggcacgaacgtcgatgaaggcaagaagatcctcgccgagagcgggctcgacctcatcaccgccgacacccttacggaagccgcgcgcaaggctgtcgaagcctgccacggcgccaagcactga

SEQ ID NO: 32 - DNA, 891 bp, Methylobacterium extorquens AM1
atgagcattctcatcgacgagaagaccccgatcctggtccagggcatcacgggcgacaagggcaccttccacgccaaggagatgatcgcctacggctcgaacgtcgtcggcggcgtcaccccgggcaagggcggcaagacccattgcggcgtgccggtgttcaacaccgtcaaggaggccgtggaggcgaccggcgccaccacctcgatcaccttcgtggcgccccccttcgcggcggacgcgatcatggaggcggccgacgccggcctcaagctcgtctgctcgatcaccgacggcatccccgctcaggacatgatgcgggtgaaacgctacctccggcgctatccgaaggagaagcgcacgatggtggtgggcccgaactgcgcgggcatcatctcgcccggcaagtcgatgctcggcatcatgcccggccacatctacctcccgggcaaggtcggcgtcatctcccgttccggcacgctgggctacgaggccgccgcgcagatgaaggagctcggcatcggcatctcgacctccgtcggcatcggcggcgatccgatcaacggctcctccttcctcgaccacctcgctctgttcgagcaggatcccgagacggaagccgtgctgatgatcggcgagatcggcgggccgcaggaggccgaggcctcggcctggatcaaggagaacttttccaagcccgtgatcggcttcgtggcgggcctcaccgcccccaagggccgccgcatggggcatgccggcgcgatcatctcggcgaccggcgacagcgccgcggagaaggccgagatcatgcgctcctatggcctgaccgtggcgcccgatccgggctccttcggcagcaccgtggccgacgtgctcgcccgcgcggcgtga

SEQ ID NO: 33 - DNA, 627 bp, Methylobacterium extorquens AM1
atggccggcaacgagacgatcgaaacattcctcgatggcctggcgagctcggccccgacccccggcggcggcggtgccgccgcgatctccggcgccatgggcgcggcgctggtctcgatggtgtgtaacctcaccatcggcaagaagaagtatgtcgaggtcgaggccgacctgaagcaggtgctggagaagtcggaaggcctgcgccgcacgctcaccggcatgatcgccgacgacgtcgaggctttcgacgcggtgatgggcgcctacgggctgccgaaaaacaccgacgaggagaaggctgcccgcgccgccaagattcaggaggcgctcaagaccgcgaccgacgtgccgctcgcctgctgccgcgtctgccgcgaggtgatcgatctggccgagatcgtcgccgagaagggcaatctcaacgtcatctcggatgccggcgtcgccgtgctctcggcctatgccggtctgcgctcggcggccctcaacgtctacgtcaacgccaagggcctcgacgaccgcgccttcgccgaggagcggctgaaggagctggaaggcctgctggccgaggcgggcgcgctcaacgagcggatctacgagacggtgaagtccaaggtaaactga

SEQ ID NO: 34 - DNA, 1314 bp, Pyrococcus horikoshii OT3
atggatattagggagataggcctgagattggttggagaagcaataaaagctgcagatccatacagggcagtcctaaatgcagttaaagtgagtgatgacaagataattgttcaaggtaaggaatttgaaatcaagggaaaggtgtacgtaatagccctggggaaagcagcatgtgaaatggcaagggccatagaggatatactcgatgttgaggatggcgttgcggtaactaagtacggttatggaaaagagttaaagaggataaaggtgattgaagcgggacacccaatacctgatgagaaatccatattgggagctaaagaggccctatcgatattgaatagagctagggaaaatgacatagttttcatcctaatttcaggtggaggttcggcactatttgagcttccagaagaaggtatttccctagaagatcttaagctaaccacggatctccttctaaagagcggggcaaagatacatgagataaatacggttagaaagcatatatcaaaggttaagggaggtaagcttgcgaagatgatcaaggggaccggaatagtactgataatttcggatgtagttggcgataacctagaagccatagcctcagggcccaccgttaaggatccaactaccttcgaagatgcgaagaggatcctggaactctacgacatctgggagaaggttcccgaaagcgttaggttacatatagagagaggattaaggggggaggtggaggaaaccctcaaggaagatctacccaatgttcacaacttcctgatagccagtaattcaatctcctgcgaggccatagccagggaagctcagaggcttgggttcaaagcatatataatgacgactactttggagggagaagccaaggatgccgggttattcataggatctatagtccaggaaatagctgaaaggggaaggccgttcgaacctccggtagttctagtgttcggtggagagaccaccgtaacaatagaaggtaaagggggaaagggagggcccaaccaagaaatagcgctgagtgctaccaggaagatctcggatctagaagccctgatcgtggccttcgacaccgatggaaccgatggtcccactgatgcggccgggggaatagtggatggaacaacgtacaagaagcttagggaaaagggaatagatgttgagaaagtattgaaagagcataactcgtatgaagccttaaagaaagttgggggcttgctattcactgggcccactggaacgaacgtcaactctattgttatagcgatagtcacatcgaaacgaggccgtacttaa

SEQ ID NO: 35 - DNA, 945 bp, Methylobacterium extorquens AM1
atgacaaagaaagtcgtcttcctcgatcgcgagtcgctcgatgcgaccgtgcgcgaattcaacttcccgcacgagtacaaggaatatgagtcgacctggacgccggaggagatcgtcgagcgccttcagggcgccgagatcgcgatgatcaacaaggtgccgatgcgcgccgacacgctgaagcagcttcccgacctgaagctgatcgcggtggctgccacgggcacggacgtcgtcgacaaggctgcggccaaggcgcagggcatcacggtcgtcaacatccgcaactacgccttcaacaccgtgcccgagcacgtggtcggcctgatgttcgcgctgcgccgggcgatcgtgccttacgccaactcggtgcgccggggcgattggaacaagtcgaagcagttctgctacttcgattacccgatctacgacatcgccggctcgacgctcggcatcatcggctacggcgcgctcggcaagtcgatcgccaagcgggctgaggccctcggcatgaaggtgctcgccttcgacgtgttcccgcaggacgggctcgtggatctcgagacgatcctgacgcaatccgacgtcatcaccctgcacgtgccgctgacccccgacaccaagaacatgatcggggccgagcagctcaagaagatgaagcgctccgcgatcctcatcaacaccgcccgcggcgggctggtggacgaggcggccctgctccaggcgctcaaggacggcaccatcggcggcgccggcttcgacgtcgtggcccaggagcccccgaaggacggcaacatcctctgcgacgccgacctgcccaacctgatcgtcaccccgcacgtggcctgggcgagcaaggaggcgatgcagatcctcgccgaccagctcgtggacaacgtcgaggccttcgtcgcgggcaagccgcagaacgtcgtcgaggcgtaa

SEQ ID NO: 36 - DNA, 867 bp, Methylobacterium extorquens AM1
atgtccaagaagctgctcttccagttcgacaccgatgccacgccgagcgtcttcgacgtcgtcgtcggctacgacggcggtgccgaccacatcaccggctacggcaacgtcacgcccgacaacgtcggcgcctatgtcgacggcacgatctacacccgcggcggcaaggagaagcagtcgacggcgatcttcgtcggcggcggcgacatggcggccggcgagcgggtgttcgaggcggtgaagaagcgcttcttcggcccgttccgcgtgtcctgcatgctggattcgaacggctccaacacgaccgccgcggcgggtgtggcgctcgtcgtcaaggcggcgggcggctcggtcaagggcaagaaggccgtcgtgctcgcgggcaccggcccggtcggcatgcgctcggcggcgctgctcgccggcgagggcgccgaggtcgtgctgtgcgggcgcaagctcgacaaggcgcaggccgcggccgattccgtgaacaagcgcttcaaggtgaacgtcaccgcggccgaaaccgcggacgacgcttcgcgcgccgaggccgtgaagggcgcccatttcgtcttcaccgccggtgcgatcggccttgaactgctgccgcaggcagcctggcagaacgagagttcgatcgagatcgtggccgactacaacgcccagccgccgctcggcatcggcgggatcgatgcgaccgacaaaggcaaggaatacggcggaaagcgcgccttcggtgcgctcggcatcggcggcttgaagctcaagctgcaccgcgcctgcatcgccaagctgttcgagtcgagcgaaggcgtcttcgacgccgaggagatctacaagctggccaaggaaatggcctga

SEQ ID NO: 37 - DNA, 1209 bp, Methylobacterium extorquens AM1
atggcggcaacgagacgtccgggacgcaaccacctgttcgttcccggcccgaccaacatcccggaccgggtgatgcgcgccatgatggtgcagtccgaggatcaccgctcggtcgatttcccgtcgctgacgaagccgctgttcgaggacaccaagaaggtgttcggctcgaccgaaggcacgatcttcctgttcccggcctccggcacgggcatctgggaatcggcgctgtccaacacgctcgcccgcggcgacaaggtgctggccgcccgcttcggccagttcagccatctctggatcgacatggcccagcgcctcggcctggacgtcgtcgtccaggaggaggagtggggcaccggcgccaagcccgagaagatcgaggaggccctgcgcgccgacaagaaccatgagatcaaggccgtcatggtggtccataacgagaccgcgaccggcgtgacctccaacatcggcgccgtgcgcaaggcgatcgacgccgccggccacccggccctgctgttcgtcgacggcgtgtcctccatcggctcgctgcccttcaaggccgacgagtggaaggtcgactgcgccatcgccggctcccagaagggcctgatgctgcccgccggcctcggcgtgatttgcgtcagccagaaggcgctcaaggccgccgagggccagtccggccgcaacgaccggctcgcccgcgtctacttcgactgggaagaccagaagaagcagaacccgaccggctacttcccctacaccccgccgctgccgctgctctacggcctgcgcgaggcgctcgcctgcctgttcgaggaaggcctggagaacgtctaccaccgccacgccgtgctcggtgaggcgacccgtcaggccgtcgcggcctggggcctgaagacctgcgccaagtcgccggagtggaactccgacaccgtcaccgccatcctggcgcccgagggtgtggacgcggccaagatcatcaagcacgcctatgtgcgctacaacctcgcgctcggcgccggcctgtcccaggtcgcgggcaaggtgttccgcatcggccacgtcggcgacctgaacgaactctcgctgctcggcgccatcgccggtgccgagatgtcgctcatcgacaacggcgtgaaggtgacccccggttcgggtgttgcggctgcctccagctacctgcgcgagaacccgctcgccaaggcttga

SEQ ID NO: 38 - DNA, 2769 bp, Methylobacterium extorquens AM1
atgaccaagacgctgcacgcccggccgtcagccgcgaccgacacgaccttcgcgccgccggtcatcaccggcacggccaccgaggacgccctcgagattctgtttcacgccctgctcgacgtggcccgccgccacgatcccgagctggaggacgtgctgcacggccgcgccgacatctcctccttcacgccggagatgctggcgcgcgcgcttcaggtgcagggcatctggtttcagctcgtctcgatcgccgagcagaacgcggccatgcgccgccgccgccacgtcgagcgcgaccagggccgtgaggcgctgaacggctccttcgctaaggttctggccgaggcctccgcccggggcatcggcccgcagcagatccacgcgctgctcaaggatctgcgcatccgcccgaccatcaccgcgcatcccaccgagggcaagcgcgtgacggtgctggagaagctgcgccggatctacctcgtgctgcgcgagctggaactgccgcgctggaccgagcgcgagcgcaacggcctgatgaacgagctgcgtgaccagatcgagctgatctggatgacgggcgagctgcatctggagaaggccaccgtcgagcgcgaggtcgcctgggggctgcacttcttcgacgagaccttgttcgagatgctgcccgagatgctgctctcgctcgaagagagcctcgcgcagtattatcccgacgagaccttcgaggtgccgccgttcttccagttcggaagctggatcggcggtgaccgcgacggcaacccctacgtcaccgccagcgtgacccgcgagacgcttcagcgcaacgcgctggcgtcgctccgccgttaccgcgacggcatcacccatctcggccgggtgctctcgatcaccgagcgctcgctgccggtgcccgagaccttccgcagcgaactcgcgcatatgctggccgagtccggcgacgcgcgcgccatcgccaaccgcaatcccggcgaggcctatcgccagttcctctcctgcgtgctgcgcaagctcgaggcgacgatcgcccgcaacaagggcgcccgctcggtcggaccggattatccgagcgccgacgggctcatcaacgatctgcgcacgctggagaaggggctggccgacgccaagtgcggcgcgctcgccaccgacatcgtgcggcccgtgcggcggatggtcgagatcttccgcttctccaccgtgcggctcgatctgcgcgagaactcgacccgcaccaccaagacgctgcacgcgctgtggaagctgcgcaacggcgaccgcgagccgccggcgctggattcgccggcctggaaggattggctgctcaccgagctggcccggccgcgcacgcccgaaacctcgttcgaggatttcgccgaccgtctgcccgacgacgcgcgcgagacgctcgccaccttcgcgctggtcggcgagatgcgcgacactctcgaccgcgaggccttcggcgccttcatcctgtcgatgacgcgctccaccgtcgacgtgctcggcgcctacctgctggccaaggaggccggcatcttcctcgacacgacgggcaccgagatctgcccgctgccgatcgtgccgctgttcgagaccatcgacgacctgcgcgccgccccggcgatcatgaaggaactgctcggcatccccgtggtgcgccgctccacccgctggcagggcggggtgcaggaagtgatgatcggctactccgattccaacaaggacggcggcttcattgcctcgaactgggagctctacaaggcgcaggtgcggctgaccacgctcggcaaccatctcggcgtgccgatcgccttcttccacggccgcggcggttcggtgagccgcggcggcgtgccgacccatcgcggcatcgcggcccagccgccgggctcgatccagggccgcttccgcatcaccgagcagggcgaagtcgtctcgttcaaatacgccaaccgcggcaccgccgcctaccagatggagctgctcgccgcctcggtgttcgagcacgccctgctctcggagggcaacggcaacggctcgcgcgccgaattcgacgacgcgctggaggcgctctcgggcgcctcgcgggcggcctatgtcaacctgctccaggccgagggcctcgtcgattacttccaggcggcgagcccgctcgacgagatctcgctgctcaatatcggctcgcgcccggcccggcgcttcggcgcgaagtcgctctcggatctgcgcgcgatcccctgggtgttcgcgtggtcgcagaaccggcacgtcatcaccggctggtacggcgtcggctcggggctgaagagcttcatcgacgtgcgcggcgaggcgggcgaagccctgctgaggcgcctgttccgggattgccgggtgttccgcctcgtcctcgacgaggtcgagaagacgctgctgatggtcgatctcgagatcgcccgcgactatgccggcctcgtcgaggacgcgggcatccgcgcgcgcatcttcgggatgatcgaggcggaatacgcgctgacgcgggagatggtcctgcgggtgtccggcgacagcgaactcgcccagcgcttcccgcaattcagcgaacgcctgcgcgggcggctgccgaccatcaatcaggtcagccgcgagcaggtcgagctgctgcgccgctaccgcagcgagaccgacgaggacaagcgcgaggcggtgaagtccgcgctgctgctgtcgatcaactgcatcgccgtcggcttcggcgcgactggataa

SEQ ID NO: 39 - DNA, 2364 bp, Clostridium butyricum
atgataagtaaaggatttagtacccaaacagaaagaataaatattttaaaggctcaaatattaaatgctaaaccatgtgttgagtcagaaagagcaatattaataacagaatcatttaaacaaacagaaggccagccagcaattttaagaagagcattggcattgaaacacatacttgaaaatatccctataacaattagagatcaagaacttatagtgggaagtttaacgaaagaaccaaggtcttcacaagtatttcctgagttttctaataaatggttacaagatgaattggatagattaaacaagagaactggagatgcattccaaatttcagaagaaagtaaagaaaaattaaaagatgtctttgagtattggaatggaaaaacaacaagtgagttagcaacttcatatatgacagaggaaacaagagaggcagtaaattgtgatgtatttactgtaggaaactactattataatggcgtaggacatgtatctgtagattatggaaaagtattaagggttggatttaatgggattataaatgaggctaaggaacaattagaaaaaagcagaagtatagatcctgattttatcaagaaagaaaaattcctaaatagtgttattatctcatgcgaagctgcaataacatacgtaaatagatatgctaaaaaggctaaagagattgcagataatacaagtgatgcaaaaagaaaagctgaattaaatgaaatagcaaaaatttgttcaaaagtttcaggagagggagctaaatctttctatgaagcatgtcaattattttggtttattcatgcaataataaatatagaatctaatggacattctatttctccagccagatttgatcaatatatgtatccatattatgaaaatgataaaaatataacagataagtttgctcaagaattaatagattgtatctggattaaattaaatgatattaataaagtaagagatgagatttcaactaaacattttggtggttatccaatgtatcaaaacttaattgttgggggtcaaaattcagaaggaaaagatgcaactaataaagtatcatatatggcattagaagcagctgtccatgtaaagttacctcagccatctttgtcagtaagaatatggaataagactccagatgaatttttgcttagagcagcagaattaactagagaagggttaggacttcctgcttattataatgatgaagttattattccagcattagtttctagaggtcttacattagaagatgcaagagactacggaataattggatgtgttgaaccacaaaagccaggaaaaacagaaggatggcatgattcagcattctttaatcttgcaagaatagtagagttaactataaattctggatttgataaaaataaacagattggacctaaaactcaaaattttgaagaaatgaaattctttgatgaattcatgaaagcttataaagctcaaatggagtattttgtaaaacatatgtgctgcgctgataattgcatagatattgcacatgcagaaagagctccattacctttcttgtcatcaatggttgataattgtatcggaaaaggaaagagccttcaagatggtggggcagaatataacttcagtggaccacaaggcgttggagtagctaatattggagattcattagttgcagttaaaaaaattgtgtttgatgaaaataagattactccttcagaattaaagaaaacattaaataatgattttaaaaattcagaagaaatacaagccttactaaaaaatgctcctaagtttggaaatgatattgatgaagttgataatttagctagagagggtgcattagtatactgtagagaagttaataaatatacaaatccaaggggaggaaattttcaaccaggattatatccatcttcaattaatgtatattttggaagcttaacaggtgctactccagatggaagaaaatctggacaaccattagctgatggggtttctccatcaagaggctgtgatgtatctggacctactgcagcttgtaattcagttagtaaattagatcattttatagcttcaaatggaactttatttaatcaaaaattccatccgtcagcattaaaaggtgataatggattaatgaatttatcatcattaataagaagttattttgatcaaaaggggtttcatgttcaatttaatgtaatagataaaaaaatattacttgcagcacaaaaaaatcctgaaaaatatcaagatttaattgttagagttgcaggatatagtgcacagttcatttctttagataaatctattcaaaatgatattattgcaagaactgaacatgttatgtaa

SEQ ID NO: 40 - protein, 556 aa, Clostridium cylindrosporum DSM 605 CLCY_4c
MKTDVQIAQEAQMKPITEVANYLGIQDDELELYGKYKAKVSLDVLERQKDKEDAKLVLVTAINPTPAGEGKTTTNVGLSMGLNKIGKRTITALREPSLGPCFGVKGGAAGGGYAQVVPMDDINLHFTGDFHAITSAHNLLAALLDNHLHQGNALNINPKKIVWKRVIDMNDRSLRNVIIGLGGNGDGFVRQAQFDITVASEIMAILCLATSMSDLKERLSKMIVAYAKDGSAVTAGQLEATGAMALLLKDAVKPNLVQTLENTPAFIHGGPFANIAHGCNSVLATKVALKLADYVVTEGGFGADLGAEKFFDIKSRFAGLKPNCVVIVATVRALKMNGGVPKTELAAENVEAVKKGVANLERHIENVAKFGVPAVVAINKFPLDTEAELKAVEDACNAKGADVVLSDVWANGGEGGVEMAKKVVEICEKNEANFAPLYDVNLSIPEKIEKIATTIYRADGVDFTSDCKKQIAELEKLGLDKMPICMAKTQYSFSDDPTLLGAPTGFRITVREVRVSAGAGFIVALTGNIMTMPGLPKVPAANGMDILESGEIIGLS

SEQ ID NO: 41 - protein, 324 aa, Methylobacterium extorquens AM1
MSFTLIQQATPRLHRSELAVPGSNPTFMEKSAASKADVIFLDLEDAVAPDDKEQARKNIIQALNDLDWGNKTMMIRINGLDTHYMYRDVVDIVEACPRLDMILIPKVGVPADVYAIDVLTTQIEQAKKREKKIGFEVLIETALGMANVEAIATSSKRLEAMSFGVADYAASTRARSTVIGGVNADYSVLTDKDEAGNRQTHWQDPWLFAQNRMLVACRAYGLRPIDGPFGDFSDPDGYTSAARRCAALGFEGKWAIHPSQIDLANEVFTPSEAEVTKARRILEAMEEAAKAGRGAVSLDGRLIDIASIRMAEALIQKADAMGGK

SEQ ID NO: 42 - protein, 390 aa, Methylobacterium extorquens AM1
MDVHEYQAKELLASFGVAVPKGAVAFSPDQAVYAATELGGSFWAVKAQIHAGARGKAGGLKLCRTYNEVRDAARDLLGKRLVTLQTGPEGKPVQRVYVETADPFERELYLGYVLDRKAERVRVIASQRGGMDIEEIAAKEPEALIQVVVEPAVGLQQFQAREIAFQLGLNIKQVSAAVKTIMNAYRAFRDCDGTMLEINPLVVTKDDRVLALDAKMSFDDNALFRRRNIADMHDPSQGDPREAQAAEHNLSYIGLEGEIGCIVNGAGLAMATMDMIKHAGGEPANFLDVGGGASPDRVATAFRLVLSDRNVKAILVNIFAGINRCDWVAEGVVKAAREVKIDVPLIVRLAGTNVDEGKKILAESGLDLITADTLTEAARKAVEACHGAKH

SEQ ID NO: 43 - protein, 296 aa, Methylobacterium extorquens AM1
MSILIDEKTPILVQGITGDKGTFHAKEMIAYGSNVVGGVTPGKGGKTHCGVPVFNTVKEAVEATGATTSITFVAPPFAADAIMEAADAGLKLVCSITDGIPAQDMMRVKRYLRRYPKEKRTMVVGPNCAGIISPGKSMLGIMPGHIYLPGKVGVISRSGTLGYEAAAQMKELGIGISTSVGIGGDPINGSSFLDHLALFEQDPETEAVLMIGEIGGPQEAEASAWIKENFSKPVIGFVAGLTAPKGRRMGHAGAIISATGDSAAEKAEIMRSYGLTVAPDPGSFGSTVADVLARAA

SEQ ID NO: 44 - protein, 440 aa, Pyrococcus horikoshii
MIAMDIREIGLRLVGEAIKAADPYRAVLNAVKVSDDKIIVQGKEFEIKGKVYVIALGKAACEMARAIEDILDVEDGVAVTKYGYGKELKRIKVIEAGHPIPDEKSILGAKEALSILNRARENDIVFILISGGGSALFELPEEGISLEDLKLTTDLLLKSGAKIHEINTVRKHISKVKGGKLAKMIKGTGIVLIISDVVGDNLEAIASGPTVKDPTTFEDAKRILELYDIWEKVPESVRLHIERGLRGEVEETLKEDLPNVHNFLIASNSISCEAIAREAQRLGFKAYIMTTTLEGEAKDAGLFIGSIVQEIAERGRPFEPPVVLVFGGETTVTIEGKGGKGGPNQEIALSATRKISDLEALIVAFDTDGTDGPTDAAGGIVDGTTYKKLREKGIDVEKVLKEHNSYEALKKVGGLLFTGPTGTNVNSIVIAIVTSKRGRT

SEQ ID NO: 45 - protein, 208 aa, Methylobacterium extorquens AM1
MAGNETIETFLDGLASSAPTPGGGGAAAISGAMGAALVSMVCNLTIGKKKYVEVEADLKQVLEKSEGLRRTLTGMIADDVEAFDAVMGAYGLPKNTDEEKAARAAKIQEALKTATDVPLACCRVCREVIDLAEIVAEKGNLNVISDAGVAVLSAYAGLRSAALNVYVNAKGLDDRAFAEERLKELEGLLAEAGALNERIYETVKSKVN

SEQ ID NO: 46 - protein, 314 aa, Methylobacterium extorquens AM1
MTKKVVFLDRESLDATVREFNFPHEYKEYESTWTPEEIVERLQGAEIAMINKVPMRADTLKQLPDLKLIAVAATGTDVVDKAAAKAQGITVVNIRNYAFNTVPEHVVGLMFALRRAIVPYANSVRRGDWNKSKQFCYFDYPIYDIAGSTLGIIGYGALGKSIAKRAEALGMKVLAFDVFPQDGLVDLETILTQSDVITLHVPLTPDTKNMIGAEQLKKMKRSAILINTARGGLVDEAALLQALKDGTIGGAGFDVVAQEPPKDGNILCDADLPNLIVTPHVAWASKEAMQILADQLVDNVEAFVAGKPQNVVEA

SEQ ID NO: 47 - protein, 288 aa, Methylobacterium extorquens AM1
MSKKLLFQFDTDATPSVFDVVVGYDGGADHITGYGNVTPDNVGAYVDGTIYTRGGKEKQSTAIFVGGGDMAAGERVFEAVKKRFFGPFRVSCMLDSNGSNTTAAAGVALVVKAAGGSVKGKKAVVLAGTGPVGMRSAALLAGEGAEVVLCGRKLDKAQAAADSVNKRFKVNVTAAETADDASRAEAVKGAHFVFTAGAIGLELLPQAAWQNESSIEIVADYNAQPPLGIGGIDATDKGKEYGGKRAFGALGIGGLKLKLHRACIAKLFESSEGVFDAEEIYKLAKEMA

SEQ ID NO: 48 - protein, 402 aa, Methylobacterium extorquens AM1
MAATRRPGRNHLFVPGPTNIPDRVMRAMMVQSEDHRSVDFPSLTKPLFEDTKKVFGSTEGTIFLFPASGTGIWESALSNTLARGDKVLAARFGQFSHLWIDMAQRLGLDVVVQEEEWGTGAKPEKIEEALRADKNHEIKAVMVVHNETATGVTSNIGAVRKAIDAAGHPALLFVDGVSSIGSLPFKADEWKVDCAIAGSQKGLMLPAGLGVICVSQKALKAAEGQSGRNDRLARVYFDWEDQKKQNPTGYFPYTPPLPLLYGLREALACLFEEGLENVYHRHAVLGEATRQAVAAWGLKTCAKSPEWNSDTVTAILAPEGVDAAKIIKHAYVRYNLALGAGLSQVAGKVFRIGHVGDLNELSLLGAIAGAEMSLIDNGVKVTPGSGVAAASSYLRENPLAKA

SEQ ID NO: 49 - protein, 922 aa, Methylobacterium extorquens AM1
MTKTLHARPSAATDTTFAPPVITGTATEDALEILFHALLDVARRHDPELEDVLHGRADISSFTPEMLARALQVQGIWFQLVSIAEQNAAMRRRRHVERDQGREALNGSFAKVLAEASARGIGPQQIHALLKDLRIRPTITAHPTEGKRVTVLEKLRRIYLVLRELELPRWTERERNGLMNELRDQIELIWMTGELHLEKATVEREVAWGLHFFDETLFEMLPEMLLSLEESLAQYYPDETFEVPPFFQFGSWIGGDRDGNPYVTASVTRETLQRNALASLRRYRDGITHLGRVLSITERSLPVPETFRSELAHMLAESGDARAIANRNPGEAYRQFLSCVLRKLEATIARNKGARSVGPDYPSADGLINDLRTLEKGLADAKCGALATDIVRPVRRMVEIFRFSTVRLDLRENSTRTTKTLHALWKLRNGDREPPALDSPAWKDWLLTELARPRTPETSFEDFADRLPDDARETLATFALVGEMRDTLDREAFGAFILSMTRSTVDVLGAYLLAKEAGIFLDTTGTEICPLPIVPLFETIDDLRAAPAIMKELLGIPVVRRSTRWQGGVQEVMIGYSDSNKDGGFIASNWELYKAQVRLTTLGNHLGVPIAFFHGRGGSVSRGGVPTHRGIAAQPPGSIQGRFRITEQGEVVSFKYANRGTAAYQMELLAASVFEHALLSEGNGNGSRAEFDDALEALSGASRAAYVNLLQAEGLVDYFQAASPLDEISLLNIGSRPARRFGAKSLSDLRAIPWVFAWSQNRHVITGWYGVGSGLKSFIDVRGEAGEALLRRLFRDCRVFRLVLDEVEKTLLMVDLEIARDYAGLVEDAGIRARIFGMIEAEYALTREMVLRVSGDSELAQRFPQFSERLRGRLPTINQVSREQVELLRRYRSETDEDKREAVKSALLLSINCIAVGFGATG

SEQ ID NO: 50 - protein, 787 aa, Clostridium butyricum
MISKGFSTQTERINILKAQILNAKPCVESERAILITESFKQTEGQPAILRRALALKHILENIPITIRDQELIVGSLTKEPRSSQVFPEFSNKWLQDELDRLNKRTGDAFQISEESKEKLKDVFEYWNGKTTSELATSYMTEETREAVNCDVFTVGNYYYNGVGHVSVDYGKVLRVGFNGIINEAKEQLEKSRSIDPDFIKKEKFLNSVIISCEAAITYVNRYAKKAKEIADNTSDAKRKAELNEIAKICSKVSGEGAKSFYEACQLFWFIHAIINIESNGHSISPARFDQYMYPYYENDKNITDKFAQELIDCIWIKLNDINKVRDEISTKHFGGYPMYQNLIVGGQNSEGKDATNKVSYMALEAAVHVKLPQPSLSVRIWNKTPDEFLLRAAELTREGLGLPAYYNDEVIIPALVSRGLTLEDARDYGIIGCVEPQKPGKTEGWHDSAFFNLARIVELTINSGFDKNKQIGPKTQNFEEMKFFDEFMKAYKAQMEYFVKHMCCADNCIDIAHAERAPLPFLSSMVDNCIGKGKSLQDGGAEYNFSGPQGVGVANIGDSLVAVKKIVFDENKITPSELKKTLNNDFKNSEEIQALLKNAPKFGNDIDEVDNLAREGALVYCREVNKYTNPRGGNFQPGLYPSSINVYFGSLTGATPDGRKSGQPLADGVSPSRGCDVSGPTAACNSVSKLDHFIASNGTLFNQKFHPSALKGDNGLMNLSSLIRSYFDQKGFHVQFNVIDKKILLAAQKNPEKYQDLIVRVAGYSAQFISLDKSIQNDIIARTEHVM

SEQ ID NO: 51 - DNA, 606 bp, Artificial sequence
  Synthetic
ggtctcaatcgaccgtggccgacgccggtcgcttctactcctatcggcgcgatggcgtgaccggccgcatggccagcctggtctggctggcggactgagcccgccgctgcctcactcgtccttgcccctggccgcctgcgcgcgctcggcttcagccttgcgtcggcggcggccgggcgtgcccatgatgtagagcaccagcgccaccggcgccatgccatacatcaggaaggtggcaacgcctgccaccacgttgtgctcggtgatcgccatcatcagcgccacgtagagccagccaatggccacgatgtacatcaaaaattcatccttctcgcctatgctctggggcctcggcagatgcgagcgctgcataccgtccggtaggtcgggaagcgtgcagtgccgaggcggattcccgcattgacagcgcgtgcgttgcaaggcaacaatggactcaaatgtctcggaatcgctgacgattcccaggtttctccggcaagcatagcgcatgcctgcagggagctgttgacaattaatcatcggctcgtataatgtgtggaattgtgagcggataacaatttcacaccggcgcgcctatgtgagacc

SEQ ID NO: 52 - DNA, 1708 bp, Artificial sequence
  Synthetic
ggtctcatatgaaaggaggtatatcgatgaaaacagatgtgcaaatcgcgcaagaagcgcaaatgaaaccaataaccgaagtcgcaaattacctgggaattcaagatgatgaactcgagctatatggaaaatacaaagcaaaagtgagcctggatgtgctcgaacggcaaaaagacaaagaagatgcaaaactcgttctcgtgactgcaataaatccaacgcctgctggtgaaggaaaaacaacaacaaatgtggggctatcaatgggcctgaacaaaatcggaaagcgaacaataactgcgctgcgtgaaccatcgctgggaccatgcttcggtgtgaaaggtggtgcagcaggaggaggatatgcacaagtggttccaatggatgacataaatctacatttcacaggtgattttcatgcaataacatccgcgcacaatctgctcgctgcgctactcgacaatcatctgcatcaaggaaatgcactgaacataaacccaaaaaaaatcgtatggaaacgagtgatcgacatgaatgaccgaagcctgcgaaatgtgataatcggcctgggaggaaatggtgatggattcgtgagacaagcacaattcgacataacagtggcatccgaaataatggcaattctatgcctcgcaacatcaatgagtgacctgaaagaacgactgagcaaaatgatcgtcgcatatgcaaaagatggaagtgcagtgactgctgggcaactggaagcaactggtgcaatggcactactactgaaagatgctgtgaagccaaatctcgttcaaacgctggaaaacacaccagcattcatacatggtgggccattcgcaaacatcgcacatggatgcaattccgtactggcaacaaaagtcgcactgaaactggctgattatgtcgtgacagaaggtggattcggtgccgacctcggtgcagaaaaatttttcgacataaaatcacgattcgccgggctgaaaccaaattgtgtggtgatcgtggcaaccgtacgagcgctgaaaatgaatggaggagttccaaaaacagaactggcagcagaaaatgtcgaagcagtgaaaaaaggagtggcaaacctggaacgccacatcgaaaatgtggcaaaattcggtgttcctgctgtggtcgcaataaacaaatttccgctcgacacagaagctgaactgaaagcagtggaagatgcatgcaatgcaaaaggtgctgatgtggtgctatctgatgtatgggcaaatggtggagaaggaggagtggaaatggcaaaaaaagtggtcgaaatatgcgaaaaaaacgaagccaatttcgcgccgctgtatgatgtgaacctgagcattcccgaaaaaatcgaaaaaatcgcaacaacaatataccgagccgatggtgtcgatttcacatccgattgcaaaaaacaaatcgccgaactggaaaaactcggcctcgacaaaatgccaatatgcatggcaaaaacacaatattcattttccgatgacccaacactactgggagcaccaacaggatttcgaataacagtacgtgaagtgagagtatctgccggagcaggattcatcgtcgcgctgactggaaacataatgacaatgccagggctaccaaaagttcccgctgcaaatggaatggacattctcgaatccggtgaaataatcgggctgagctgatgagtgagacc

SEQ ID NO: 53 - DNA, 1431 bp, Artificial sequence
  Synthetic
ggtctcatgagaaaggaggtatatcgatggacattcgagaaatcggccttcgactggtcggagaagcaataaaagcagctgacccataccgtgctgtgctgaatgccgtgaaagtgagtgatgacaaaataatcgtgcaaggaaaagaattcgaaataaaaggaaaagtatatgtgatcgcgctcggaaaagccgcatgtgaaatggcacgtgcaatcgaagatatactggatgtggaagatggtgtcgctgtgacaaaatatggatatggaaaagagctgaaacgaataaaagtgatcgaagccggccatccaatacccgatgaaaaatcaatactcggtgcaaaagaagcactgagcattctgaatcgagcacgtgaaaatgacatcgtattcattctgatatctggtggtggaagtgcactattcgaacttcccgaagaaggaatatcgctggaagatctgaaactgacaaccgacctactactgaaatccggagcaaaaattcacgaaatcaacaccgtgcgaaagcacatatcaaaagtgaaaggtggaaaactcgccaaaatgataaaaggaaccggaatcgtgctgatcatatccgatgtggtgggtgacaacctcgaagcaatcgcatctggaccaacagtgaaagacccaacaacattcgaagatgcaaaacgaattctcgaactatatgacatatgggaaaaagttcctgaatcagttcggctgcacatcgaacgagggctgcgtggagaagtcgaagaaacactgaaagaagatctaccaaatgttcacaattttctgatcgcatcaaattcaatatcatgtgaagcaatcgcgcgagaagcgcaacggctcggattcaaagcatacataatgacaacaacgctcgaaggtgaagcaaaagatgccgggctattcatcggaagcatcgtacaagaaatcgctgaacgaggacggccattcgaaccacctgtggttctcgtattcggtggtgaaacaaccgtgacaatcgaaggaaaaggaggaaaaggtggcccaaaccaagaaatcgcactatctgcaacgcgaaaaatatcagacctggaagcgctgatcgtcgcattcgacacagatggaactgatggcccaactgatgcagctggtggaatcgtcgatggaacaacatacaaaaagctgcgagaaaaaggaatcgatgtcgaaaaagtgctgaaagagcacaattcatatgaagcgctgaaaaaagtgggtggcctactattcacagggccaaccggaacaaatgtgaattcaatcgtgatcgcaatcgtgacatcaaaacgtggacgaacatgatcgcacaggtgccggaaaaagaaaaacgggacgtatcggaggatacgtcccgtttttttatggcctctggccagccggcgtacttgagacc

SEQ ID NO: 54 - DNA, 216 bp, Artificial sequence
  Synthetic
ggtctcaatcgcctgcaggatcaccagctcaccgtctttcattgccatacggaattcggatccgtgatgtgcagcttggtccgcacttaagggattgcttatacaggggctaagaatatctgaatttaccttatgtgggtgggcttatataattgcatcaacgcagcagccaagacgctcaaccacgcaaggagacaggcgcgcctatgtgagacc

SEQ ID NO: 55 - DNA, 1210 bp, Artificial sequence
  Synthetic
ggtctcatatgaaaggaggtatatcgatggatgtacatgaataccaagcaaaagagcttctggcatcattcggagtcgctgttccaaaaggagctgtggcattttcacctgatcaagccgtatatgcagcaacagaactgggtggatcattttgggcagtgaaagcgcaaattcatgctggtgcgcgaggaaaagccggtggaataaaactatgccgaacatacaatgaagtgcgagatgctgcgcgtgaccttctcggaaaacgactggtgacactgcaaacaggccccgaaggaaagccagttcaacgagtatatgtggaaacagcagatccattcgaacgagaactatatctcggatatgttctggaccgaaaagcagaacgtgttcgtgtgatcgcatcacaacgaggtggaatggacatcgaagaaatcgccgcaaaagaacctgaagcgctgattcaagtcgtcgtcgaaccagctgtcgggcttcaacaatttcaagcacgtgaaatcgcatttcagctggggctgaacataaagcaagtgagtgccgccgtgaaaacaataatgaatgcataccgagcatttcgagattgtgatggaacaatgctggaaataaacccgctcgtggtgacaaaagatgatcgagtgctcgcactggatgcaaaaatgagcttcgatgacaatgcactatttcgacggcgaaacatcgcagacatgcatgacccatcgcaaggtgatccgcgtgaagcgcaagcagccgaacacaatctatcatacatcggcctggaaggagaaatcggatgcatcgtgaatggtgcaggcctggcaatggcaacaatggacatgataaaacatgcaggaggtgaaccagcaaattttctggatgtgggaggaggtgcatcaccagatcgtgtcgcaaccgcatttcgcctggtactgagtgaccgaaatgtgaaagcaatactcgtgaacatattcgcaggaataaaccgatgtgattgggtggcagaaggtgtcgtgaaagctgcacgagaagtgaaaatcgatgttccactgatcgtacggctggccggaacaaatgtggatgaaggaaaaaaaattctggccgaatccggactcgatctgataacagctgacacgctgactgaagctgcgcgaaaagctgtcgaagcatgccatggagcaaaacattgatgagtgagacc

SEQ ID NO: 56 - DNA, 928 bp, Artificial sequence
  Synthetic
ggtctcatgagaaaggaggtatatcgatgagcattctgatcgatgaaaaaacgccaattctcgtgcaaggaataaccggcgacaaaggaacatttcatgccaaagaaatgatcgcatatggaagcaatgtggtgggaggagtgacacctggaaaaggaggaaaaacacattgtggagttcctgtattcaacactgtgaaagaagccgtcgaagcaaccggagcaacaacatcaataacattcgtcgcgccgccattcgccgcagatgcaataatggaagctgccgatgcagggctgaaactcgtatgcagcataactgatggaataccagcgcaagacatgatgcgtgtgaaacgatacctgcgccgatatccaaaagaaaaacgaacaatggtggtcgggccaaattgtgctggaataatatcgcccggaaaatcaatgctgggaataatgcccggacacatatacctgccaggaaaagtgggagtgatatcgcgaagtggaacactcggatatgaagccgccgcgcaaatgaaagaactgggaatcggaatatcaacatccgtcggaatcggtggtgacccaataaatggatcatcatttctcgaccatctcgcgctattcgaacaagaccctgaaaccgaagcagtgctgatgatcggtgaaatcggaggaccgcaagaagcagaagcatctgcatggataaaagaaaatttttcaaaacccgtgatcggattcgtggccgggctgactgcaccaaaagggcgccgaatggggcatgcaggagcaataatatcagcaactggagattccgcagccgaaaaagctgaaataatgagatcatatggactgactgtcgcgcccgatcccggatcattcggaagcactgtggcagatgtactggcacgagctgcataaggagtgagacc

SEQ ID NO: 57 - DNA, 1618 bp, Artificial sequence
  Synthetic
ggtctcaggagaaaggaggtatatcgatgagcttcacgctgatacagcaagcaacgccacgcctgcaccgaagtgaactggcagtgccaggaagcaacccaacattcatggaaaaatctgctgcatcaaaagccgatgtgatatttctggatctcgaagatgcagtcgcacctgatgacaaagaacaagcgcgaaaaaacataatacaagcactgaatgatctggattggggaaacaaaacaatgatgattcgaataaatgggctcgacacgcattacatgtatcgagatgtggtggacatcgtcgaagcatgcccgcgactcgacatgatactgataccaaaagtcggtgtaccagctgatgtatatgcaatcgatgtactgacaacgcaaatcgaacaagcaaaaaaacgagaaaaaaaaatcggattcgaagtactgatcgaaacagcgctcggaatggcaaatgtggaagcaatcgcaacatcatcaaaacgcctcgaagcaatgagtttcggagtggcagattatgccgcatcaacgcgagcgcgaagcactgtgatcggtggtgtgaatgctgattattccgttctgacagacaaagatgaagccggaaatcggcaaacgcattggcaagatccatggctattcgcgcaaaatcgaatgctggtggcatgccgagcatatggactgcgaccaatcgatggaccattcggtgatttttcagatcccgatggatacacatccgctgcgcggcgatgtgctgcactgggattcgaaggaaaatgggcaattcatccatcacaaatcgacctcgcaaatgaagtattcacgccatccgaagcagaagtgacaaaagcgcgccgaatactggaagcaatggaagaagccgcaaaagcaggacgaggtgccgtatcactcgatgggcggctgatcgacatcgcatcaattcgaatggccgaagcgctgattcaaaaagctgatgcaatgggtggaaaatgatacttgagacggcatctagctcgtctcaatcgatacctcacgcaaaacaagaaaaccggccgggaaagtcccggccggtttttctggtcacccgccctgccctggccctgccggcctggttcaaccagtcggcagccggcgctggcgcccgcgtattgcggtgcagccagcgcggcgcacaaggcggcgggcgtttcgtttcgccgcccgtttcgcgggccgtcaaggcccgcgaatcgtttctgcccgcgcggcattcctcgctttttgcgccaattcaccgggttttccttaagccccgtcgcttttcttagtgccttgttgggcatagaatcagggcagcggcgcagccagcaccatgttcgtgcagcgcggccctcgcgggggcgaggctgcaggccgccacgcgcagccatgcgcgaacgggccaccagatggccggcacgacaacaagcagatggcgcgggcgataccgatttgcgcactgcaccccatgcggtgcagcagcgcgcaaacagcgatgacacaaggacagagcaccgatggccacgaccaaaaaaggcgcagagcgactgatcaaaaagtatccgaaccgtaggctctacgtacttgagacc

SEQ ID NO: 58 - DNA, 1395 bp, Artificial sequence
  Synthetic
ggtctcaatcgcgtctcatactatactggaattgccagctggggcgccctctggtaaggttgggaagccctgcaaagtaaactggatggctttcttgccgccaaggatctgatggcgcaggggatcaagatctgatcaagagacaggatgaggatcgtttaaaggaggtatatcgatggccgccacccggaggcctggcaggaatcatttattcgtacctgggcctacgaacatacctgaccgcgtcatgagggccatgatggtgcaatctgaggatcaccggagcgttgattttccaagtttgacgaagcctttatttgaggacacgaagaaggtttttggttcgactgagggcaccatttttttatttcctgcttctggcactggcatatgggagagcgctttatcgaacactttggctcgtggtgataaggttttggctgcccgttttgggcaattttcgcatttatggattgacatggcgcaaaggcttgggcttgacgtcgtcgtgcaggaggaggagtggggtactggggcgaagcctgagaagattgaggaagctttaagggctgataagaatcatgagattaaggccgtcatggtcgtgcataatgaaaccgcgactggcgttacttcgaacattggtgccgtaaggaaggccattgacgccgctgggcatcctgctttgcttttcgttgatggcgtaagcagcattggatctttgccttttaaggctgatgaatggaaggttgactgtgccattgctggttcgcagaagggtttgatgctgccggctgggcttggcgtcatatgcgtatcgcagaaagctttgaaggccgctgaggggcaatctggaaggaatgaccgtttagctcgcgtgtattttgactgggaggatcagaagaagcagaatcctactgggtattttccgtatacgccgcctttacctttattgtatggtttgcgtgaagctttggcatgtttgtttgaggaggggcttgagaacgtgtatcatcggcacgccgtgcttggtgaggcaacaaggcaggccgtggctgcatggggtttgaaaacatgtgcgaaatcacctgagtggaattctgataccgtaacggccatcctcgcgcctgagggcgttgacgctgcgaagatcattaagcatgcgtacgtgaggtataatttggcgcttggtgctggtttatcgcaggttgctgggaaagttttccgcattgggcacgttggtgatttgaatgaattgagtttgcttggggccatcgctggggctgagatgagtttgattgataatggcgtgaaggttacacctggttctggcgttgccgctgcatcatcgtatttgcgtgagaatcctttggcgaaggcatgatatgtgagacc

SEQ ID NO: 59 - DNA, 982 bp, Artificial sequence
  Synthetic
ggtctcatatgaaaggaggtatatcgatgacaaaaaaagtggtatttctggaccgcgaatcgctcgatgcaaccgtgcgtgaattcaattttccgcatgaatacaaagaatacgaatcaacatggacgcccgaagaaatcgtcgaacggcttcaaggtgccgaaatcgcaatgataaacaaagtaccaatgcgtgcagacacgctgaaacaacttcctgacctgaaactgatcgccgtcgctgcaactggaactgatgtggtggacaaagcagctgcaaaagcgcaaggaataacagtcgtgaacattcgaaattatgcattcaacactgtaccagaacatgtcgtcgggctgatgttcgcgcttcgacgtgcaatcgttccatatgcaaattccgtgcgacgaggtgattggaacaaatcaaaacaattttgctatttcgattacccaatatatgacatcgccggaagcacactcggaataatcggatatggagcgctgggaaaatcaatcgcaaaacgtgccgaagcactgggaatgaaagtactggcattcgatgtatttccgcaagatgggctggtcgatctcgaaacaattctgacacaatcagatgtgataacactgcatgtaccgctgacgccagacacaaaaaacatgatcggagctgaacaactgaaaaaaatgaaacgaagtgcaatactgataaacaccgcacgaggaggactcgtggatgaagccgcgctacttcaagcgctgaaagatggaacaatcggaggagctggattcgatgtcgtggcacaagaaccaccaaaagatggaaacattctatgtgatgcagatcttccaaacctgatcgtgacgccgcatgtcgcatgggcatcaaaagaagcaatgcaaatactggccgatcaactcgtcgacaatgtggaagcattcgtcgcaggaaaaccgcaaaatgtggtcgaagcatgatgagtgagacc

SEQ ID NO: 60 - DNA, 904 bp, Artificial sequence
  Synthetic
ggtctcatgagaaaggaggtatatcgatgagcaaaaaactgctatttcaattcgacacagatgcaacgccatccgtattcgatgtggtggtgggatatgatggaggagctgaccacataaccggatatggaaatgtgacgcccgacaatgtgggtgcatatgtggatggaacaatatacacgcgtggtggaaaagaaaaacaatcaaccgcaatattcgtgggaggaggagacatggcagcaggagaacgtgtattcgaagccgtgaaaaaacgatttttcgggccatttcgagtatcatgcatgctggattcaaatggaagcaacacaacagccgctgctggagtcgcgctcgtcgtgaaagctgcaggaggaagtgtgaaaggaaaaaaagctgtcgttctcgctggaaccggacctgtcggaatgagaagtgctgcactgctcgccggagaaggtgcagaagtggtgctatgtggacgaaaactggacaaagcacaagcagcagcagattctgtgaacaaacgattcaaagtgaatgtgactgctgccgaaactgctgatgatgcatcacgtgccgaagcagtgaaaggtgcacatttcgtattcactgccggtgcaatcggcctcgaactacttccgcaagctgcatggcaaaatgaatcatcaatcgaaatcgtcgccgattacaatgcgcaaccaccgctcggaatcggtggaatcgatgcaacagacaaaggaaaagaatatggaggaaaacgagcattcggagcactgggaatcggaggactgaagctgaagctacaccgtgcatgcatcgcaaagctattcgaatcatccgaaggagtattcgatgccgaagaaatatacaaactggcaaaagaaatggcataaggagtgagacc

SEQ ID NO: 61 - DNA, 763 bp, Artificial sequence
  Synthetic
ggtctcaggagaaaggaggtatatcgatggcaggaaatgaaacaatcgaaacatttctggatggcctcgcatcatctgcgccaacacctggaggtggaggagcagccgcaatatctggtgcaatgggagccgcactcgtgagcatggtatgcaatctgacaatcggaaaaaaaaaatatgtggaagtggaagccgatctgaaacaagtactggaaaaatccgaagggctgcgacgaacgctgacaggaatgatcgccgatgatgtcgaagcattcgatgcagtgatgggagcatatggactaccaaaaaacacagatgaagaaaaagctgcacgagcagcaaaaattcaagaagcactgaaaaccgcaaccgatgtaccgctcgcatgctgccgagtatgccgagaagtgatcgatctggctgaaatcgtggcagaaaaaggaaatctgaatgtgatatccgatgcaggagtggcagtgctgagtgcatatgccgggcttcgaagtgctgcactgaatgtatatgtgaatgcaaaaggcctcgatgatcgagcattcgcagaagaacgcctgaaagaactcgaaggcctactcgctgaagccggagcgctgaatgaacgaatatatgaaaccgtgaaatcaaaagtgaattgattccggaattcgccgacacaaaaaaaccggtcctggggaccggtttttttgcgccttgcgcgggttcgataccgcgccgcacagctcttggctgagacgtacttgagacc

SEQ ID NO: 62 - DNA, 3089 bp, Artificial sequence
  Synthetic
ggtctcaatcgcgtctcatggcatatcaccagctcaccgtctttcattgccatacggaattcggatccgtgatgtgcagcttggtccgcacttaagggattgcttatacaggggctaagaatatctgaattgacattatgtgggtgggcttatataattgcatcaacgcagcagccaagacgctcaaccacgcaaggagacaaaaggaggtatatcgatgaccaagaccttgcacgcccgtccgtcggcagcaaccgataccaccttcgccccaccagtgatcacgggcaccgccactgaggatgccttggaaattttgttccacgcccttctggacgttgcccgtcgccacgatccggaattggaggacgtgctgcatggccgtgccgatatctcgtcgttcaccccagaaatgttagcccgcgccttacaggtgcagggcatctggttccagctggtgtcgatcgccgagcagaacgcagccatgcgtagacgtcgtcatgttgagcgcgatcaaggccgtgaagccttgaacggcagcttcgccaaggttttggcagaagcctcagcccgcggcattggcccacagcaaatccatgccctgttgaaggatctgcgcattagaccgaccattaccgcccacccgactgaaggcaagcgcgtgactgttctggaaaagctgcgtcgtatttacctggtgctgcgcgaattggagttgccgcgctggaccgagcgcgaacgtaatggcctgatgaacgagctgcgcgaccagatcgagctgatctggatgactggcgaattgcacttggaaaaggccaccgtggagcgcgaggtggcctggggcttgcactttttcgacgaaaccctgttcgagatgctgccggagatgctgttgtcgttggaggagagcctggcccaatactacccggacgaaaccttcgaggtgccgccgttctttcagttcggctcgtggatcggtggcgacagggatggcaatccatacgtgaccgcaagcgtgacgcgtgaaactctgcaacgtaatgccctggcctcgttgcgtcgctaccgtgatggcatcacccacctgggtcgtgtgctgtcgatcaccgagagatcgctgccggttccagaaactttccgctcggagctggcccatatgttggccgaatcaggcgatgcgcgtgccatcgccaaccgcaatccaggtgaggcctatcgccagtttctgtcgtgcgttttacgcaagctggaggcaactatcgcacgcaacaaaggcgcccgttcggttggcccggactacccgagcgcagacggcttgattaatgatttgcgtaccctggagaagggtttggccgacgccaaatgcggtgcattggccaccgacattgttcgcccggtgcgccgcatggttgaaattttccgcttcagcaccgtgcgcttggacctgcgtgaaaattcgacccgcaccaccaagaccctgcatgcattgtggaagttgcgcaacggcgatcgtgaaccgccggcactggactcgccagcctggaaagattggttgttgaccgaactggcccgtccacgtaccccggaaacttcgttcgaagatttcgccgatcgcttgccggacgatgcccgtgaaaccctggcgaccttcgccttagtgggtgagatgcgcgataccttggacagagaggcattcggtgccttcatcctgtcgatgacccgctcgaccgttgacgtgctgggcgcctacctgttggccaaagaagccggtattttcttggataccaccggcaccgagatctgcccgctgccgatcgtgccgctgtttgaaaccattgacgacttgcgtgccgccccggcaatcatgaaggaactgcttggcatcccggtggtgcgtagatcaacccgctggcaaggcggtgtgcaggaagtgatgatcggctactcggactcgaacaaggacggcggcttcatcgcctcgaactgggagctgtacaaggcccaggttcgcttgaccaccttgggcaaccatttgggtgtgccgattgccttctttcacggtcgcggcggtagtgtgtcacgtggtggcgttccgacccatcgtggcattgcagcacaaccgccgggttcgattcagggtcgctttaggattaccgagcagggcgaggtggtgtcgttcaagtacgcaaaccgtggcaccgcagcctatcagatggagctgcttgccgcatcggtgtttgagcatgcactgctttcggaaggcaacggcaacggctcgcgcgcagaatttgacgatgccttagaagcattgtcgggtgccagccgcgccgcatatgtgaatctgctgcaggccgaaggcctggttgactatttccaggccgccagtccgctggatgaaatctcgctgttgaacatcggctcacgtccagcacgccgctttggcgccaaatcgttgtcggacctgagggccatcccatgggtgtttgcctggtcgcagaatcgccatgtgatcaccggctggtatggtgtgggttcgggcctgaaatcgtttatcgacgtgcgcggtgaggccggcgaggcattgctgcgtcgcttgtttcgtgactgccgtgttttccgcctggtgttggacgaggtggaaaagaccctgttgatggtggatctggaaatcgcacgcgactacgccggcttggtggaggatgccggtatccgcgcccgcatcttcggcatgatcgaagccgaatacgcactgacccgtgaaatggtgttgcgtgtgtcaggtgattcggaactggcgcagcgcttcccacaattctcggagcgcttgcgcggtcgcctgccaaccattaaccaggtgtcacgcgaacaggttgagctgctgcgtaggtaccgttcggaaaccgacgaggacaagagagaggcggtgaagtcggccctgctgttgagcatcaactgcatcgccgttggctttggcgcgaccggctgatactttgtcttcgcatctagctgaagacaaatcgaaaaccgcgccttcggggcgcggtttttttattgccggcgtcagcgcatcgtgagacgtacttgagacc

SEQ ID NO: 63 - DNA, 2423 bp, Artificial sequence
  Synthetic
ggtctcaatcgcgtctcatactaaaggaggtatatcgatgatttcgaagggtttttcgacgcaaactgagcgcattaacatcctgaaggcgcagatcctgaacgcgaagccatgcgttgagagtgagagggccatcctcataactgagagttttaagcagactgaggggcagcctgccatccttcgtcgcgctttggctttgaagcacatccttgagaacatacccattaccattcgtgatcaggaactcatcgttggcagtttaacgaaggaaccccggagttcgcaggtttttcctgaattcagtaataaatggctgcaggatgagcttgatcggctgaataaaaggactggtgacgcttttcagataagtgaggaatcgaaggagaaattgaaggatgtttttgagtactggaatgggaaaacgacatctgaattggctacttcgtacatgactgaggaaacccgtgaagccgtgaactgtgacgtttttaccgttgggaactactactataatggcgttgggcacgtttccgttgactatgggaaggttttgagggttggttttaatggcattattaatgaagcgaaggagcagcttgagaaatccaggagcattgatcctgatttcattaagaaggagaaattcctgaacagcgtcatcatttcatgtgaggccgccattacgtacgtgaacaggtatgcgaagaaggcgaaggaaattgctgataatacttctgacgcgaagaggaaggctgaattgaatgagattgcgaagatatgttcgaaggtgagtggtgagggggcgaagagtttctatgaggcctgtcagcttttctggttcattcatgccatcattaacattgaatcgaatgggcattccatatcacccgcccgttttgatcagtacatgtatccgtactatgagaatgacaagaacatcactgataaattcgcgcaggagctcattgactgcatatggatcaagttgaatgacatcaataaggttcgtgatgagatttccacgaagcattttggtgggtatcccatgtatcagaatttgatcgttggtgggcagaacagtgaggggaaggatgctacgaataaggtatcgtacatggcccttgaggcggccgtgcacgtgaaattgccgcaaccatcgctatccgttcgcatctggaataagacgcctgatgaattcctgctccgggccgctgagctaactcgtgaggggcttgggctgcccgcgtactataatgatgaggtcatcataccggcgctcgtaagtcgtgggcttacgcttgaggacgctcgtgactatggcatcattgggtgcgttgaaccgcagaaacctgggaaaactgagggctggcatgattcggctttttttaacctggcccgcatcgttgagctgaccatcaattccggttttgataagaacaagcagattgggccgaaaacgcagaattttgaggagatgaaattttttgacgaattcatgaaggcgtataaggcgcaaatggagtatttcgtgaagcatatgtgctgcgctgataactgcattgacattgcgcatgctgaaagggcaccgctgccttttttatccagcatggttgataactgtattgggaaggggaaatctttgcaggatggtggggctgagtataatttttctgggccgcagggcgttggcgtggcgaacattggtgattcgctcgtcgccgtgaagaagatcgtttttgatgagaataagatcacgccgagtgaattgaagaaaactttgaataacgattttaagaacagtgaggagattcaggcgctgctgaagaacgcgccgaaatttgggaatgacattgatgaagttgataaccttgcccgtgagggtgcgcttgtgtactgccgtgaggtgaataagtatacgaatccccgtggtgggaattttcagcctgggctgtatccatcgagcattaacgtgtattttggttctttaactggggctacacctgatggtcggaaaagtgggcagccgctggctgatggcgtatcaccatctcgtggatgtgacgtcagtggacccactgctgcatgtaattccgtttcgaagcttgaccatttcattgcttcgaatggaacgctttttaatcagaaattccatccaagtgcgctgaagggtgataatgggctcatgaatttgagcagcctcattcgttcgtattttgaccagaagggtttccacgtgcaatttaacgtcattgataagaagattttgctggcggcgcagaagaatcctgagaagtatcaggacctcattgtccgcgtggctgggtattctgcgcaattcatatcgcttgataaaagcattcagaatgatatcatcgcccgcactgagcacgtcatgtgaatcgtgagacgtacttgagacc

SEQ ID NO: 64 - DNA, 2425 bp, Artificial sequence
  Synthetic
ggtctcaatcggaagacaatactaaaggaggtatatcgatgatatcaaaaggattcagcacgcaaaccgaacgaatcaacattctgaaagcacaaattctgaatgcaaaaccatgtgtggaatccgaacgagcaattctgataacagaatcattcaagcaaactgaagggcaaccagcaatacttcggcgagcgctggcactgaaacacatactggaaaacataccaataacaattcgagatcaagaactgatcgtgggaagcctgacaaaagaaccgcgatcatcgcaagtatttccagaattttcaaacaaatggcttcaagatgaactggaccgactgaacaaacgaaccggagatgcatttcaaatatccgaagagtcaaaagaaaagctgaaagatgtattcgaatattggaatggcaaaacaacatccgaactcgccacatcatacatgaccgaagaaacgcgagaagcagtgaattgtgatgtattcacagtcggaaattattattacaatggagtggggcatgtgagtgtcgattatggaaaagttctacgagtcggattcaatggcataataaatgaagcaaaagagcagctggaaaaatcgcgcagcatcgaccctgatttcatcaaaaaagaaaaattcctgaattccgtgataatctcatgcgaagccgccataacatacgtgaaccgatatgcaaagaaagccaaagaaatcgctgacaacacatcagatgcaaagcgaaaagcagaactgaatgaaatcgccaaaatatgcagcaaagtatccggtgaaggtgcaaaatcattttacgaagcatgccagctattttggttcattcacgcaataataaacatcgaatcaaacgggcacagcatatcgcccgcacgattcgatcaatacatgtacccatattatgagaatgacaaaaacataaccgacaaattcgcgcaagagctgatcgactgcatatggatcaagctgaatgacataaacaaagttcgtgatgaaataagcacaaaacatttcggaggatacccaatgtaccaaaacctgatcgtcggtgggcaaaattccgaaggaaaagatgcaacaaacaaagtgagctacatggcactcgaagctgccgtgcatgtgaaactgccacaaccatcactatctgtgcgaatatggaacaaaacaccagatgaatttcttctgcgtgccgctgaactgacgcgtgaagggctgggactacccgcatattacaatgatgaagtgataatacctgcactggtgagccgtgggctgacgctcgaagatgcacgtgattatggaataatcggatgtgtcgaaccacaaaaaccaggaaaaaccgaaggatggcatgattccgcatttttcaacctggcacgaatcgtcgaactgacaataaattccggattcgacaaaaacaagcaaatcggccccaagacgcaaaatttcgaagaaatgaaattcttcgatgaattcatgaaagcgtacaaagcgcaaatggaatatttcgtgaagcacatgtgctgtgcagacaactgcatcgacatcgcgcatgctgaacgagcgccgcttccatttctatcatcaatggtggacaattgcatcggaaaaggaaaatcactacaagatggaggtgccgaatacaatttttccggaccgcaaggtgtgggtgtcgcaaacatcggagattcactggtggctgtgaaaaaaatcgtattcgatgagaacaaaataacgccatccgagctgaaaaaaacgctgaacaacgatttcaaaaactctgaagaaatacaagcgctgctgaaaaatgcaccaaaattcggaaatgacatcgatgaagtcgacaatctggcacgtgaaggagcactcgtatattgccgagaagtgaacaaatacacaaacccgcgtggtggaaattttcagccagggctatacccatcatcaataaatgtatatttcggaagcctgaccggagcaacacctgatggacgaaaatctggccaaccgctggcagatggtgtgagcccatcacgtggatgcgatgtatctggaccaaccgcagcatgcaattccgtatcaaaactggatcatttcatcgcatcaaatggaacactattcaatcaaaaatttcatccatccgcgctgaaaggagacaatgggctgatgaacctatcatcgctgatacgatcatatttcgaccaaaaaggatttcatgtgcaattcaatgtgatcgacaaaaaaatactgctggcagcgcaaaaaaaccccgaaaaataccaagatctgatcgtacgtgtcgcaggatattctgcgcaattcatatcactggacaaatcaattcaaaatgacataatcgcacgaacagaacatgtgatgtgaatcgttgtcttctacttgagacc

SEQ ID NO: 65 - DNA, 590 bp, Artificial sequence
  Synthetic
ggtctcaatcgaccgtggccgacgccggtcgcttctactcctatcggcgcgatggcgtgaccggccgcatggccagcctggtctggctggcggactgagcccgccgctgcctcactcgtccttgcccctggccgcctgcgcgcgctcggcttcagccttgcgtcggcggcggccgggcgtgcccatgatgtagagcaccagcgccaccggcgccatgccatacatcaggaaggtggcaacgcctgccaccacgttgtgctcggtgatcgccatcatcagcgccacgtagagccagccaatggccacgatgtacatcaaaaattcatccttctcgcctatgctctggggcctcggcagatgcgagcgctgcataccgtccggtaggtcgggaagcgtgcagtgccgaggcggattcccgcattgacagcgcgtgcgttgcaaggcaacaatggactcaaatgtctcggaatcgctgacgattcccaggtttctccggcaagcatagcgcatggagctgttgacaattaatcatcggctcgtataatgtgtggaattgtgagcggataacaatttcacacctatgagagacc

SEQ ID NO: 66 - DNA, 2050 bp, Artificial sequence
  Synthetic
ggtctcatatgaaaggaggtatatcgatgtcgctggagctgaaggagaaggagtcggagctgccgttcgacgagcagatcatcaacgacaagtggcgctcgaagtacaccccgatcgacgcctacttcaagttccaccgccagaccgtggagaacctggagtcgttctgggagtcggtggccaaggagctggagtggttcaagccgtgggacaaggtgctggatgcctcaaacccgccgttctacaagtggttcgtgggcggtcgcctgaacctgtcgtacttggccgtggaccgccatgtgaaaacctggcgcaagaacaagctggccatcgaatgggaaggcgaaccggtggacgaaaacggttacccgaccgaccgccgcaagctgacctactacgacctgtaccgcgaggtgaaccgcgtggcctacatgctgaagcagaacttcggcgtgaagaagggcgacaagatcaccttgtacctgccgatggtgccggaactgccgatcaccatgttggccgcctggcgcatcggtgccatcacctcggtggtgttctcaggcttttcagccgacgcattggcagaacgcatcaacgactcgcaatcgcgcattgtgattaccgcagacggtttctggcgtcgtggccgtgtggtgcgcctgaaggaagtggtggacgcagccctggaaaaggccaccggtgttgagtcagttattgttctgccgcgtctgggcctgaaggacgtgccgatgaccgaaggtcgcgattactggtggaacaagctgatgcagggcatcccgccgaacgcctacatcgaaccggaaccggttgagtcggaacacccgtcgttcattctgtatacctcgggcactaccggcaagccgaagggcatcgtgcatgacaccggtggctgggccgtgcatgtgtacgccactatgaagtgggtgttcgacatccgcgacgacgacattttctggtgcaccgccgatatcggctgggtgaccggccattcgtacgtggtgttgggcccgctgttgatgggtgcaaccgaggtgatttacgaaggtgccccggactatccgcagccggatcgctggtggtcgatcatcgaacgctatggcgtgaccatcttctacaccagcccgaccgccatccgcatgttcatgcgctacggcgaggaatggccacgtaagcacgacctgtcgaccttgcgcatcatccactcagtgggcgaaccgattaacccagaagcctggcgctgggcctaccgcgtgctgggcaatgagaaggtggccttcggctcaacctggtggatgactgaaaccggcggcattgtgatttcacatgcaccgggcctgtacctggtgccaatgaaaccgggtaccaacggtccaccgctgccgggcttcgaggtggacgtggtggatgaaaacggtaatccggcaccaccaggcgtgaagggctacctggtgatcaagaagccttggccgggcatgttgcacggcatctggggcgacccggaacgctacatcaagacctactggtcgcgcttcccaggcatgttctacgccggcgattacgccatcaaggacaaggacggctacatctgggtgctgggccgtgccgacgaagtgattaaggtggcgggccaccgcttgggtacttatgagttggagtcggccctgatctcgcatccggcagttgccgaatcagccgtggtgggtgtgccagatgcaatcaaaggcgaggtgccgatcgccttcgttgtgctgaagcagggtgttgccccgtcggacgagttgcgtaaggaattgcgtgaacacgttcgccgcaccattggcccgatcgccgaaccggcccagatcttcttcgtgaccaagctgccaaagacccgctcgggcaagatcatgcgtcgcttgttgaaagccgttgccaccggtgcaccgctgggcgacgtgaccaccttggaggacgaaaccagcgtggaagaagccaagcgcgcctatgaagaaatcaaggccgagatggcccgcacctgatgagagagacc

SEQ ID NO: 67 - DNA, 1584 bp, Artificial sequence
  Synthetic
ggtctcatgagaaaggaggtatatcgatggccaaggagatctacatcgcaggcgaatggcgcttgggccgcggcgccgtgatccaatcgctgttcccggccgatcaatcggtgaacgccgaactgtcgaccgcaactttggaggacgtgaacgaagccatcgagaaggcagatcaggcgtggcgccagccgtcgtggcgcaattcgttgccgcatgagcgcgcccgcatcctgtacaaggttgccgacatcatcgaagcacgcgtggacgaactggccaagctgcaaacccgtgacaacggtaagccgttgaccgatactcgcggcttggtgatgagcgcagcagccactgcacgctacgtggccgccgcctgcgaaaccctgaacgatgagttgaccacccagagagccccggatttcatgaccatgtcggtgcacgagccagtgggcgtggtggcagccatcaccccgtggaactcgccaattgcatcagaagtgcagaagctggccccagccttagcagccggcaacgccgtggtgctgaagccggcagaagccacctcgctgatcgccctggaattggccaagatcttcgaagaagccggcctgccaaagggcctgttgagcgtgctggtgggccgcggttcgatcatcggcgacgccatcgcacagcatccgctggttcgcaagatttcattcaccggcggtaccaccaccggccgtcacctggcacacatcgccgccgacaagctgattaccaccagcctggagctgggcggtaagtcaccgaccatcgttctgccagatgccgatgtggaactggccgccaagggcgtggcctatggcattttttcgtcggccggccaggcctgcatcgcaggctcgcgcttgttcatccactcgtcgctgtacgaccagttcctgacccgcctggtggaaatcactaagggcctgcgcgtgggccatccggaacaagccggtgtgcacttgggcccgctggttaacgataaacacctccagtcggtggaccgttacgtgcagctggccaagtcggaaggcggccaagtgctgatcggcggcgaagccctgaccaccggtgactatgccaagggttcgtactacctgccgaccatcatcaccggcctgaacaactcggcccagacctgccaggaggagatcttcggcccggtgctggtggtgatgaagtacgacaacgagcaggacctgatcgcccaggcaaatgacagctgcttcggccttgccgccggtatctggaccgaatcataccgcaaggcatggcgcatcgcccgcgccttggaagtgggcaccgtgtggatcaacacctacaagaagttctcgatctcggccccgttcggcggcttcaaggactcgggcatcggtcgcgagaagggccgcctgggcatcctgagctacatgcagcagaagagcatctacatgggcctgaacgagcagccgaacccgtggtgcgactgatcgcacaggtgccggaaaaagaaaaacgggacgtatcggaggatacgtcccgtttttttatggcctctggccagccggcgtactagagacc

SEQ ID NO: 68 - DNA, 200 bp, Artificial sequence
  Synthetic
ggtctcaatcgatcaccagctcaccgtctttcattgccatacggaattcggatccgtgatgtgcagcttggtccgcacttaagggattgcttatacaggggctaagaatatctgaatttaccttatgtgggtgggcttatataattgcatcaacgcagcagccaagacgctcaaccacgcaaggagacatatgagagacc

SEQ ID NO: 69 - DNA, 667 bp, Artificial sequence
  Synthetic
ggtctcatatgaaaggaggtatatcgatggccctgacccagatggccctggactcgctggacttcgacgccaccgtggccctggccgaaaaggtggcaccgcatgtggacatcctggagatcggcaccccgtgcatcaagcacaacggcatcaagctgctggaaaccctgcgcgccaagttcccgaacaacaagatcctggtggacctcaagaccatggacgccggcttctacgaggccgaaccgttctacaaggccggcgccgacatcaccaccgtgctgggcgtggccgatctgggcaccatcaagggcgtgatcgacgccgccaacaagtacggcaagaaggcccagatcgacctgatcaacgtgggcgataaggccgcccgcaccaaggaggtggcaaagctgggcgcccatatcatcggcgtgcataccggcctggatcagcaagccgccggccaaactccgttcgcagatctggccaccgtgaccggcctgaatttgggcctggaagtgtcggtggcaggcggcgtgaagccggccactgttgcccaagtgaaagacgccggcgcaaccatcatcgttgcaggagccgccatctatggtgccgcagatccggccgccgcagccgccgaaatcaccggcctggccaagtgatgagagagacc

SEQ ID NO: 70 - DNA, 571 bp, Artificial sequence
  Synthetic
ggtctcatgagaaaggaggtatatcgatgcaccagaagctgatcatcgacaagatcagcggcatcctggccgccaccgatgccggctatgacgccaagctgaccgccatgctggatcaggcatcgcgcatctttgtggcaggcgcaggccgttcaggcctggtggccaagttcttcgccatgcgcctgatgcacggcggctacgacgtgttcgtggtgggcgagatcgtgaccccgtcgatccgcaagggcgacctgctgatcgtgatctcgggctcgggcgaaaccgaaaccatgctggccttcaccaagaaggccaaggaacagggcgcctcgatcgccctgatctcgacccgcgactcgtcgtcgttgggcgatctggccgactcggtgttccgcatcggctcgccggaactgttcggcaaggtggtgggcatgccgatgggcaccgtgttcgagctgtcgaccctgctgttcctggaggccaccatctcgcacatcatccacgagaagggcatcccggaggaggagatgcgcacccgccacgccaacctggagtgaggatagagacc

SEQ ID NO: 71 - DNA, 1574 bp, Artificial sequence
  Synthetic
ggtctcaggataaaggaggtatatcgatgatcaagaaaatcggcgttctgacctcgggcggcgatgccccaggcatgaatgccgccattcgcggcgtggtgcgctcagccctgaccgaaggcctggaagtgatgggcatttacgacggctacctgggcctgtacgaggaccgcatggtgcagctggaccgctattcggtgtcggacatgatcaaccgcggcggcacctttctgggctcggcccgctttccggagttccgcgacgagaacatccgcgccgtggcgatcgagaacctgaagaagcgtggcatcgatgccctggtggtgatcggcggtgacggctcgtacatgggcgccatgcgtctgaccgaaatgggcttcccgtgcatcggtctgccgggcaccatcgacaacgacatcaagggcaccgactacaccatcggcttcttcaccgccctgtcgaccgtggtggaggccatcgatcgcctgcgcgacacctcgtcgtcgcatcagcgcatctcggtggtggaagtgatgggccgctattgcggcgatctgactctggcagccgccatcgccggtggctgcgaattcgtggtggtgccggaagtggagttcagccgcgaggacctggtgaacgagatcaaggccggcatcgccaagggcaagaagcacgccatcgtggccatcaccgagcacatgtgcgacgtggacgagctggcccacttcatcgaaaaggaaaccggccgcgagactcgcgccaccgtgctgggccatattcagcgcggtggctcaccggtgccgtatgatcgtatcttagcctcgcgcatgggcgcatatgccatcgacctgctgctggccggctatggcggccgctgcgtgggcatccagaacgagcagctggtgcaccacgacatcatcgacgccatcgagaacatgaagcgcccgttcaagggcgactggctggactgcgccaagaagctgtactgaatacctcacgcaaaacaagaaaaccggccgggaaagtcccggccggtttttctggtcacccgccctgccctggccctgccggcctggttcaaccagtcggcagccggcgctggcgcccgcgtattgcggtgcagccagcgcggcgcacaaggcggcgggcgtttcgtttcgccgcccgtttcgcgggccgtcaaggcccgcgaatcgtttctgcccgcgcggcattcctcgctttttgcgccaattcaccgggttttccttaagccccgtcgcttttcttagtgccttgttgggcatagaatcagggcagcggcgcagccagcaccatgttcgtgcagcgcggccctcgcgggggcgaggctgcaggccgccacgcgcagccatgcgcgaacgggccaccagatggccggcacgacaacaagcagatggcgcgggcgataccgatttgcgcactgcaccccatgcggtgcagcagcgcgcaaacagcgatgacacaaggacagagcaccgatggccacgaccaaaaaaggcgcagagcgactgatcaaaaagtatccgaaccgtaggctctacgtactagagacc

SEQ ID NO: 72 - DNA, 2052 bp, Artificial sequence
  Synthetic
ggtctcatatgaaaggaggtatatcgatgcgcactcgccgtgagttagccaacgccattcgcgccttgagcatggatgccgtgcagaaggcaaatagcggccatccaggcgccccgatgggcatggcagacatcgccgaagtgctgtggaatgactacttgtgccataacccggccaacccgaagtggccgaaccgcgatcgcttcgtgctgagcaacggccacggctcgatgctgatctattcgctgctgcacctgaccggctacgatctgccgatcgaggaactgcagaacttccgccagctgcacagcaagaccccgggccatcctgagtacggctacaccccaggcgtggaaactaccaccggcccattgggtcagggtattaccaatgccgtgggcatggccttggccgaacgcaccctggcaggccaattcaaccgcccaggtcataccatcgtggatcactacacctacgtgtttctgggcgacggctgcctgatggaaggcatctcgcacgaggcctgctcgctggccggcagcatgaagctgggcaagctgatcgccttctacgacgacaacaacatctcgatcgacggcgaagtgcgtggccatggcggcgtgccaggctggttcatggatgacaccccgcgtcgctttgaagcctacggttggcacgtgattccgaaggttgacggccatgacccggatgccgtgaaggccgccattgaggaggcacgcgcagtgaccgacaagccatcgttaatctgctgccagaccatcatcggctggggctcaccgaacaagcagggcaaagaagattgccacggcgcagccctgggtaccgacgaagtggccctgacccgtgagaatatcggctggtcgcacccgccgttcgaaattccggccgatatctacgaagcctggtcagcacgcgaaactggcgccaaggcggaaggcgaatggaacgaccgcttcgagaactaccgccgcgaattcccggaattggccgccgaattcgaacgccgtatggccggtgaactgccacgcgactgggccgaaaaatcggcagccttcgtggcccaagtgaacgagaaggccgaaaccatcgcctcgcgcaaggcatcgcagaatgccttgaacggctttggcccgttgttgccggaaatcttgggcggctcggccgacttggccggtagcaatctgaccctgtggagcggctgcaaagacgtgaacgcccctggtcatgacggcaactacgtgtactacggcgtgcgcgagttcggcatgtcggccatcatgaacggcatcgccctgcatggcggcttcaagccgtacggcgccaccttcctgatgtttagcgagtacgcccgcaacgccctgcgtatgagcgccctgatgaagatcccggtgatctacgtgtacacccacgacagcatcggcctgggtgaagatggcccgactcatcagccggtggaacagaccgccaccctgcgcttgatcccgaatatgcaggtttggcgtccgtgcgacgccgttgaatcagccgttgcctggaagtgcgcaatcgaacgccgtgatggcccgtcggccttgatcttctcgcgccagaatctgccacatatgccgcgtaccccggaacagattgccgccatcgccaaaggcggctatgtgctggttgattgcaccggcaccccagacctgatcctgatggccactggctcggaagtagaattggcggtgaaggcagccgaggcactgaccgccaagggccgtcaagtgagggtggtgtcgatggcctcgaccaatgttttcgacgcccaggaaccggcctatcgtgattcagtgttgccgccagccgtgacccgtcgcttggccattgaagccggcgtgtcagacggctggtggaagtacgttggctcgcacggcaaagtgattggtttggaccgctttggcgaatcggccccggcaggcgtgctgttcaagacctttggcttcaccgtggacaacgtggtgggccaggccgaggccctgttctgacctcacagagacc

SEQ ID NO: 73 - DNA, 2066 bp, Artificial sequence
  Synthetic
ggtctcatcacccgacaccaccccatggccctgatctcgctgcgccagctgctggaccacgccgccgagcacggctacggcctgccggccttcaacgtgaacaacatggagcagatcaaggccatcatggaagccgcatcggcagtggatgccccggtgattctgcagggttcggcaggcgcccgtacctacgccggcgaacctttcttgcgtcatctggttcgtgccgccatcgagatgtacccgcacatcccggtgtgcatgcatcaggaccacggcgcaagcccggccgtgtgcatccgctcgatccaatcgggcttctcgtcggtgatgatggacggcagcctgctggaggacatgaaaaccccggccagctacgcctacaacgtggaaaccacccgcaaggtggtggagatggcacatgcctgcggtgtttcggtggaaggcgagctgggctgcctgggctcgctggaaactggccgtgccggtaaggaggatggtcatggtgccgaaggcgaactggaccactcgctgttgctgaccgacccggacgaagccgccgacttcgtgcgtcagacccaagttgacgccctggccatcgccatcggcacttcgcatggtgcctacaagttcacccgcaagccgaccggccaggtgctgcgtatcgaccgtgtgaaggccatccaccagcgcattccgaccatccacctggtgatgcacggctcgtcgagcgtgccggaagagtgggcccagatgatcaacgactacggcggcgacatcggccagacctacggcgttccggtggaagagatcgtggaaggcatccgccacggcgtgcgcaaggtgaatatcgacaccgacctgcgcatcgcctcgtacggcgccatgcgccgtttcatggtggaggaccgcaagaatttcgacccgcgcaagctgtacaaggccgcccagaccgccatgactgccatctgccgtgcacgctatgaagcattcggcgccgccggccaagccgccaagattaagccgctgcgtctggaagatatgtcgctggcctacgcccagggcaagctggacccgatcgtgcgctgacgcggtgggccgaacgggcgccgggaggcggacgtgaaagcgtccggcaacggaacgcaagcgatgcaattggaaaaacaaggggggcgatcccgcaacccgccccgccgagcttttgtacccaactgaccagacttatgggggaaaccacgtgaagaaaaacggctgaccctgcgaaagggcgggctgcattgacagcggccccctcactctttacagttgggaaattgtgcgtttagcaataccccataactcctaaggcttaagcccgacttttttctctctaccttacttttttgatcggaggagatctcatggcccgcccgctgatccagctggccctggacaccctggatatcccgcagaccctgaagctggcctcgctgaccgccccgtacgtggacatcttcgagatcggcaccccgtcgatcaagcacaacggcatcgccctggtgaaggagttcaagaagcgcttcccgaacaagctgctgctggtggacctcaagaccatggacgccggcgaatatgaagccaccccgttctttgccgccggcgccgatatcaccaccgtgctgggtgtggccggcctggccaccatcaagggcgtgatcaacgccgccaacaagcataacgccgaggtgcaagtggacctgatcaacgtgccggataaggccgcctgcgcccgcgaatcggcaaaggcaggcgcccaaatcgtgggcatccataccggtctggacgcccaagcagcaggccagactccattcgccgatctgcaggccatcgcaaagctgggcctgccagtgcgcatctcggtggccggtggcatcaaggcatcgaccgcccagcaggtcgtgaaaaccggcgcaaatattatcgtggtgggcgccgccatctacggcgccgcctcgccagccgacgcagcacgcgaaatctatgaacaggtggtggccgccagcgcctgatcgggaatagagacc

SEQ ID NO: 74 - DNA, 1815 bp, Artificial sequence
  Synthetic
ggtctcagaatttgcatgcaccagaagctgatcatcgacaagatctcgggcatcctggccgccaccgacgcaggctatgatgccaaactgaccgccatgctggaccaggccagccgcatctttgtggcaggcgcaggccgttcaggcctggtggccaagttcttcgccatgcgcctgatgcacggcggctacgacgtgttcgtggtgggcgagatcgtgaccccgtcgatccgcaagggcgacctgctgatcgtgatcagcggctcgggcgaaaccgaaaccatgctggccttcaccaagaaggccaaggaacagggcgcctcgatcgccctgatctcgacccgcgattcgtcgtcgctgggcgatctggccgactcggtgttccgcatcggctcgccggaactgttcggcaaggtggtgggcatgccgatgggcaccgtgttcgagctgtcgaccctgctgttcctggaggccaccatctcgcacatcatccacgagaagggcatcccggaggaggagatgcgcacccgccacgccaacctggagtgagaagaaggcaaagcctgtggcagtcctcccttctgcccgggctcttgcccttcctatggacccggcgctcgacgccccggactcgaaccgaatccgagcggccagaggcgggcaacgatgctagggcaaaggccagcagagcacggcgtcgataaggtgccaccgtatgaagcccgtgtaggaaaataaataaagtcccgaccggggaacattttcgtgattgctggcagcacccaatactttaagaaatctttaggagtccaccatgtcgaagaacctgctggaccagctgcgcgaggtgaccgtggtggtggccgacaccggcgacatcgaggcaatcgagaagttcaagccgcgcgatgccaccaccaacccgtcgctgattaccgccgccgcccagatgccgcagtaccaggatatcgtggacgataccttgaagggtgccagacagaccctgggccctggtgcctcggcagcccaagtggccaaccgcgcattcgaccgcctggccgtgtcgtttggcttgaagatcctgcagatcatcgagggccgtgtgagcaccgaggtggacgcccgcctgagctacgacaccgaaggcaccatcgagaaggcccgcgagatcatcaagcagtacgaagccgccggcgtgagcaaggagcgcgtgctgatcaagatcgccgccacctgggagggcattgaagccgcggccgtgctggagaaggaaggcatccactgcaacctgaccctgctgttcggcctgcatcaggccatcgcctgcgccgagaacggcatcaccctgatcagcccgttcgtgggccgcatcctggactggtacaagaaggacaccggccgcgagtcgtacgccccgcatgaagatccgggcgtgctgtcggtgacccagatctacaactactacaagaagttcggctacaagaccgaggtgatgggcgcctcgttccgcaacatcggcgagatcaccgaactggccggctgcgatctgttgaccattgccccgagcctgctggcggaactgcaagcaaccgaaggcgaactgccgcgcaagctggacccggccaaggccaaggactacccgatcgagaagatccacgtgaacaagtacaccttcgacaagatgcacgccgagaaccgcatggccaccgaaaagctggaggaaggcatccagggcttcaccaaggccctggagcagctggaaaagctgctggccgaccgcctggcccacctggaagccgcctgatgagagagacc

SEQ ID NO: 75 - DNA, 1888 bp, Artificial sequence
  Synthetic
ggtctcatgagaaaggaggtatatcgatggccgcccgcaacgccttctatgcccaatcgggcggcgtgactgccgtgatcaacgcatcagcctgcggcgtgctggaaactgcccgccagtatccggatcgcatcggcaccgtgtatgccggtcgcaacggcattgtgggcgccttgaccgaggatctgatcgataccggccaggagtcggccgaagccattgccgcactgcgccatactccgtcgggcgcattcggctcgtgcagatacaagctgaagggcctggaggagaaccgcgcccagtacgagcgcctgatcgaagtgttccgcgcccacgacattggctacttcttctacaacggcggcggcgactcggccgatacctgcctgaaggtgtcgcagctgtcggaaaagctgggctacccattgcaggccgtgcacatccccaagaccgtggacaacgacctgccgatcaccgactgctgcccgggcttcggcagcgtggccaaatatatcgccgtgtcggtgcgcgaggcctcgttcgatgttcgctcgatggccgcaacctcgacctgcatctttgtgctggaagttatgggccgccatgcaggctggattgcggccgccggcggcttggcctcggacgaacgccacgaactggcactggtgatcctgttcccagaacaagtgttcgatccggaacgcttccttcgcgccgtggatgagaaggtgcgctcgcacggctactgctcggtggtggtgtcagaaggcatccgcggtgcagatggccgctttgtggccgagagtggctcacgtgatgttttcggccatgcccgtctgggtggcgtggccccggttattgccgacctgatcaaggaacgcctgggctacaagtaccactgggcagtggccgactacctgcagcgtgcagcccgccatattgcgtcgcgcactgacgtggaacaggcctatgcagtgggcaaggccggtgtggagatggccctgaagggtctgtcggcagttatgccggccattgtgcgcacctcggactcgccgtaccgctgggaaatcactgccgccagcctggcagaagtggccaacgtggagaagaagatgccgctggagttcatcagcgccgacggcttcggcatcaccgaagcatgccgccgctatctgcgtccgctgatcgaaggcgaagattatccgccgtacgcaggcggcctgccagactacgtgaccctgtgcaacgtggccgtgccgaagaagctggccgcctcgttctcggtgtgattccggaattcgccgacacaaaaaaaccggtcctggggaccggtttttttgcgccttgcgcgggttcgataccgcgccgcacagctctcctgccggcctggttcaaccagtcggcagccggcgctggcgcccgcgtattgcggtgcagccagcgcggcgcacaaggcggcgggcgtttcgtttcgccgcccgtttcgcgggccgtcaaggcccgcgaatcgtttctgcccgcgcggcattcctcgctttttgcgccaattcaccgggttttccttaagccccgtcgcttttcttagtgccttgttgggcatagaatcagggcagcggcgcagccagcaccatgttcgtgcagcgcggccctcgcgggggcgaggctgcaggccgccacgcgcagccatgcgcgaacgggccaccagatggccggcacgacaacaagcagatggcgcgggcgataccgatttgcgcactgcaccccatgcggtgcagcagcgcgcaaacagcgatgacacaaggacagagcaccgatggccacgaccaaaaaaggcgcagagcgactgatcaaaaagtatccgaaccgtaggctctacgtactagagacc

SEQ ID NO: 76 - DNA, 590 bp, Artificial sequence; identical to SEQ ID NO: 65
  Synthetic

SEQ ID NO: 77 - DNA, 1117 bp, Artificial sequence
  Synthetic
ggtctcatatgaaaggaggtatatcgatgatcctgatgcgccgcaccctgaaggccgccatcctgggcgccaccggcctggtgggcatcgagtatgtgcgcatgctgtcgaaccatccgtacatcaagccggcatacttggccggcaagggctcggttggcaagccgtatggcgaagtggtgcgctggcagaccgtgggccaggtgccgaaggaaatcgccgacatggagatcaagccgaccgacccgaagctgatggacgacgtggacatcatcttctcgccgctgccgcaaggcgccgcaggcccggtggaagaacagttcgccaaggaaggcttcccggttatctcgaactcgccggaccaccgctttgatccggacgtgccgctgctggtgccggagctgaacccgcacaccatctcgctgattgacgaacagcgcaagcgccgtgaatggaagggctttatcgtgaccaccccactgtgcaccgcccagggtgccgccatcccgctgggcgccatcttcaaggactacaagatggacggcgccttcatcaccaccatccaatcgctgtcgggcgcaggctatccgggcatcccgtcgctggacgtggttgacaatatcctgccgctgggcgacggctacgacgccaagaccatcaaggagatcttccgcatcctgtcggaagtgaagcgcaacgtggacgagccgaagctggaggacgtgtcgttggccgccaccacccatcgcatcgccaccatccacggccactacgaggtgctgtacgtgagcttcaaggaggaaaccgccgccgagaaggtgaaggaaaccctggagaacttccgcggcgagccgcaggacctgaagctgccgaccgccccgtcgaagccgatcatcgtgatgaatgaagatacccgcccccaggtgtacttcgaccgctgggccggcgacatcccgggcatgtcggtggtggtgggcagactgaagcaagtgaacaagcgcatgatccgcctggtgagcttgatccataataccgtgcgcggcgcggccggcggcggcatcctggccgccgagctgttggtggaaaagggctacatcgagaagtgatgagagagacc

SEQ ID NO: 78 - DNA, 1368 bp, Artificial sequence
  Synthetic
ggtctcatgagaaaggaggtatatcgatgatcaaccgccgctacgagctgttcaaggacgtgtcggacgccgactggaacgactggcgctggcaagtgcgcaaccgcatcgaaaccgtggaggagctgaagaagtacatcccgctgaccaaggaggaggaagagggcgtggcccagtgcgtgaagtcgctgcgcatggccatcaccccgtactacctgagcctgatcgacccgaacgatccgaacgacccggtgcgcaagcaggccatcccgactgccctggagctgaataaggccgccgcagacctggaagatccgctgcatgaagataccgactcgccggtgccgggcctgacccatcgctatccggatcgcgtgctgttgctgatcaccgacatgtgctcgatgtactgccgccattgcacccgccgtcgcttcgccggccagtcggacgactcgatgccgatggaacgcatcgataaggccatcgactacatccgcaacaccccgcaagtgcgcgacgtgctgctgtcgggcggcgacgccctgctggtgagcgacgaaaccctggagtacatcatcgccaaactgcgcgaaatcccgcacgtggagatcgtgcgcatcggctcgcgcaccccggtggtgctgccgcagcgcatcactccggaactggtgaacatgctgaagaagtaccacccggtgtggctgaacacccacttcaaccatccgaacgagatcactgaagagtcgacccgcgcctgccaactgctggccgacgccggcgtgccgctgggtaaccagtcggtgttgctgcgtggcgttaacgactgcgtgcatgtgatgaaggagctggtgaacaagctggtgaagatccgcgtgcgcccgtactacatctaccagtgcgacctgtcgctgggcctggagcacttccgcaccccggtgtcgaagggcatcgaaatcatcgaaggtctgcgcggtcacacctcgggttattgcgtgccaaccttcgtggtggatgccccgggcggcggcggcaagaccccggtgatgccgaactatgtgatctcgcagtcgcacgacaaggtgatcctgcgcaacttcgagggcgtgatcaccacctactcggagccgatcaactacaccccgggctgcaactgcgatgtgtgcaccggcaagaagaaggtgcacaaggtgggcgtggccggcctgttgaatggcgagggcatggccttggagccggtgggcctggaacgcaacaagcgccacgtgcaggagtgatcgcacaggtgccggaaaaagaaaaacgggacgtatcggaggatacgtcccgtttttttatggcctctggccagccggcgtactagagacc

SEQ ID NO: 79 - DNA, 1512 bp, Artificial sequence
  Synthetic
ggtctcaatcgcgtctcatactatactggaattgccagctggggcgccctctggtaaggttgggaagccctgcaaagtaaactggatggctttcttgccgccaaggatctgatggcgcaggggatcaagatctgatcaagagacaggatgaggatcgtttaaaggaggtatatcgatgaacgacttcagcaacccgagcgtgtcggcacgcggcgccttctgggccccattcactccgatgcgccagttccagcagcagccaatgatgttcgcatcggcagatggcatgcactacaccaccaccgacggccgccgcgttctggacgcaatggccggtctgtggtgtgtgaatgccggtcatggtcagccgaccattgtggaagccattagagaagccgccggccgcttggatttcgtgtcgtcgttcaagatgtcgcatccgcaggccctggaaatggccgagcgcctgattgacatcagcccggccggcatggagcaggtctttttcaccaactcgggttcggaggcagtggacaccgccctgaagattgcccgcgcatatcatcaggccagaggcgattgccgccgcaccaagttcatcggccgcgcgaaaggctatcatggcatgggcttcggcggtttgtcagtgtcgggcatcggtcgccagaagcgcgatttcggccctctgctgggtgaagtgtcgcacttgccgttgccgtacgatgcgggcatgcgcttctcagtgggccagccgcaacaaggcgcaagctacgccgatgcattgacccagctgctggatatccaagacccatcgaccgtggccgccgtgatcgtggaaccagtgaccggtagcggcggcgtttatgccccgccgcaaggctatttacagcgcctgcgtgaaatctgcacccgccatggcgtgctgctgatcttcgacgaagttatcactggcttcggccgcgtgggcgccccgtttgcagcccaagccttcggcgttttgccagatttgattaccaccgccaaaggcctgactaacggcgcggtgccaatgggcggcgtgctggtgagcggcgccgtgtacgaagcattcatggccggcccgcaaaatgtgatcgagctgatgcacggctatacctactcggcccacccgttggcctgtgccgccggcttggccaccttggaagtgcatcgcgaactgggcatcaaccaacatgtgaacgccgtgtcgggtctgtggcagtcgaccgcactggccctgcaaggcattggcgccgtgctggatgtgcgtgcaatcggcttgttatgcgccgttgaactggaaccacgccctggttttccaggcgcccgcggctcggaagtggcacagtggtgcttcgataacggcgttctggttcgcggctcaggcgacaccatcgttattagcccgccgctggtgatctcgccgcaggagatcgcccaggtgttcgaaaccctggcctcggccttgaagcacgtggcctgatatgagagacc

SEQ ID NO: 80 - DNA, 2416 bp, Artificial sequence
  Synthetic
ggtctcatatgaaaggaggtatatcgatggacgaccagctgaagcagtcggccctggacttccacgaattcccggtgccaggcaagattcaggtgtcaccgaccaagccgttggccactcagcgtgatttagccctggcctattcaccgggtgtggccgccccgtgtttggaaatcgagaaggacccgctgaaggcctacaagtataccgcccgcggcaatctggttgccgtgatctcgaatggcaccgccgttttgggtctgggcaacatcggtgcgttggcaggtaagccggtgatggagggcaagggcgtgctgttcaagaagttcgccggcatcgacgtgttcgatatcgaggtggacgagctggacccggacaagttcattgaggtggttgccgccctggagccgaccttcggcggcatcaacctggaggatatcaaggccccagagtgcttctacatcgagcagaagctgcgcgagcgcatgaacatcccggtgttccacgacgaccagcacggcaccgcgatcatttcgaccgccgccatcctgaatggtttgcgcgtggtggaaaagaacatttcggacgtgcgcatggttgtgtcgggtgccggcgccgcggccattgcctgcatgaacctgttggtggcgctgggtttgcagaagcacaacatcgtggtttgcgactcaaaaggcgtgatctaccagggccgcgagccgaatatggccgaaaccaaggccgcctatgccgtggtggacgacggcaagcgtaccctggacgatgtgatcgaaggtgcagatatcttcctgggctgctcaggcccgaaggtgctgacccaggagatggtgaagaagatggcccgcgccccgatgatcttggccttggccaatccggaaccagaaatcctgccgccacttgccaaggaggttcgtccagatgccatcatctgcaccggccgcagcgactacccgaaccaggtgaacaacgtgctgtgcttcccattcatcttccgtggcgccttggacgtgggcgcaaccgccatcaacgaggagatgaagttggccgccgttcgcgccattgcagaactggcccacgcagaacaatcggaagtggtggcatcggcctatggcgaccaggatttgtcgttcggcccggagtacatcatcccgaaaccttttgacccgcgtttgatcgtgaagatcgccccggccgtggccaaagccgccatggaatcaggcgtggcaacccgcccgatcgcagacttcgacgtgtacattgacaagctgaccgagttcgtgtacaagaccaacctgttcatgaagccgatcttctcgcaggcccgcaaggcaccgaagcgcgttgtgctgccggaaggtgaagaagcccgcgtgttgcatgccactcaggaattggttaccctgggtctggcaaagccgatcctgattggccgcccgaacgttatcgagatgcgcatccagaagctgggcctgcagatcaaggccggcgtggacttcgagatcgtgaacaacgagtcggacccgcgcttcaaggagtactggaccgagtacttccagatcatgaagcgtcgcggcgttacccaggagcaggcgcagcgtgcactgatcagcaacccgaccgtgatcggcgcaattatggtgcaacgtggtgaagcagacgccatgatctgcggcaccgtgggcgactaccacgagcacttctcggtggtgaagaacgtgttcggctaccgtgacggcgttcataccgcaggcgccatgaatgccctgctgctgccgtcgggcaataccttcatcgccgacacctacgtgaatgacgaaccggacgccgaagagctggccgagattaccttgatggccgccgaaaccgtgcgtcgtttcggcattgagccacgtgtggccctgctgtcgcactcaaacttcggcagctcggactgcccatcgtcgagcaaaatgcgccaagccctggaattggtgcgcgaacgtgccccagaactgatgatcgacggcgagatgcatggcgacgccgcccttgtggaagccatccgtaatgaccgtatgccggactcgagcctgaagggttcggccaacatcctggtgatgccgaacatggaagccgcccgcatcagctacaatctgctgcgcgtgtcgtcgtcagaaggcgtgaccgtgggccctgttctgatgggtgttgccaaaccggttcatgtgttgaccccaattgcctcggtgcgccgcattgtgaacatggtggccctggcggtggtggaggcgcagacccaaccgctgtgattccggaattcgccgacacaaaaaaaccggtcctggggaccggtttttttgcgccttgcgcgggttcgataccgcgccgcacagctcttggctgagacgtactagagacc

SEQ ID NO: 81 - DNA, 2010 bp, Artificial sequence
  Synthetic
ggtctcaatcgcgtctcatggcatatcaccagctcaccgtctttcattgccatacggaattcggatccgtgatgtgcagcttggtccgcacttaagggattgcttatacaggggctaagaatatctgaattgacattatgtgggtgggcttatataattgcatcaacgcagcagccaagacgctcaaccacgcaaggagacaaaaggaggtatatcgatggccaagatgcgcgccgtggacgccgcgatgtacgtgttggagaaggagggcattaccactgccttcggcgtgccgggcgccgccatcaacccattctattcggccatgcgtaagcacggcggtatccgtcacatcctggcccgtcatgtggagggtgcctcgcatatggcagaaggctatactcgcgcaaccgccggtaacatcggcgtgtgtttgggcacctcgggtccggccggtaccgacatgattaccgccctgtactcggcctcggcagattcgatcccgatcctgtgcattactggtcaggccccacgtgcccgcttgcacaaggaggactttcaggcggtggacatcgaggccattgccaagccggtgagcaaaatggccgtgaccgtgcgcgaggcagcattggttccgcgcgtgttgcaacaggcattccatttgatgcgtagtggccgcccgggcccggttctggtggatctgccgttcgacgttcaggttgccgaaatcgagttcgacccggacatgtacgaaccgctgccggtgtataagccggcagccagccgcatgcagatcgagaaggccgtggaaatgctgatccaggccgaaagaccggtgatcgtggccggtggcggcgtgattaatgccgacgccgccgcactgttgcagcagttcgccgagttgacctcagtgccagtgattccgaccttgatgggctggggctgcatcccggacgatcatgagctgatggccggcatggtgggacttcagaccgcccaccgctatggtaacgccaccttgttggcctcggacatggtgttcggcatcggcaaccgcttcgccaaccgccacaccggctcggtggagaagtacaccgagggccgcaagatcgtgcacattgacatcgagccgactcagatcggccgcgtgttgtgtccggacctgggtatcgtgtcggacgccaaggccgccctgaccctgctggtggaagttgcccaagaaatgcagaaggccggtcgtctgccgtgccgtaaggaatgggtggccgactgccagcagcgcaagcgtaccttgttgcgcaagacccacttcgacaacgtgccggtgaagccgcagcgcgtgtatgaggaaatgaataaggcgttcggccgcgacgtgtgctacgtgaccaccatcggcctgtcgcagatcgccgccgcccagatgctgcatgtgttcaaggatcgtcactggatcaactgcggtcaggcaggcccgctgggttggaccattccggcagccttgggcgtgtgcgcagccgatccgaagcgtaatgttgtggccatctcgggcgatttcgacttccagttcctgatcgaggagctggccgtgggcgcccagttcaacatcccgtacatccatgtgctggtgaacaacgcctacctgggcctgatccgccagagccagcgcgccttcgacatggactactgcgtgcagctggccttcgagaacatcaactcgtcggaggtgaacggctacggcgtggaccacgtgaaggtggccgaaggcctgggctgcaaggccattcgcgtgtttaagccggaggatattgccccggccttcgaacaggccaaggccctgatggcccaataccgcgtgccggtggtggtggaagtgatcctggagcgcgtgaccaacatctcgatgggctcggagctggacaacgtgatggagttcgaggacatcgccgacaacgccgccgacgccccgaccgagacttgcttcatgcactacgagtgatatgagagacc

SEQ ID NO: 82 - DNA, 922 bp, Artificial sequence
  Synthetic
ggtctcatatgaaaggaggtatatcgatgaaggtgggcttcatcggcctgggcatcatgggcaagccgatgagcaagaacctgctgaaggccggctattcgcttgtggtggccgatcgcaatccggaagccatcgccgatgtgatcgccgcaggcgccgaaaccgcctcgactgccaaggcaatcgccgagcagtgcgacgtgatcatcaccatgctgccgaactcgccgcacgtgaaggaagtggccctgggcgaaaacggcatcatcgaaggcgccaagccgggcaccgtgctgatcgatatgagctcgatcgccccgctggcctcgcgcgaaatctcggaagccctgaaggccaagggcatcgatatgttggatgccccggtgtcgggtggcgaaccgaaggccatcgacggcaccctgtcggtgatggtgggcggcgacaaggccatcttcgacaagtactacgacctgatgaaggccatggcgggctcggtggtgcataccggcgagatcggcgccggcaatgtgaccaagctggccaaccaagtgatcgtggccctgaacatcgccgccatgtcggaggcactgaccctggccaccaaggccggtgtgaatccggatctggtgtaccaagcaatccgcggcggtctggccggctcgactgttctggacgccaaggccccgatggtgatggaccgcaacttcaagccgggctttcgcatcgacctgcacatcaaggacctggccaacgccctggatacctcgcatggcgttggcgcacagctgccgctgaccgccgccgttatggaaatgatgcaggccctgcgtgccgatggtttgggcactgccgaccattcggcactggcctgctactacgagaagctggcgaaggtggaggtgacccgctgatgagagagacc

SEQ ID NO: 83 - DNA, 1241 bp, Artificial sequence
  Synthetic
ggtctcatgagaaaggaggtatatcgatgaagatcgtgatcgccccggactcgttcaaggagtcgctgtcggccgagaagtgctgccaggccatcaaggccggcttctcgaccctgtttccggatgcaaactacatctgtctgccgatcgccgacggcggcgaaggcaccgtggatgccatggtggccgcaaccggcggcaatattgtgaccttggaagtgtgcggtccgatgggcgagaaggtgaacgccttctacggcctgaccggcgatggcaagaccgccgtgatcgaaatggcagccgcctcgggcttgatgctggtggcaccggaaaagcgtaatccgctgctggcctcgtcgttcggcaccggcgaactgatccgccatgcactggacaacgacatccgccacatcatcctgggcatcggtggttcggcaaccgtggatggcggcatgggtatggcccaggccctgggtgtgcgttttttagatgccgacggccaagccttggccgccaatggcggcaacttggcccgtgtggccagcatcgagatggacgaatgcgatccgcgcctggccaactgccatatcgaagtggcctgcgacgttgataacccgttggtgggtgcacgcggcgcggcagccgtgtttggcccacaaaagggcgccaccccggaaatggtggaagagctggaacagggcctgcagaattacgcacgcgttctgcagcagcagaccgaaatcaacgtgtgccagatggccggtggtggcgccgcaggcggcatgggcattgccgccgcagtgttcctgaatgccgacatcaagccgggcatcgaaatcgtgctgaacgccgtgaatctggcccaagcagtgcagggcgccgccttggtgatcactggcgaaggcagaatcgactcgcagaccgccggtggcaaagccccactgggtgtggcatcagtggccaagcagttcaacgtgccggtgatcggcatcgcgggtgtgttgggcgatggcgtggaagtggtgcatcaatacggcatcgacgccgtgttctcgatcctgccacgcctggccccgctggccgaagttctggccagcggcgaaaccaacctgttcaattcggcccgcaatatcgcctgcgccatcaagatcggccagggcatcaagaactgaaaaaccgcgccttcggggcgcggtttttttattgccggcgtcagcgcatcgtgagacgtactagagacc

SEQ ID NO: 84 - DNA, 626 bp, Artificial sequence
  Synthetic
gaagacaagcttttgactataggagttcctccatgccagaccagaccgatcccgccttcgaactcgcgcgctgcgccgagcagtccgccctgcgcatttggctgcgctgggaagaatcgcgccagatgttgctcaggctccgcctcaaggccctggacgaggacataaaaggcacccggcaggcgctcgcccgcgtcaacgatgcgccagactggaacgccttccatggcctgcccgctcagattctggccgcgcaggtcgagcgcaacgccgcacatgcacgcgacattggcggtctttacatagaaatggcctcggccttcatggagcacctgcgcgagtcgaccgaagcctggcaggactaccagcaaagggtcatggtggagggaggcggctacgcgccctttgcggaatcggcgcgcgaaatgttcgacaacgtcggccgcctgatggccgtccctaccccaggcgcgaaggccccggcgcgccccgccggcgcgtcacggagctgatcgcacaggtgccggaaaaagaaaaacgggacgtatcggaggatacgtcccgtttttttatggcctctggccagccggcgcctgcaggatcgcaggcgcgcctactttgtcttc

SEQ ID NO: 85 - DNA, 491 bp, Artificial sequence
  Synthetic
gaagacaatggcatacctcacgcaaaacaagaaaaccggccgggaaagtcccggccggtttttctggtcacccgccctgccctggcctcttgttcactgtgctgcgcctggcaaccttgcgagcgggcaaactttcgggtagccggatcgaacggaaacatgccgtcggacacgggaaatgccggccgacgcctaaggaactcattccagaaaccccgcgcaagctggtggagtgttcggttttgtcccacatcccgctccctggcagcagccgaaccgcaccgcacctcacttcatcgcatcgccgaggcgctgcggcaatatcagtgcgacatcaggaccggcactgtcatcgactcaagcaacgtgcgggtgacaccgccaaggaccagctcgtgcacccggctgtgcccgtaggcccccatgacgatcaggtcagcatttcggtccgcggcgtgtgagagtaacatctggccgacctcaaagtcttc

SEQ ID NO: 86 - DNA, 626 bp, Artificial sequence
  Synthetic
gaagacaagcttagccttgccacgcacaaaggttttctggctcgttcaagatgccttgaaggtgatgtgctgacgaacatcgttggcccacctctagggaaactggggcttgttaccaaagaaattggtgaggtcaatattaatcaagcaattgcgttatttcgaccaaccgaacaactactgccaaagttcctattaatctggcttagtagctcaatctcgcagtcttggctgaggaaccgagccaagcagacgtcgggacaagtgaatctgaccctcgctctatgccaggagcttcctctacctcggatgacgatcaatgagcaacaggcaatcgttgaccgagttgatgccgcgcaggaacaaatctggtgtgaggaggaactgatccgaaagatgcgacttgagaaatctggccttatggatgacctcctcaccggccgcgtccgcgtcaagccgcagctggcggaaaccaaacaagcagggagcgcctgatgggctgggagcttgaatcgcacaggtgccggaaaaagaaaaacgggacgtatcggaggatacgtcccgtttttttatggcctctggccagccggcgcctgcaggatcgcaggcgcgcctactttgtcttc

SEQ ID NO: 87 - DNA, 597 bp, Artificial sequence
  Synthetic
gaagacaatggcatacctcacgcaaaacaagaaaaccggccgggaaagtcccggccggtttttctggtcacccgccctgccctggaatatatgcttccgcaacctcaggtcccctggggtccagtggggaccctcaggctttcgggctctacccgggcgagggggcgtcctgcctcaggcttccgtggctgctgaagagcgagcaaaaggcttgttgtaagcaatgcccctagacgcacactcgtagccgacagagtaaaatgtctgtcgttcttcgagtgcgaactattgagcacatcgctggcgcgatccgtgcgcagctccactatgaccaacaagactagagatcgcgatgttcgggcggccgtattccggaaggtgctcgccgaacatgttgctgaccccaatgtgctggttgttgaagagctcggcctgcagcacggcttgtgccgtgttgacattgcagtagtgaatggatttctgcacggctacgaactgaaaagcgatgctgacacgctggaacgcttaccagcgcaagttgaagcctattcgaaagcaatggatagggcgaccttggtggtgggactcaaagtcttc

SEQ ID NO: 88 - DNA, 1726 bp, Artificial sequence
  Synthetic
gtagatgaagacaatactcctgcaggatactggaattgccagctggggcgccctctggtaaggttgggaagccctgcaaagtaaactggatggctttcttgccgccaaggatctgatggcgcaggggatcaagatctgatcaagagacaggatgaggatcgtttggcgcgccaaaggaggtatatcgatgcccgaatctagactacaacgcttagccaatctcaaaatcggcaccccgcagcagctgcggcgcacctcgatcattggcaccatcggccccaagaccaactcctgcgaagcgatcaccgccctgcgcaaggccggcctcaacattatccgcctcaacttctcccacggcagctacgaattccatcagagcgtgatcgaaaacgcggtcaagtccgagcaacagtttccgggccgcccgctggccatcgccctggacaccaagggcccggaaatccggaccggccgcaccctgaacgaccaggacctgtatattccggtggaccaccagatgatcttcacgaccgacgcgtcgttcgcgaacacctccaatgacaagattatgtacattgactacgcgaacctcacgaaggtcatcgtcccgggccgcttcatttatgtcgacgacggcatcctgagctttaaggtgctgcaaattatcgatgagtcgaacctccgcgtgcaggccgtgaactccggctatatcgcctcgcacaagggtgtcaacctccccaacacggatgtggatctgccgccgctgagcgccaaggacatgaaggacctccagtttggcgtgcgcaacggcatccacatcgtgttcgcgagctttatccgtaccagcgaagatgtcctgtccatccgcaaggccctgggctccgagggccaggacattaagatcatctccaagattgagaaccagcagggcctggataacttcgacgaaatcctggaagtgaccgacggcgtcatgatcgcccgcggtgatctgggcatcgaaatcctcgcccccgaggtcctggccatccagaagaagctgatcgcgaagtgcaacctggccggcaagccggtcatttgcgccacccagatgctggatagcatgacccataatccgcgtcccacccgcgcggaagtgtccgatgtgggcaacgccgtcctggatggcgcggactgcgtcatgctgtcgggtgaaacggccaagggcgattaccccgtgaatgccgtgaatatcatggcggcgacggccctgatcgccgagtcgaccatcgcgcacctggccctgtacgacgacctccgtgacgccacgcccaagccgacctcgacgacggaaacggtggcggccgccgcgaccgcggccatcctggaacaagacggtaaggccatcgtggtgctgagcaccaccggcaataccgcccgtctgctgtcgaagtaccgcccgtcgtgcccgatcatcctggtcacccggcacgcccgcaccgcgcgcatcgcccacctgtatcgcggcgtgtttcccttcctgtacgagcccaagcggctggacgactggggcgaggacgtccaccgccgcctgaagttcggcgtggaaatggcccgctccttcggcatggtggataacggcgacaccgtggtgagcatccaaggcttcaagggcggcgtgggccattcgaacacgctgcgtatcagcacggtcggccaggagttctgaatcgttgtcttctagttg

(19) United States
(12) Patent Application Publication
(10) Pub. No.: US 2018/0002704 A1
(43) Pub. Date: Jan. 4, 2018

(54) SYNTHETIC CARBON FIXATION PATHWAYS

(71) Applicant: INVISTA NORTH AMERICA S.A.R.L., Wilimington, DE (US)

(72) Inventors: Achuthanunni Chokkathukalam, Redcar (GB); Alex Van Eck Conradie, Cleveland (GB); Ramdane Haddouche, Redcar (GB); Satnam Surae, Redcar (GB); Katherine Louise Tibbles, Stockton on Tees (GB)

(21) Appl. No.: 15/637,489

(22) Filed: Jun. 29, 2017

Related U.S. Application Data
(60) Provisional application No. 62/357,361, filed on Jun. 30, 2016.

(51) Int. Cl. C12N 15/52; C12P 7/40; C12N 9/02; C12N 9/90; C12N 9/88; C12N 9/10; C12N 9/04; C12N 9/00

(52) U.S. Cl. CPC C12N 15/52; C12Y 602/01036; C12N 9/0006; C12Y 404/01001; C12Y 403/01019; C12Y 403/01006; C12Y 401/01031; C12Y 208/03; C12Y 203/03005; C12Y 203/01054; C12Y 103/01084; C12Y 102/01043; C12Y 102/0101; C12Y 102/01002; C12Y 101/99007; C12Y 101/01027; C12P 7/40; C12N 9/93; C12N 9/90; C12N 9/88; C12N 9/13; C12N 9/1029; C12N 9/1025; C12N 9/001; C12N 9/0008; C12Y 503/01027

(57) ABSTRACT

The present disclosure relates to methods for more efficiently recycling reduced electron carriers in a hydrogen-oxidizing microorganism with an operable Calvin-Benson cycle; synthetic carbon fixation pathways that recycle reduced electron carriers more efficiently than the Calvin-Benson cycle, such as methods for enzymatically converting carbon dioxide to formate and assimilating the resulting formate into central carbon metabolism; methods for producing biochemical products; and recombinant hosts utilizing one or more synthetic carbon fixation pathways.

This patent application claims the benefit of priority from U.S. Provisional Application Ser. No. 62/357,361, filed Jun. 30, 2016, teachings of which are herein incorporated by reference in their entirety.

### FIELD

The present disclosure relates to methods for more efficiently recycling reduced electron carriers in a hydrogen-oxidizing microorganism with an operable Calvin-Benson cycle, comprising attenuating the Calvin-Benson cycle in the microorganism and fixing carbon more efficiently via alternative carbon fixation pathways, including synthetic carbon fixation pathways described herein.

The disclosure also relates to synthetic, non-naturally occurring carbon fixation pathways that recycle reduced electron carriers more efficiently than the Calvin-Benson cycle, such as methods for enzymatically converting carbon dioxide to formate and assimilating the resulting formate into central carbon metabolism. The disclosure provides methods for enzymatically converting pyruvate or 2-oxobutyrate to formate using a protein having formate C-acetyltransferase activity. The disclosure also relates to methods for enzymatically converting carbon dioxide to formate using a protein having reductive NADP/NADPH-dependent formate dehydrogenase activity. The disclosure further relates to methods for assimilating formate into central carbon metabolism via acetyl-CoA or glycerone phosphate using one or more anaplerotic enzymes such as pyruvate carboxylase, phosphoenolpyruvate carboxylase, the malic enzyme, and isocitrate dehydrogenase.

### BACKGROUND

Fixation of inorganic carbon into biomass in autotrophic organisms such as plants and microorganisms is one of nature's predominant biochemical processes, supplying the carbon building blocks necessary to sustain life. In addition, biological carbon fixation represents a means to generate biofuels or other chemical commodities utilizing renewable solar energy.

The reductive pentose phosphate cycle, also known as the Calvin-Benson or Calvin-Benson-Bassham cycle, is used by a significant majority of autotrophic organisms for carbon dioxide assimilation. Key genes associated with the Calvin-Benson cycle include, but are not limited to, cbbS and cbbL, which encode the small and large subunits of key enzyme ribulose-1,5-bisphosphate carboxylase/oxygenase (RubisCO), respectively.

While the ubiquitous Calvin-Benson cycle is kinetically and thermodynamically favorable, its key carboxylase enzyme RubisCO is known to be catalytically slow and inefficient. Moreover, the oxygenation reaction associated with RubisCO produces toxic phosphoglycolate. To date, attempts to reengineer RubisCO to improve Calvin-Benson cycle efficiency have achieved limited success.

Alternative carbon fixation pathways to the Calvin-Benson cycle may provide another method for improving carbon fixation efficiency. Alternative autotrophic mechanisms in nature have been identified and elucidated. For example, the reductive tricarboxylic acid (rTCA) cycle, the oxygen-sensitive reductive acetyl-CoA (rAcCoA) pathway, the 3-hydroxypropionate cycle, the 3-hydroxypropionate/4-hydroxybutyrate cycle, and the dicarboxylate/4-hydroxybutyrate cycle are alternative natural metabolic pathways known to perform carbon fixation.

Some analyses indicate that these alternative carbon fixation pathways may utilize donated electrons from H₂ more efficiently and require fewer ATP per carbon fixed relative to the Calvin-Benson cycle. As a non-limiting example, the Calvin-Benson pathway requires 5 NADPH electron donors and 7 ATP to synthesize one pyruvate molecule. In contrast, the rTCA cycle requires 2 ferredoxin pairs, 3 NADPH, and only 2 ATP to synthesize one pyruvate molecule, and the glycine synthase pathway (serine hydroxymethytransferase) utilizes 5 NADPH electron donors and 2 ATP to synthesize one pyruvate molecule. Resources needed for the synthesis of pyruvate are based on Table 1 of Bar-Even et al. (Bar-Even, Arren, Elad Noor, and Ron Milo. Journal of Experimental Botany (2011)), which assumes pyrophosphate is hydrolyzed into two phosphates. Accordingly, microorganisms engineered to utilize natural or synthetic alternative carbon fixation pathways may utilize donated electrons from H₂ more efficiently and require fewer ATP per carbon fixed relative to a microorganism with an operable Calvin-Benson cycle. More efficient recycling of donated electrons in the host microorganism may facilitate more efficient production of biofuels or other chemical commodities utilizing renewable solar energy.

Cupriavidus necator H16 has potential for industrial chemical production. It is metabolically flexible, able to grow on CO₂, and can divert carbon flux into a storage pathway as a biopolymer; this ability to redirect carbon flux could be manipulated to produce the chemicals of interest (Brigham et al. (2012). Manipulation of Ralstonia eutropha carbon storage pathways to produce useful bio-based products. In Reprogramming microbial metabolic pathways (pp. 343-366). Springer Netherlands; Müller et al. (2013) Applied and environmental microbiology, 79(14), 4433-4439). However, C. necator uses the Calvin-Benson-Bassham (CBB) cycle for carbon fixation (Pohlmann et al. (2006) Nature biotechnology, 24(10), 1257-1262), which is slow and inefficient due to the limitations of the carbon fixation enzyme, Rubisco (Andersson, I. (2008) Journal of experimental botany, 59(7), 1555-1568; Boyle, N. R., & Morgan, J. A. (2011) Metabolic engineering, 13(2), 150-158)). There are multiple carbon fixation pathways found in nature (Bar-Even, A., Noor, E., & Milo, R. (2011) Journal of experimental botany, err417; Hügler, M., & Sievert, S. M. (2011) Marine Science, 3), and even more synthetic pathways have been proposed (Bar-Even et al. (2010) Proceedings of the National Academy of Sciences, 107(19), 8889-8894; Siegel et al. (2015) Proceedings of the National Academy of Sciences, 112(12), 3704-3709), which are calculated to be more efficient, and use different carbon fixation enzymes. C. necator could benefit from a more efficient fixation pathway, to make production of useful chemicals from CO₂ more financially viable.

There is a need for recombinant hosts which fix carbon at a greater rate and with better energy efficiency than strains using the endogenous CBB cycle.

### SUMMARY OF THE DISCLOSURE

The present disclosure relates to methods for more efficiently recycling reduced electron carriers in a hydrogen-oxidizing microorganism with an operable Calvin-Benson cycle, comprising attenuating the Calvin-Benson cycle in the microorganism and fixing carbon more efficiently via an alternative carbon fixation pathway.

In general, alternative carbon fixation pathways are naturally found in organisms that live in unusual or extreme environments such as members of the thermophilic bacterial phylum Aquificae, methanogens, and green sulfur bacteria. In contrast, according to this disclosure, synthetic carbon fixation pathways incorporating features of alternative natural metabolic pathways that perform carbon fixation may be constructed within canonical laboratory and industrial hydrogen-oxidizing microorganisms such as Cupriavidus necator or Rhodobacter capsulatus that have an operable Calvin-Benson cycle to more efficiently recycle donated electrons from H₂.

Some embodiments of the disclosure relate to attenuating the Calvin-Benson cycle in a hydrogen-oxidizing microorganism with an operable Calvin-Benson cycle by attenuating the cbbL and/or cbbS gene in the microorganism. In some further embodiments, native formate metabolism in the microorganism is inhibited by attenuating one or more of the native fdsG, fdsB, fdsA, fdsC, fdsD genes.

These methods may allow for more efficient production of biofuels or other chemical commodities utilizing renewable solar energy.

The present disclosure also relates to synthetic carbon fixation pathways that recycle reduced electron carriers more efficiently than the Calvin-Benson cycle, such as methods for enzymatically converting carbon dioxide to an intermediate such as formate, oxalate, succinate, malate, isocitrate, or acetate and assimilating the resulting intermediate into central carbon metabolism. Processes for converting carbon dioxide to an intermediate such as formate, oxalate, succinate, isocitrate, or acetate may include natural biochemical pathways or combinations of natural biochemical pathways. In addition, processes for assimilating the resulting intermediate into central carbon metabolism may include natural biochemical pathways or combinations of natural biochemical pathways.

Some embodiments of the disclosure relate to said methods, wherein said intermediate is formate. In some embodiments, pyruvate or 2-oxobutyrate is enzymatically converted to formate using a protein having formate C-acetyltransferase activity. In some embodiments, β-alanine, lactate, 3-hydroxypropionate, or homoserine serves as a central precursor leading to formate.

Some additional embodiments of the disclosure relate to said methods, wherein carbon dioxide is enzymatically converted to formate using a protein having reductive NADP/NADPH-dependent formate dehydrogenase activity. In some embodiments, the partial pressures of CO₂ and H₂ are increased to promote the enzymatic conversion of CO₂ to formate using a protein having reductive NADP/NADPH-dependent formate dehydrogenase activity.

Some additional embodiments of the disclosure relate to said methods, wherein formate is assimilated into central carbon metabolism via acetyl-CoA or glycerone phosphate using one or more anaplerotic enzymes such as a pyruvate carboxylase, a PEP carboxylase, a malic enzyme, and an isocitrate dehydrogenase.

Some embodiments of the disclosure relate to recombinant host comprising at least one exogenous nucleic acid encoding a methylisocitrate lyase and an anaplerotic enzyme.

In one nonlimiting embodiment, the disclosure relates to a method of producing formate in a recombinant host, said method comprising enzymatically converting 2-methyl-isocitrate to pyruvate in said recombinant host using a protein having methylisocitrate lyase activity; and enzymatically converting pyruvate to formate in said recombinant host using a protein having formate C-acetyltransferase activity.

In one nonlimiting embodiment, this method further comprises enzymatically converting β-alanine to β-alanyl-CoA using a protein having CoA-transferase activity classified under EC 2.8.3.-; and enzymatically converting β-alanyl-CoA to acrylol-CoA using a protein having acrylyl-CoA reductase activity. In one nonlimiting embodiment, the protein having acrylyl-CoA reductase activity is classified under EC 1.3.1.84. In these nonlimiting embodiments, the recombinant host may overexpress one or more genes encoding at least one protein having the activity of at least one enzyme selected from: a 2-methylisocitrate dehydratase, a methylisocitrate lyase, a succinate dehydrogenase (quinone), a fumarate reductase (quinol), a fumarate hydratase, a malate dehydrogenase, a 2-methylisocitrate dehydratase, a 2-methylcitrate synthase, an acrylyl-CoA reductase (NADPH), a β-alanyl-CoA:ammonia lyase, a glutamate dehydrogenase, a CoA-transferase, an alanine transaminase, a β-alanine pyruvate aminotransferase, a formate C-acetyltransferase, a malonyl-CoA reductase (malonate semialdehyde-forming), an alanine-oxo-acid transaminase, and an acetyl-CoA carboxylase.

In another nonlimiting embodiment, the method further comprises enzymatically converting 3-hydroxy-propanoate to 3-hydroxy-propanoyl-CoA using a protein having 3-hydroxypropionyl-CoA synthase activity and a protein having CoA-transferase activity; and enzymatically converting 3-hydroxy-propanoyl-CoA to acrylol-CoA using a protein having β-alanyl-CoA ammonia-lyase activity. In this nonlimiting embodiment, the protein having 3-hydroxypropionyl-CoA synthase activity may be classified under EC 6.2.1.36. Further, in these nonlimiting embodiments, the protein having β-alanyl-CoA ammonia-lyase activity may be classified under EC 4.3.1.6. In any of these nonlimiting embodiments, the recombinant host may overexpress one or more genes encoding at least one protein having the activity of at least one enzyme selected from: a 2-methylisocitrate dehydratase, a methylisocitrate lyase, a succinate dehydrogenase (quinone), a fumarate reductase (quinol), a fumarate hydratase, a malate dehydrogenase, a 2-methylisocitrate dehydratase, a 2-methylcitrate synthase, an acrylyl-CoA reductase (NADPH), a CoA-transferase, an alanine transaminase, a formate C-acetyltransferase, a malonyl-CoA reductase (malonate semialdehyde-forming), a 3-hydroxypropionate dehydrogenase, a 3-hydroxypropionyl-CoA synthase, an enoyl-CoA hydratase, and an acetyl-CoA carboxylase.

In any of the above described nonlimiting embodiments, the protein having CoA-transferase activity may be classified under EC 2.8.3.-.

Further, in any of the above described nonlimiting embodiments, the protein having methylisocitrate lyase activity may be classified under EC 2.3.3.5.

In another nonlimiting embodiment, the disclosure relates to a method of producing formate in a recombinant host, said method comprising enzymatically converting lactate to pyruvate in said recombinant host using a protein having L-lactate dehydrogenase activity and a protein having lactate-malate transhydrogenase activity; and enzymatically converting pyruvate to formate in said recombinant host using a protein having formate C-acetyltransferase activity. In this nonlimiting embodiment, the protein having L-lactate dehydrogenase activity may be classified under EC 1.1.1.27. Further, in these nonlimiting embodiments, the protein having lactate-malate transhydrogenase activity may be classified under EC 1.1.99.7. In any of these nonlimiting embodiments, the recombinant host may overexpress one or more genes encoding at least one protein having the activity of at least one enzyme selected from: an enoyl-CoA hydratase, a lactoyl-CoA dehydratase, a propionate CoA-transferase, a 3-hydroxypropionate dehydrogenase, a malonyl-CoA reductase (malonate semialdehyde-forming), an acetyl-CoA carboxylase, a formate C-acetyltransferase, a lactate-malate transhydrogenase, and a L-lactate dehydrogenase.

In another nonlimiting embodiment, the disclosure relates to a method of producing formate in a recombinant host, said method comprising enzymatically converting L-homoserine to 2-oxobutyrate in said recombinant host using a protein having threonine ammonia-lyase activity and a protein having cystathionine γ-lyase activity; and enzymatically converting 2-oxobutyrate to formate in said recombinant host using a protein having formate C-acetyltransferase activity. In this nonlimiting embodiment, the protein having threonine ammonia-lyase activity may be classified under EC 4.3.1.19. Further, in these nonlimiting embodiments, the protein having cystathionine γ-lyase activity may be classified under EC 4.4.1.1. In these nonlimiting embodiments, the recombinant host may overexpress one or more genes encoding at least one protein having the activity of at least one enzyme depicted in FIG. 6.

In any of the above-described embodiments, the protein having formate C-acetyltransferase activity may be classified under EC 2.3.1.54.

In any of the above-described embodiments, the recombinant host may overexpress one or more genes encoding at least one or more proteins having the activity of at least one enzyme selected from: a threonine ammonia-lyase, a cystathionine γ-lyase, a formate C-acetyltransferase, a 2-methylcitrate synthase, a 2-methylcitrate dehydratase, a 2-methylisocitrate dehydratase, a methylisocitrate lyase, a succinate dehydrogenase (quinone), a fumarate reductase (quinol), a fumarate hydratase, a malate dehydrogenase, a malate dehydrogenase (oxaloacetate-decarboxylating), an acetyl-CoA carboxylase, an aspartate kinase, an aspartate-semialdehyde dehydrogenase, a malate dehydrogenase, and a glutamate dehydrogenase.

In another nonlimiting embodiment, the disclosure relates to a method of producing formate in a recombinant host, said method comprising enzymatically converting CO₂ to formate in said recombinant host using a protein having reductive NADP/NAPDH-dependent formate dehydrogenase activity. In this embodiment, the protein having reductive NADP/NAPDH-dependent formate dehydrogenase activity may be classified under EC 1.2.1.43 or EC 1.2.1.2.

In another nonlimiting embodiment, the disclosure relates to a method of producing β-D-fructofuranose 6 phosphate in a recombinant host, said method comprising enzymatically converting formyl-CoA and NADH to formaldehyde and NAD⁺ in said recombinant host using a protein having acetaldehyde dehydrogenase activity; enzymatically converting D-ribulose 5-phosphate and formaldehyde to hexulose 6-phosphate in said recombinant host using a protein having phosphoenolpyruvate carboxylase activity; and enzymatically converting hexulose 6-phosphate to β-D-fructofuranose 6 phosphate in said recombinant host using a protein having 6-phospho-3-hexuloisomerase activity. In this nonlimiting embodiment, the method may further comprise enzymatically converting formate, adenosine triphosphate, and succinyl-CoA to formyl-CoA, adenosine diphosphate, Pi, and succinate in said recombinant host using a protein having formyl-CoA transferase activity and a protein having acetate-CoA ligase activity. In these nonlimiting embodiments, the protein having acetate-CoA ligase may be classified under EC 6.2.1.1, the protein having formyl-CoA transferase activity may be classified under EC 2.8.3.16, the protein having acetaldehyde dehydrogenase activity may be classified under EC 1.2.1.10, the protein having phosphoenolpyruvate carboxylase activity may be classified under EC 4.1.1.31, and the protein having 6-phospho-3-hexuloisomerase activity may be classified under EC 5.3.1.27.

In any of the above nonlimiting embodiments, the recombinant host may comprise an attenuation of one or more of the following genes: cbbL, cbbS, fdsG, fdsB, fdsA, fdsC, and fdsD.

In any of the above nonlimiting embodiments, the recombinant host may be a hydrogen-oxidizing microorganism. In these nonlimiting embodiments, the hydrogen-oxidizing microorganism may have an operable Calvin-Benson cycle. In these embodiments, the Calvin-Benson cycle in the recombinant host may be at least partially attenuated by increasing the partial pressure of both CO₂ and H₂ in the surrounding environment.

In another nonlimiting embodiment, the disclosure relates to a method of producing a biochemical product in a recombinant host, wherein one or more of the methods for producing formate as disclosed herein are intermediate steps in the process.

In any of the above nonlimiting embodiments, the production of formate may be increased by increasing the partial pressure of both CO₂ and H₂ in fermentation conditions.

In any of the above nonlimiting embodiments, the method may result in more efficient utilization of H₂ as an electron donor relative to the Calvin-Benson cycle.

In any of the above nonlimiting embodiments, the method may result in more efficient carbon fixation relative to the Calvin-Benson cycle.

In another nonlimiting embodiment, the disclosure relates to a recombinant host comprising at least one exogenous nucleic acid encoding a methylisocitrate lyase and an anaplerotic enzyme. In one nonlimiting embodiment, the anaplerotic enzyme of this recombinant host is a pyruvate carboxylase, a phosphoenolpyruvate carboxylase, a malic enzyme, or an isocitrate dehydrogenase. In one nonlimiting embodiment, this recombinant host further comprises one or more of the following exogenous enzymes: 2-methylcitrate dehydratase, a methylisocitrate lyase, a succinate dehydrogenase (quinone), a fumarate reductase (quinol), a fumarate hydratase, a malate dehydrogenase, a 2-methylisocitrate dehydratase, a 2-methylcitrate synthase, an acrylyl-CoA reductase (NADPH), a β-alanyl-CoA:ammonia lyase, a glutamate dehydrogenase, a CoA-transferase, an alanine transaminase, a β-alanine pyruvate aminotransferase, a formate C-acetyltransferase, a malonyl-CoA reductase (malonate semialdehyde-forming), an acetyl-CoA carboxylase, an enoyl-CoA hydratase, a 3-hydroxypropionyl-CoA synthase, a lactoyl-CoA dehydratase, a propionate CoA-transferase, a L-lactate dehydrogenase, a lactate-malate transhydrogenase, a 3-hydroxypropionate dehydrogenase, a threonine ammonia-lyase, a cystathionine γ-lyase, a homoserine dehydrogenase, an aspartate-semialdehyde dehydrogenase, a malate dehydrogenase (oxaloacetate-decarboxylating), an aspartate kinase, a formate-tetrahydrofolate ligase, a methenyltetrahydrofolate cyclohydrolase, a glycine hydroxymethyltransferase, a serine-glyoxylate transaminase, a hydroxypyruvate reductase, a glycerate dehydrogenase, a glycerate 2-kinase, a phosphopyruvate hydratase, a phosphoenolpyruvate carboxylase, a malate-CoA ligase, a malyl-CoA lyase, a pyruvate kinase, a pyruvate carboxylase, a succinyl-CoA-L-malate CoA-transferase, a pyruvate synthase, a tartronate-semialdehyde synthase, an oxidoreductase with NAD(+) or NADP(+) as acceptor, a glycerate 3-kinase, a phosphoglycerate mutase (2,3-diphosphoglycerate-independent), a phosphoglycerate mutase (2,3-diphosphoglycerate-dependent), a pyruvate, phosphate dikinase, a pyruvate, water dikinase, a hydroxypyruvate isomerase, a 2-dehydro-3-deoxyglucarate aldolase, a 5-dehydro-4-deoxyglucarate dehydratase, a 2, 5-dioxovalerate dehydrogenase, an acetate-CoA ligase, a formyl-CoA transferase, an aldehyde-alcohol dehydrogenase, a 6-phospho-3-hexuloisomerase, a 6-phosphofructokinase, a fructose-bisphosphate aldolase, a transketolase, a transaldolase, a ribulose-phosphate 3-epimerase, a ribose-5-phosphate isomerase, a fructose-6-phosphate phosphoketolase, and a phosphate acetyltransferase. In any of these nonlimiting embodiments, the recombinant host may comprise an attenuation of one or more of the following genes: cbbL, cbbS, fdsG, fdsB, fdsA, fdsC, and fdsD. Further, in any of these nonlimiting embodiment, the recombinant host may overexpress one or more genes encoding at least one protein having the activity of at least one enzyme depicted in FIGS. 3 to 12. In any of these nonlimiting embodiments, the recombinant host may overexpress one or more genes encoding at least one protein having the activity of at least one enzyme selected from: a 2-methylcitrate dehydratase, a methylisocitrate lyase, a succinate dehydrogenase (quinone), a fumarate reductase (quinol), a fumarate hydratase, a malate dehydrogenase, a 2-methylisocitrate dehydratase, a 2-methylcitrate synthase, an acrylyl-CoA reductase (NADPH), a β-alanyl-CoA:ammonia lyase, a glutamate dehydrogenase, a CoA-transferase, an alanine transaminase, a β-alanine pyruvate aminotransferase, a formate C-acetyltransferase, a malonyl-CoA reductase (malonate semialdehyde-forming), an acetyl-CoA carboxylase, an enoyl-CoA hydratase, a 3-hydroxypropionyl-CoA synthase, a lactoyl-CoA dehydratase, a propionate CoA-transferase, a L-lactate dehydrogenase, a lactate-malate transhydrogenase, a 3-hydroxypropionate dehydrogenase, a threonine ammonia-lyase, a cystathionine γ-lyase, a homoserine dehydrogenase, an aspartate-semialdehyde dehydrogenase, a malate dehydrogenase (oxaloacetate-decarboxylating), an aspartate kinase, a formate-tetrahydrofolate ligase, a methenyltetrahydrofolate cyclohydrolase, a glycine hydroxymethyltransferase, a serine-glyoxylate transaminase, a hydroxypyruvate reductase, a glycerate dehydrogenase, a glycerate 2-kinase, a phosphopyruvate hydratase, a phosphoenolpyruvate carboxylase, a malate-CoA ligase, a malyl-CoA lyase, a pyruvate kinase, a pyruvate carboxylase, a succinyl-CoA-L-malate CoA-transferase, a pyruvate synthase, a tartronate-semialdehyde synthase, an oxidoreductase with NAD(+) or NADP(+) as acceptor, a glycerate 3-kinase, a phosphoglycerate mutase (2, 3-diphosphoglycerate-independent), a phosphoglycerate mutase (2,3-diphosphoglycerate-dependent), a pyruvate, phosphate dikinase, a pyruvate, water dikinase, a hydroxypyruvate isomerase, a 2-dehydro-3-deoxyglucarate aldolase, a 5-dehydro-4-deoxyglucarate dehydratase, a 2,5-dioxovalerate dehydrogenase, an acetate-CoA ligase, a formyl-CoA transferase, an aldehyde-alcohol dehydrogenase, a 6-phospho-3-hexuloisomerase, a 6-phosphofructokinase, a fructose-bisphosphate aldolase, a transketolase, a transaldolase, a ribulose-phosphate 3-epimerase, a ribose-5-phosphate isomerase, a fructose-6-phosphate phosphoketolase, and a phosphate acetyltransferase. In any of these nonlimiting embodiments, the recombinant host may be a hydrogen-oxidizing microorganism. In these embodiments, the hydrogen-oxidizing microorganism may have an operable Calvin-Benson cycle.

In another nonlimiting embodiment, the disclosure relates to a method for more efficiently recycling reduced electron carriers in a recombinant host comprising providing at least one microorganism capable of hydrogen oxidation, wherein the microorganism has an operable Calvin-Benson cycle; attenuating the Calvin Benson cycle in said microorganism; and utilizing the donated electrons more efficiently than the microorganism having a Calvin Benson cycle. In this nonlimiting embodiment, the recombinant host may more efficiently fix carbon than an otherwise identical microorganism utilizing the Calvin-Benson cycle for carbon fixation.

In yet another nonlimiting embodiment, the disclosure relates to a method for more efficiently fixing carbon in a recombinant host comprising providing at least one microorganism capable of hydrogen oxidation, wherein the microorganism has an operable Calvin-Benson cycle; attenuating the Calvin Benson cycle in said microorganism; and fixing carbon more efficiently than the attenuated reductive pentose phosphate pathway. In this nonlimiting embodiment, the method may further comprise any of the methods for formate production described herein.

In any of the above described nonlimiting embodiments, the hydrogen-oxidizing microorganism with an operable Calvin-Benson cycle may be selected from Cupriavidus necator, Hydrogenovibrio marinus, Rhodobacter capsulatus, Rhodobacter sphaeroides, Rhodospirillum rubrum, Thiobacillus ferrooxidans, and Xanthobacter flavus.

### BRIEF DESCRIPTION OF THE DRAWINGS

The descriptions below are provided by way of explanation. The disclosures of the figures are not limited to the descriptions below. Black arrows represent where a cycle turns only once, whereas dashed arrows represent where a portion of a cycle or pathway is utilized more than once.

FIG. 1 is a schematic of two exemplary biochemical pathways, summarizing the energetics association with H2 as an electron donor for the synthesis of reducing equivalents.

FIG. 2 is a schematic of exemplary biochemical pathways leading to formate synthesis via (1) reductive formate dehydrogenase, (2) acetyl-CoA carboxylase, or (3) pyruvate dehydrogenase.

FIG. 3 is a schematic of exemplary biochemical pathways leading to formate synthesis via a modified acetyl-CoA carboxylase, β-alanine, and methylcitrate cycle.

FIG. 4 is a schematic of exemplary biochemical pathways leading to formate synthesis via a modified acetyl-CoA carboxylase, 3-hydroxypropionate and methylcitrate cycle.

FIG. 5 is a schematic of exemplary biochemical pathways leading to formate synthesis via acetyl-CoA carboxylase and lactate.

FIG. 6 is a schematic of exemplary biochemical pathways leading to formate synthesis via homoserine and the methylcitrate cycle.

FIG. 7 is a schematic of exemplary biochemical pathways leading to acetyl-CoA synthesis via formate and a modified serine cycle.

FIG. 8 is a schematic of exemplary biochemical pathways leading to acetyl-CoA synthesis using formate and a combination of the reductive TCA cycle and glyoxylate degradation.

FIG. 9 is a schematic of exemplary biochemical pathways leading to acetyl-CoA synthesis using formate and a combination of the reductive TCA cycle and serine cycle.

FIG. 10 is a schematic of exemplary biochemical pathways leading to acetyl-CoA synthesis using formate and a combination of the reductive TCA cycle and serine cycle.

FIG. 11 is a schematic of exemplary biochemical pathways leading to glycerone phosphate synthesis by assimilating formate via the RUMP cycle.

FIG. 12 is a schematic of exemplary biochemical pathways leading to acetyl-CoA synthesis by assimilating formate via a modified RUMP cycle.

FIG. 13 is a schematic of synthetic carbon fixation pathway similar to that of FIG. 7 referred to herein as P1. Enzyme activities that are definitely not found in the C. necator genome and require insertion are italicized; enzyme activities that may not exist or may need upregulating are underlined.

FIG. 14 is a schematic of synthetic carbon fixation pathway similar to that of FIG. 9 referred to herein as P2. The alternative two genes required for P2 pathway are emboldened, whereas genes inserted for the various P1 strategies are italicized or underlined.

FIG. 15 is a schematic of synthetic carbon fixation pathway similar to that of FIG. 12 referred to herein as P5. Enzyme activities that are definitely not found in the C. necator genome and require insertion are italicized; enzyme activities that may not exist or may need upregulating are underlined.

FIG. 16 is a schematic of synthetic carbon fixation pathway referred to herein as P10. Pfl is shown in bold. Enzyme activities that are definitely not found in the C. necator genome and require insertion are italicized; enzyme activities that may not exist or may need upregulating are underlined.

FIGS. 17A and 17B show growth of wild type Cupriavidus necator in INV-2 media containing potassium or sodium formate equivalent to 2.7 g/L formic acid. FIG. 17B shows the same data as FIG. 17A for formate media at a closer scale.

FIG. 18 shows formate consumption of wild type Cupriavidus necator in INV-2 media containing potassium or sodium formate equivalent to 2.7 g/L formic acid.

FIGS. 19A and 19B show growth of deletion strains of Cupriavidus necator in INV-2 media containing sodium formate equivalent to 1.35 g/L formic acid. FIG. 19B shows same data as FIG. 19A at a closer scale.

FIG. 20 shows formate consumption of deletion strains of Cupriavidus necator in INV-2 media containing sodium formate equivalent to 1.35 g/L formic acid.

### DETAILED DESCRIPTION

Provided herein are enzymes, synthetic pathways (i.e., pathways that do not naturally take place in nature), cultivation strategies, feedstocks, host microorganisms, and attenuations of the host microorganism's biochemical network for more efficient recycling of reduced electron carriers in the host relative to the native Calvin-Benson cycle. More efficient recycling of reduced electron carriers in the host microorganism may facilitate more efficient production of biofuels or other chemical commodities utilizing renewable solar energy.

### Definitions and Abbreviations

As used above, and throughout the description, the following terms, unless otherwise indicated, shall be understood to have the following meanings. Unless otherwise defined, all technical and scientific terms used herein have the same meaning as commonly understood by one of ordinary skill in the art to which the disclosure pertains.

Unless clearly indicated otherwise, use of the terms “a,” “an,” “at least one,” and the like refers to one or more.

As used herein, the term “naturally” refers to an unengineered state, such as, for example, the state of a microorganism in its native environment.

As used herein in reference to a nucleic acid or protein in a host, the term “exogenous” refers to a nucleic acid or protein not naturally found in the host. Exogenous nucleic acids may contain nucleic acid subsequences or fragments of nucleic acid sequences that are found in nature provided the nucleic acid as a whole does not exist in the host. For example, a nucleic acid molecule containing a genomic DNA sequence within an expression vector is non-naturally-occurring nucleic acid and thus is exogenous to a host cell once introduced into the host. A naturally-occurring protein or nucleic acid can be exogenous to a particular host microorganism.

As used herein in reference to a nucleic acid or protein in a host, the term “endogenous” refers to a nucleic acid or protein naturally found in the host.

As used herein, the term “anaplerotic enzyme” refers to an enzyme used to replenish a depleted metabolic cycle or pathway intermediate.

As used herein, the term “central precursor” refers to any metabolite in a metabolic pathway shown herein leading to formate synthesis or assimilation of formate into central carbon metabolism via acetyl-CoA or glycerone phosphate.

As used herein, the term “central metabolite” refers to a metabolite that is produced in all microorganisms to support growth.

As used herein, the term “central carbon metabolism” refers to the conversion of sugars into central metabolites produced in all microorganisms to support growth. Non-limiting examples of pathways involved in central carbon metabolism in some microorganisms include the Embden-Meyerhof-Parnas (EMP) pathway of glycolysis, the Calvin-Benson cycle, and the citric acid cycle.

As used herein, the phrases “engineered pathway” and the like refer to biochemical pathways that do not occur in nature and/or biochemical pathways in a host microorganism that does not naturally express all of the enzymes catalyzing the reactions within the pathway but has been engineered such that all of the enzymes within the pathway are expressed in the host.

As used herein, the term “engineered” in reference to a host microorganism refers to manipulation of the organism's genome and/or expression of a recombinant DNA or RNA construct. Non-limiting examples of manipulation to the organism's genome may include removal or silencing of a gene and introduction of an exogenous nucleic acid.

As used herein, the term “attenuation” refers to the downregulation of gene and/or protein expression relative to the gene and/or protein's expression level in a naturally occurring microorganism and downregulation of enzymatic activity. Non-limiting examples of attenuation include reduction in mRNA transcription of a gene (e.g., due to the presence of a repressor), gene removal, and use of enzyme inhibitors.

As used herein, the phrase “microorganism with an operable Calvin-Benson cycle” refers to a microorganism that naturally utilizes the Calvin-Benson cycle, alone or in combination with alternative carbon fixation pathways, to fix inorganic carbon. A microorganism with an operable Calvin-Benson cycle naturally expresses all of the enzymes catalyzing the reactions within the Calvin-Benson cycle.

As used herein, the phrase “efficiently recycling reduced electron carriers” refers to biochemical pathways that utilize fewer reduced electron carriers per fixed carbon than the Calvin-Benson cycle.

As used herein, the phrase “efficient carbon fixation” refers to biochemical pathways that utilize fewer total reduced electron carriers and ATP molecules per fixed carbon than the Calvin-Benson cycle.

### Recombinant Hosts

In one embodiment, recombinant host microorganisms described herein include hydrogen oxidizing microorganisms. Hydrogen-oxidizing microorganisms are physiologically defined on the basis of their ability to utilize H₂ as an electron donor and include bacteria from different taxonomic units. Hydrogen-oxidizing microorganisms include facultative autotrophs, as well as microorganisms that may grow under completely heterotrophic or mixotrophic conditions. For example, hydrogen-oxidizing microorganisms include fermentative organisms, photosynthetic prokaryotes, aerobes, anaerobes, autotrophs, and heterotrophs.

Non-limiting examples of hydrogen oxidizing microorganisms include Alcaligenes eutrophus, Alcaligenes latus, Alcaligenes paradoxus, Alcaligenes ruhlandii, Alcaligenes lactus, Alcaligenes paradoxus, Aquaspirillum autotrophicum, Bacillus schlegelii, Cupriavidus necator, Derxia gummosa, Flavobacterium autothermophilum, Helicobacter pylori, Hydrogenobacter thermophilus, Hydrogenovibrio marinus, Hydrogenomonas facilis, Hydrogenomonas eutropha, Microcyclus aquaticus, Microcyclus ebruneus, Parcoccus denitrificans, Pseudomonas carboxydovorans, Pseudomonas facilis, Pseudomonas flava, Pseudomonas pseudoflava, Pseudomonas hydrogenovora, Pseudomonas hydrogenothermophila, Pseudomonas palleronii, Pseudomonas saccharophila, Pseudomonas thermophila, Renobacter vacuolatum, Rhizobium japonicum, Rhodobacter capsulatus, Rhodospirillum rubrum, Seliberia carboxyhydrogena, Flavobacterium autothermophilum, Paracoccus denitrificans, Xanthobacter autotrophicus, Xanthobacter flavus, Mycobacterium gordonae, Nocardia autotrophica, Nocardia opaca, and Wautersia eutropha.

Some names recited herein reflect alternative names given to one microorganism (e.g., Hydrogenomonas eutropha and Cupriavidus necator).

Tables 1 and 2 of Vignais et al. (Vignais, Paulette M., and Bernard Billoud. “Occurrence, classification, and biological function of hydrogenases: an overview.” Chemical Reviews, 107.10 (2007): 4206-4272), which are hereby incorporated by reference to the extent they disclose hydrogen-oxidizing organisms known to those skilled in the field, disclose the taxonomic classification of organisms in which hydrogen metabolism has been studied or hydrogenase genes have been identified.

In addition, recombinant host microorganisms described herein include microorganisms with an operable Calvin-Benson cycle. Non-limiting examples of microorganisms with an operable Calvin-Benson cycle include some cyanobacteria (e.g., Synechococcus, Anacytis, and Anabaena), some purple non-sulfur bacteria (e.g., Rhodobacter, Rhodospirillum, and Rhodopseudomonas), some purple sulfur bacteria (e.g., Chromatium), some hydrogen-oxidizing bacteria (e.g., Ralstonia, including renamed Cupriavidus necator, and Hydrogenovibrio), and some other chemoautotrophs (e.g., Thiobacillus). It will be understood by one of ordinary skill in the art that some cyanobacteria, purple non-sulfur bacteria, purple sulfur bacteria, and other chemoautotrophs are capable of hydrogen oxidization, in addition to the hydrogen-oxidizing bacteria belonging to genera such as Ralstonia and Hydrogenovibrio.

In some embodiments, recombinant host microorganisms include hydrogen-oxidizing microorganisms with an operable Calvin-Benson cycle. Non-limiting examples of hydrogen-oxidizing microorganisms with an operable Calvin-Benson cycle include Cupriavidus necator, Hydrogenovibrio marinus, Rhodobacter capsulatus, Rhodobacter sphaeroides, Rhodospirillum rubrum, Thiobacillus ferrooxidans, and Xanthobacter flavus.

### Attenuation of the Calvin-Benson Cycle

In some embodiments, a recombinant host comprises an attenuation of one or more genes associated with the Calvin-Benson cycle. In some embodiments, the one or more genes are selected from the cbb genes.

Attenuation of the Calvin-Benson cycle has been demonstrated in several organisms, including Cupriavidus necator H16 and Rhodobacter capsulatus. For example, Paoli et al. (Paoli, George C., Padungsri Vichivanives, and F. Robert Tabita. “Physiological control and regulation of the Rhodobacter capsulatus cbb operons.” Journal of Bacteriology 180.16 (1998): 4258-4269) have described the production of a RubisCO (cbbLS cbbM)-deficient strain of Rhodobacter capsulatus. Paoli et al. demonstrated that that R. capsulatus strains with disruptions in both the cbbL and cbbM genes could not grow autotrophically. Moreover, disruption in the cbbP in R. capsulatus resulted in strains that did not synthesize form II RubisCO.

In addition, Satagopan and Tabita have described the production of a RubisCO-deletion strain of Ralstonia eutropha H16 (also known as Cupriavidus necator H16). See Satagopan, Sriram, and F. Robert Tabita. “RubisCO selection using the vigorously aerobic and metabolically versatile bacterium Ralstonia eutropha.” The FEBS Journal (2016)). This RubisCO-deletion strain, which was constructed by precisely deleting the cbbL and cbbS genes from both operons in the wild-type strain H16, is unable to support CO2-dependent aerobic growth.

In some embodiments, a recombinant host comprises an attenuation of one or more genes associated with native formate metabolism. In some embodiments, the one or more genes are selected from the fds operon.

Attenuation of the native formate metabolism has been demonstrated in, for example, Oh, Jeong-I I, and Botho Bowien. “Structural analysis of the fds operon encoding the NAD+-linked formate dehydrogenase of Ralstonia eutropha.” Journal of Biological Chemistry 273.41 (1998): 26349-26360.

In some embodiments, a recombinant host comprises an attenuation of one or more of the following genes associated with the Calvin-Benson cycle: cbbA, cbbE, cbbG, cbbL, cbbM, cbbP, cbbR, cbbS, and cbbX.

In some embodiments, a recombinant host comprises an attenuation of the cbbL gene (e.g., GenBank Gene ID 10921831 in Cupriavidus necator N-1 or GenBank Gene ID 9003408 in Rhodobacter capsulatus SB 1003). In some embodiments, a recombinant host comprises an attenuation of the cbbS gene (e.g., GenBank Gene ID 10921830 in C. necator N-1 or GenBank Gene ID 9003407 in R. capsulatus SB 1003). In some embodiments, a recombinant host comprises an attenuation of each of the cbbL and cbbS genes.

In some embodiments, a recombinant host comprises an attenuation of the cbbR gene (e.g., GenBank Gene ID 10921832 in C. necator N-1). In some embodiments, a recombinant host comprises an attenuation of the cbbR_{I} gene (e.g., GenBank Gene ID 9003409 in R. capsulatus SB 1003). In some embodiments, a recombinant host comprises an attenuation of the cbbR_{II} gene (e.g., GenBank Gene ID 9004658 in R. capsulatus SB 1003). In some embodiments, a recombinant host comprises an attenuation of the cbbR_{I} gene and the cbbR_{II} gene.

In some embodiments, a recombinant host comprises an attenuation of the cbbR gene and the cbbL gene. In some embodiments, a recombinant host comprises an attenuation of the cbbR gene and the cbbS gene. In some embodiments, a recombinant host comprises an attenuation of the cbbR gene, the cbbL gene, and the cbbS gene.

In some embodiments, a recombinant host comprises an attenuation of the cbbM gene (e.g., GenBank Gene ID 9004652 in R. capsulatus SB 1003). In some embodiments, a recombinant host comprises an attenuation of the cbbM gene and the cbbL gene. In some embodiments, a recombinant host comprises an attenuation of the cbbM gene and the cbbS gene. In some embodiments, a recombinant host comprises an attenuation of the cbbM gene, the cbbL gene, and the cbbS gene.

In some embodiments, a recombinant host comprises an attenuation of the cbbP gene (e.g., GenBank Gene ID 10921825 in C. necator N-1 or GenBank Gene ID 9004656 in R. capsulatus SB 1003). In some embodiments, a recombinant host comprises an attenuation of the cbbP gene and the cbbL gene. In some embodiments, a recombinant host comprises an attenuation of the cbbP gene and the cbbS gene. In some embodiments, a recombinant host comprises an attenuation of the cbbP gene, the cbbL gene, and the cbbS gene.

In some embodiments, a recombinant host comprises an attenuation of the cbbR gene and the cbbM gene.

In some embodiments, a recombinant host comprises an attenuation of one or more of the following genes associated with native formate metabolism: fdsG, fdsB, fdsA, fdsC, and fdsD. In some embodiments, a recombinant host comprises an attenuation of fdsG (e.g., GenBank Gene ID 10917038 in C. necator N-1). In some embodiments, a recombinant host comprises an attenuation of fdsA (e.g., GenBank Gene ID 10917040 in C. necator N-1). In some embodiments, a recombinant host comprises an attenuation of fdsC (e.g., GenBank Gene ID 10917041 in C. necator N-1). In some embodiments, a recombinant host comprises an attenuation of fdsD (e.g., GenBank Gene ID 10917042 in C. necator N-1).

In some embodiments, a recombinant host comprises an attenuation of cbbL, cbbS, and one or more of fdsG, fdsB, fdsA, fdsC, and fdsD.

In some embodiments, a recombinant host comprises an attenuation of cbbLp, cbbSp, and one or more of fdsG, fdsB, fdsA, fdsC, and fdsD.

In some embodiments, a recombinant host comprises an attenuation of one or more enzymes associated with the Calvin-Benson cycle and/or native formate metabolism.

In some embodiments, a recombinant host comprises an attenuation of one or more of the enzymes phosphoribulokinase, ribulose-1,5-bisphosphate carboxylase/oxygenase and NAD⁺-reducing formate dehydrogenase.

In some embodiments, the recombinant host comprises an attenuation of phosphoribulokinase (e.g., GenBank Accession No. KUE89983.1 in C. necator or GenBank Accession No. AAC32306.1 in R. capsulatus SB 1003).

In some embodiments, the recombinant host comprises an attenuation of ribulose-1,5-bisphosphate carboxylase/oxygenase (e.g., GenBank Accession No. CDG15352.1 in C. necator or GenBank Accession No. AAC37141.1 in R. capsulatus SB 1003). In some embodiments, the recombinant host comprises an attenuation of Form I ribulose-1,5-bisphosphate carboxylase/oxygenase. In some embodiments, the recombinant host comprises an attenuation of Form II ribulose-1,5-bisphosphate carboxylase/oxygenase. In some embodiments, the recombinant host comprises an attenuation of Form I ribulose-1,5-bisphosphate carboxylase/oxygenase and Form II ribulose-1,5-bisphosphate carboxylase/oxygenase.

In some embodiments, the recombinant host comprises an attenuation of NAD⁺-reducing formate dehydrogenase. In some embodiments, the recombinant host comprises an attenuation of ribulose-1,5-bisphosphate carboxylase/oxygenase and NAD⁺-reducing formate dehydrogenase. In some embodiments, the recombinant host comprises an attenuation of phosphoribulokinase and NAD⁺-reducing formate dehydrogenase. In some embodiments, the recombinant host comprises an attenuation of phosphoribulokinase, NAD⁺-reducing formate dehydrogenase, and ribulose-1,5-bisphosphate carboxylase/oxygenase.

In some embodiments, a recombinant host comprises an attenuation of one or both of the LysR-type transcriptional activators CbbR_{I} and CbbR_{II}.

In some embodiments, a recombinant host comprises an attenuation of the LysR-type transcriptional activator CbbR_{I} (e.g., GenBank Accession No. AAC32308.1 in R. capsulatus SB 1003).

In some embodiments, a recombinant host comprises an attenuation of the LysR-type transcriptional activator CbbR_{II} (e.g., GenBank Accession No. AAC32304.1 in R. capsulatus SB 1003).

In some embodiments, a recombinant host comprises an attenuation of the LysR-type transcriptional activator CbbR_{I} and the LysR-type transcriptional activator CbbR_{II}.

Attenuation strategies include, but are not limited to, the use of transposons, homologous recombination, mutagenesis, enzyme inhibitors, and RNAi interference.

For example, Thomason et al. describes the use of homologous recombination in gene attenuation. See Thomas et al., “Recombineering: genetic engineering in bacteria using homologous recombination.” Current Protocols in Molecular Biology (2007): 1-16.

For example, Agarwal et al. describe the use of double-stranded RNA-mediated interference (RNAi) to silence gene expression in a range of organisms. See Agrawal, Neema, et al. “RNA interference: biology, mechanism, and applications.” Microbiology and Molecular Biology Reviews 67.4 (2003): 657-685.

In some embodiments, attenuation strategies may remove a gene, decrease gene expression, or inactivate an enzyme.

For example, in some embodiments, DL-glyceraldehyde, a small molecule inhibitor, is used to inhibit one or more enzymes associated with the Calvin-Benson cycle. DL-glyceraldehyde has previously been shown to inhibit Calvin-Benson cycle activity (see, e.g., Khetkorn, Wanthanee, et al. “Redirecting the electron flow towards the nitrogenase and bidirectional Hox-hydrogenase by using specific inhibitors results in enhanced H2 production in the cyanobacterium Anabaena siamensis TISTR 8012.” Bioresource Technology 118 (2012): 265-271.).

For example, in some embodiments, DL-glyceraldehyde may inhibit ribulose 1,5-bisphosphate carboxylase (e.g., GenBank Accession No. KUE89989.1 in C. necator).

In some embodiments, attenuation of the Calvin-Benson cycle and utilization of a synthetic pathway described herein in a host microorganism results in more efficient recycling of donated electron carriers and more efficient carbon fixation.

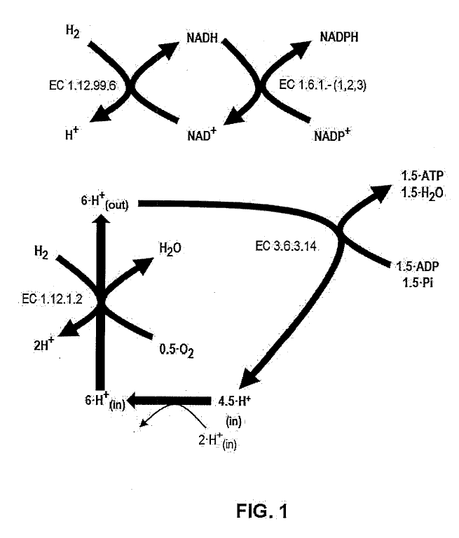

FIG. 1 demonstrates exemplary natural biochemical pathways summarizing the energetics associated with H₂ as electron donor for the synthesis of reducing equivalents.

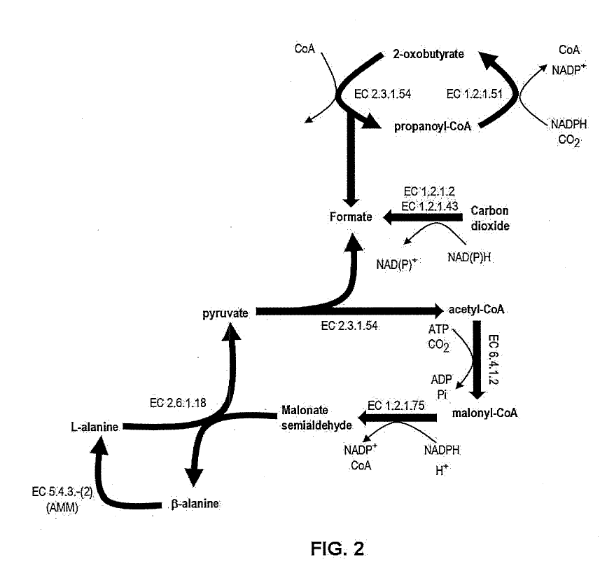

FIG. 2 demonstrates exemplary natural biochemical pathways leading to formate synthesis. In some embodiments, natural pathways leading to formate synthesis, such as, e.g., the pathways outlined in FIG. 2, are attenuated. For example, in some embodiments, a recombinant host comprises an attenuation of the enzyme pyruvate dehydrogenase.

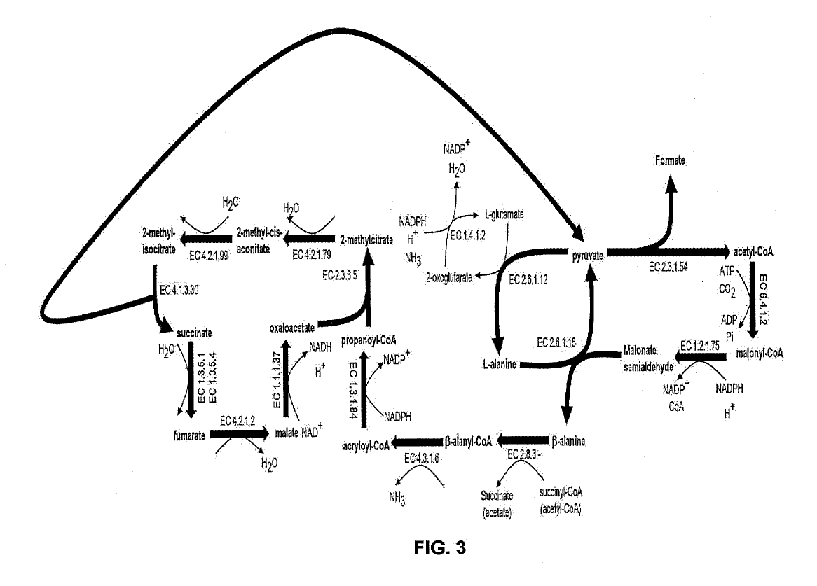

The remaining figures provide novel synthetic pathways for the synthesis of formate and the assimilation of formate into central carbon metabolism. In some embodiments, the novel synthetic pathways provided in FIG. 3-12 may be used by a recombinant host as alternatives to the Calvin-Benson cycle, resulting in more efficient recycling of donated electron carriers and more efficient carbon fixation.

### Enzymes

As used herein, references to a particular enzyme mean a protein having the activity of the particular enzyme.

Many of the enzymes described herein catalyze reversible reactions, and the reaction of interest may be the reverse of the described reaction. The schematic pathways shown in FIGS. 1 to 12 illustrate the reaction of interest for each of the intermediates.

In some embodiments, a recombinant host may include one or both exogenous enzymes selected from a methylisocitrate lyase and a formate dehydrogenase.

In some embodiments, a recombinant host may overexpress one or more genes encoding: an methylisocitrate lyase and/or a formate dehydrogenase.

In some embodiments, a recombinant host may include a methylisocitrate lyase and an anaplerotic enzyme. In some further embodiments, the anaplerotic enzyme is selected from a pyruvate carboxylase, a phosphoenolpyruvate carboxylase, a malic enzyme, and an isocitrate dehydrogenase.

In some embodiments, a recombinant host may over express one or more genes encoding: a methylisocitrate lyase and/or an anaplerotic enzyme. In some further embodiments, the anaplerotic enzyme is selected from a pyruvate carboxylase, a phosphoenolpyruvate carboxylase, a malic enzyme, and an isocitrate dehydrogenase.

In some embodiments, a recombinant host may include one or more exogenous enzymes selected from a 2-methylcitrate dehydratase, a methylisocitrate lyase, a succinate dehydrogenase (quinone), a fumarate reductase (quinol), a fumarate hydratase, a malate dehydrogenase, a 2-methylisocitrate dehydratase, a 2-methylcitrate synthase, an acrylyl-CoA reductase (NADPH), a β-alanyl-Cokammonia lyase, a glutamate dehydrogenase, a CoA-transferase, an alanine transaminase, a β-alanine pyruvate aminotransferase, a formate C-acetyltransferase, a malonyl-CoA reductase (malonate semialdehyde-forming), an acetyl-CoA carboxylase, an enoyl-CoA hydratase, a 3-hydroxypropionyl-CoA synthase, a lactoyl-CoA dehydratase, a propionate CoA-transferase, a L-lactate dehydrogenase, a lactate-malate transhydrogenase, a 3-hydroxypropionate dehydrogenase, a threonine ammonia-lyase, a cystathionine γ-lyase, a homoserine dehydrogenase, an aspartate-semialdehyde dehydrogenase, a malate dehydrogenase (oxaloacetate-decarboxylating), an aspartate kinase, a formate-tetrahydrofolate ligase, a methenyltetrahydrofolate cyclohydrolase, a glycine hydroxymethyltransferase, a serine-glyoxylate transaminase, a hydroxypyruvate reductase, a glycerate dehydrogenase, a glycerate 2-kinase, a phosphopyruvate hydratase, a phosphoenolpyruvate carboxylase, a malate-CoA ligase, a malyl-CoA lyase, a pyruvate kinase, a pyruvate carboxylase, a succinyl-CoA-L-malate CoA-transferase, a pyruvate synthase, a tartronate-semialdehyde synthase, an oxidoreductase with NAD(+) or NADP(+) as acceptor, a glycerate 3-kinase, a phosphoglycerate mutase (2, 3-diphosphoglycerate-independent), a phosphoglycerate mutase (2,3-diphosphoglycerate-dependent), a pyruvate, phosphate dikinase, a pyruvate, water dikinase, a hydroxypyruvate isomerase, a 2-dehydro-3-deoxyglucarate aldolase, a 5-dehydro-4-deoxyglucarate dehydratase, a 2,5-dioxovalerate dehydrogenase, an acetate-CoA ligase, a formyl-CoA transferase, an aldehyde-alcohol dehydrogenase, a 6-phospho-3-hexuloisomerase, a 6-phosphofructokinase, a fructose-bisphosphate aldolase, a transketolase, a transaldolase, a ribulose-phosphate 3-epimerase, a ribose-5-phosphate isomerase, a fructose-6-phosphate phosphoketolase, and a phosphate acetyltransferase.

For example, in some embodiments, a recombinant host may include one or more exogenous enzymes selected from a 2-methylcitrate dehydratase, a methylisocitrate lyase, a succinate dehydrogenase (quinone), a fumarate reductase (quinol), a fumarate hydratase, a malate dehydrogenase, a 2-methylisocitrate dehydratase, a 2-methylcitrate synthase, an acrylyl-CoA reductase (NADPH), a β-alanyl-CoA:ammonia lyase, a glutamate dehydrogenase, a CoA-transferase, an alanine transaminase, a β-alanine pyruvate aminotransferase, a formate C-acetyltransferase, a malonyl-CoA reductase (malonate semialdehyde-forming), and an acetyl-CoA carboxylase.

In some embodiments, a recombinant host may include one or more exogenous enzymes selected from a enoyl-CoA hydratase and a 3-hydroxypropionyl-CoA synthase.

In some embodiments, a recombinant host may include one or more exogenous enzymes selected from a lactoyl-CoA dehydratase, a propionate CoA-transferase, a L-lactate dehydrogenase, a lactate-malate transhydrogenase, and a 3-hydroxypropionate dehydrogenase.

In some embodiments, a recombinant host may include one or more exogenous enzymes selected from a threonine ammonia-lyase, a cystathionine γ-lyase, a homoserine dehydrogenase, an aspartate-semialdehyde dehydrogenase, a malate dehydrogenase (oxaloacetate-decarboxylating), and an aspartate kinase.

In some embodiments, a recombinant host may include one or more exogenous enzymes selected from a formate-tetrahydrofolate ligase, a methenyltetrahydrofolate cyclohydrolase, a glycine hydroxymethyltransferase, a serine-glyoxylate transaminase, a hydroxypyruvate reductase, a glycerate dehydrogenase, a glycerate 2-kinase, a phosphopyruvate hydratase, a phosphoenolpyruvate carboxylase, a malate-CoA ligase, a malyl-CoA lyase, a pyruvate kinase, a pyruvate carboxylase, a succinyl-CoA-L-malate CoA-transferase, and a pyruvate synthase.

In some embodiments, a recombinant host may include one or more exogenous enzymes selected from a tartronate-semialdehyde synthase, an oxidoreductase with NAD(+) or NADP(+) as acceptor, a glycerate 3-kinase, a phosphoglycerate mutase (2,3-diphosphoglycerate-independent), a phosphoglycerate mutase (2,3-diphosphoglycerate-dependent), a pyruvate, phosphate dikinase, and a pyruvate, water dikinase.

In some embodiments, a recombinant host may include one or more exogenous enzymes selected from a hydroxypyruvate isomerase, a 2-dehydro-3-deoxyglucarate aldolase, a 5-dehydro-4-deoxyglucarate dehydratase, and a 2,5-dioxovalerate dehydrogenase.

In some embodiments, a recombinant host may include one or more exogenous enzymes selected from an acetate-CoA ligase, a formyl-CoA transferase, an aldehyde-alcohol dehydrogenase, a 6-phospho-3-hexuloisomerase, a 6-phosphofructokinase, a fructose-bisphosphate aldolase, a transketolase, a transaldolase, a ribulose-phosphate 3-epimerase, a ribose-5-phosphate isomerase, a fructose-6-phosphate phosphoketolase, and a phosphate acetyltransferase.

In some embodiments, a recombinant host may overexpress one or more genes encoding: a 2-methylcitrate dehydratase, a methylisocitrate lyase, a succinate dehydrogenase (quinone), a fumarate reductase (quinol), a fumarate hydratase, a malate dehydrogenase, a 2-methylisocitrate dehydratase, a 2-methylcitrate synthase, an acrylyl-CoA reductase (NADPH), a β-alanyl-CoA:ammonia lyase, a glutamate dehydrogenase, a CoA-transferase, an alanine transaminase, a β-alanine pyruvate aminotransferase, a formate C-acetyltransferase, a malonyl-CoA reductase (malonate semialdehyde-forming), an acetyl-CoA carboxylase, an enoyl-CoA hydratase, a 3-hydroxypropionyl-CoA synthase, a lactoyl-CoA dehydratase, a propionate CoA-transferase, a L-lactate dehydrogenase, a lactate-malate transhydrogenase, a 3-hydroxypropionate dehydrogenase, a threonine ammonia-lyase, a cystathionine γ-lyase, a homoserine dehydrogenase, an aspartate-semialdehyde dehydrogenase, a malate dehydrogenase (oxaloacetate-decarboxylating), an aspartate kinase, a formate-tetrahydrofolate ligase, a methenyltetrahydrofolate cyclohydrolase, a glycine hydroxymethyltransferase, a serine-glyoxylate transaminase, a hydroxypyruvate reductase, a glycerate dehydrogenase, a glycerate 2-kinase, a phosphopyruvate hydratase, a phosphoenolpyruvate carboxylase, a malate-CoA ligase, a malyl-CoA lyase, a pyruvate kinase, a pyruvate carboxylase, a succinyl-CoA-L-malate CoA-transferase, a pyruvate synthase, a tartronate-semialdehyde synthase, an oxidoreductase with NAD(+) or NADP(+) as acceptor, a glycerate 3-kinase, a phosphoglycerate mutase (2,3-diphosphoglycerate-independent), a phosphoglycerate mutase (2,3-diphosphoglycerate-dependent), a pyruvate, phosphate dikinase, a pyruvate, water dikinase, a hydroxypyruvate isomerase, a 2-dehydro-3-deoxyglucarate aldolase, a 5-dehydro-4-deoxyglucarate dehydratase, a 2, 5-dioxovalerate dehydrogenase, an acetate-CoA ligase, a formyl-CoA transferase, an aldehyde-alcohol dehydrogenase, a 6-phospho-3-hexuloisomerase, a 6-phosphofructokinase, a fructose-bisphosphate aldolase, a transketolase, a transaldolase, a ribulose-phosphate 3-epimerase, a ribose-5-phosphate isomerase, a fructose-6-phosphate phosphoketolase, and/or a phosphate acetyltransferase.

For example, in some embodiments, a recombinant host may overexpress one or more genes encoding: a 2-methylcitrate dehydratase, a methylisocitrate lyase, a succinate dehydrogenase (quinone), a fumarate reductase (quinol), a fumarate hydratase, a malate dehydrogenase, a 2-methylisocitrate dehydratase, a 2-methylcitrate synthase, an acrylyl-CoA reductase (NADPH), a β-alanyl-CoA:ammonia lyase, a glutamate dehydrogenase, a CoA-transferase, an alanine transaminase, a β-alanine pyruvate aminotransferase, a formate C-acetyltransferase, a malonyl-CoA reductase (malonate semialdehyde-forming), and/or an acetyl-CoA carboxylase.

In some embodiments, a recombinant host overexpress one or more genes encoding: a enoyl-CoA hydratase and/or a 3-hydroxypropionyl-CoA synthase.

In some embodiments, a recombinant host may overexpress one or more genes encoding: a lactoyl-CoA dehydratase, a propionate CoA-transferase, a L-lactate dehydrogenase, a lactate-malate transhydrogenase, and/or a 3-hydroxypropionate dehydrogenase.

In some embodiments, a recombinant host may overexpress one or more genes encoding: a threonine ammonia-lyase, a cystathionine γ-lyase, a homoserine dehydrogenase, an aspartate-semialdehyde dehydrogenase, a malate dehydrogenase (oxaloacetate-decarboxylating), and/or an aspartate kinase.

In some embodiments, a recombinant host may overexpress one or more genes encoding: a formate-tetrahydrofolate ligase, a methenyltetrahydrofolate cyclohydrolase, a glycine hydroxymethyltransferase, a serine-glyoxylate transaminase, a hydroxypyruvate reductase, a glycerate dehydrogenase, a glycerate 2-kinase, a phosphopyruvate hydratase, a phosphoenolpyruvate carboxylase, a malate-CoA ligase, a malyl-CoA lyase, a pyruvate kinase, a pyruvate carboxylase, a succinyl-CoA-L-malate CoA-transferase, and/or a pyruvate synthase.

In some embodiments, a recombinant host may overexpress one or more genes encoding: a tartronate-semialdehyde synthase, an oxidoreductase with NAD(+) or NADP(+) as acceptor, a glycerate 3-kinase, a phosphoglycerate mutase (2,3-diphosphoglycerate-independent), a phosphoglycerate mutase (2,3-diphosphoglycerate-dependent), a pyruvate, phosphate dikinase, and/or a pyruvate, water dikinase.

In some embodiments, a recombinant host may overexpress one or more genes encoding: a hydroxypyruvate isomerase, a 2-dehydro-3-deoxyglucarate aldolase, a 5-dehydro-4-deoxyglucarate dehydratase, and/or a 2,5-dioxovalerate dehydrogenase.

In some embodiments, a recombinant host may overexpress one or more genes encoding: from an acetate-CoA ligase, a formyl-CoA transferase, an aldehyde-alcohol dehydrogenase, a 6-phospho-3-hexuloisomerase, a 6-phosphofructokinase, a fructose-bisphosphate aldolase, a transketolase, a transaldolase, a ribulose-phosphate 3-epimerase, a ribose-5-phosphate isomerase, a fructose-6-phosphate phosphoketolase, and/or a phosphate acetyltransferase.

Within an engineered pathway, the enzymes can be from a single source, i.e., from one species or genera, or from multiple sources, i.e., different species or genera. Nucleic acids encoding the enzymes described herein have been identified from various organisms and are readily available in publicly available databases such as GenBank or EMBL.

Any of the enzymes described herein can have at least 70% sequence identity (homology) (e.g., at least 75%, 80%, 85%, 90%, 91%, 92%, 93%, 94%, 95%, 96%, 97%, 98%, 99%, or 100%) to the amino acid sequence of the corresponding wild-type enzyme. Sequence identity may be determined on the basis of the mature enzyme (e.g., with any signal sequence removed) or on the basis of the immature enzyme (e.g., with any signal sequence included).

The percent identity (homology) between two amino acid sequences can be determined as follows. First, the amino acid sequences are aligned using the BLAST 2 Sequences (Bl2seq) program from the stand-alone version of BLASTZ containing BLASTP version 2.0.14. This stand-alone version of BLASTZ can be obtained from the U.S. government's National Center for Biotechnology Information web site (www.ncbi.nlm.nih.gov). Instructions explaining how to use the Bl2seq program can be found in the readme file accompanying BLASTZ. Bl2seq performs a comparison between two amino acid sequences using the BLASTP algorithm. To compare two amino acid sequences, the options of Bl2seq are set as follows: -i is set to a file containing the first amino acid sequence to be compared (e.g., C:\seql.txt); -j is set to a file containing the second amino acid sequence to be compared (e.g., C:\seq2.txt); -pis set to blastp; -o is set to any desired file name (e.g., C:\output.txt); and all other options are left at their default setting. For example, the following command can be used to generate an output file containing a comparison between two amino acid sequences: C:\Bl2seq -i c:\seq 1.txt -j c:\seq2.txt -p blastp -o c:\output.txt. If the two compared sequences share homology (identity), then the designated output file will present those regions of homology as aligned sequences. If the two compared sequences do not share homology (identity), then the designated output file will not present aligned sequences. Similar procedures can be following for nucleic acid sequences except that blastn is used.

Once aligned, the number of matches is determined by counting the number of positions where an identical amino acid residue is presented in both sequences. The percent identity (homology) is determined by dividing the number of matches by the length of the full-length polypeptide amino acid sequence followed by multiplying the resulting value by 100. It is noted that the percent identity (homology) value is rounded to the nearest tenth. For example, 78.11, 78.12, 78.13, and 78.14 is rounded down to 78.1, while 78.15, 78.16, 78.17, 78.18, and 78.19 is rounded up to 78.2. It also is noted that the length value will always be an integer.

It will be appreciated that a number of nucleic acids can encode a polypeptide having a particular amino acid sequence. The degeneracy of the genetic code is well known to the art; i.e., for many amino acids, there is more than one nucleotide triplet that serves as the codon for the amino acid. For example, codons in the coding sequence for a given enzyme can be modified such that optimal expression in a particular species (e.g., bacteria or fungus) is obtained, using appropriate codon bias tables for that species.

Functional fragments of any of the enzymes described herein can also be used in the methods described herein. The term “functional fragment” as used herein refers to a peptide fragment of a protein that has at least 25% (e.g., at least: 30%; 40%; 50%; 60%; 70%; 75%; 80%; 85%; 90%; 95%; 98%; 99%; 100%; or even greater than 100%) of the activity of the corresponding mature, full-length, wild-type protein. The functional fragment can generally, but not always, be comprised of a continuous region of the protein, wherein the region has functional activity.

Engineered hosts can naturally express none or some (e.g., one or more, two or more, three or more, four or more, five or more, or six or more) of the enzymes of the pathways described herein. Thus, a pathway within an engineered host can include all exogenous enzymes, or can include both endogenous and exogenous enzymes. A pathway within an engineered host can also include recombinant versions of endogenous enzymes placed under control of appropriate heterologous promoters (i.e., promoters with which the endogenous enzymes are not naturally associated with). Endogenous genes of the engineered hosts also can be disrupted to prevent the formation of undesirable metabolites or prevent the loss of intermediates in the pathway through other enzymes acting on such intermediates. Engineered hosts can be referred to as recombinant hosts or recombinant host cells.

In some embodiments, a recombinant host may include nucleic acids encoding one or more of a 2-methylcitrate dehydratase, a methylisocitrate lyase, a succinate dehydrogenase (quinone), a fumarate reductase (quinol), a fumarate hydratase, a malate dehydrogenase, a 2-methylisocitrate dehydratase, a 2-methylcitrate synthase, an acrylyl-CoA reductase (NADPH), a β-alanyl-CoA:ammonia lyase, a glutamate dehydrogenase, a CoA-transferase, an alanine transaminase, a β-alanine pyruvate aminotransferase, a formate C-acetyltransferase, a malonyl-CoA reductase (malonate semialdehyde-forming), an acetyl-CoA carboxylase, an enoyl-CoA hydratase, a 3-hydroxypropionyl-CoA synthase, a lactoyl-CoA dehydratase, a propionate CoA-transferase, a L-lactate dehydrogenase, a lactate-malate transhydrogenase, a 3-hydroxypropionate dehydrogenase, a threonine ammonia-lyase, a cystathionine γ-lyase, a homoserine dehydrogenase, an aspartate-semialdehyde dehydrogenase, a malate dehydrogenase (oxaloacetate-decarboxylating), an aspartate kinase, a formate-tetrahydrofolate ligase, a methenyltetrahydrofolate cyclohydrolase, a glycine hydroxymethyltransferase, a serine-glyoxylate transaminase, a hydroxypyruvate reductase, a glycerate dehydrogenase, a glycerate 2-kinase, a phosphopyruvate hydratase, a phosphoenolpyruvate carboxylase, a malate-CoA ligase, a malyl-CoA lyase, a pyruvate kinase, a pyruvate carboxylase, a succinyl-CoA-L-malate CoA-transferase, a pyruvate synthase, a tartronate-semialdehyde synthase, an oxidoreductase with NAD(+) or NADP(+) as acceptor, a glycerate 3-kinase, a phosphoglycerate mutase (2, 3-diphosphoglycerate-independent), a phosphoglycerate mutase (2,3-diphosphoglycerate-dependent), a pyruvate, phosphate dikinase, a pyruvate, water dikinase, a hydroxypyruvate isomerase, a 2-dehydro-3-deoxyglucarate aldolase, a 5-dehydro-4-deoxyglucarate dehydratase, a 2,5-dioxovalerate dehydrogenase, an acetate-CoA ligase, a formyl-CoA transferase, an aldehyde-alcohol dehydrogenase, a 6-phospho-3-hexuloisomerase, a 6-phosphofructokinase, a fructose-bisphosphate aldolase, a transketolase, a transaldolase, a ribulose-phosphate 3-epimerase, a ribose-5-phosphate isomerase, a fructose-6-phosphate phosphoketolase, and a phosphate acetyltransferase.

For example, in some embodiments, a recombinant host may include nucleic acids encoding one or more of a 2-methylcitrate dehydratase, a methylisocitrate lyase, a succinate dehydrogenase (quinone), a fumarate reductase (quinol), a fumarate hydratase, a malate dehydrogenase, a 2-methylisocitrate dehydratase, a 2-methylcitrate synthase, an acrylyl-CoA reductase (NADPH), a β-alanyl-CoA:ammonia lyase, a glutamate dehydrogenase, a CoA-transferase, an alanine transaminase, a β-alanine pyruvate aminotransferase, a formate C-acetyltransferase, a malonyl-CoA reductase (malonate semialdehyde-forming), and an acetyl-CoA carboxylase.

In some embodiments, a recombinant host may include nucleic acids encoding one or more of enoyl-CoA hydratase and a 3-hydroxypropionyl-CoA synthase.

In some embodiments, a recombinant host may include nucleic acids encoding one or more of a lactoyl-CoA dehydratase, a propionate CoA-transferase, a L-lactate dehydrogenase, a lactate-malate transhydrogenase, and a 3-hydroxypropionate dehydrogenase.

In some embodiments, a recombinant host may include nucleic acids encoding one or more of a threonine ammonia-lyase, a cystathionine γ-lyase, a homoserine dehydrogenase, an aspartate-semialdehyde dehydrogenase, a malate dehydrogenase (oxaloacetate-decarboxylating), and an aspartate kinase.

In some embodiments, a recombinant host may include nucleic acids encoding one or more of a formate-tetrahydrofolate ligase, a methenyltetrahydrofolate cyclohydrolase, a glycine hydroxymethyltransferase, a serine-glyoxylate transaminase, a hydroxypyruvate reductase, a glycerate dehydrogenase, a glycerate 2-kinase, a phosphopyruvate hydratase, a phosphoenolpyruvate carboxylase, a malate-CoA ligase, a malyl-CoA lyase, a pyruvate kinase, a pyruvate carboxylase, a succinyl-CoA-L-malate CoA-transferase, and a pyruvate synthase.

In some embodiments, a recombinant host may include nucleic acids encoding one or more of a tartronate-semialdehyde synthase, an oxidoreductase with NAD(+) or NADP(+) as acceptor, a glycerate 3-kinase, a phosphoglycerate mutase (2,3-diphosphoglycerate-independent), a phosphoglycerate mutase (2,3-diphosphoglycerate-dependent), a pyruvate, phosphate dikinase, and a pyruvate, water dikinase.

In some embodiments, a recombinant host may include nucleic acids encoding one or more of a hydroxypyruvate isomerase, a 2-dehydro-3-deoxyglucarate aldolase, a 5-dehydro-4-deoxyglucarate dehydratase, and a 2,5-dioxovalerate dehydrogenase.

In some embodiments, a recombinant host may include nucleic acids encoding one or more of an acetate-CoA ligase, a formyl-CoA transferase, an aldehyde-alcohol dehydrogenase, a 6-phospho-3-hexuloisomerase, a 6-phosphofructokinase, a fructose-bisphosphate aldolase, a transketolase, a transaldolase, a ribulose-phosphate 3-epimerase, a ribose-5-phosphate isomerase, a fructose-6-phosphate phosphoketolase, and a phosphate acetyltransferase.

In some embodiments, a 2-methylisocitrate dehydratase may be classified under EC 4.2.1.99, such as aconitate hydratase B encoded by acnB from Escherichia coli K-12 (e.g., RefSeq Accession No. NP_414660.1), aconitate hydratase A encoded by citB from Bacillus subtilis 168 (e.g., RefSeq Accession No. NP_389683.1), or aconitate hydratase A encoded by acnM from C. necator (e.g., UniProtKB Accession No. Q937N8.1).

In some embodiments, a methylisocitrate lyase may be classified under EC 4.1.3.30, such as 2-methylisocitrate lyase encoded by prpB from E. coli K-12 (e.g., RefSeq Accession No. NP_414865.1), 2-methylisocitrate lyase encoded by prpB from Salmonella typhimurium LT2 (e.g., RefSeq Accession No. NP_459363.1), or isocitrate lyase 1 encoded by icl1 from Mycobacterium tuberculosis ATCC 35801 (e.g., RefSeq Accession No. YP_177728.1).

In some embodiments, a succinate dehydrogenase (quinone) may be classified under EC 1.3.5.1, such as succinate dehydrogenase encoded by sdhA/B from E. coli K-12 (e.g., RefSeq Accession No. NP_415251.1 and NP_415252.1), succinate dehydrogenase encoded by sdhA/B from B. subtilis 168 (e.g., RefSeq Accession No. NP_390722.2 and NP_390721.1), or succinate dehydrogenase encoded by sdhA/B from Rickettsia prowazekii Madrid E (e.g., RefSeq Accession No. NP_220520.1 and NP_220438.1).

In some embodiments, a fumarate reductase (quinol) may be classified under EC 1.3.5.4, such as fumarate reductase encoded by frdA from E. coli K-12 (e.g., RefSeq Accession No. NP_418578.1), fumarate reductase encoded by frdA from Heliobacter pylori J99 (e.g., GenBank Accession No. AAD05762.1), or fumarate reductase encoded by frdA from Mycobacterium bovis ATCC BAA-935 (e.g., RefSeq Accession No. NP_855230.1).

In some embodiments, a fumarate hydratase may be classified under EC 4.2.1.2, such as fumarate hydratase encoded by fumA/C from E. coli K-12 (e.g., RefSeq Accession No. NP_416129.1 and NP_416128.1), fumarate hydratase encoded by fumA/C from M. tuberculosis ATCC 25618 (e.g., RefSeq Accession No. NP_215614.1), or fumarate hydratase encoded by fumC from B. subtilis 168 (e.g., RefSeq Accession No. NP_391184.1).

In some embodiments, a malate dehydrogenase may be classified under EC 1.1.1.37, such as malate dehydrogenase encoded by mdh from E. coli K-12 (e.g., RefSeq Accession No. NP_417703.1), malate dehydrogenase encoded by mdh from Corynebacterium glutamicum ATCC 13032 (e.g., RefSeq Accession No. YP_226625.1), or malate dehydrogenase encoded by mdh from B. subtilis 168 (e.g., RefSeq Accession No. NP_390790.1).

In some embodiments, a 2-methylcitrate dehydratase may be classified under EC 4.2.1.79, such as 2-methylcitrate dehydratase encoded by prpD from E. coli K-12 (e.g., RefSeq Accession No. NP_414868.1), 2-methylcitrate dehydratase encoded by prpD from M. tuberculosis ATCC 25618 (e.g., GenBank Accession No. KLL06634.1), or 2-methylcitrate dehydratase encoded by prpD from C. necator (e.g., RefSeq Accession No. WP_013952326.1).

In some embodiments, a 2-methylcitrate synthase may be classified under EC 2.3.3.5, such as 2-methylcitrate synthase encoded by prpC from E. coli K-12 (e.g., RefSeq Accession No. NP_414867.1), 2-methylcitrate synthase encoded by prpC from C. necator (e.g., UniProtKB Accession No. Q937N9), or 2-methylcitrate synthase encoded by mmgD from B. subtilis 168 (e.g., RefSeq Accession No. NP_390294.1).

In some embodiments, an acrylyl-CoA reductase (NADPH) may be classified under EC 1.3.1.84, such as acrylyl-CoA reductase encoded by acul from E. coli K-12 (e.g., RefSeq Accession No. NP_417719.1), acrylyl-CoA reductase encoded by acul from Rhodobacter sphaeroides ATCC 17023 (e.g., RefSeq Accession No. YP_351476.1), or acrylyl-CoA reductase encoded by acul from Ruegeria pomeroyi (e.g., RefSeq Accession No. WP_011047645.1).

In some embodiments, a β-alanyl-CoA:ammonia lyase may be classified under EC 4.3.1.6, such as β-alanyl-CoA:ammonia lyase 2 encoded by acl2 from Clostridium propionicum (e.g., UniProtKB Accession No. Q6KC22).

In some embodiments, a glutamate dehydrogenase may be classified under EC 1.4.1.2 or EC 1.4.1.4, such as NAD(P)-specific glutamate dehydrogenase encoded by gdhA from Prevotella ruminicola (e.g., RefSeq Accession No. WP_013064508.1) or NAD(P)-specific glutamate dehydrogenase encoded by gdhA from E. coli K-12 (e.g., RefSeq Accession No. NP_416275.1).

In some embodiments, a CoA-transferase may be classified under EC 2.8.3.-, such as formyl-CoA:oxalate CoA-transferase encoded by frc from E. coli K-12 (e.g., UniProt Accession No. P69902.1).

In some embodiments, an alanine transaminase may be classified under EC 2.6.1.2, such as glutamate-pyruvate aminotransferase encoded by alaA or alaC from E. coli K-12 (e.g., RefSeq Accession No. NP_416880.1).

In some embodiments, a β-alanine pyruvate aminotransferase may be classified under EC 2.6.1.18, such as β-alanine pyruvate aminotransferase encoded by bauA from Pseudomonas aeruginosa (e.g., GenBank Accession No. BAR65000.1).

In some embodiments, a formate C-acetyltransferase may be classified under EC 2.3.1.54, such as formate acetyltransferase encoded by pfl from Clostridium butyricum (e.g., RefSeq Accession No. WP_043853189.1), formate acetyltransferase encoded by pfl from Clostridium pasteurianum (e.g., UniProtKB Accession No. Q46266.1), or formate acetyltransferase encoded by pflB/D/ybiW from E. coli K-12 (e.g., UniProtKB Accession No. P09373.2, P32674.1, and P75793.1).

In some embodiments, a malonyl-CoA reductase (malonate semialdehyde-forming) may be classified under EC 1.2.1.75, such as malonyl-CoA reductase encoded by mcr from Sulfolobus tokodaii (e.g., UniProtKB Accession No. Q96YK1.1) or malonyl-CoA reductase encoded by Msed_0709 from Metallosphaera sedula ATCC 51363 (e.g., UniProtKB Accession No. A4YEN2.1).

In some embodiments, an acetyl-CoA carboxylase may be classified under EC 6.4.1.2, such as acetyl-CoA carboxylase carboxyl transferase encoded by accA/C/D from E. coli K-12 (e.g., RefSeq Accession No. NP_414727.1, NP_417722.1, and NP_416819.1), acetyl-CoA carboxylase carboxyl transferase encoded by accA/C1/C2/D from C. necator H16 (e.g., UniProtKB Accession No. Q0KCA7.1, Q0KF85, Q0K6X4, and Q0K8H8), or acetyl-CoA carboxylase carboxyl transferase encoded by accA/C1/C2/D from B. subtilis 168.

In some embodiments, an enoyl-CoA hydratase may be classified under EC 4.2.1.17, such as fatty acid oxidation complex subunit alpha encoded by fadB from E. coli K-12 (e.g., RefSeq Accession No. NP_418288.1), fatty acid oxidation complex subunit alpha encoded by fadB from Pseudomonas fluorescens (e.g., RefSeq Accession No. WP_014339362.1), or fatty acid oxidation complex subunit alpha encoded by fadB from Yersinia pestis (e.g., RefSeq Accession No. YP_002348645.1).

In some embodiments, a 3-hydroxypropionyl-CoA synthase may be classified under EC 6.2.1.36, such as 3-hydroxypropionyl-CoA synthase encoded by Msed_1456 from M. sedula ATCC 51363 (e.g., UniProtKB Accession No. A4YGR1.1) or 3-hydroxypropionyl-CoA synthase encoded by STK_07830 from S. tokodaii DSM 16993 (e.g., GenBank Accession No. BAB65795.1).

In some embodiments, a lactoyl-CoA dehydratase may be classified under EC 4.2.1.54, such as lactoyl-CoA dehydratase encoded by lcdA/B from C. propionicum (e.g., UniProtKB Accession No. G3KIM4.1 and G3KIM3.1).

In some embodiments, a propionate CoA-transferase may be classified under EC 2.8.3.1, such as propionate CoA-transferase encoded by pct from C. propionicum (e.g., RefSeq Accession No. YP_765248.1), propionate CoA-transferase encoded by pct from Listeria welshimeri (e.g., RefSeq Accession No. YP_850387.1), or propionate CoA-transferase encoded by pct from Peptoniphilus indolicus ATCC 29427 (e.g., GenBank Accession No. EGY80526.1).

In some embodiments, a L-lactate dehydrogenase may be classified under EC 1.1.1.27, such as L-lactate dehydrogenase encoded by ldh from B. subtilis 168 (e.g., GenBank Accession No. NP_388187.2), L-lactate dehydrogenase encoded by Ldh1/2 from Lactobacillus plantarum (e.g., RefSeq Accession No. WP_003646532.1 and WP_003644108.1, L-lactate dehydrogenase encoded by ldh from Plasmodium falciparum (e.g., GenBank Accession No. ABA46355.1), or L-lactate dehydrogenase encoded by vpar_0498 from V. parvula (e.g., RefSeq Accession No. WP_012864037.1).

In some embodiments, a lactate-malate transhydrogenase may be classified under EC 1.1.99.7, such as lactate-malate transhydrogenase from Veillonella parvula.

In some embodiments, a 3-hydroxypropionate dehydrogenase may be classified under EC 1.1.1.59, such as D-mannonate oxidoreductase encoded by uxuB from E. coli K-12 (e.g., RefSeq Accession No. NP_418743.1).

In some embodiments, a 3-hydroxypropionate dehydrogenase (NADP+) may be classified under EC 1.1.1.298, such as NADP-dependent 3-hydroxy acid dehydrogenase encoded by ydfG from E. coli K-12 (e.g., UniProtKB Accession No. P39831.2).

In some embodiments, a threonine ammonia-lyase may be classified under EC 4.3.1.19, such as threonine dehydratase encoded by tdcB from E. coli K-12 (e.g., RefSeq Accession No. NP_417587.1), threonine dehydratase encoded by ilvA from C. glutamicum ATCC 13032 (e.g., RefSeq Accession No. WP_003862033.1), or threonine dehydratase encoded by ilvA from B. subtilis 168 (e.g., RefSeq Accession No. NP_390060.1).

In some embodiments, a cystathionine γ-lyase may be classified under EC 4.4.1.1, such as cystathionine γ-lyase encoded by mccB from B. subtilis (e.g., RefSeq Accession No. WP_{—} 003229810.1) or cystathionine β-lyase encoded by mccB from Staphylococcus aureus (e.g., RefSeq Accession No. WP_001036647.1).

In some embodiments, a homoserine dehydrogenase may be classified under EC 1.1.1.3, such as homoserine dehydrogenase encoded by thrA from E. coli K-12 (e.g., RefSeq Accession No. NP_414543.1) or homoserine dehydrogenase encoded by horn from B. subtilis 168 (e.g., RefSeq Accession No. NP_391106.1).

In some embodiments, an aspartate-semialdehyde dehydrogenase may be classified under EC 1.2.1.11, such as aspartate-semialdehyde dehydrogenase encoded by asd from E. coli K-12 (e.g., RefSeq Accession No. NP_417891.1), aspartate-semialdehyde dehydrogenase encoded by asd from M. tuberculosis ATCC 25618 (e.g., GenBank Accession No. AAQ75346.1), or aspartate-semialdehyde dehydrogenase encoded by asd from Pseudomonas aeruginosa (e.g., RefSeq Accession No. NP_251807.1).

In some embodiments, a malate dehydrogenase (oxaloacetate-decarboxylating) may be classified under EC 1.1.1.40, such as NADP-dependent malic enzyme encoded by maeB from E. coli K-12 (e.g., RefSeq Accession No. NP_416958.1).

In some embodiments, an aspartate kinase may be classified under EC 2.7.2.4, such as lysine-sensitive aspartokinase 3 encoded by lysC from E. coli K-12 (e.g., RefSeq Accession No. NP_418448.1), aspartokinase 2 encoded by lysC from B. subtilis 168 (e.g., RefSeq Accession No. NP_390725.1), or aspartokinase encoded by askfrom M. tuberculosis ATCC 35801 (e.g., RefSeq Accession No. NP_218226.1).

In some embodiments, a formate-tetrahydrofolate ligase may be classified under EC 6.3.4.3, such as formate tetrohydrofolate ligase encoded by fhs from Clostridium cylindrosporum (e.g., GenBank Accession No. KMT22112.1), formate tetrahydrofolate ligase encoded by fhs from S. aureus (e.g., GenBank Accession No. AKJ17614.1), or formate tetrahydrofolate ligase encoded by fhs from Lactobacillus delbrueckii (e.g., RefSeq Accession No. WP_052933655.1).

In some embodiments, a methylenetetrahydrofolate dehydrogenase (NADP⁺) may be classified under EC 1.5.1.5, such as bifunctional protein encoded by folD from Methylobacterium extorquens (e.g., UniProtKB Accession No. Q9X7F6.1) or bifunctional protein encoded by folD from Salmonella enterica (e.g., GenBank Accession No. GAR69529.1).

In some embodiments, a methenyltetrahydrofolate cyclohydrolase may be classified under EC 3.5.4.9, such as bifunctional protein encoded by folD from Methylobacterium extorquens (e.g., UniProtKB Accession No. Q9X7F6.1) or bifunctional protein encoded by folD from Salmonella enterica (e.g., GenBank Accession No. GAR69529.1).

In some embodiments, a glycine hydroxymethyltransferase may be classified under EC 2.1.2.1, such as serine hydroxymethyltransferase encoded by glyA from E. coli K-12 (e.g., RefSeq Accession No. NP_417046.1) or serine hydroxymethyltransferase encoded by glyA from S. typhimurium LT2 (e.g., RefSeq Accession No. NP_461490.1).

In some embodiments, a serine-glyoxylate transaminase may be classified under EC 2.6.1.45, such as serine-glyoxylate aminotransferase encoded by sgaA from H. methylovorum (e.g., UniProtKB Accession No. 008374.2) or serine-glyoxylate aminotransferase encoded by sgaA from M. extorquens ATCC 14718 (e.g., UniProtKB Accession No. P55819.2).

In some embodiments, a hydroxypyruvate reductase may be classified under EC 1.1.1.81, such as hydroxypyruvate reductase A encoded by ghrA from E. coli K-12 (e.g., RefSeq Accession No. NP_415551.2).

In some embodiments, a glycerate dehydrogenase may be classified under EC 1.1.1.29, such as glycerate dehydrogenase encoded by hprA from M. extorquens (e.g., RefSeq Accession No. WP_012253363.1).

In some embodiments, a glycerate 2-kinase may be classified under EC 2.7.1.165, such as glycerate kinase encoded by gck from Hyphomicrobium methylovorum, glycerate 2-kinase encoded by gck from S. tokodaii DSM 16993 (e.g., UniProtKB Accession No. Q96YZ3.1), or glycerate 2-kinase encoded by gck from Pyrococcus horikoshii (e.g., UniProtKB Accession No. 058231.1).

In some embodiments, a phosphopyruvate hydratase may be classified under EC 4.2.1.11, such as enolase encoded by eno from E. coli K-12 (e.g., RefSeq Accession No. NP_417259.1), enolase 1 encoded by enol from S. cerevisiae (e.g., RefSeq Accession No. NP_011770.3), or enolase encoded by eno from Plasmodium falciparum (e.g., GenBank Accession No. AAA18634.1).

In some embodiments, a phosphoenolpyruvate carboxylase may be classified under EC 4.1.1.31, such as phosphoenolpyruvate carboxylase encoded by ppc from E. coli K-12 (e.g., RefSeq Accession No. NP_418391.1), phosphoenolpyruvate carboxylase encoded by ppcA from Clostridium perfringens (e.g., RefSeq Accession No. WP_011590671.1), or phosphoenolpyruvate carboxylase encoded ppc from Rhodopseudomonas palustris (e.g., RefSeq Accession No. WP_011157278.1).

In some embodiments, a malate-CoA ligase may be classified under EC 6.2.1.9, such as malate-CoA ligase encoded by mtkA/B from M. extorquens (e.g., RefSeq Accession No. WP_015822351.1 and WP_003597632.1).

In some embodiments, a malyl-CoA lyase may be classified under EC 4.1.3.24, such as L-malyl-CoA/β-methylmalyl-CoA ligase encoded by mcl1 from Rhodobacter capsulatus (e.g., UniProtKB Accesion No. B6E2X2.1) or L-malyl-CoA/β-methylmalyl-CoA ligase encoded by mclA from Chloroflexus aurantiacus (e.g., UniProtKB Accession No. A9WC35.1).

In some embodiments, a pyruvate kinase may be classified under EC 2.7.1.40, such as pyruvate kinase encoded by pyk1 from S. cerevisiae (e.g., RefSeq Accession No. NP_009362.1) or pyruvate kinase encoded by pykA/F from E. coli K-12 (e.g., RefSeq Accession No. NP_416368.1 and NP_416191.1).

In some embodiments, a pyruvate carboxylase may be classified under EC 6.4.1.1, such as pyruvate carboxylase encoded by pyc1/2 from S. cerevisiae (e.g., RefSeq Accession No. NP_011453.1 and NP_009777.1) or pyruvate carboxylase encoded by pycA from B. subtilis 168 (e.g., RefSeq Accession No. NP_389369.1).

In some embodiments, a succinyl-CoA-L-malate CoA-transferase may be classified under EC 2.8.3.22, such as succinyl-CoA-L-malate-CoA transferase encoded by smtA/B from Chloroflexus aurantiacus (e.g., GenBank Accession No. ABF14400.1 and ABF14399.1).

In some embodiments, a pyruvate synthase may be classified under EC 1.2.7.1, such as pyruvate synthase encoded by por from Desulfovibrio africanus (e.g., UniProtKB Accession No. P94692.1).

In some embodiments, a tartronate-semialdehyde synthase may be classified under EC 4.1.1.47, such as glyoxylate carboligase encoded by gcl from E. coli K-12 (e.g., RefSeq Accession No. NP_415040.1).

In some embodiments, a oxidoreductase with NAD(+) or NADP(+) as acceptor may be classified under EC 1.1.1.-, such as 2-hydroxy-3-oxopropionate reductase encoded by garR from E. coli K-12 (e.g., RefSeq Accession No. NP_417594.3).

In some embodiments, a glycerate 3-kinase may be classified under EC 2.7.1.31, such as glycerate kinase II encoded by g/xK from E. coli K-12 (e.g., RefSeq Accession No. NP_415047.1).

In some embodiments, a phosphoglycerate mutase (2,3-diphosphoglycerate-independent) may be classified under EC 5.4.2.12, such as 2,3-bisphosphoglycerate-independent phosphoglycerate mutase encoded by gpml from E. coli K-12 (e.g., GenBank Accession No. AMC96500.1).

In some embodiments, a phosphoglycerate mutase (2,3-diphosphoglycerate-dependent) may be classified under EC 5.4.2.11, such as phosphoglycerate mutase encoded by gpml from S. cerevisiae (e.g., RefSeq Accession No. NP_012770.1).

In some embodiments, a pyruvate, phosphate dikinase may be classified under EC 2.7.9.1, such as pyruvate, phosphate dikinase encoded by ppdK from Cenarchaeum symbiosum (e.g., GenBank Accession No. ABK77107.1).

In some embodiments, a pyruvate, water dikinase may be classified under EC 2.7.9.2, such as phosphoenolpyruvate synthase encoded by ppsA from E. coli K-12 (e.g., RefSeq Accession No. NP_416217.1), phosphoenolpyruvate synthase encoded by ppsA from H. pylori (e.g., RefSeq Accession No. NP_416217.1), or phosphoenolpyruvate synthase encoded by ppsA from P. aeruginosa (e.g., RefSeq Accession No. WP_003098065.1).

In some embodiments, a hydroxypyruvate isomerase may be classified under EC 5.3.1.22, such as hydroxypyruvate isomerase encoded by hyi from E. coli K-12 (e.g., RefSeq Accession No. NP_415041.1).

In some embodiments, a 2-dehydro-3-deoxyglucarate aldolase may be classified under EC 4.1.2.20, such as 5-keto-4-deoxy-D-glucarate aldolase encoded by garL from E. coli K-12 (e.g., RefSeq Accession No. NP_417595.1) or 5-keto-4-deoxy-D-glucarate aldolase encoded by garL from S. typhimurium LT2 (e.g., RefSeq Accession No. NP_462162.1).

In some embodiments, a 5-dehydro-4-deoxyglucarate dehydratase may be classified under EC 4.2.1.41, such as probable 5-dehydro-4-deoxyglucarate dehydrogenase encoded by ybcC from B. subtilis 168 (e.g., UniProtKB Accession No. P42235.2) or 5-dehydro-4-deoxyglucarate dehydrogenase encoded by H16_80131 from C. necator (e.g., RefSeq Accession No. WP_011616367.1).

In some embodiments, a 2,5-dioxovalerate dehydrogenase may be classified under EC 1.2.1.26, such as α-ketoglutaric semialdehyde dehydrogenase encoded by araE from Azospirillum brasilense (e.g., UniProtKB Accession No. Q1JUP4.1).

In some embodiments, an acetate-CoA ligase may be classified under EC 6.2.1.1, such as acetyl-CoA synthetase encoded by acsA from P. aerophilum (e.g., UniProtKB Accession No. 093730.2), acetyl-CoA synthetase encoded by acs from S. typhimurium LT2 (e.g., RefSeq Accession No. NP_463140.1), or acetyl-CoA synthetase encoded by acs from E. coli K-12 (e.g., RefSeq Accession No. NP_418493.1).

In some embodiments, a formyl-CoA transferase may be classified under EC 2.8.3.16, such as formyl-CoA:oxalate CoA-transferase encoded by frc from E. coli K-12 (e.g., RefSeq Accession No. NP_416875.1), formyl-CoA:oxalate CoA-transferase encoded by frc from R. palustris CGA009 (e.g., UniProtKB Accession No. Q6N8F8.2), or formyl-CoA:oxalate CoA-transferase encoded by frc from Oxalobacter formigenes. (e.g., RefSeq Accession No. WP_005880857.1)

In some embodiments, an aldehyde-alcohol dehydrogenase may be classified under EC 1.2.1.10, such as aldehyde-alcohol dehydrogenase encoded by mhpF or adhE from E. coli K-12 (e.g., RefSeq Accession No. NP_414885.1 or NP_415757.1).

In some embodiments, a 6-phospho-3-hexuloisomerase may be classified under EC 5.3.1.27, such as 3-hexulose-6-phosphate isomerase encoded by rmpB from Methylomonas aminofaciens (e.g., UniProtKB Accession No. Q9S0X3.1), 3-hexulose-6-phosphate isomerase encoded by rmpB from Mycobacterium gastri (e.g., UniProtKB Accession No. Q9LBW5.1), or 3-hexulose-6-phosphate isomerase encoded by hxlB from B. subtilis 168 (e.g., RefSeq Accession No. NP_388227.1).

In some embodiments, a 6-phosphofructokinase may be classified under EC 2.7.1.11, such as 6-phosphofructokinase I encoded by pfkA from E. coli K-12 (e.g., RefSeq Accession No. NP_418351.1).

In some embodiments, a fructose-bisphosphate aldolase may be classified under EC 4.1.2.13, such as fructose-bisphosphate aldolase B encoded by fbaB from C. necator (e.g., GenBank Accession No. AEI75959.1) or fructose-bisphosphate aldolase B encoded by fbaA from E. coli K-12 (e.g., RefSeq Accession No. NP_417400.1).

In some embodiments, a transketolase may be classified under EC 2.2.1.1, such as transketolase encoded by tktA from E. coli K-12 (e.g., RefSeq Accession No. YP_026188.1) or transketolase encoded by tkt from M. tuberculosis ATCC 25618 (e.g., RefSeq Accession No. NP_215965.1).

In some embodiments, a transaldolase may be classified under EC 2.2.1.2, such as transaldolase encoded by talB from E. coli K-12 (e.g., RefSeq Accession No. NP_414549.1), transaldolase encoded by tal from B. subtilis 168 (e.g., RefSeq Accession No. NP_391592.3), or transaldolase encoded by tal from M. tuberculosis ATCC 25618 (e.g., RefSeq Accession No. NP_215964.1).

In some embodiments, a ribulose-phosphate 3-epimerase may be classified under EC 5.1.3.1, such as ribulose-phosphate 3-epimerase encoded by rpe1 from S. cerevisiae (e.g., RefSeq Accession No. NP_012414.1) or ribulose-phosphate 3-epimerase encoded by rpe from E. coli K-12 (e.g., RefSeq Accession No. NP_417845.1).

In some embodiments, a ribose-5-phosphate isomerase may be classified under EC 5.3.1.6, such as ribose-5-phosphate isomerase A encoded by rpiA from E. coli K-12 (e.g., RefSeq Accession No. NP_417389.1).

In some embodiments, a fructose-6-phosphate phosphoketolase may be classified under EC 4.1.2.22, such as xylulose-5-phosphate/fructose-6-phosphate phosphoketolase encoded by xfp from Bifidobacterium animalis (e.g., GenBank Accession No. BAF37975.1).

In some embodiments, a phosphate acetyltransferase may be classified under EC 2.3.1.8, such as phosphate acetyltransferase encoded by pta from E. coli K-12 (e.g., RefSeq Accession No. NP_416800.1), phosphate acetyltransferase encoded by pta from M. tuberculosis ATCC 25618 (e.g., RefSeq Accession No. NP_214922.1), or phosphate acetyltransferase encoded by pta from P. aeruginosa (e.g., RefSeq Accession No. NP_249526.1).

### Biochemical Networks

As used herein, references to a particular enzyme mean a protein having the activity of the particular enzyme.

The reactions of the pathways described herein can be performed in one or more host strains (a) naturally expressing one or more, but not all, relevant enzymes, (b) genetically engineered to express one or more relevant enzymes, or (c) naturally expressing one or more relevant enzymes and genetically engineered to express one or more relevant enzymes. Alternatively, relevant enzymes can be extracted from the above types of host cells and used in a purified or semi-purified form. Moreover, such extracts include lysates (e.g., cell lysates) that can be used as sources of relevant enzymes. In the methods provided herein, all the biochemical steps can be performed in host cells, all the steps can be performed using extracted enzymes, or some of the steps can be performed in cells and others can be performed using extracted enzymes.

Metabolic pathway engineering has successfully been utilized by several groups to produce chemical commodities via fermentation processes. For example, recombinant strains expressing multiple exogenous genes and utilizing multi-step pathways not native to the strains have been developed. Recent advances in metabolic pathway engineering are summarized in, e.g., Chotani, Gopal, et al. “The commercial production of chemicals using pathway engineering.” Biochimica et Biophysica Acta (BBA)-Protein Structure and Molecular Enzymology 1543.2 (2000): 434-455, Blombach, Bastian, and Bernhard J. Eikmanns. “Current knowledge on isobutanol production with Escherichia coli, Bacillus subtilis and Corynebacterium glutamicum.” Bioengineered Bugs 2.6 (2011): 346-350., and Adkins, Jake, et al. “Engineering microbial chemical factories to produce renewable tiomonomers” Synthetic Biology Applications in Industrial Microbiology (2014): 31.

For example, Rathnasingh et al. developed a novel recombinant Escherichia coli SH254 strain that can produce 3-hydroxypropionic acid from glycerol via two consecutive enzymatic reactions. To develop the novel strains, Rathnasingh et al. inserted two plasmids, one encoding 5 exogenous genes utilized in the enzymatic reactions, into an Escherichia coli SH254 strain. See Rathnasingh, Chelladurai, et al. “Development and evaluation of efficient recombinant Escherichia coli strains for the production of 3-hydroxypropionic acid from glycerol.” Biotechnol Bioeng 104.4 (2009): 729-739.

In addition, Martin et al. engineered the expression of a synthetic amorpha-4,11-diene synthase gene and the mevalonate isoprenoid pathway from Saccharomyces cerevisiae in Escherichia coli. See Martin, Vincent J J, et al. “Engineering a mevalonate pathway in Escherichia coli for production of terpenoids.” Nature Biotechnology 21.7 (2003): 796-802.

The following paragraphs exemplify novel synthetic pathways for carbon fixation including the synthesis of formate followed by assimilation of formate into central carbon metabolism. The synthetic carbon fixation pathways incorporate features of alternative natural metabolic pathways that perform carbon fixation. The synthetic pathways may be constructed within canonical laboratory and industrial hydrogen-oxidizing microorganisms such as Cupriavidus necator or Rhodobacter capsulatus that have an operable Calvin-Benson cycle to more efficiently recycle donated electrons from H₂.

### Formate Synthesis Via Acetyl-CoA Carboxylase, β-Alanine, and Methylcitrate Cycle

In one aspect, formate is synthesized using an acetyl-CoA carboxylase, β-alanine, and the methylcitrate cycle. See, e.g., FIG. 3.

In some embodiments, formate and acetyl-CoA may be synthesized from 2-methyl-isocitrate by conversion of 2-methyl-isocitrate to pyruvate and succinate by a methylisocitrate lyase classified, for example, under EC 4.1.3.30, such as the gene product of prpB from E. coli K-12; followed by conversion of pyruvate to formate and acetyl-CoA by a formate C-acetyltransferase classified, for example, under EC 2.3.1.54, such as the gene product of pfl from Clostridium butyricum.

In some embodiments, formate and acetyl-CoA may be synthesized from pyruvate synthesized from malonate semialdehyde and L-alanine by conversion of malonate semialdehyde and L-alanine to pyruvate by a β-alanine pyruvate aminotransferase classified, for example, under EC 2.6.1.18, such as the gene product of bauA from P. aeruginosa; followed by conversion of pyruvate to formate and acetyl-CoA by a formate C-acetyltransferase classified, for example, under EC 2.3.1.54, such as the gene product of pfl from Clostridium butyricum.

In some embodiments, acetyl-CoA formed as described above, ATP, and CO₂ are converted to malonyl-CoA, ADP, and Pᵢ by an acetyl-CoA carboxylase classified, for example, under EC 6.4.1.2, such as the gene product of accA/C/D from E. coli K-12; followed by conversion of malonyl-CoA formed as described above, NADPH, and 1-1⁺ to malonate semialdehyde, NADP⁺, and CoA by a malonyl-CoA reductase (malonate semialdehyde-forming) classified, for example, under EC 1.2.1.75, such as the gene product of mcr from Sulfolobus tokodaii; followed by conversion of malonate semialdehyde and L-alanine to β-alanine and pyruvate by a β-alanine pyruvate aminotransferase classified, for example, under EC 2.6.1.18, such as the gene product of bauA from Pseudomonas aeruginosa. In some embodiments, the pyruvate produced from the conversion of malonate semialdehyde and L-alanine may be converted to formate and acetyl-CoA as described above.

In some embodiments, L-alanine and 2-oxoglutarate may be synthesized from pyruvate and L-glutamate by conversion of pyruvate and L-glutamate to L-alanine and 2-oxoglutarate by an alanine transaminase classified, for example, under EC 2.6.1.2, such as the gene product of alaA from E. coli K-12 (e.g., UniProtKB Accession No. P0A959.1). In some embodiments, malonate semialdehyde and L-alanine synthesized from pyruvate and L-glutamate as described above are converted to β-alanine and pyruvate as described above. In some embodiments, NADPH, H⁺, NH₃, and 2-oxoglutarate formed as described above are converted to L-glutamate, NADP⁺, and H₂O by a glutamate dehydrogenase classified, for example, under EC 1.4.1.2 or EC 1.4.1.4 such as the gene product of gdhA from Prevotella ruminicola.

In some embodiments, β-alanine formed as described above and succinyl-CoA are converted to succinate and β-alanyl-CoA by a CoA-transferase classified, for example, under EC 2.8.3.-, such as the gene product of frc from E. coli K-12. Alternatively, β-alanine formed as described above and acetyl-CoA is converted to acetate and β-alanyl-CoA by a CoA-transferase classified, for example, under EC 2.8.3.-, such as the gene product of frc from E. coli K-12.

In some embodiments, β-alanyl-CoA formed as described above is converted to acryloyl-CoA and NH₃ by a β-alanyl-CoA:ammonia lyase classified, for example, under EC 4.3.1.6, such as the gene product of alt from Clostridium propionicum; followed by conversion of acryloyl-CoA and NADPH to NADP⁺ and propanoyl-CoA by a acrylyl-CoA reductase (NADPH) classified, for example, under EC 1.3.1.84, such as the gene product of acul from E. coli K-12.

In some embodiments, propanoyl-CoA formed as described above is converted to 2-methylcitrate by conversion of propanoyl-CoA and oxaloacetate to 2-methylcitrate by 2-methylcitrate synthase a classified, for example, under EC 2.3.3.5, such as the gene product of prpC from E. coli K-12.

In some embodiments, oxaloacetate converted along with propanoyl-CoA to 2-methylcitrate by a 2-methylcitrate synthase classified, for example, under EC 2.3.3.5, such as the gene product of prpC from E. coli K-12, is formed by conversion of 2-methylcitrate to 2-methyl-cis-aconitate and H₂O by a 2-methylcitrate dehydratase classified, for example, under EC 4.2.1.79, such as the gene product of prpD from E. coli K-12; followed by conversion of 2-methyl-cis-aconitate and H₂O to 2-methyl-isocitrate by a 2-methylisocitrate dehydratase classified, for example, under EC 4.2.1.99 such as the gene product of acnB from E. coli K-12; followed by conversion of 2-methyl-isocitrate to pyruvate and succinate by a methylisocitrate lyase classified, for example, under EC 4.1.3.30, such as the gene product of prpB from E. coli K-12; followed by conversion of succinate and H₂O to fumarate by a succinate dehydrogenase (quinone) classified, for example, under EC 1.3.5.1, such as the gene product of sdhA/B from E. coli K-12, or a fumarate reductase (quinol) classified, for example, under EC 1.3.5.4, such as the gene product of frdA from E. coli K-12; followed by conversion of fumarate to malate and H₂O by a fumarate hydratase classified, for example, under EC 4.2.1.2, such as the gene product of fumA/C from E. coli K-12; followed by conversion of malate and NAD⁺ to oxaloacetate, NADH, and H⁺ by a malate dehydrogenase classified, for example, under EC 1.1.1.37, such as the gene product of mdh from E. coli K-12.

### Formate Synthesis Via acetyl-CoA carboxylase, 3-hydroxypropanoate, and Methylcitrate Cycle

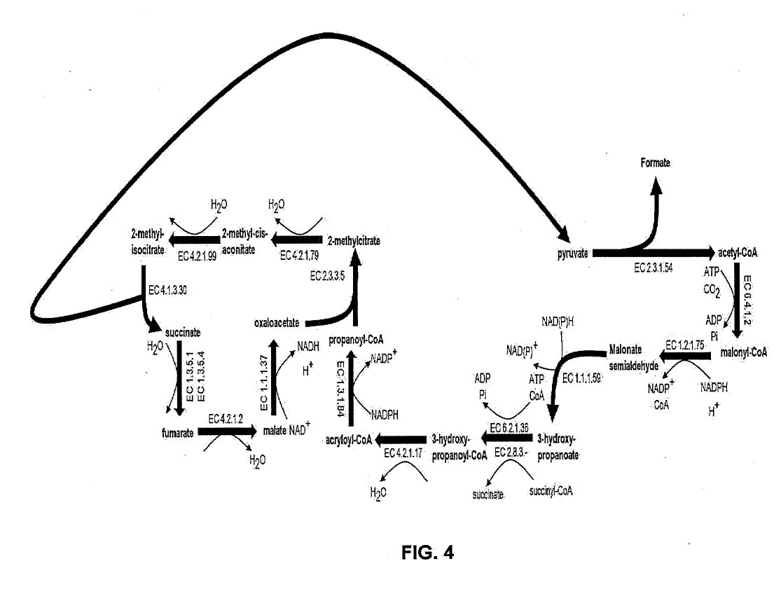

In one aspect, formate is synthesized using an acetyl-CoA carboxylase, 3-hydroxypropanoate, and the methylcitrate cycle. See, e.g., FIG. 4.

In some embodiments, formate and acetyl-CoA may be synthesized from 2-methyl-isocitrate by conversion of 2-methyl-isocitrate to pyruvate and succinate by a methylisocitrate lyase classified, for example, under EC 4.1.3.30, such as the gene product of prpB from E. coli K-12; followed by conversion of pyruvate to formate and acetyl-CoA by a formate C-acetyltransferase classified, for example, under EC 2.3.1.54, such as the gene product of pfl from Clostridium butyricum.

In some embodiments, acetyl-CoA formed as described above, ATP, and CO₂ are converted to malonyl-CoA, ADP, and Pᵢ by an acetyl-CoA carboxylase classified, for example, under EC 6.4.1.2, such as the gene product of accA/C/D from E. coli K-12; followed by conversion of malonyl-CoA formed as described above, NADPH, and H⁺ to malonate semialdehyde, NADP⁺, and CoA by a malonyl-CoA reductase (malonate semialdehyde-forming) classified, for example, under EC 1.2.1.75, such as the gene product of mcr from Sulfolobus tokodaii; followed by conversion of malonate semialdehyde and NADPH to NADP⁺ and 3-hydroxypropanoate by a 3-hydroxypropionate dehydrogenase classified, for example, under EC 1.1.1.59 or EC 1.1.1.298, such as the gene product of uxuB from E. coli K-12 or the gene product of ydfG from E. coli K-12.

In some embodiments, 3-hydroxypropanoate formed as described above, ATP, CoA, and succinyl-CoA are converted to succinate, 3-hydroxy-propanoyl-CoA, ADP, and Pᵢ by a CoA-transferase classified, for example, under EC 2.8.3.-, such as the gene product of frc from E. coli K-12, and a 3-hydroxypropionyl-CoA synthase classified, for example, under EC 6.2.1.36, such as the gene product of Msed_1456 from M. sedula ATCC 51363. In some embodiments, 3-hydroxy-propanoyl-CoA formed as described above is converted to acryloyl-CoA and H₂O by an enoyl-CoA hydratase classified, for example, under EC 4.2.1.17, such as the gene product of fadB from E. coli K-12; followed by conversion of acryloyl-CoA and NADPH to NADP⁺ and propanoyl-CoA by an acrylyl-CoA reductase (NADPH) classified, for example, under EC 1.3.1.84, such as the gene product of acul from E. coli K-12; followed by conversion of propanoyl-CoA and oxaloacetate to 2-methylcitrate by 2-methylcitrate synthase a classified, for example, under EC 2.3.3.5, such as the gene product of prpC from E. coli K-12.

In some embodiments, oxaloacetate converted along with propanoyl-CoA to 2-methylcitrate by a 2-methylcitrate synthase classified, for example, under EC 2.3.3.5, such as the gene product of prpC from E. coli K-12, is formed by conversion of 2-methylcitrate to 2-methyl-cis-aconitate and H₂O by a 2-methylcitrate dehydratase classified, for example, under EC 4.2.1.79, such as the gene product of prpD from E. coli K-12; followed by conversion of 2-methyl-cis-aconitate and H₂O to 2-methyl-isocitrate by a 2-methylisocitrate dehydratase classified, for example, under EC 4.2.1.99 such as the gene product of acnB from E. coli K-12; followed by conversion of 2-methyl-isocitrate to pyruvate and succinate by a methylisocitrate lyase classified, for example, under EC 4.1.3.30, such as the gene product of prpB from E. coli K-12; followed by conversion of succinate and H₂O to fumarate by a succinate dehydrogenase (quinone) classified, for example, under EC 1.3.5.1, such as the gene product of sdhA/B from E. coli K-12, or a fumarate reductase (quinol) classified, for example, under EC 1.3.5.4, such as the gene product of frdA from E. coli K-12; followed by conversion of fumarate to malate and H₂O by a fumarate hydratase classified, for example, under EC 4.2.1.2, such as the gene product of fumA/C from E. coli K-12; followed by conversion of malate and NAD⁺ to oxaloacetate, NADH, and H⁺ by a malate dehydrogenase classified, for example, under EC 1.1.1.37, such as the gene product of mdh from E. coli K-12.

### Formate Synthesis Via Acetyl-CoA Carboxylase and Lactate

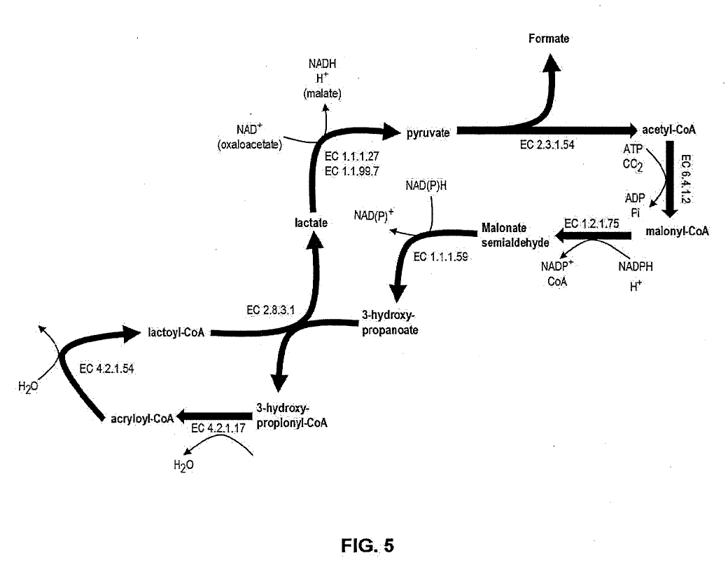

In one aspect, formate is synthesized using acetyl-CoA carboxylase and lactate. See, e.g., FIG. 5.

In some embodiments, formate and acetyl-CoA may be synthesized from lactate by conversion of lactate, NAD⁺, and oxaloacetate to pyruvate, NADH, H⁺, and malate by a L-lactate dehydrogenase classified, for example, under EC 1.1.1.27, such as the gene product of ldh from B. subtilis, and a lactate-malate transhydrogenase classified, for example, under EC 1.1.99.7, such as lactate-malate transhydrogenase from Veillonella parvula; followed by conversion of pyruvate to formate and acetyl-CoA by a formate C-acetyltransferase classified, for example, under EC 2.3.1.54, such as the gene product of pfl from Clostridium butyricum.

In some embodiments, lactate used in the synthesis of formate as described above may be synthesized from acetyl-CoA formed as described above, ATP, and CO₂ are converted to malonyl-CoA, ADP, and Pᵢ by an acetyl-CoA carboxylase classified, for example, under EC 6.4.1.2, such as the gene product of accA/C/D from E. coli K-12; followed by conversion of malonyl-CoA formed as described above, NADPH, and H⁺ to malonate semialdehyde, NADP⁺, and CoA by a malonyl-CoA reductase (malonate semialdehyde-forming) classified, for example, under EC 1.2.1.75, such as the gene product of mcr from Sulfolobus tokodaii; followed by conversion of malonate semialdehyde and NADPH to NADP⁺ and 3-hydroxypropanoate by a 3-hydroxypropionate dehydrogenase classified, for example, under EC 1.1.1.59 or EC 1.1.1.298, such as the gene product of uxuB from E. coli K-12 or the gene product of ydfG from E. coli K-12; followed by conversion of 3-hydroxypropanoate and lactoyl-CoA to lactate and 3-hydroxy-propionyl-CoA by a propionate CoA-transferase classified, for example, under EC 2.8.3.1, such as the gene product of pct from C. propionicum.

In some embodiments, lactoyl-CoA used in the synthesis of lactate as described above may be synthesized by the conversion of 3-hydroxy-propionyl-CoA formed as described above to acryloyl-CoA and H₂O by an enoyl-CoA hydratase classified, for example, under EC 4.2.1.17, such as the gene product of fadB from E. coli K-12; followed by conversion of acryloyl-CoA and H₂O to lactoyl-CoA by a lactoyl-CoA dehydratase classified, for example, under EC 4.2.1.54, such as the gene product of IcdA/B from C. propionicum.

### Formate Synthesis Via Homoserine and Methylcitrate Cycle

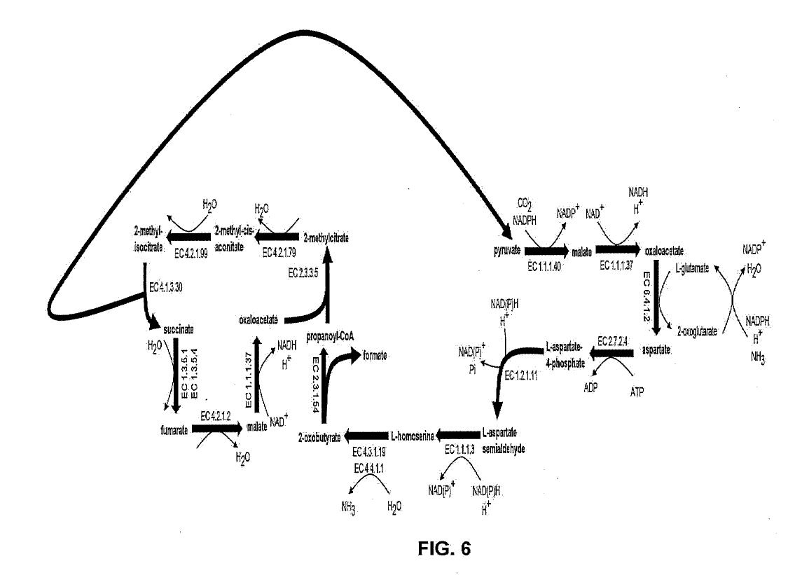

In one aspect, formate is synthesized using a homoserine and the methylcitrate cycle. See, e.g., FIG. 6.

In some embodiments, formate may be synthesized from L-homoserine by conversion of L-homoserine and H₂O to 2-oxobutyrate and NH₃ by a threonine ammonia-lyase classified, for example, under EC 4.3.1.19, such as the gene product of tdcB from E. coli K-12, and a cystathionine γ-lyase classified, for example, under EC 4.4.1.1, such as the gene product of mccB from B. subtilis; followed by the conversion of 2-oxobutyrate to formate and propanoyl-CoA by a formate C-acetyltransferase classified, for example, under EC 2.3.1.54, such as the gene product of pfl from Clostridium butyricum.

In some embodiments, propanoyl-CoA produced as described above and oxaloacetate are converted by 2-methylcitrate by a 2-methylcitrate synthase classified, for example, under EC 2.3.3.5, such as the gene product of prpC from E. coli K-12.

In some embodiments, oxaloacetate converted along with propanoyl-CoA to 2-methylcitrate by a 2-methylcitrate synthase classified, for example, under EC 2.3.3.5, such as the gene product of prpC from E. coli K-12, is formed by conversion of 2-methylcitrate to 2-methyl-cis-aconitate and H₂O by a 2-methylcitrate dehydratase classified, for example, under EC 4.2.1.79, such as the gene product of prpD from E. coli K-12; followed by conversion of 2-methyl-cis-aconitate and H₂O to 2-methyl-isocitrate by a 2-methylisocitrate dehydratase classified, for example, under EC 4.2.1.99 such as the gene product of acnB from E. coli K-12; followed by conversion of 2-methyl-isocitrate to pyruvate and succinate by a methylisocitrate lyase classified, for example, under EC 4.1.3.30, such as the gene product of prpB from E. coli K-12; followed by conversion of succinate and H₂O to fumarate by a succinate dehydrogenase (quinone) classified, for example, under EC 1.3.5.1, such as the gene product of sdhA/B from E. coli K-12, or a fumarate reductase (quinol) classified, for example, under EC 1.3.5.4, such as the gene product of frdA from E. coli K-12; followed by conversion of fumarate to malate and H₂O by a fumarate hydratase classified, for example, under EC 4.2.1.2, such as the gene product of fumA/C from E. coli K-12; followed by conversion of malate and NAD⁺ to oxaloacetate, NADH, and H⁺ by a malate dehydrogenase classified, for example, under EC 1.1.1.37, such as the gene product of mdh from E. coli K-12.

In some embodiments, pyruvate formed as described above, 00₂, and NADPH are converted to malate and NAD⁺ by a malate dehydrogenase (oxaloacetate-decarboxylating) classified, for example, under EC 1.1.1.40, such as the gene product of maeB from E. coli K-12; followed by conversion of malate and NAD⁺ to oxaloacetate, NADH, and H⁺ by a malate dehydrogenase classified, for example, under EC 1.1.1.37, such as the gene product of mdh from E. coli K-12; followed by conversion of oxaloacetate and L-glutamate to aspartate and 2-oxoglutarate by an acetyl-CoA carboxylase classified, for example, under EC 6.4.1.2, such as the gene product of accA/C/D from E. coli K-12; followed by conversion of aspartate and ATP to ADP and L-aspartate-4-phosphate by an aspartate kinase classified, for example, under EC 2.7.2.4, such as the gene product of lysC from E. coli K-12; followed by the conversion of L-aspartate-4-phosphate, NADPH, and H⁺ to NADP⁺, Pᵢ, and L-aspartate by an aspartate-semialdehyde dehydrogenase classified, for example, under EC 1.2.1.11, such as the gene product of asd from E. coli K-12; followed by conversion of NADPH, H⁺, and L-aspartate semialdehyde to NADP+ and L-homoserine by a malate dehydrogenase classified, for example, by EC 1.1.1.3, such as the gene product of mdh from E. coli K-12.

In some embodiments, L-homoserine produced as described above is used to synthesize formate as described above.

In some embodiments, 2-oxoglutarate produced as described above is recycled to L-glutamate by conversion of 2-oxoglutarate, NADPH, H⁺, and NH₃ to H₂O, NADP⁺, and L-glutamate.

### Acetyl-CoA Synthesis Via Formate and a Modified Serine Cycle

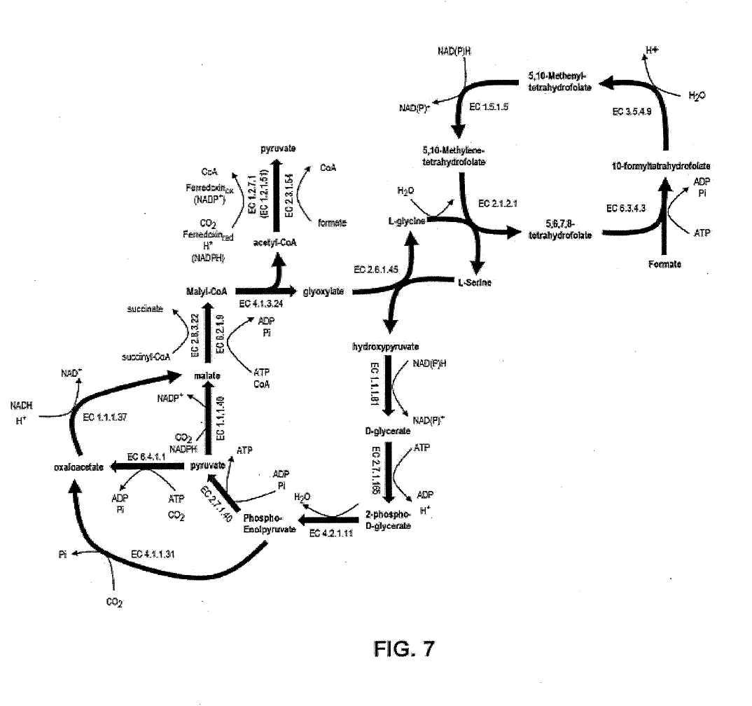

In one aspect, acetyl-CoA is synthesized using a formate and a modified serine cycle. See, e.g., FIG. 7.

In some embodiments, L-serine may be synthesized from formate by conversion of formate, 5,6,7,8-tetrahydrofolate, and ATP to ADP, Pᵢ, and 10-formyletetrahydrofolate by a formate-tetrahydrofolate ligase classified, for example, under EC 6.3.4.3, such as the gene product of fhs from C. cylindrosporum; followed by conversion of 10-formyltetrahydrofolate and H₂O to H⁺ and 5,10-methenyl-tetrahydrofolate by a methenyltetrahydrofolate cyclohydrolase classified, for example, under EC 3.5.4.9, such as the gene product of folD from M. extorquens; followed by conversion of 5,10-methenyl-tetrahydrofolate and NADPH to NADP⁺ and 5,10-methylene-tetrahydrofolate by a methylenetetrahydrofolate dehydrogenase (NADP⁺) classified, for example, under EC 1.5.1.5, such as the gene product of folD from M. extorquens; followed by conversion of L-glycine, H₂O, and 5-methylene-tetrahydrofolate to L-serine and 5,6,7,8-tetrahydrofolate by a glycine hydroxymethyltransferase classified, for example, under EC 2.1.2.1, such as the gene product of glyA from E. coli K-12. In some embodiments, 5,6,7,8-tetrahydrofolate is converted to formate by a formate-tetrahydrofolate ligase classified, for example, under EC 6.3.4.3, such as the gene product of fhs from C. cylindrosporum.

In some embodiments, L-serine formed as described above and glyoxylate are converted to L-glycine and hydroxypyruvate by a serine-glyoxylate aminotransferase classified, for example, under EC 2.6.1.45, such as the gene product of sgaA from H. methylovorum. In some embodiments, the L-glycine produced by the conversion of L-serine and glyoxylate is used to produce L-serine as described above.

In some embodiments, hydroxypyruvate formed as described above and NADPH are converted to NADP⁺ and D-glycerate by a hydroxypyruvate reductase classified, for example, under EC 1.1.1.81, such as the gene product of hprA from M. extorquens or by a glycerate dehydrogenase classified, for example, under EC 1.1.1.29, such as the gene product of hprA from M. extorquens; followed by conversion of D-glycerate and ATP to ADP, H⁺, and 2-phospho-D-glycerate by a glycerate 2-kinase classified, for example, under EC 2.7.1.165, such as the gene product of gck from H. methylovorum; followed by conversion of 2-phospho-D-glycerate to phosphoenolpyruvate and H₂O by a phosphopyruvate hydratase classified, for example, under EC 4.2.1.11, such as the gene product of eno from E. coli K-12; followed by the conversion of phospho-enolpyruvate, ADP, and Pᵢ to pyruvate by a pyruvate kinase classified, for example, under EC 2.7.1.40, such as the gene product of pyk1 from S. cerevisiae.

In some embodiments, pyruvate formed as described above is converted to malate by conversion of pyruvate, NADPH, and CO₂ to malate and NAD⁺ by a malate dehydrogenase (oxaloacetate-decarboxylating) classified, for example, under EC 1.1.1.40, such as the gene product of maeB from E. coli K-12.

In some embodiments, oxaloacetate is formed from pyruvate formed as described above by conversion of CO₂, ATP, and pyruvate to ADP, Pᵢ, and oxaloacetate by a pyruvate carboxylase classified, for example, under EC 6.4.1.1, such as the gene product of pyc1/2 from S. cerevisiae.

In some embodiments, phosphoenolpyruvate formed as described above and CO₂ are converted to oxaloacetate and Pᵢ by a malate dehydrogenase (oxaloacetate-decarboxylating) classified, for example, under EC 4.1.1.31, such as the gene product of maeB from E. coli K-12.

In some embodiments, oxaloacetate produced as described above is converted to malate by conversion of oxaloacetate, NADH, and H⁺ to NAD⁺ and malate by a malate dehydrogenase classified, for example, under EC 1.1.1.37, such as the gene product of mdh from E. coli K-12.

In some embodiments, malate produced as described above is converted to malyl-CoA by conversion of succinyl-CoA, succinate, ATP, and CoA to ADP, Pᵢ, succinate, and malyl-CoA by a succinyl-CoA-L-malate CoA-transferase classified, for example, under EC 2.8.3.22, such as the gene product of smtA/B from C. aurantiacus, and a malate-CoA ligase classified, for example, under EC 6.2.1.9, such as the gene product of mtkA/B from M. extorquens; followed by conversion of malyl-CoA to glyoxylate and acetyl-CoA by a malyl-CoA lyase classified, for example, under EC 4.1.3.24, such as the gene product of mcl1 from R. capsulatus; followed by conversion of acetyl-CoA, CO2, ferredoxin_{red}, NADPH, and formate to 2 CoA, ferredoxinₒₓ, NADP⁺, and pyruvate by a pyruvate synthase classified, for example, under EC 1.2.7.1, such as the gene product of por from D. africanus, and a formate C-acetyltransferase classified, for example, under EC 2.3.1.54, such as the gene product of pfl from C. butyricum.

In some embodiments, glyoxylate formed as described above is used in the synthesis of L-serine as described above.

### Acetyl-CoA Synthesis Via Formate and a Combination of the Reductive TCA Cycle and Glyoxylate Degradation

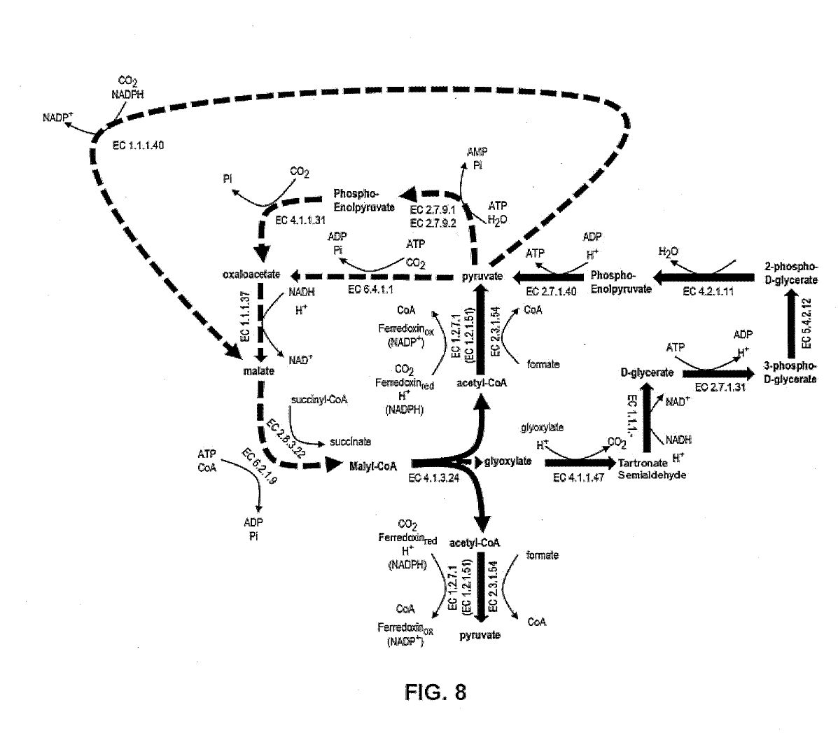

In one aspect, acetyl-CoA is synthesized using formate and a combination of the reductive TCA cycle and glyoxylate degradation. See, e.g., FIG. 8.

In some embodiments, oxaloacetate is formed from pyruvate by conversion of CO₂, ATP, and pyruvate to ADP, Pᵢ, and oxaloacetate by a pyruvate carboxylase classified, for example, under EC 6.4.1.1, such as the gene product of pyc1/2 from S. cerevisiae. In some embodiments, this step is repeated more than one time (e.g., at least two times).

In some embodiments, malyl-CoA is synthesized from pyruvate by the conversion of pyruvate, ATP, and H₂O to AMP, Pᵢ, and phosphoenolpyruvate by a pyruvate, phosphate dikinase classified, for example, under EC 2.7.9.1, such as the gene product of ppdK from C. symbiosum and a pyruvate, water dikinase classified, for example, under EC 2.7.9.2, such as the gene product of ppsA from E. coli K-12. In some embodiments, this step is repeated more than one time (e.g., at least two times).

In some embodiments, phosphoenolpyruvate formed as described above and CO₂ are converted to oxaloacetate and Pᵢ by a malate dehydrogenase (oxaloacetate-decarboxylating) classified, for example, under EC 4.1.1.31, such as the gene product of maeB from E. coli K-12. In some embodiments, this step is repeated more than one time (e.g., at least two times).

In some embodiments, oxaloacetate produced as described above is converted to malate by conversion of oxaloacetate, NADH, and H⁺ to NAD⁺ and malate by a malate dehydrogenase classified, for example, under EC 1.1.1.37, such as the gene product of mdh from E. coli K-12. In some embodiments, this step is repeated more than one time (e.g., at least two times).

In some embodiments, malate is synthesized from pyruvate by conversion of pyruvate, CO₂, and NAPDH to malate and NADP+ by a malate dehydrogenase (oxaloacetate-decarboxylating) classified, for example, under EC 1.1.1.40, such as the gene product of maeB from E. coli K-12. In some embodiments, this step is repeated more than one time (e.g., at least two times).

In some embodiments, malate produced as described above is converted to malyl-CoA by conversion of succinyl-CoA, succinate, ATP, and CoA to ADP, Pᵢ, succinate, and malyl-CoA by a succinyl-CoA-L-malate CoA-transferase classified, for example, under EC 2.8.3.22, such as the gene product of smtA/B from C. aurantiacus, and a malate-CoA ligase classified, for example, under EC 6.2.1.9, such as the gene product of mtkA/B from M. extorquens; followed by conversion of malyl-CoA to glyoxylate and acetyl-CoA by a malyl-CoA lyase classified, for example, under EC 4.1.3.24, such as the gene product of mcl1 from R. capsulatus. In some embodiments, these steps are repeated more than one time (e.g., at least two times).

In some embodiments, pyruvate is synthesized from acetyl-CoA produced as described above by conversion of acetyl-CoA, CO₂, ferredoxin_{red}, NADPH, and formate to 2 CoA, ferredoxinₒₓ, NADP⁺, and pyruvate by a pyruvate synthase classified, for example, under EC 1.2.7.1, such as the gene product of por from D. africanus, and a formate C-acetyltransferase classified, for example, under EC 2.3.1.54, such as the gene product of pfl from C. butyricum. In some embodiments, pyruvate synthesized from acetyl-CoA is used to produce oxaloacetate, malate, or phosphoenolpyruvate as described above.

In some embodiments, glyoxylate formed as described above is used in the synthesis of pyruvate by the conversion of glyoxylate and H⁺ to CO₂ and tartonate semialdehyde by a tartronate-semialdehyde synthase classified, for example, under EC 4.1.1.47, such as the gene product of gcl from E. coli K-12; followed by the conversion of tartonate semialdehyde, NADH, and H⁺ to NAD⁺ and D-glycerate by an oxidoreductase with NAD(+) or NADP(+) as acceptor classified, for example, under EC 1.1.1.-, such as the gene product of garR from E. coli K-12; followed by conversion of D-glycerate and ATP to ADP, H⁺, and 3-phopho-D-glycerate by a glycerate 3-kinase classified, for example, under EC 2.7.1.31, such as the gene product of glxK from E. coli K-12; followed by conversion of 3-phospho-D-glycerate to 2-phospho-D-glycerate by a phosphoglycerate mutase (2,3-diphosphoglycerate-independent) classified, for example, under EC 5.4.2.12, such as the gene product of gmpl from E. coli K-12; followed by conversion of 2-phospho-D-glycerate to phosphoenolpyruvate and H₂O; followed by conversion of phosphoenolpyruvate, ADP, and H⁺ to pyruvate and ATP by a pyruvate kinase classified, for example, under EC 2.7.1.40, such as the gene product of pykAIF from E. coli K-12. In some embodiments, pyruvate synthesized from glyoxylate is used to produce oxaloacetate, malate, or phosphoenolpyruvate as described above.

### Acetyl-CoA Synthesis Via Formate and a Combination of the Reductive TCA Cycle and the Serine Cycle

In one aspect, acetyl-CoA is synthesized using formate and a combination of the reductive TCA cycle and the serine cycle. See, e.g., FIG. 9.

In some embodiments, oxaloacetate is formed from pyruvate by conversion of CO₂, ATP, and pyruvate to ADP, Pᵢ, and oxaloacetate by a pyruvate carboxylase classified, for example, under EC 6.4.1.1, such as the gene product of pyc1/2 from S. cerevisiae. In some embodiments, this step is repeated more than one time (e.g., at least two times).

In some embodiments, malyl-CoA is synthesized from pyruvate by the conversion of pyruvate, ATP, and H₂O to AMP, Pᵢ, and phosphoenolpyruvate by a pyruvate, phosphate dikinase classified, for example, under EC 2.7.9.1, such as the gene product of ppdK from C. symbiosum and a pyruvate, water dikinase classified, for example, under EC 2.7.9.2, such as the gene product of ppsA from E. coli K-12. In some embodiments, this step is repeated more than one time (e.g., at least two times).

In some embodiments, phosphoenolpyruvate formed as described above and CO₂ are converted to oxaloacetate and Pᵢ by a malate dehydrogenase (oxaloacetate-decarboxylating) classified, for example, under EC 4.1.1.31, such as the gene product of maeB from E. coli K-12. In some embodiments, this step is repeated more than one time (e.g., at least two times).

In some embodiments, oxaloacetate produced as described above is converted to malate by conversion of oxaloacetate, NADH, and H⁺ to NAD⁺ and malate by a malate dehydrogenase classified, for example, under EC 1.1.1.37, such as the gene product of mdh from E. coli K-12. In some embodiments, this step is repeated more than one time (e.g., at least two times).

In some embodiments, malate is synthesized from pyruvate by conversion of pyruvate, CO₂, and NAPDH to malate and NADP+ by a malate dehydrogenase (oxaloacetate-decarboxylating) classified, for example, under EC 1.1.1.40, such as the gene product of maeB from E. coli K-12. In some embodiments, this step is repeated more than one time (e.g., at least two times).

In some embodiments, malate produced as described above is converted to malyl-CoA by conversion of succinyl-CoA, succinate, ATP, and CoA to ADP, Pᵢ, succinate, and malyl-CoA by a succinyl-CoA-L-malate CoA-transferase classified, for example, under EC 2.8.3.22, such as the gene product of smtA/B from C. aurantiacus, and a malate-CoA ligase classified, for example, under EC 6.2.1.9, such as the gene product of mtkA/B from M. extorquens; followed by conversion of malyl-CoA to glyoxylate and acetyl-CoA by a malyl-CoA lyase classified, for example, under EC 4.1.3.24, such as the gene product of mcl1 from R. capsulatus. In some embodiments, these steps are repeated more than one time (e.g., at least two times).

In some embodiments, pyruvate is synthesized from acetyl-CoA produced as described above by conversion of acetyl-CoA, CO₂, ferredoxin_{red}, NADPH, and formate to 2 CoA, ferredoxinₒₓ, NADP⁺, and pyruvate by a pyruvate synthase classified, for example, under EC 1.2.7.1, such as the gene product of por from D. africanus, and a formate C-acetyltransferase classified, for example, under EC 2.3.1.54, such as the gene product of pfl from C. butyricum. In some embodiments, pyruvate synthesized from acetyl-CoA is used to produce oxaloacetate, malate, or phosphoenolpyruvate as described above.

In some embodiments, L-serine may be synthesized from formate by conversion of formate, 5,6,7,8-tetrahydrofolate, and ATP to ADP, Pᵢ, and 10-formyletetrahydrofolate by a formate-tetrahydrofolate ligase classified, for example, under EC 6.3.4.3, such as the gene product of fhs from C. cylindrosporum; followed by conversion of 10-formyltetrahydrofolate and H₂O to H⁺ and 5,10-methenyl-tetrahydrofolate by a methenyltetrahydrofolate cyclohydrolase classified, for example, under EC 3.5.4.9, such as the gene product of folD from M. extorquens; followed by conversion of 5,10-methenyl-tetrahydrofolate and NADPH to NADP⁺ and 5,10-methylene-tetrahydrofolate by a methylenetetrahydrofolate dehydrogenase (NADP⁺) classified, for example, under EC 1.5.1.5, such as the gene product of folD from M. extorquens; followed by conversion of L-glycine, H₂O, and 5-methylene-tetrahydrofolate to L-serine and 5,6,7,8-tetrahydrofolate by a glycine hydroxymethyltransferase classified, for example, under EC 2.1.2.1, such as the gene product of glyA from E. coli K-12. In some embodiments, 5,6,7,8-tetrahydrofolate is converted to formate by a formate-tetrahydrofolate ligase classified, for example, under EC 6.3.4.3, such as the gene product of fhs from C. cylindrosporum.

In some embodiments, L-serine formed as described above and glyoxylate formed as described above are used in the synthesis of pyruvate by the conversion of L-serine and glyoxylate to L-glycine and hydroxypyruvate by a serine-glyoxylate aminotransferase classified, for example, under EC 2.6.1.45, such as the gene product of sgaA from H. methylovorum; followed by conversion of hydroxypyruvate and NADPH to NADP⁺ and D-glycerate by a hydroxypyruvate reductase classified, for example, under EC 1.1.1.81, such as the gene product of hprA from M. extorquens or by a glycerate dehydrogenase classified, for example, under EC 1.1.1.29; followed by conversion of D-glycerate and ATP to ADP, H⁺, and 2-phospho-D-glycerate by a glycerate 2-kinase classified, for example, under EC 2.7.1.165, such as the gene product of gck from H. methylovorum; followed by conversion of 2-phospho-D-glycerate to phosphoenolpyruvate and H₂O by a phosphopyruvate hydratase classified, for example, under EC 4.2.1.11, such as the gene product of eno from E. coli K-12; followed by the conversion of phospho-enolpyruvate, ADP, and Pᵢ to pyruvate by a pyruvate kinase classified, for example, under EC 2.7.1.40, such as the gene product of pyk1 from S. cerevisiae.

In some embodiments, pyruvate formed as described above is used in the synthesis of phophoenolpyruvate or oxaloacetate as described above.

In some embodiments, the L-glycine produced by the conversion of L-serine and glyoxylate is used to produce L-serine as described above.

### Acetyl-CoA Synthesis Via Formate and a Combination of the Reductive TCA Cycle and the Serine Cycle

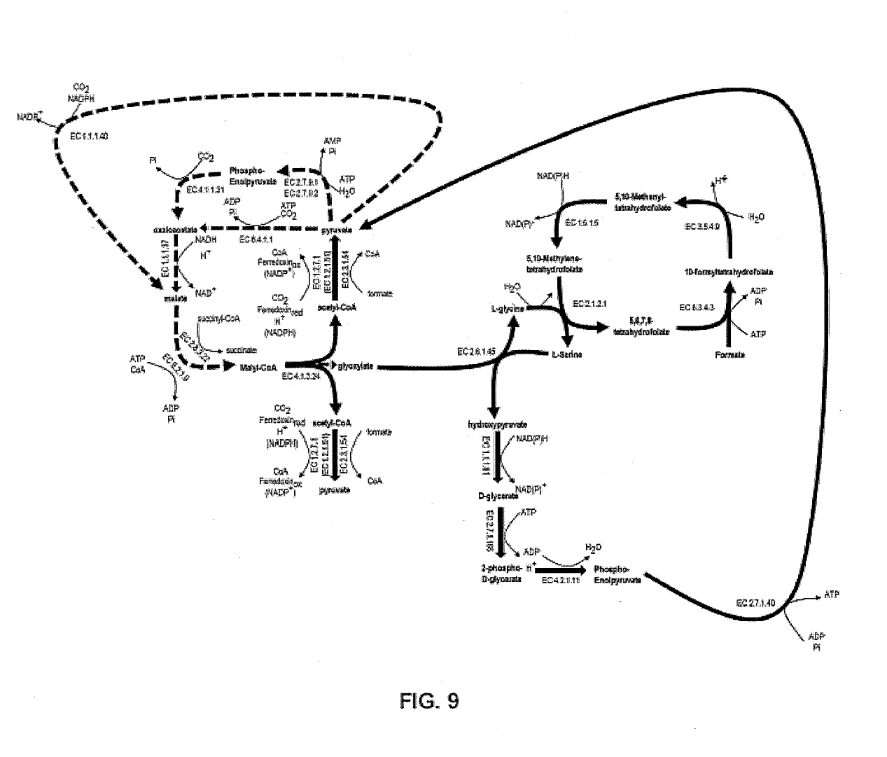

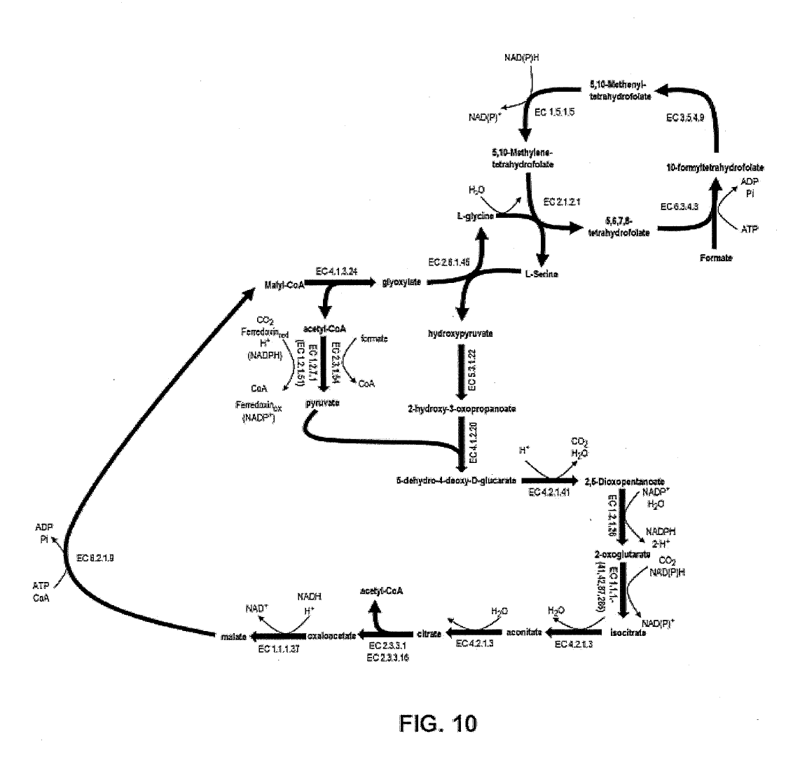

In one aspect, acetyl-CoA is synthesized using formate and a combination of the reductive TCA cycle and the serine cycle. See, e.g., FIG. 10.

In some embodiments, L-serine may be synthesized from formate by conversion of formate, 5,6,7,8-tetrahydrofolate, and ATP to ADP, Pᵢ, and 10-formyletetrahydrofolate by a formate-tetrahydrofolate ligase classified, for example, under EC 6.3.4.3, such as the gene product of fhs from C. cylindrosporum; followed by conversion of 10-formyltetrahydrofolate and H₂O to H⁺ and 5,10-methenyl-tetrahydrofolate by a methenyltetrahydrofolate cyclohydrolase classified, for example, under EC 3.5.4.9, such as the gene product of folD from M. extorquens; followed by conversion of 5,10-methenyl-tetrahydrofolate and NADPH to NADP⁺ and 5,10-methylene-tetrahydrofolate by a methylenetetrahydrofolate dehydrogenase (NADP⁺) classified, for example, under EC 1.5.1.5, such as the gene product of folD from M. extorquens; followed by conversion of L-glycine, H₂O, and 5-methylene-tetrahydrofolate to L-serine and 5,6,7,8-tetrahydrofolate by a glycine hydroxymethyltransferase classified, for example, under EC 2.1.2.1, such as the gene product of glyA from E. coli K-12. In some embodiments, 5,6,7,8-tetrahydrofolate is converted to formate by a formate-tetrahydrofolate ligase classified, for example, under EC 6.3.4.3, such as the gene product of fhs from C. cylindrosporum.

In some embodiments, L-serine formed as described above may be used to synthesize 2-hydroxy-3-oxopropanoate by the conversion of L-serine and glyoxylate to hydroxypyruvate and L-glycine by a serine-glyoxylate transaminase classified, for example, under EC 2.6.1.45, such as the gene product of sgaA from H. methylovorum; followed by the conversion of hydroxypyruvate to 2-hydroxy-3-oxopropanoate by a hydroxypyruvate isomerase classified, for example, under EC 5.3.1.22, such as the gene product of hyi from E. coli K-12.

In some embodiments, 2-hydroxy-3-oxopropanoate formed as described above may be used to synthesize 2-oxoglutarate by conversion of 2-hydroxy-3-oxopropanoate and pyruvate to 5-dehydro-4-deoxy-D-glucarate by a 2-dehydro-3-deoxyglucarate aldolase classified, for example, under EC 4.1.2.20, such as the gene product of garL from E. coli K-12; followed by conversion of 5-dehydro-4-deoxy-D-glucarate and H⁺ to CO₂, H₂O, and 2,5-dioxopentanoate by a 5-dehydro-4-deoxyglucarate dehydratase classified, for example, under EC 4.2.1.41, such as the gene product of ybcC from B. subtilis; followed by conversion of 2,5-dioxopentanoate, NADP⁺, and H₂O to NADP, 2H⁺, and 2-oxoglutarate by a 2,5-dioxovalerate dehydrogenase classified, for example, under EC 1.2.1.26, such as the gene product of araE from A. brasilense.

In some embodiments, 2-oxoglutarate formed as described above may be used to synthesize malyl-CoA by conversion of 2-oxoglutarate, CO₂, and NADPH to NADP⁺ and isocitrate by an oxidoreductase with NAD(+) or NADP(+) as acceptor classified, for example, under EC 1.1.1.-, such as the gene product of garR from E. coli K-12; followed by conversion of isocitrate to aconitate and H₂O by an aconitate hydratase classified, for example, under EC 4.2.1.3, such as the gene product of acnB from E. coli K-12 (e.g., RefSeq Accession No. NP_414660.1); followed by conversion of H₂O and aconitate to citrate by an aconitate hydratase classified, for example, under EC 4.2.1.3; followed by conversion of citrate to acetyl-CoA and oxaloacetate by a citrate (Si)-synthase classified, for example, under EC 2.3.3.1, such as the gene product of gltA from E. coli K-12 (e.g., RefSeq Accession No. NP_415248.1), and a citrate synthase classified, for example, under EC 2.3.3.16, such as the gene product of CIT1 from S. cerevisiae S288c (e.g., RefSeq Accession No. NP_014398.1); followed by conversion of oxaloacetate, NADH, and H^{.+} to malate and NAD⁺ by a malate dehydrogenase classified, for example, under EC 1.1.1.37, such as the gene product of mdh from E. coli K-12; followed by conversion of malate, ATP, and COA to ADP, Pᵢ, and malyl-CoA by a malate-CoA ligase classified, for example, under EC 6.2.1.9, such as the gene product of mtkA/B from M. extorquens.

In some embodiments, pyruvate used in the synthesis of 5-dehydro-4-deoxy-D-glucarate is formed by the conversion of malyl-CoA formed as described above by conversion of malyl-CoA to glyoxylate by a malyl-CoA lyase classified, for example, under EC 4.1.3.24, such as the gene product of mcl1 from R. capsulatus; followed by conversion of acetyl-CoA, CO₂, ferredoxin_{red}, NADPH, and formate to 2 CoA, ferredoxinₒₓ, NADP⁺, and pyruvate by a pyruvate synthase classified, for example, under EC 1.2.7.1, such as the gene product of por from D. africanus, and a formate C-acetyltransferase classified, for example, under EC 2.3.1.54, such as the gene product of pfl from C. butyricum.

In some embodiments, glyoxylate formed as described above is used in the synthesis of L-glycine and hydroxypyruvate as described above.

### Glycerone Phosphate Synthesis Via Formate and the RUMP Cycle

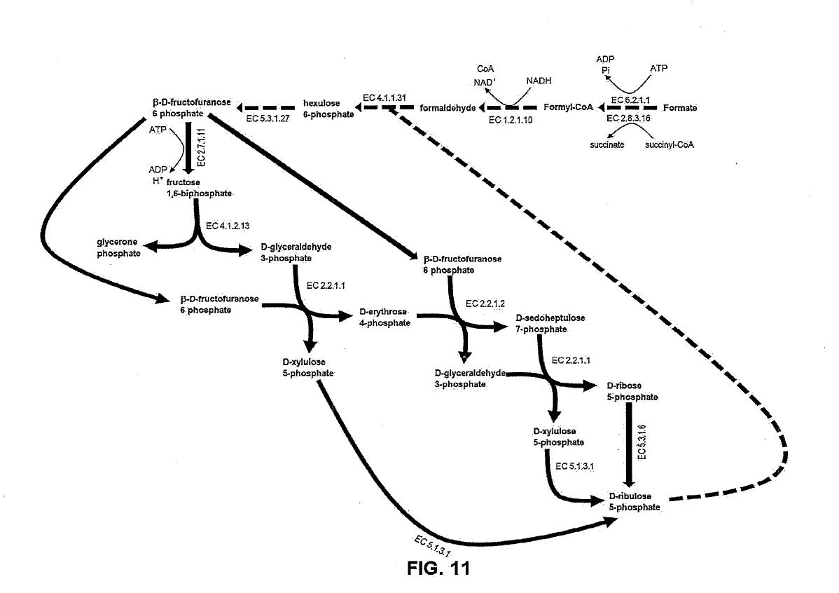

In one aspect, glycerone phosphate is synthesized using formate and the RUMP cycle. See, e.g., FIG. 11.

In some embodiments, hexulose 6-phosphate is synthesized from formate by conversion of formate, succinyl-CoA, and ATP to ADP, Pᵢ, and succinate by an acetate-CoA ligase classified, for example, under EC 6.2.1.1, such as the gene product of acsA from P. aerophilum, and a formyl-CoA transferase classified, for example, under EC 2.8.3.16, such as the gene product of frc from E. coli K-12; followed by conversion of formyl-CoA and NADH to CoA, NAD⁺, and formaldehyde by an aldehyde-alcohol dehydrogenase classified, for example, under EC 1.2.1.10, such as the gene product of frc from E. coli K-12; followed by conversion of formaldehyde and D-ribulose 5-phosphate to hexulose 6-phosphate by a phosphoenolpyruvate carboxylase classified under, for example, EC 4.1.1.31, such as the gene product of ppc from E. coli K-12. In some embodiments, these steps are repeated more than one time (e.g., at least two times).

In some embodiments, hexulose 6-phosphate formed as described above is converted to β-D-fructofuranose 6 phosphate by a 6-phospho-3-hexuloisomerase classified under EC 5.3.1.27, such as the gene product of rmpB from M. aminofaciens. In some embodiments, this step is repeated more than one time (e.g., at least two times).

In some embodiments, β-D-fructofuranose 6 phosphate formed as described above is converted to fructose 1,6-biphosphate by conversion of β-D-fructofuranose 6 phosphate and ATP to ADP, H⁺, and fructose 1,6-biphosphate by a 6-phosphofructokinase classified, for example, under EC 2.7.1.11, such as the gene product of pfkA from E. coli K-12; followed by conversion of fructose 1,6-biphosphate to glycerone phosphate and D-glyceraldehyde 3-phosphate by a fructose-bisphosphate aldolase classified, for example, under EC 4.1.2.13, such as the gene product of cbbAC from C. necator, followed by conversion of D-glyceraldehyde 3-phosphate and β-D-fructofuranose 6 phosphate to D-xylulose 5-phosphate and D-erythrose 4-phosphate by a transketolase classified, for example, under EC 2.2.1.1, such as the gene product of tktA from E. coli K-12; followed by conversion of D-xylulose 5-phosphate to D-ribulose 5-phosphate by a ribulose-phosphate 3-epimerase classified, for example, under EC 5.1.3.1, such as the gene product of rpe1 from S. cerevisiae.

In some embodiments, D-erythrose 4-phosphate formed as described above and β-D-fructofuranose 6 phosphate formed as described are converted to D-glyceraldehyde 3-phosphate and D-sedoheptulose 7-phosphate by a transaldolase classified, for example, under EC 2.2.1.2, such as the gene product of talB from E. coli K-12; followed by conversion of D-glyceraldehyde 3-phosphate and D-sedoheptulose 7-phosphate to D-xylulose 5-phosphate and D-ribose 5-phosphate by a ribose-5-phosphate isomerase classified under EC 5.3.1.6, such as the gene product of rpiA from E. coli K-12; followed by conversion of D-xylolose 5-phosphate to D-ribulose 5-phosphate by a ribulose-phosphate 3-epimerase classified, for example, under EC 5.1.3.1, such as the gene product of rpe1 from S. cerevisiae, and conversion of D-ribose 5-phosphate to D-ribulose 5-phosphate by a ribose-5-phosphate isomerase classified, for example, under EC 5.3.1.6, such as the gene product of rpiA from E. coli K-12.

In some embodiments, D-ribulose 5-phosphate formed as described above is used in the synthesis of hexulose 6-phosphate as described above.

### Acetyl-CoA Synthesis Via Formate and a Modified RUMP Cycle

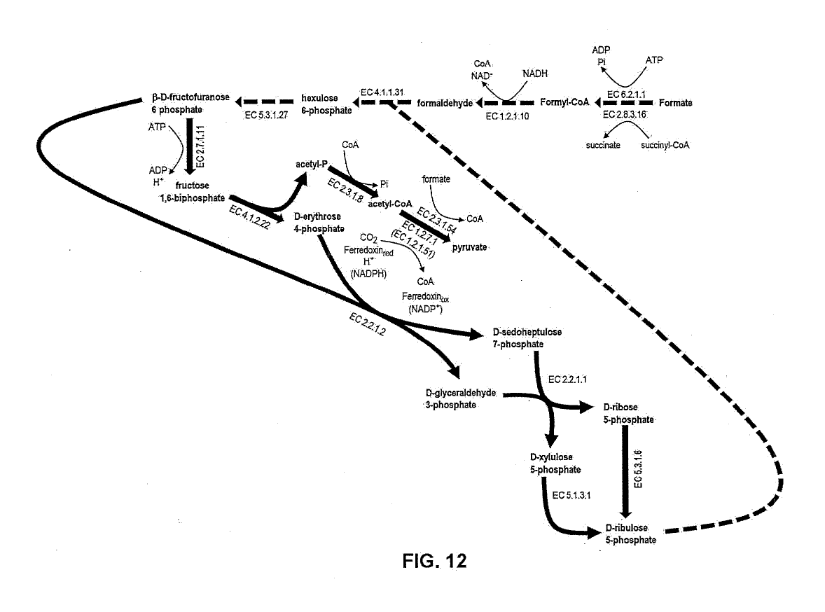

In one aspect, acetyl-CoA is synthesized using formate and a modified RUMP cycle. See, e.g., FIG. 12.

In some embodiments, hexulose 6-phosphate is synthesized from formate by conversion of formate, succinyl-CoA, and ATP to ADP, Pᵢ, and succinate by an acetate-CoA ligase classified, for example, under EC 6.2.1.1, such as the gene product of acsA from P. aerophilum, and a formyl-CoA transferase classified, for example, under EC 2.8.3.16, such as the gene product of frc from E. coli K-12; followed by conversion of formyl-CoA and NADH to CoA, NAD⁺, and formaldehyde by an aldehyde-alcohol dehydrogenase classified, for example, under EC 1.2.1.10, such as the gene product of frc from E. coli K-12; followed by conversion of formaldehyde and D-ribulose 5-phosphate to hexulose 6-phosphate by a phosphoenolpyruvate carboxylase classified, for example, under EC 4.1.1.31, such as the gene product of ppc from E. coli K-12. In some embodiments, these steps are repeated more than one time (e.g., at least two times).

In some embodiments, hexulose 6-phosphate formed as described above is converted to β-D-fructofuranose 6 phosphate by a 6-phospho-3-hexuloisomerase classified, for example, under EC 5.3.1.27, such as the gene product of rmpB from M. aminofaciens. In some embodiments, this step is repeated more than one time (e.g., at least two times).

In some embodiments, β-D-fructofuranose 6 phosphate formed as described above is converted to fructose 1,6-biphosphate by conversion of ATP and β-D-fructofuranose 6 phosphate to ADP, H⁺, and fructose 1,6-biphosphate by a 6-phosphofructokinase classified, for example, under EC 2.7.1.11, such as the gene product of pfkA from E. coli K-12; followed by conversion of fructose 1,6-biphosphate to acetyl-P and D-erythrose 4-phosphate by a fructose-6-phosphate phosphoketolase classified, for example, under EC 4.1.2.22, such as the gene product of xfp from Bifidobacterium animalis sp. Lactis.

In some embodiments, D-erythrose 4-phosphate formed as described above and β-D-fructofuranose 6 phosphate formed as described are converted to D-glyceraldehyde 3-phosphate and D-sedoheptulose 7-phosphate by a transaldolase classified, for example, under EC 2.2.1.2, such as the gene product of talB from E. coli K-12; followed by conversion of D-glyceraldehyde 3-phosphate and D-sedoheptulose 7-phosphate to D-xylulose 5-phosphate and D-ribose 5-phosphate by a ribose-5-phosphate isomerase classified, for example, under EC 5.3.1.6, such as the gene product of rpiA from E. coli K-12; followed by conversion of D-xylolose 5-phosphate to D-ribulose 5-phosphate by a ribulose-phosphate 3-epimerase classified, for example, under EC 5.1.3.1, such as the gene product of rpe1 from S. cerevisiae, and conversion of D-ribose 5-phosphate to D-ribulose 5-phosphate by a ribose-5-phosphate isomerase classified, for example, under EC 5.3.1.6, such as the gene product of rpiA from E. coli K-12.

In some embodiments, D-ribulose 5-phosphate formed as described above is used in the synthesis of hexulose 6-phosphate as described above.

In some embodiments, pyruvate is synthesized by acetyl-P formed as described above by the conversion of acetyl-P and CoA to Pᵢ and acetyl-CoA by a phosphate acetyltransferase classified, for example, under EC 2.3.1.8, such as the gene product of pta from E. coli K-12; followed by conversion of acetyl-CoA and formate to CoA and pyruvate by a formate C-acetyltransferase classified, for example, under EC 2.3.1.54, such as the gene product of by pfl from C. butyricum, and a pyruvate synthase classified, for example, under EC 1.2.7.1, such as the gene product of por from D. africanus, or a pyruvate dehydrogenase (NADP⁺) classified, for example, under EC 1.2.1.51, such as the gene product of CMU_012830 from Cryptosporidium muris RN66 (e.g., RefSeq Accession No. XP_002140958.1).

### Additional Embodiments

Further provided herein are methods involving less than all the steps described for all the above pathways. Such methods can involve, for example, one, two, three, four, five, six, seven, eight, nine, ten, eleven, twelve or more of such steps. Where less than all the steps are included in such a method, the first, and in some embodiments the only step can be any one of the steps listed.

Furthermore, recombinant hosts described herein can include any combination of the above enzymes such that one or more of the steps, e.g., one, two, three, four, five, six, seven, eight, nine, ten, or more of such steps, can be performed within a recombinant host.

For example, in one aspect, the disclosure provides a method of producing formate in a recombinant host, said method comprising enzymatically converting 2-methyl-isocitrate to pyruvate and succinate in said recombinant host using a protein having methylisocitrate lyase activity and enzymatically converting pyruvate to formate and acetyl-CoA in said recombinant host using a protein having formate C-acetyltransferase activity.

In some embodiments, the protein having methylisocitrate lyase activity is classified under EC 4.1.3.30.

In some embodiments, the protein having formate C-acetyltransferase activity is classified under EC 2.3.1.54.

In another aspect, the disclosure provides a method of producing formate in a recombinant host, said method comprising enzymatically converting malonate semialdehyde and L-alanine to pyruvate in said recombinant host using a protein having β-alanine pyruvate aminotransferase activity and enzymatically converting pyruvate to formate and acetyl-CoA in said recombinant host using a protein having formate C-acetyltransferase classified.

In some embodiments, the protein having β-alanine pyruvate aminotransferase activity is classified under EC 2.6.1.18.

In some embodiments, the protein having formate C-acetyltransferase activity is classified under EC 2.3.1.54.

In another aspect, the disclosure provides a method of producing β-alanine in a recombinant host, said method comprising enzymatically converting acetyl-CoA, ATP, and CO₂ to malonyl-CoA, ADP, and Pᵢ in said recombinant host using a protein having acetyl-CoA carboxylase activity; enzymatically converting malonyl-CoA, NADPH, and H⁺ to malonate semialdehyde, NADP⁺, and CoA in said recombinant host using a protein having malonyl-CoA reductase (malonate semialdehyde-forming) activity; and enzymatically converting malonate semialdehyde and L-alanine to β-alanine and pyruvate in said recombinant host using a protein having β-alanine pyruvate aminotransferase activity.

In some embodiments, the protein having acetyl-CoA carboxylase is classified under EC 6.4.1.2.

In some embodiments, the protein having malonyl-CoA reductase (malonate semialdehyde-forming) activity is classified under EC 1.2.1.75.

In some embodiments, the protein having β-alanine pyruvate aminotransferase is classified under EC 2.6.1.18.

In another aspect, the disclosure further provides a method of producing propanoyl-CoA in a recombinant host, said method comprising enzymatically converting β-alanine and succinyl-CoA to succinate and β-alanyl-CoA in said recombinant host using a protein having a CoA-transferase activity; enzymatically converting β-alanyl-CoA to acryloyl-CoA and NH₃ in said recombinant host using a protein having β-alanyl-CoA:ammonia lyase activity; and enzymatically converting acryloyl-CoA and NADPH to NADP⁺ and propanoyl-CoA in said recombinant host using a protein having acrylyl-CoA reductase (NADPH) activity.

In some embodiments, the protein having CoA-transferase activity is classified under 2.8.3.-.

In some embodiments, the protein having β-alanyl-CoA:ammonia lyase activity is classified under EC 4.3.1.6.

In some embodiments, the protein having acrylyl-CoA reductase (NADPH) activity is classified under EC 1.3.1.84.

In another aspect, the disclosure provides a method of producing 2-methylcitrate in a recombinant host, said method comprising enzymatically converting acetyl-CoA, ATP, and CO₂ to malonyl-CoA, ADP, and Pᵢ in said recombinant host using a protein having acetyl-CoA carboxylase activity; enzymatically converting malonyl-CoA, NADPH, and H⁺ to malonate semialdehyde, NADP⁺, and CoA in said recombinant host using a protein having malonyl-CoA reductase (malonate semialdehyde-forming) activity; enzymatically converting malonate semialdehyde and NADPH to NADP⁺ and 3-hydroxypropanoate in said recombinant host using a protein having 3-hydroxypropionate dehydrogenase activity; enzymatically converting 3-hydroxypropanoate ATP, CoA, and succinyl-CoA to succinate, 3-hydroxy-propanoyl-CoA, ADP, and Pᵢ in said recombinant host using a protein having 3-hydroxypropionyl-CoA synthase activity; enzymatically converting 3-hydroxy-propanoyl-CoA to acryloyl-CoA and H₂O in said recombinant host using a protein having enoyl-CoA hydratase activity; enzymatically converting acryloyl-CoA and NADPH to NADP⁺ and propanoyl-CoA in said recombinant host using a protein having acrylyl-CoA reductase (NADPH) activity; and enzymatically converting propanoyl-CoA and oxaloacetate to 2-methylcitrate in said recombinant host using a protein having 2-methylcitrate synthase activity.

In some embodiments, the protein having CoA-transferase activity is classified under EC 2.8.3.-.

In some embodiments, the protein having malonyl-CoA reductase (malonate semialdehyde-forming) activity is classified under EC 1.2.1.75.

In some embodiments, the protein having 3-hydroxypropionate dehydrogenase activity is classified under EC 1.1.1.59.

In some embodiments, the protein having 3-hydroxypropionate dehydrogenase activity is classified under EC 1.1.1.298.

In some embodiments, the protein having CoA-transferase activity is classified under EC 6.4.1.2.

In some embodiments, the protein having 3-hydroxypropionyl-CoA synthase activity is classified under EC 6.2.1.36.

In some embodiments, the protein having enoyl-CoA hydratase activity is classified under EC 4.2.1.17.

In some embodiments, the protein having acrylyl-CoA reductase (NADPH) activity is classified under EC 1.3.1.84.

In some embodiments, the protein having 2-methylcitrate synthase activity is classified under EC 2.3.3.5.

In another aspect, the disclosure provides a method of producing formate in a recombinant host, said method comprising enzymatically converting lactate, NAD⁺, and oxaloacetate to pyruvate, NADH, H⁺, and malate in said recombinant host using a protein having L-lactate dehydrogenase activity and a protein having lactate-malate transhydrogenase activity; and enzymatically converting pyruvate to formate and acetyl-CoA in said recombinant host using a protein having formate C-acetyltransferase activity.

In some embodiments, the protein having L-lactate dehydrogenase activity is classified under EC 1.1.1.27.

In some embodiments, the protein having lactate-malate transhydrogenase activity is classified under EC 1.1.99.7.

In some embodiments, the protein having formate C-acetyltransferase activity is classified under EC 2.3.1.54.

In another aspect, the disclosure provides a method of producing lactate in a recombinant host, said method comprising enzymatically converting acetyl-CoA, ATP, and CO₂ to malonyl-CoA, ADP, and Pᵢ using a protein having acetyl-CoA carboxylase activity; enzymatically converting malonyl-CoA, NADPH, and H⁺ to malonate semialdehyde, NADP⁺, and CoA in said recombinant host using a protein having malonyl-CoA reductase (malonate semialdehyde-forming) activity; enzymatically converting malonate semialdehyde and NADPH to NADP⁺ and 3-hydroxypropanoate in said recombinant host using a protein having 3-hydroxypropionate dehydrogenase activity; and enzymatically converting 3-hydroxypropanoate and lactoyl-CoA to lactate and 3-hydroxy-propionyl-CoA in said recombinant host using a protein having propionate CoA-transferase activity.

In some embodiments, the protein having acetyl-CoA carboxylase activity is classified under EC 6.4.1.2.

In some embodiments, the protein having malonyl-CoA reductase (malonate semialdehyde-forming) activity is classified under EC 1.2.1.75.

In some embodiments, the protein having 3-hydroxypropionate dehydrogenase activity is classified under EC 1.1.1.59.

In some embodiments, the protein having 3-hydroxypropionate dehydrogenase activity is classified under EC 1.1.1.298.

In some embodiments, the protein having propionate CoA-transferase is classified under EC 2.8.3.1.

In another aspect, the disclosure provides a method of producing formate in a recombinant host, said method comprising enzymatically converting homoserine and H₂O to 2-oxobutyrate and NH₃ in said recombinant host using a protein having threonine ammonia-lyase activity and a protein having cystathionine γ-lyase activity; and enzymatically converting 2-oxobutyrate to formate and propanoyl-CoA in said recombinant host using a protein having formate C-acetyltransferase activity.

In some embodiments, the protein having threonine ammonia-lyase activity is classified under EC 4.3.1.19.

In some embodiments, the protein having cystathionine γ-lyase activity is classified under EC 4.4.1.1.

In some embodiments, the protein having formate C-acetyltransferase activity is classified under EC 2.3.1.54.

In another aspect, the disclosure provides a method of producing L-serine in a recombinant host, said method comprising enzymatically converting formate, 5,6,7,8-tetrahydrofolate, and ATP to ADP, Pᵢ, and 10-formyletetrahydrofolate in said recombinant host using a protein having formate-tetrahydrofolate ligase activity; enzymatically converting 10-formyltetrahydrofolate and H₂O to H⁺ and 5,10-methenyl-tetrahydrofolate in said recombinant host using a protein having methenyltetrahydrofolate cyclohydrolase activity; enzymatically converting 5,10-methenyl-tetrahydrofolate and NADPH to NADP⁺ and 5,10-methylene-tetrahydrofolate in said recombinant host using a protein having methylenetetrahydrofolate dehydrogenase (NADP⁺) activity; and enzymatically converting L-glycine, H₂O, and 5-methylene-tetrahydrofolate to L-serine and 5,6,7,8-tetrahydrofolate in said recombinant host using a protein having glycine hydroxymethyltransferase activity.

In some embodiments, the protein having formate-tetrahydrofolate ligase activity is classified under EC 6.3.4.3.

In some embodiments, the protein having methenyltetrahydrofolate cyclohydrolase activity is classified under EC 3.5.4.9.

In some embodiments, the protein having methylenetetrahydrofolate dehydrogenase (NADP⁺) activity is classified under EC 1.5.1.5.

In some embodiments, the protein having glycine hydroxymethyltransferase activity is classified under EC 2.1.2.1.

In another aspect, the disclosure provides a method of producing 2-hydroxy-3-oxopropanoate in a recombinant host, said method comprising enzymatically converting L-serine and glyoxylate to hydroxypyruvate and L-glycine in said recombinant host using a protein having serine-glyoxylate transaminase activity and enzymatically converting hydroxypyruvate to 2-hydroxy-3-oxopropanoate in said recombinant host using a protein having hydroxypyruvate isomerase activity.

In some embodiments, the protein having serine-glyoxylate transaminase activity is classified under EC 2.6.1.45.

In some embodiments, the protein having hydroxypyruvate isomerase activity is classified under EC 5.3.1.22.

In another aspect, the disclosure provides a method of producing 2-oxoglutarate in a recombinant host, said method comprising enzymatically converting 2-hydroxy-3-oxopropanoate and pyruvate to 5-dehydro-4-deoxy-D-glucarate in said recombinant host using a protein having 2-dehydro-3-deoxyglucarate aldolase activity; enzymatically converting 5-dehydro-4-deoxy-D-glucarate and H⁺ to CO₂, H₂O, and 2,5-dioxopentanoate in said recombinant host using a protein having 5-dehydro-4-deoxyglucarate dehydratase activity; and enzymatically converting 2,5-dioxopentanoate, NADP⁺, and H₂O to NADP, 2H⁺, and 2-oxoglutarate in said recombinant host using a protein having 2,5-dioxovalerate dehydrogenase activity.

In some embodiments, the protein having 2-dehydro-3-deoxyglucarate aldolase activity is classified under EC 4.1.2.20.

In some embodiments, the protein having 5-dehydro-4-deoxyglucarate dehydratase activity is classified under EC 4.2.1.41.

In some embodiments, the protein having 2,5-dioxovalerate dehydrogenase activity is classified under EC 1.2.1.26.

In another aspect, the disclosure provides a method of producing hexulose 6-phosphate in a recombinant host, said method comprising enzymatically converting formate, succinyl-CoA, and ATP to ADP, Pᵢ, and succinate in said recombinant host using a protein having acetate-CoA ligase activity and a protein having formyl-CoA transferase activity; enzymatically converting formyl-CoA and NADH to CoA, NAD⁺, and formaldehyde in said recombinant host using a protein having aldehyde-alcohol dehydrogenase activity; and enzymatically converting formaldehyde and D-ribulose 5-phosphate to hexulose 6-phosphate in said recombinant host using a protein having phosphoenolpyruvate carboxylase activity.

In some embodiments, the protein having acetate-CoA ligase activity is classified under EC 6.2.1.1.

In some embodiments, the protein having formyl-CoA transferase activity is classified under EC 2.8.3.16.

In some embodiments, the protein having aldehyde-alcohol dehydrogenase activity is classified under EC 1.2.1.10.

In some embodiments, the protein having phosphoenolpyruvate carboxylase activity is classified under EC 4.1.1.31.

In some embodiments, hexulose 6-phosphate formed as described above is enzymatically converted to β-D-fructofuranose 6 phosphate in said recombinant host using a protein having 6-phospho-3-hexuloisomerase activity. In some embodiments, the protein having 6-phospho-3-hexuloisomerase activity is classified under EC 5.3.1.27.

In some embodiments, the disclosure provides a method of producing D-erythrose 4-phosphate in a recombinant host, said method comprising enzymatically converting ATP and β-D-fructofuranose 6 phosphate to ADP, H⁺, and fructose 1,6-biphosphate in said recombinant host using a protein having 6-phosphofructokinase and enzymatically converting fructose 1,6-biphosphate to acetyl-P and D-erythrose 4-phosphate in said recombinant host using a protein having fructose-6-phosphate phosphoketolase activity.

In some embodiments, the protein having 6-phosphofructokinase activity is classified under EC 2.7.1.11.

In some embodiments, the protein having fructose-6-phosphate phosphoketolase activity is classified under EC 4.1.2.22.

Also provided herein are host cells of hydrogen-oxidizing bacteria genetically engineered to express one or more (e.g., two, three, four, five, six, seven, eight, nine, 10, 11, 12 or more) recombinant forms of any of the enzymes recited in the document. For example, the host cells can contain exogenous nucleic acids encoding enzymes catalyzing one or more of the steps of any of the pathways described herein. Alternatively, all the steps can be performed using extracted enzymes, or some of the steps can be performed in cells and others can be performed using extracted enzymes.

For example, in some embodiments, a recombinant host may include one or more exogenous nucleic acids encoding one or more proteins having the activity of a 2-methylisocitrate dehydratase, a methylisocitrate lyase, a succinate dehydrogenase (quinone), a fumarate reductase (quinol), a fumarate hydratase, a malate dehydrogenase, a 2-methylisocitrate dehydratase, a 2-methylcitrate synthase, an acrylyl-CoA reductase (NADPH), a β-alanyl-CoA:ammonia lyase, a glutamate dehydrogenase, a CoA-transferase, an alanine transaminase, a β-alanine pyruvate aminotransferase, a formate C-acetyltransferase, a malonyl-CoA reductase (malonate semialdehyde-forming), an alanine-oxo-acid transaminase, or an acetyl-CoA carboxylase.

In some embodiments, a recombinant host may include one or more exogenous nucleic acids encoding one or more proteins having the activity of a 2-methylisocitrate dehydratase, a methylisocitrate lyase, a succinate dehydrogenase (quinone), a fumarate reductase (quinol), a fumarate hydratase, a malate dehydrogenase, a 2-methylisocitrate dehydratase, a 2-methylcitrate synthase, an acrylyl-CoA reductase (NADPH), a CoA-transferase, an alanine transaminase, a formate C-acetyltransferase, a malonyl-CoA reductase (malonate semialdehyde-forming), a 3-hydroxypropionate dehydrogenase, a 3-hydroxypropionyl-CoA synthase, an enoyl-CoA hydratase, or an acetyl-CoA carboxylase.

In some embodiments, a recombinant host may include one or more exogenous nucleic acids encoding one or more proteins having the activity of an enoyl-CoA hydratase, a lactoyl-CoA dehydratase, a propionate CoA-transferase, a 3-hydroxypropionate dehydrogenase, a malonyl-CoA reductase (malonate semialdehyde-forming), an acetyl-CoA carboxylase, a formate C-acetyltransferase, a lactate-malate transhydrogenase, or a L-lactate dehydrogenase.

In some embodiments, a recombinant host may include one or more exogenous nucleic acids encoding one or more proteins having the activity of a threonine ammonia-lyase, a cystathionine γ-lyase, a formate C-acetyltransferase, a 2-methylcitrate synthase, a 2-methylcitrate dehydratase, a 2-methylisocitrate dehydratase, a methylisocitrate lyase, a succinate dehydrogenase (quinone), a fumarate reductase (quinol), a fumarate hydratase, a malate dehydrogenase, a malate dehydrogenase (oxaloacetate-decarboxylating), an acetyl-CoA carboxylase, an aspartate kinase, an aspartate-semialdehyde dehydrogenase, a malate dehydrogenase, or a glutamate dehydrogenase.

In some embodiments, a recombinant host may include one or more exogenous nucleic acids encoding one or more proteins having the activity of a formate-tetrahydrofolate ligase, a methenyltetrahydrofolate cyclohydrolase, a methylenetetrahydrofolate dehydrogenase (NADP⁺), a glycine hydroxymethyltransferase, a formate-tetrahydrofolate ligase, a serine-glyoxylate aminotransferase, a hydroxypyruvate reductase, a glycerate 2-kinase, a phosphopyruvate hydratase, a pyruvate kinase, a malate dehydrogenase (oxaloacetate-decarboxylating), a pyruvate carboxylase, a malate dehydrogenase, a succinyl-CoA-L-malate CoA-transferase, a malate-CoA ligase, a malyl-CoA lyase, a pyruvate synthase, or a formate C-acetyltransferase.

In some embodiments, a recombinant host may include one or more exogenous nucleic acids encoding one or more proteins having the activity of a pyruvate, phosphate dikinase, a pyruvate, water dikinase, a malate dehydrogenase (oxaloacetate-decarboxylating), a pyruvate carboxylase, a malate dehydrogenase, a succinyl-CoA-L-malate CoA-transferase, a malate-CoA ligase, a malyl-CoA lyase, a pyruvate synthase, a tartronate-semialdehyde synthase, an oxidoreductase with NAD(+) or NADP(+) as acceptor, a glycerate 3-kinase, a phosphoglycerate mutase (2,3-diphosphoglycerate-independent), or a pyruvate kinase.

In some embodiments, a recombinant host may include one or more exogenous nucleic acids encoding one or more proteins having the activity of a pyruvate carboxylase, a pyruvate, phosphate dikinase, a pyruvate, water dikinase, a malate dehydrogenase (oxaloacetate-decarboxylating), a malate dehydrogenase, a succinyl-CoA-L-malate CoA-transferase, a malate-CoA ligase, a malyl-CoA lyase, a pyruvate synthase, a formate C-acetyltransferase, a formate-tetrahydrofolate ligase, a methenyltetrahydrofolate cyclohydrolase, a methylenetetrahydrofolate dehydrogenase (NADP⁺), a glycine hydroxymethyltransferase, a formate-tetrahydrofolate ligase, a serine-glyoxylate aminotransferase, a hydroxypyruvate reductase, a glycerate dehydrogenase, a glycerate 2-kinase, a phosphopyruvate hydratase, or a pyruvate kinase.

In some embodiments, a recombinant host may include one or more exogenous nucleic acids encoding one or more proteins having the activity of a formate-tetrahydrofolate ligase, a methenyltetrahydrofolate cyclohydrolase, a methylenetetrahydrofolate dehydrogenase (NADP⁺), a glycine hydroxymethyltransferase, a formate-tetrahydrofolate ligase, a serine-glyoxylate transaminase, a hydroxypyruvate isomerase, a 2-dehydro-3-deoxyglucarate aldolase, a 5-dehydro-4-deoxyglucarate dehydratase, a 2,5-dioxovalerate dehydrogenase, an oxidoreductase with NAD(+) or NADP(+) as acceptor, an aconitate hydratase, a citrate (Si)-synthase, a citrate synthase, a malate dehydrogenase, a malate-CoA ligase, a malyl-CoA lyase, a pyruvate synthase, or a formate C-acetyltransferase. In some embodiments, a recombinant host may include one or more exogenous nucleic acids encoding one or more proteins having the activity of an acetate-CoA ligase, a formyl-CoA transferase, an aldehyde-alcohol dehydrogenase, a phosphoenolpyruvate carboxylase, a 6-phospho-3-hexuloisomerase, a 6-phosphofructokinase, a fructose-bisphosphate aldolase, a transketolase, a ribulose-phosphate 3-epimerase, a transaldolase, a ribose-5-phosphate isomerase, a ribulose-phosphate 3-epimerase, and a ribose-5-phosphate isomerase.

In some embodiments, a recombinant host may include one or more exogenous nucleic acids encoding one or more proteins having the activity of an acetate-CoA ligase, a formyl-CoA transferase, an aldehyde-alcohol dehydrogenase, a phosphoenolpyruvate carboxylase, a 6-phospho-3-hexuloisomerase, a 6-phosphofructokinase, a fructose-6-phosphate phosphoketolase, a transaldolase, a ribose-5-phosphate isomerase, a ribulose-phosphate 3-epimerase, a ribose-5-phosphate isomerase, a phosphate acetyltransferase, a formate C-acetyltransferase, a pyruvate synthase, or a pyruvate dehydrogenase (NADP⁺).

In some embodiments, the enzymes in the pathways described herein can be gene dosed, i.e., overexpressed, into the resulting genetically modified organism via episomal or chromosomal integration approaches.

In some embodiments, the hydrogen-oxidizing microorganism has an operable Calvin-Benson cycle, wherein the Calvin-Benson cycle is attenuated to direct inorganic carbon to one of the pathways described herein. Attenuation strategies include, but are not limited to, the use of transposons, homologous recombination, mutagenesis, enzyme inhibitors, and RNAi interference.

### Cultivation Strategies

In some embodiments, a recombinant host is a hydrogen-oxidizing microorganism with an attenuated Calvin-Benson cycle utilizing at least one synthetic biochemical pathway described herein. In some embodiments, the recombinant host more efficiently recycles reduced electron carriers and more efficiently fixes carbon relative to the an otherwise identical hydrogen-oxidizing microorganism utilizing the Calvin-Benson cycle for carbon fixation. In some embodiments, the recombinant host more efficiently produces biofuels or other chemical commodities utilizing renewable solar energy relative to an otherwise identical hydrogen-oxidizing microorganism utilizing the Calvin-Benson cycle for carbon fixation.

In some embodiments, the recombinant host is a hydrogen-oxidizing microorganism such as Alcaligenes eutrophus, Alcaligenes latus, Alcaligenes paradoxus, Alcaligenes ruhlandii, Alcaligenes lactus, Alacligenes paradoxus, Aquaspirillum autotrophicum, Bacillus schlegelii, Cupriavidus necator, Derxia gummosa, Flavobacterium autothermophilum, Helicobacter pylori, Hydrogenobacter thermophilus, Hydrogenovibrio marinus, Hydrogenomonas facilis, Hydrogenomonas eutropha, Microcyclus aquaticus, Microcyclus ebruneus, Parcoccus denitrificans, Pseudomonas carboxydovorans, Pseudomonas facilis, Pseudomonas flava, Pseudomonas pseudoflava, Pseudomonas hydrogenovora, Pseudomonas hydrogenothermophila, Pseudomonas palleronii, Pseudomonas saccharophila, Pseudomonas thermophila, Renobacter vacuolatum, Rhizobium japonicum, Rhodospirillum rubrum, Seliberia carboxyhydrogena, Flavobacterium autothermophilum, Paracoccus denitrificans, Xanthobacter autotrophicus, Xanthobacter flavus, Mycobacterium gordonae, Nocardia autotrophica, or Nocardia opaca.

In some embodiments, the recombinant host is a hydrogen-oxidizing microorganism with an operable Calvin-Benson cycle selected from Cupriavidus necator, Hydrogenovibrio marinus, Rhodobacter capsulatus, Rhodobacter sphaeroides, Rhodospirillum rubrum, Thiobacillus ferrooxidans, and Xanthobacter flavus.

In some embodiments, a biofuel or other chemical commodity may be produced by providing a host microorganism with an attenuated Calvin-Benson cycle utilizing at least one synthetic biochemical pathway described herein and culturing the provided microorganism with a culture media containing a suitable carbon source as described herein. In some embodiments, the culture media and/or culture conditions can be such that the microorganisms grow to an adequate density and produce a biofuel or other chemical commodity efficiently.

In some embodiments, a cell retention strategy using, for example, ceramic hollow fiber membranes may be employed to achieve and maintain a high cell density during either fed-batch or continuous fermentation.

In some embodiments, a continuous culture incorporating cell retention is utilized such that the biomass in the vessel reaches a concentration above that possible in a chemostat with a similar nutrient feed rate. The biomass concentration is achieved by subjecting the effluent stream to a biomass separation process using a cross-flow membrane) and returning a portion of the concentrated biomass to the growth biomass.

In some embodiments, fermentation is divided into three main phases. The first phase is a growth in batch mode with pH control (6 hours) which allowed for growth using an initial charge of a carbon source. The second phase is performed in continuous-retention mode, where high cell growth is achieved and sustained. The growth may be monitored in real time measuring the Oxygen Uptake Rate and controlled by continuous bleeding. A third phase, for inducible constructs, comprised induction after reaching a steady state, by spiking an inducer in the vessel and the different feeds.

The initial charge containing a carbon source and appropriate antibiotics is prepared and filtrated to fermenters. The bioreactors may be operated at a temperature of 30° C. and a pH from 6.80 to 7 (depending on the culture). The fermentation startup is performed in batch or continuous-retention conditions to allow initial accumulation of biomass. After startup, the fermenters are operated at the desired dilution rate.

The pH may be controlled by the automatic addition of Ammonium Hydroxide Solution, 10% through DCU peristaltic pumps. The dissolved oxygen percentage (pO₂) [%] was controlled by a single-level cascade of agitation.

Example fermentation control parameters for a 1 L scale bioreactor operation are provided in Table 1. STP stands for standard temperature and pressure.

TABLE 1
Example fermentation control parameters.

| | | | Heterologous | Lithotrophic |
|---|---|---|---|---|
| Control | | | Growth | Growth |
| parameter | Unit | Batch phase | phase | phase |
| Aeration feed | [vvm] | 0.5 | 0.5 | 0 |
| rate | | | | |
| Agitation | [rpm] | Control DO > | Control DO > | Maximum |
| | | 20[%] sat | 20[%] sat | |
| O₂ feed rate @ | [vvm] | 0 | 0 | Manipulated |
| STP | | | | variable of |
| | | | | DO |
| | | | | controller |
| H₂ feed rate @ | [vvm] | 0 | 0 | 0.9 |
| STP | | | | |
| CO₂ feed | [vvm] | 0 | 0 | 0.05 |
| rate @ STP | | | | |
| pH | [—] | 6.8 | 6.8 | 6.8 |
| DO controller | [%] sat | Manual | Manual | Manipulated |
| | | control | control | variable of |
| | | | | Off-gas |
| | | | | controller. |
| Off-gas O2 | [%] | Not | Not | 3.5[%] (v/v) |
| concentration | (v/v) | controlled | controlled | |
| Cross flow | [rpm] | 120 | 120 | 120 |
| Temperature | [° C.] | 30 | 30 | 30 |

In some embodiments, for large-scale production processes, any method can be used such as those described elsewhere (Manual of Industrial Microbiology and Biotechnology, 2nd Edition, Editors: A L. Demain and J. E. Davies, ASM Press; and Principles of Fermentation Technology, P. F. Stanbury and A Whitaker, Pergamon). Briefly, a large tank (e.g., a 100 gallon, 200 gallon, 500 gallon, or more tank) containing an appropriate culture medium is inoculated with a particular microorganism. After inoculation, the microorganism is incubated to allow biomass to be produced. Once a desired biomass is reached, the broth containing the microorganisms can be transferred to a second tank. This second tank can be any size. For example, the second tank can be larger, smaller, or the same size as the first tank. Typically, the second tank is larger than the first such that additional culture medium can be added to the broth from the first tank. In addition, the culture medium within the second tank can be the same as, or different from, that used in the first tank. Once transferred, the microorganisms can be incubated to allow for the production of a biofuel or other chemical commodity.

In some embodiments, a cultivation strategy is used to achieve anaerobic, microaerobic, or aerobic cultivation conditions.

In some embodiments, the cultivation strategy includes limiting nutrients, such as limiting nitrogen, phosphate, or oxygen.

In some embodiments, the principal carbon source fed to the fermentation in the synthesis of a biofuel or other chemical commodity in a hydrogen-oxidizing microorganism with an attenuated Calvin-Benson cycle utilizing at least one synthetic biochemical pathway described herein can derive from biological or non-biological feedstocks.

In some embodiments, the biological feedstock can be or can be derived from monosaccharides, disaccharides, lignocellulose, hemicellulose, cellulose, lignin, levulinic acid and formic acid, triglycerides, glycerol, fatty acids, agricultural waste, condensed distillers' solubles, or municipal waste. Efficient catabolism of these biological feedstocks has been demonstrated in many microorganisms.

In some embodiments, the principal carbon source is a commercially available growth medium. Some non-limiting examples include Nutrient broth (Sigma: N7519), dextrose-free TSB (Sigma: T3938), LB medium (Sigma: L3022), or 2xYT medium (Sigma: Y2377).

In some embodiments, the principal carbon source is the example growth medium described in Table 2.

TABLE 2
Shake flask media, adjusted to pH = 6.6 [-] using 1[M] NaOH.

| Media component | Unit | Concentration |
|---|---|---|
| (NH₄)₂SO₄ | [g/kg] | 1.25 |
| MgSO₄•7H₂O | [g/kg] | 0.5 |
| CaCl₂•2H₂O | [mg/kg] | 10 |
| FeSO₄•7H₂O | [mg/kg] | 50 |
| K₂SO₄ | [g/kg] | 0.3 |
| (NaPO₃)₆ (CAS 68915-31-1) | [g/kg] | 0.85 |
| Trace metal solution (see Table 3) | [mL/kg] | 10 |
| Antifoam 204 | [mL/kg] | 0.5 |
| Fructose | [g/kg] | 2.5 |
| H₂O (Natural pH ~4) | [mL] | Make-up |

TABLE 3
Trace metal stock solution

| Media component | Unit | Concentration |
|---|---|---|
| ZnSO₄•7H₂O | [mg/L] | 100 |
| MnCl₂•4H₂O | [mg/L] | 30 |
| H₃BO₃ | [mg/L] | 300 |
| CoCl₂•6H₂O | [mg/L] | 200 |
| NiSO₄•6H₂O | [mg/L] | 25 |
| Na₂MoO₄•2H₂O | [mg/L] | 30 |
| CuSO₄•5H₂O | [mg/L] | 15 |
| H₂O (pH = 2 with HCl) | [mL] | 1000 |

In some embodiments, the cultivation strategy includes the use of an initial charge medium for fermentation as described in Table 4.

TABLE 4
Initial charge media, adjusted to pH = 6.6 [-] in fermenter
using 12[%] (v/v) NH₃(aq).

| Media component | Unit | Concentration |
|---|---|---|
| (NH₄)₂SO₄ | [g/kg] | 1.25 |
| MgSO₄•7H₂O | [g/kg] | 0.5 |
| CaCl₂•2H₂O | [mg/kg] | 10 |
| FeSO₄•7H₂O | [mg/kg] | 50 |
| K₂SO₄ | [g/kg] | 0.3 |
| (NaPO₃)₆ (CAS 68915-31-1) | [g/kg] | 0.85 |
| Trace metal solution (see Table 3) | [mL/kg] | 10 |
| Antifoam 204 | [mL/kg] | 0.5 |
| H₂O (Natural pH ~4) | [mL] | Make-up |

In some embodiments, the cultivation strategy includes the use of a medium composition for a sterile nutrient fermentation as described in Table 5.

TABLE 5
Media composition for sterile nutrient feed

| Media component | Unit | Concentration |
|---|---|---|
| (NH₄)₂SO₄ | [g/kg] | 1.75 |
| MgSO₄•7H₂O | [g/kg] | 0.7 |
| CaCl₂•2H₂O | [mg/kg] | 15 |
| FeSO₄•7H₂O | [mg/kg] | 65 |
| K₂SO₄ | [g/kg] | 0.3 |
| (NaPO₃)₆ (CAS 68915-31-1) | [g/kg] | 0.85 |
| Trace metal solution (see Table 3) | [mL/kg] | 15 |
| Antifoam 204 (fed separately in | [mL/kg] | 0.5 |
| Lanzatech fermenter) | | |
| H₂O (Natural pH ~4) | [mL] | Make-up |

For example, the efficient catabolism of crude glycerol stemming from the production of biodiesel has been demonstrated in several microorganisms such as Escherichia coli, Cupriavidus necator, Pseudomonas oleavorans, Pseudomonas putida, and Yarrowia lipolytica (Lee et al., Appl. Biochem. Biotechnol., 2012, 166: 1801-1813; Yang et al., Biotechnology for Biofuels, 2012, 5: 13; Meijnen et al., Appl. Microbial. Biotechnol., 2011, 90:885-893).

For example, the efficient catabolism of lignocellulosic-derived levulinic acid has been demonstrated in several organisms such as Cupriavidus necator and Pseudomonas putida in the synthesis of 3-hydroxyvalerate via the precursor propanoyl-CoA (Jaremko and Yu, 2011, supra; Martin and Prather, J Biotechnol., 2009, 139:61-67).

For example, the efficient catabolism of lignin-derived aromatic compounds such as benzoate analogues has been demonstrated in several microorganisms such as Pseudomonas putida and Cupriavidus necator (Bugg et al., Current Opinion in Biotechnology, 2011, 22, 394-400; Perez-Pantoja et al., FEMS Microbial. Rev., 2008, 32, 736-794). The efficient utilization of agricultural waste such as olive mill waste water has been demonstrated in several microorganisms, including Yarrowia lipolytica (Papanikolaou et al., Bioresour. Technol., 2008, 99(7):2419-2428).

For example, the efficient utilization of fermentable sugars such as monosaccharides and disaccharides derived from cellulosic, hemicellulosic, cane and beet molasses, cassava, corn, and other agricultural sources has been demonstrated for several microorganisms, such as Escherichia coli, Corynebacterium glutamicum and Lactobacillus delbrueckii, and Lactococcus lactis (see, e.g., Hermann et al, J Biotechnol., 2003, 104:155-172; Wee et al., Food Technol. Biotechnol., 2006, 44(2): 163-172; Ohashi et al., J Bioscience and Bioengineering, 1999, 87(5):647-654).

For example, the efficient utilization of furfural, derived from a variety of agricultural lignocellulosic sources, has been demonstrated for Cupriavidus necator (Li et al., Biodegradation, 2011, 22:1215-1225).

In some embodiments, the non-biological feedstock can be or can be derived from natural gas, syngas, CO₂/H₂, methanol, ethanol, benzoate, non-volatile residue (NVR) or a caustic wash waste stream from cyclohexane oxidation processes, or terephthalic acid/isophthalic acid mixture waste streams. Efficient catabolism of these non-biological feedstocks has been demonstrated in many microorganisms.

For example, the efficient catabolism of ethanol has been demonstrated for Clostridium kluyveri (Seedorf et al., Proc. NatL Acad Sci. USA, 2008, 105(6) 2128-2133). The efficient catabolism of CO₂ and H₂, which may be derived from natural gas and other chemical and petrochemical sources, has been demonstrated for Cupriavidus necator (Prybylski et al., Energy, Sustainability and Society, 2012, 2:11).

For example, the efficient catabolism of syngas has been demonstrated for numerous microorganisms, such as Clostridium ljungdahlii and Clostridium autoethanogenum (Kopke et al., Applied and Environmental Microbiology, 2011, 77(15):5467-5475).

For example, the efficient catabolism of the non-volatile residue waste stream from cyclohexane processes has been demonstrated for numerous microorganisms, such as Deljtia acidovorans and Cupriavidus necator (Ramsay et al., Applied and Environmental Microbiology, 1986, 52(1):152-156).

In some embodiments, the host microorganism's tolerance to high concentrations of one or more central precursors described herein is improved through continuous cultivation in a selective environment.

### Strain Construction

To create and test a strain with a heterologous carbon fixation pathway, the current carbon fixation pathway must be inactivated, along with the formate dehydrogenase enzyme as the synthetic pathways often require formate. It should be sufficient to delete Rubisco to remove the ability to fix carbon. The Rubisco genes, cbbL and cbbS, are present in two locations in the C. necator H16 genome, in two cbb operons found on the megaplasmid and chromosome 2 (Kusian, B., & Bowien, B. (1997). Organization and regulation of cbb CO2 assimilation genes in autotrophic bacteria. FEMS microbiology reviews, 21(2), 135-155; Pohlmann, A., Fricke, W. F., Reinecke, F., Kusian, B., Liesegang, H., Cramm, R., . . . & Strittmatter, A. (2006). Nature biotechnology, 24(10), 1257-1262). Both operons contain several other genes involved in the CBB cycle. These genes could be involved in the synthetic pathways (e.g. transketolase in the P5 pathway) or function elsewhere in metabolism depending on growth conditions (Friedrich, C. G., Friedrich, B., & Bowien, B. (1981). Microbiology, 122(1), 69-78), so the Rubisco deletions should not disrupt normal expression of the rest of the operon. The operon on Chromosome 2 is preceded by the cbbR gene, which controls expression of both operons, as the operon on the megaplasmid is preceded by a non-functional version of cbbR (Bowien, B., & Kusian, B. (2002) Archives of microbiology, 178(2), 85-93). The cbbR gene similarly should not be disrupted by the deletions, as not only is it involved in expressing the operon, but also it is required for full expression of the Rbc mutant promoters used in some of the inserted pathways.

Cupriavidus necator has a soluble cytoplasmic formate dehydrogenase, which supports autotrophic growth on formate, as well as a number of membrane-bound formate dehydrogenases that feed directly into the electron transport chain and do not support growth (Friedebold, J. O. R. G., & Bowien, B. (1993) Journal of bacteriology, 175(15), 4719-472; Pohlmann, A., Fricke, W. F., Reinecke, F., Kusian, B., Liesegang, H., Cramm, R., . . . & Strittmatter, A. (2006) Nature biotechnology, 24(10), 1257-1262; Cramm, R. (2008) Journal of molecular microbiology and biotechnology, 16(1-2), 38-52). To remove the ability to grow on formate, it should be sufficient to delete the soluble formate dehydrogenase. This enzyme oxidises formate, producing CO₂ and reducing equivalents that feed into the CBB cycle (Friedrich, C. G., Bowien, B., & Friedrich, B. (1979) Microbiology, 115(1), 185-192). The fdsGBACD operon produces the soluble formate dehydrogenase, and is found on Chromosome 1 (Oh, J. I., & Bowien, B. (1998) Journal of Biological Chemistry, 273(41), 26349-26360). This deletion should remove the ability to grow on formate, though membrane-bound formate dehydrogenases may still be able to metabolise it.

Growth on formate depends on the CBB cycle as well as the formate dehydrogenase activity (Friedrich, C. G., Bowien, B., & Friedrich, B. (1979) Microbiology, 115(1), 185-192). Deletion of Rubisco would consequently remove the ability to grow on formate as well as fix carbon. Deletion of soluble formate dehydrogenase was completed first to allow the possibility of testing of that strain for the loss of the ability to grow on formate. The cbbLS genes were then deleted first from the plasmid, then the chromosome, and the chromosomal cbbR gene confirmed.

A retrosynthesis algorithm was used to produce potential synthetic carbon fixation pathways. Thirteen pathways referred to as P1 through P13 were proposed by the algorithm (named P1-P13). These pathways plus several naturally existing pathways were then further analysed against the original CBB pathway. Pathways were ranked on the following criteria:

1-maximum theoretical yield of isopropanol per hydrogen consumed, assuming no production of biomass or maintenance costs, an assumption with a yield which is unachievable in real life, but is comparable across the pathways, including the native CBB pathway;

2-thermodynamic feasibility based upon likely physiological conditions with reactions with an overly positive ΔG value (e.g. more than +15 kJ/mol) being considered unfavourable, and the overall cycle having a negative ΔG; and

3-metabolic compatibility, meaning how well the pathway would integrate with the existing metabolism of Cupriavidus necator with pathways with fewer heterologous enzymes being preferred.

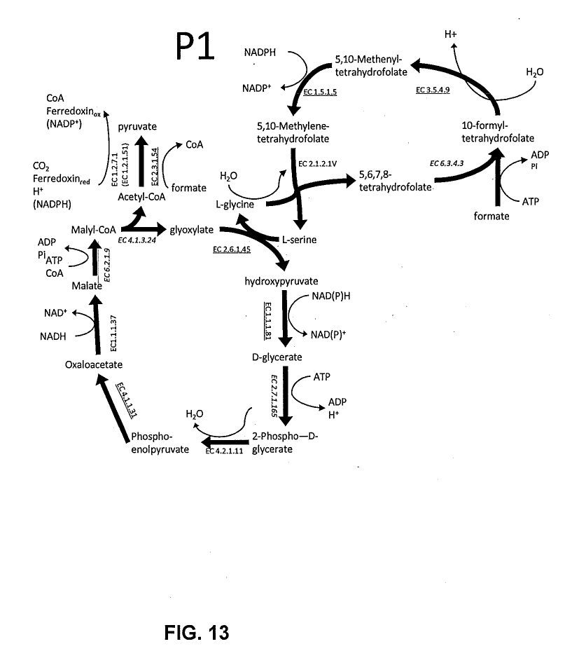

Pathways that scored highly are listed in Table 6 and are depicted in FIGS. 13 through 16.

TABLE 6
Summary of Pathway Details

| | | | # heterologous | |
|---|---|---|---|---|
| | Yield | Cumulative ΔG | enzymes | |
| | (Isopropanol/ | (least favourable | required: | |
| | H₂) | individual | minimal stragegy | |
| Name of | (compared | reaction | (“safest” | Formate |
| pathway | to CBB) | ΔG) | strategy) | dependent |
| CBB | 0.196 (1) | −125.6 (+18.4) | 0 | No |
| P1 | 0.261 (1.33) | −141.2 (+6.5) | 4 (9) | Yes |
| P2 | 0.275 (1.40) | −98.8 (+6.5) | 5 (10) | Yes |
| P5 | 0.275 (1.40) | −11.6 (+15.3) | 5 (8) | Yes |
| P10 | 0.256 (1.30) | −134.5 (+6.8) | 4 (9) | No |

Some of the enzymes that would be necessary for each pathway were not found in the Cupriavidus necator genome. Accordingly, these genes need to be included in the the pathway construct. A number of other enzymes were annotated in the genome, but with lower confidence so they may be misannotated, or their expression level may not support flux through the pathway. These genes may also need to be inserted in the construct, and are highlighted in lighter blue.

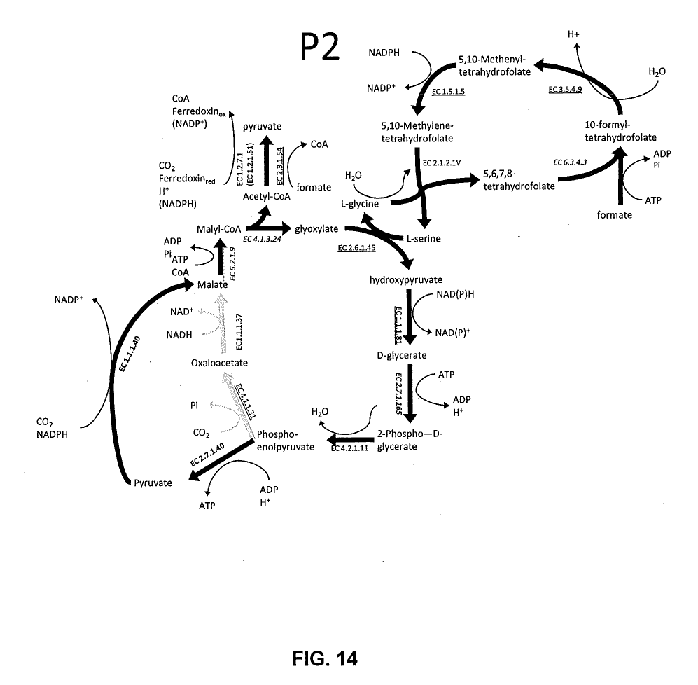

Pathways P1 and P2 are based on the serine cycle, the pathway that methylotrophic bacteria use to assimilate C1 compounds. Both take in formate via THF cycle and attach it to glycine, then regenerate the glycine by transamination with glyoxylate, generating hydroxypyruvate (FIG. 13 & FIG. 14). The difference is that in P1, glyoxylate is regenerated via a cycle including fixation of carbon by the PEP carboxylase and in P2 the carbon is fixed by the malic enzyme. P2 is slightly more energy efficient than P1 (see Table 6).

For pathway P1, enzymes not found in the Cupriavidus necator genome which need to be inserted include EC 6.3.4.3, EC 4.1.3.24, EC 6.2.1.9 and EC 2.7.1.165. Enzymes which may need to be inserted due to misannotation or low expression levels include EC 1.5.1.5, EC 3.5.4.9, EC 2.3.1.54, EC 2.6.1.45, EC 1.1.1.81 and EC 4.1.1.31.

The P2 pathway is largely identical to P1 apart from two steps which include the carbon fixation step of the cycle. The P2 pathway includes most of the same gene requirements as the P1 pathway, with two additional genes required for the alternative CO₂ fixation route (FIG. 14). Both these genes are proposed to be present in Cupravidus necator, however their expression levels are unknown. P2 strains are created from the equivalent P1 strain by insertion of the two additional genes. The additional two genes required which need to be inserted for the P2 pathway include EC 1.1.1.40 and 2.7.1.40.

Pfl (EC 2.3.1.54), is not necessary for the function of either P1 or P2, but may increase the fixation of carbon if it works in that context. Accordingly, this gene was included as another optional gene for each pathway

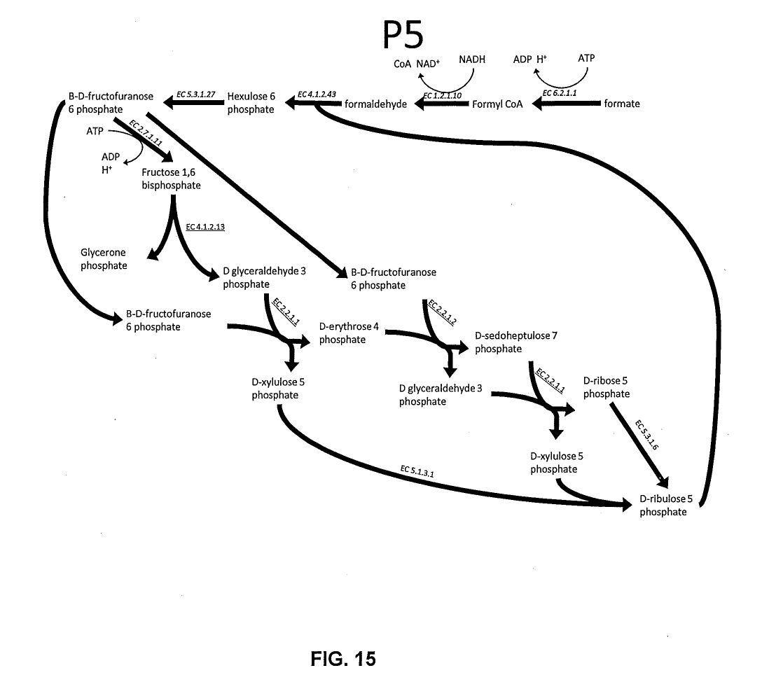

P5 is a variation on the ribulose monophosphate pathway (RuMP pathway) used by methanotrophic bacteria (Kato, N., Yurimoto, H., & Thauer, R. K. (2006) Bioscience, biotechnology, and biochemistry, 70(1), 10-21). In this pathway (FIG. 15), the five-carbon compound ribulose-5-P and the one-carbon formaldehyde are combined to generate the six-carbon hexulose-6-p. Ribulose-5-p is regenerated by pentose phosphate enzymes while the acquired carbon enters the metabolism as DHAP (glycerone phosphate). The upper part of the pathway generates formaldehyde from formate, so this pathway is formate-dependent, and there is no other CO₂ fixing step. For pathway P5, enzymes not found in the Cupriavidus necator genome which need to be inserted include EC 5.3.1.27, EC 4.1.2.43, EC 1.2.1.10, EC 6.2.1.1 and EC 2.7.1.11. Enzymes which may need to be inserted due to misannotation or low expression levels include EC 4.1.2.13, EC 2.2.1.1 and EC 2.2.1.2. Two strategies were developed for constructing P5. The optional genes for P5 are taken from a single operon in Methylococcus capsulatus, and the operon structure is conserved, so the safe strategy for including these extra genes is referred to as P5-operon.

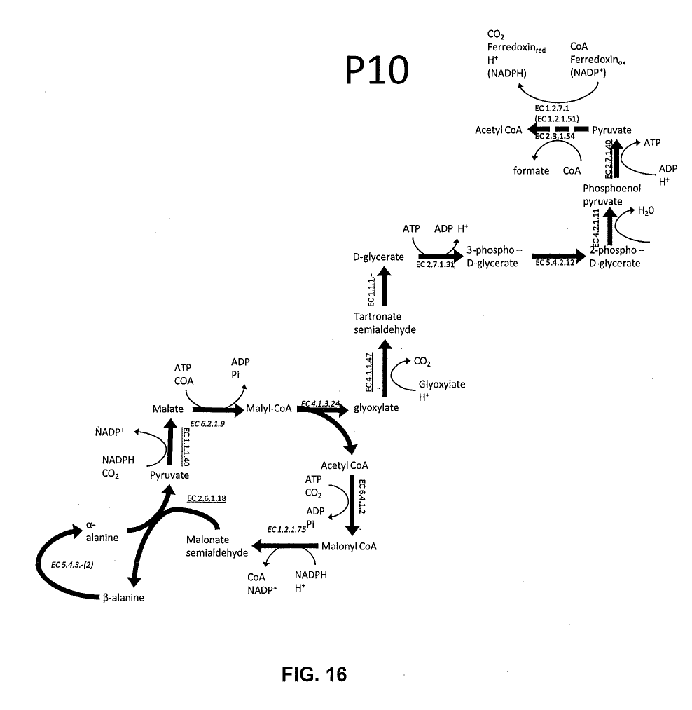

P10 is a pathway involving acetyl-coA carboxylase and glycerol degradation (FIG. 16). There are two carbon fixation steps, using the acetyl-CoA carboxylase, and malic enzyme, in a cycle that generates glyoxylate. This enters the metabolism via glyoxylate assimilation native to Cupriavidus necator, releasing one carbon for every four fixed. This pathway is formate-independent. Accordingly, enzyme Pfl (EC 2.3.1.54) may or may not be required. Optional additional of this enzyme in the pathway is indicated in FIG. 16 by a dashed arrow. For pathway P10, enzymes not found in the Cupriavidus necator genome which need to be inserted include EC 6.2.1.9, EC 4.1.3.24, EC 5.4.3.-(2) and 1.2.1.75. Enzymes which may need to be inserted due to misannotation or low expression levels include EC 2.7.1.40, 2.7.1.31, 1.1.1- and 4.1.1.47.

The remaining set of genes for each pathway are known to be in the Cupriavidus necator genome and are likely or known to be active. These genes are not included in the insertion strategies. This gives the strategy of multiple strains per pathway: a minimal strains containing only the definitely necessary genes (PX.A), and some “safer” strains (PX.B-D) that include the possibly absent or under-expressed genes. For each pathway, a subset of strains is constructed.

In one nonlimiting embodiment, the pathways are assembled in suicide vectors using Goldengate and Goldenbraid methods, and inserted into the genome at the phaCAB locus, phaB2C2 locus, and A0006 locus replacing the native genes. The proposed test for the efficiency of the carbon fixation pathways was production of biomass, which may be affected in some conditions by the presence or absence of the PHA genes. A control strain will be made which will be the base strain in which phaCAB is deleted with no insertion. The phaB2C2 and A0006 deletions are not expected to have a further effect on biomass production so they were not included in the control.

Enzymes required for each pathway construct showing gene names are depicted in Table 7. Arrows indicate the insertion vectors by which each strain is derived from its parent strain.

The specific genes chosen for each enzyme activity are described in more detail in the Examples as well as methods for production of these pathway constructs and their use in production of formate.

### EXAMPLES

The following examples are intended for illustration purposes only and should not be construed as limiting the scope of the disclosure or the claims appended hereto in any way.

### Example 1: Attenuation of the Calvin-Benson Cycle

RubisCO is the key carboxylase enzyme of the Calvin-Benson cycle. Attenuation of the Calvin-Benson cycle may be achieved by attenuating expression of genes associated with RubisCO in a host microorganism with an operable Calvin-Benson cycle.

A RubisCO (cbbLS cbbM)-deficient strain of Rhodobacter capsulatus may be synthesized as previously described in Paoli et al. (Paoli, George C., Padungsri Vichivanives, and F. Robert Tabita. “Physiological control and regulation of the Rhodobacter capsulatus cbb operons.” Journal of Bacteriology 180.16 (1998): 4258-4269), which is incorporated herein by reference to the extent it discloses methods of producing a RubisCO-deficient strain of R. capsulatus. In brief and as described by Paoli et al., photoautotrophic cultures of R. capsulatus are grown anaerobically at 1.5% CO₂ and 98.5% H₂ in Ormerod's medium as previously described (see, e.g., Ormerod, John G., Kari S. Ormerod, and Howard Gest. “Light-dependent utilization of organic compounds and photoproduction of molecular hydrogen by photosynthetic bacteria; relationships with nitrogen metabolism.” Archives of Biochemistry and Biophysics 94.3 (1961): 449-463) supplemented with 1 μg of thiamine/ml and 0.4% DL-malate (see, e.g., Falcone, D. L., and F. R. Tabita. 1991. Expression of endogenous and foreign ribulose 1,5-bisphosphate carboxylase-oxygenase (RubisCO) genes in a RubisCO deletion mutant of Rhodobacter sphaeroides. J. Bacteriol. 173:2099-2108).

Routine DNA manipulations, including plasmid preparation, restriction endonuclease digestion, agarose gel electrophoresis, fragment ligation, and bacterial transformation, are performed by standard methods to prepare a RubisCO deficient strain. For example, as described by Paoli et al., plasmid pJP5603 derivatives are conjugated into R. capsulatus SB1003 by using E. coli S17-1 λpir, a suicide vector previously described by Penfold and Pemberton. See Penfold, Robert J., and John M. Pemberton. “An improved suicide vector for construction of chromosomal insertion mutations in bacteria.” Gene 118.1 (1992): 145-146. Homologous recombination of the plasmid-borne disrupted gene into the wild-type copy in the chromosome is forced because pJP5603 does not replicate in R. capsulatus. For complementation of mutant strains, plasmids are conjugated into R. capsulatus by triparental matings on filter pads using the helper plasmid pRK2013 as described by Paoli et al.

In addition and as described by Paoli et al., a 4.7-kb BamHI fragment, containing the R. capsulatus cbbLS genes, is cloned from pRKFIP into pUC1813. The resulting plasmid, pUC1813::FIB, lacks any EcoRI sites in the multiple cloning region so that the 639-bp EcoRI fragment within cbbL may be removed and replaced by the spectinomycin resistance (Sp^{r}) gene from pHP45Ω. The 6.5-kb BamHI fragment containing the disrupted gene is moved from pUC1813::FIΩ to pJP5603, resulting in plasmid pJP::FIΩ. Plasmid pJP::FIΩ is mobilized into R. capsulatus SB1003 from E. coli S17-1 λpir.

In addition and as described by Paoli et al., the 2-kb SalI fragment encoding the R. capsulatus cbbM gene is cloned from plasmid pK18FIIS2-I into plasmid pUC1318. The resulting construct, pUC1318FII, lacks HindIII sites within its multiple cloning region. To generate a Km^{r} cassette with flanking HindIII sites, a 1.4-kb SalI fragment encoding the Tn5 Km^{r} gene is cloned from pUC1318K into plasmid pUC1813, generating pUC1813K. The 650-bp HindIII fragment within the cbbM gene in vector pUC1318FII is removed and replaced by theHindIII fragment containing the Tn5 Km^{r} gene from plasmid pUC1813K. The resulting cbbM deletion fragment is cloned as an Xbal fragment into plasmid pTC5603, yielding pTC::FIIKm. E. coli S17-1 λpir is used to mobilize pTC::FIIKm into R. capsulatus SB1003 with Plasmid pJP::F10 already mobilized to construct a strain lacking genes for both forms of RubisCO.

To confirm attenuation, RubisCO activity may be measured as ribulose 1,5-bisphosphate-dependent ¹⁴CO₂ fixation into acid-stable 3-phosphoglycerate using methods known in the art (see, e.g., Gibson J L, Falcone D L, Tabita F R. Nucleotide sequence, transcriptional analysis and expression of genes encoded within the form I CO₂ fixation operon of Rhodobacter sphaeroides. J Biol Chem.1991; 266:14646-14653.) for samples grown to late log phase (A₆₆₀=0.9 to 1.2). If 3-phosphoglycerate containing ¹⁴C is not produced or is produced at a much slower rate than in wild-type R. capsulatus, then RubiscCO activity in the organism has been attenuated.

To confirm attenuation of the Calvin-Benson cycle itself, the ability of the deletion strain to grow photoauthrophically in the absence of an alternate electron acceptor may be assessed as described by Paoli et al. Under photoautotrophic growth conditions, where CO₂ functions as the sole carbon source, the Calvin-Benson cycle provides nearly all cellular carbon. If the Calvin-Benson cycle has been attenuated, the deletion strain will be unable to grow in the absence of an alternate electron acceptor.

In brief, mutant strains are grown under photoautotrophic conditions on solid media. Photoautotrophic growth conditions have been described previously. See, e.g., Paoli, George C., et al. “Rhodobacter capsulatus genes encoding form I ribulose-1, 5-bisphosphate carboxylase/oxygenase (cbbLS) and neighbouring genes were acquired by a horizontal gene transfer.” Microbiology 144.1 (1998): 219-227 and Paoli, George C., et al. “Expression of the cbbLcbbS and cbbM genes and distinct organization of the cbb Calvin cycle structural genes of Rhodobacter capsulatus.” Archives of Microbiology 164.6 (1995): 396-405. For example, photoautotrophic cultures of engineered R. capsulatus and wild-type R. capsulatus are grown anaerobically at 1.5% CO₂ and 98.5% H₂ in Ormerod's medium with 1 μg of thiamine/ml and 30 mM ammonia.

Similarly, a RubisCO-deletion strain of Ralstonia eutropha H16 (also known as Cupriavidus necator H16) may be prepared as previously described by Satagopan and Tabita (see Satagopan, Sriram, and F. Robert Tabita. “RubisCO selection using the vigorously aerobic and metabolically versatile bacterium Ralstonia eutropha.” The FEBS Journal (2016)), which is incorporated by reference herein in the extent it discloses a method of producing a RubisCO-deletion strain of C. necator.

### Example 2: Formate Synthesis and Assimilation Via a Modified acetyl-CoA carboxylase, 3-hydroxypropionate and Methylcitrate Cycle and a Modified RUMP Cycle in C. necator

C. necator is known to possess a 2-methylcitrate cycle II. See Brämer, Christian O., and Alexander Steinbüchel. “The methylcitric acid pathway in Ralstonia eutropha: new genes identified involved in propionate metabolism.” Microbiology 147.8 (2001): 2203-2214. Because C. necator naturally possesses a 2-methylcitrate cycle II, exogenous enzymes may be introduced into C. necator to utilize its natural methylcitrate cycle in the production of formate, for example, by utilizing the exemplary synthetic pathway shown in FIG. 4.

To produce a C. necator strain utilizing a novel carbon fixation pathway via a modified acetyl-CoA carboxylase, 3-hydroxypropionate, and methylcitrate cycle as shown in FIG. 4 and a modified RUMP cycle in place of the native Calvin-Benson cycle as shown in FIG. 5, a RubisCO deficient strain of C. necator is prepared as described by Satagopan and Tabita (include reference).

To facilitate efficient synthesis of formate via the novel synthetic pathway shown in FIG. 5, the following genes are knocked out in the RubisCO deficient C. necator strain using methods known in the art: fdsG (e.g., GenBank Gene ID 10917038 in C. necator N-1), fdsA (e.g., GenBank Gene ID 10917040 in C. necator N-1), fdsB (e.g., GenBank Gene ID 10917039 in C. necator N-1), fdsC (e.g., GenBank Gene ID 10917041 in C. necator N-1), and fdsD (e.g., GenBank Gene ID 10917042 in C. necator N-1).

The region of chromosome 1 containing fdsABCDG with flanking Xbal restriction sites may be amplified from C. necator and cloned into a pUC10 plasmid. Use of the pUC10 plasmid was previously described by Satagopan and Tabita. Site-directed mutagenesis may be used to introduce an MfeI or a ClaI restriction site immediately 5′ of the fdsB start codon and a SpeI site immediate 3′ of the fdsD stop codon. The plasmids may then be used to transfer the entire region into suicide-vector constructs for homologous recombinant, resulting in reduced formate metabolism.

Formate synthesis may be evaluated by loading 20 to 50 μL of culture filtrate onto a HPX-87H ion-exclusion high-pressure liquid chromatography column (Bio-Rad Laboratories, Richmond, Calif.). The solvent is 0.01 N H₂SO₄ at a flow rate of 0.6 mL/min. Formate synthesis may then be quantified by measuring absorbance at 210 nm with a UV monitor (model 1305; Bio-Rad Laboratories). Higher absorbance is expected to correlate with higher levels of formate synthesis. It is anticipated that absorbance at 210 nm will be considerably lower for the engineered strain's culture filtrate relative to wild-type because less formate should be produced and secreted by the engineered strain.

In one embodiment, methods known in the art are then used to insert genes corresponding to the following enzymes associated with the synthetic pathways shown in FIGS. 4 and 12 into the RubisCO and formate synthesis (fdsABCDG)-deficient C. necator formate acetyltransferase encoded by pflB/D/ybiW (GenBank Gene ID 945514, 948454, and 945444) from E. coli K-12 (GenBank Accession No. ALI38381.1, SEQ ID NO: 1); D-mannonate oxidoreductase encoded by uxuB (GenBank Gene ID 946795) from E. coli K-12 (RefSeq Accession No. NP_418743.1, SEQ ID NO: 2); 3-hydroxypropionyl-CoA synthase encoded by Msed_1456 (GenBank Gene ID 5104826) from M. sedula ATCC 51363 (RefSeq Accession No. WP_048060101.1, SEQ ID NO: 3); formyl-CoA:oxalate CoA-transferase encoded by frc (GenBank Gene ID 946842) from E. coli K-12 (RefSeq Accession No. NP_416875.1, SEQ ID NO: 4).

The following enzymes associated with the synthetic pathways shown in FIGS. 4 and 12 and FIGS. 13-16 are naturally expressed in C. necator: 2-methylcitrate synthase (RefSeq Accession No. WP_011615747.1; SEQ ID NO:5); 2-methylcitrate dehydratase (RefSeq Accession No. WP_011616750.1; SEQ ID NO:6); aconitate hydratase B (RefSeq Accession No. WP_013952213.1; SEQ ID NO:7); methylisocitrate lyase (RefSeq Accession No. WP_013956854.1; SEQ ID NO:8); acetyl-CoA carboxylase carboxyl transferase (RefSeq Accession No. WP_010813183.1; SEQ ID NO:9, WP_010814637.1; SEQ ID NO:10, and WP_010808961.1; SEQ ID NO:11); fructose-bisphosphate aldolase B (GenBank Accession No. AEI75959.1; SEQ IE NO:12); enoyl-CoA hydratase (RefSeq Accession No. WP_011616888.1; SEQ ID NO:13); acryloyl-CoA reductase (RefSeq Accession No. WP_013956251.1; SEQ ID NO:14); malate dehydrogenase (RefSeq Accession No. WP_010814614.1; SEQ ID NO:15); fumarate hydratase (RefSeq Accession No. WP_013957339.1; SEQ ID NO:16 and WP_013951812.1; SEQ ID NO:17); succinate dehydrogenase (RefSeq Accession No. WP_010814618.1; SEQ ID NO:18, KUE85836.1; SEQ ID NO:19, KUE85833.1; SEQ ID NO:20); acetyl-coenzyme A synthetase (RefSeq Accession No. WP_011615709.1; SEQ ID NO:21); acetaldehyde dehydrogenase (RefSeq Accession No. WP_013952197.1; SEQ ID N0:22); phosphoenolpyruvate carboxylase (RefSeq Accession No. WP_013957758.1; SEQ ID NO:23); phosphate acetyltransferase (RefSeq Accession No. WP_013953470.1; SEQ ID NO:24); transketolase (RefSeq Accession No. WP_041228130.1; SEQ ID NO:25); transaldolase (RefSeq Accession No. WP_013957205.1; SEQ ID NO:26); ribulose-phosphate 3-epimerase (RefSeq Accession No. WP_013953007.1; SEQ ID NO:27); and ribose 5-phosphate isomerase A (RefSeq Accession No. WP_013957204.1; SEQ ID NO:28).

As previously described, examples of microorganisms carrying multiple exogenous enzymes and having successfully engineered metabolic pathways have been demonstrated by several groups, including engineered E. coli, P. aeruginosa, C. necator, and R. capsulatus utilizing synthetic pathways to produce chemical commodities.

Once these genes are introduced, the engineered C. necator strain is able to utilize the synthetic formate synthesis pathway shown in FIG. 4 and the synthetic formate assimilation pathway (a modified RuMP pathway) shown in FIG. 12.

To determine if engineered C. necator more efficiently performs carbon fixation relative to wild-type C. necator, biomass generation in the engineered C. necator relative to wild-type C. necator may be examined by comparing the absorbance at 660 nm (A₆₆₀) for both strains over time. Both strains may be cultured in chemoautotrophic conditions in either liquid minimal medium bubbled with 2.5% or 10% CO₂, 50% air (10.5% O₂), balanced with H₂.

A₆₆₀ provides a measure of growth in a culture, with higher A₆₆₀ correlating with higher concentration of microorganism (i.e., a faster growth rate for pre-stationary phase cultures). If the engineered C. necator more efficiently fixes carbon related to the wild-type strain, it is anticipated that the strain will produce more carbon building blocks and grow more rapidly than the wild-type strain.

In addition, carbon fixation efficiency in the engineered C. necator strain may be investigated by measuring the rate of carbon dioxide fixation and the percentage converted to biomass. It is anticipated that the engineered C. necator strain will fix carbon dioxide at a similar or higher rate than the wild-type strain and that a higher percentage of the fixed carbon dioxide will be converted to biomass.

To measure carbon dioxide fixation, an infrared sensor (Vaisala GMT) and a cultivation vessel coupled with sensors for the measurement of carbon dioxide and oxygen in the inlet and outlet gases may be utilized as described by Sydney et al. See Sydney, Eduardo Bittencourt, et al. “Potential carbon dioxide fixation by industrially important microalgae.” Bioresource Technology 101.15 (2010): 5892-5896. In the inlet, carbon dioxide flow is monitored by a rotameter and measured by a thermal dispersion mass flow sensor (Aalborg GFM), while oxygen flow is monitored by a rotameter and its concentration in the air measured by an electrochemical sensor (Alphasense O2-A2). In the outlet, total flow is measured by a mass flow sensor (Aalborg model GFM), the percentage of carbon dioxide by an infrared sensor (Vaisala GMT) and the percentage of oxygen by an O2-A2 sensor. These sensors are all connected to Novus model N1100 controllers. Data acquisition occurs at 15 min intervals by Laquis software.

A blank trial, using only sterile media in the vessel, should be run for 5 days with data acquisition in order to define sensors baselines for O₂ and CO₂ to be used as basis to calculate carbon dioxide consumption.

The determination of carbon dioxide fixation is done based on the CO₂ consumption profile. The trapezoidal method was used in order to integrate the curves (CO₂ cons g/h and CO₂ base line). The areas obtained are subtracted and the difference between them corresponding to the total amount carbon dioxide consumed.

Biomass may be analyzed by methods described by Sydney et al. In brief, after a pre-determined period of time (e.g., one week), the cells are removed from culture by centrifugation (3600 rpm for 20 min), washed (distilled water), recentrifuged again, and dried at 60° C. until constant weight. The dried biomass is analyzed from carbohydrates, proteins, and lipids.

Lipids are determined by extraction with methanol:chloroform 1:1 followed by a liquid-liquid extraction with hexane. The phenol-sulfuric method is used for total carbohydrate determination, and the Lowry method is used for protein determination. The biomass composition in terms of percentage protein, carbohydrate, and lipid may then be determined by comparing the measured amounts to the dried biomass weight. Carbon composition may then be measured by estimating that proteins are 45% carbon, carbohydrates are 40% carbon, and lipids are 87% carbon.

The % of CO₂ converted to biomass may then be calculated by comparing the amount of carbon fixed based on the CO₂ consumption profile (where CO2 is 27% carbon by mass) to the total weigh of carbon in the dried biomass.

### Example 3: In Silico Analysis of Synthetic Carbon Fixation Pathways

Some hydrogen-oxidizing microorganisms (e.g., C. necator strains) can naturally assimilate CO₂ through the Calvin-Benson cycle when growing under lithoautotrophic conditions. However, it has been shown that this carbon fixation pathway is less efficient compared to other alternative carbon fixation pathways. Alternative synthetic autotrophic pathways were analyzed to determine efficient pathways for carbon fixation that could be implemented in C. necator.

While the analysis was based only upon reaction stoichiometry and mass balances, this In Silico analysis is considered in the field to be a reliable predictor of metabolic capabilities. Stoichiometry based models enable efficient calculation of maximum theoretical yields for any desired product under defined conditions. Thus, maximum theoretical yields for isopropanol production under lithoautotrophic conditions were determined for each proposed pathway using the native Calvin-Benson cycle as a reference and then ranked according to the isopropanol yield per H₂ consumed (Y_{ipOH/H2}). In addition, a thermodynamic feasibility analysis of these pathways was performed to identify possible thermodynamic bottlenecks in pathways.

Metabolic fluxes were calculated under autotrophic conditions (i.e., H₂/CO₂) in C. necator using parsimonious flux balance analysis (pFBA) considering no biomass formation and no ATP consumption for cell maintenance. To evaluate the most efficient pathways, the amount of flux through the hydrogen uptake reaction was set to a fixed value, while the oxygen and CO₂ uptake rates were basically limited by the amount of energy assimilated from hydrogen. Maximum isopropanol production was chosen as the objective function. The energy required to convert CO₂ into isopropanol was defined as the ratio of carbon flux maximizing the production of isopropanol (with units [C-mol/h]) per H2 consumed (with units [mol/h]). The CO₂ and O₂ assimilation fluxes were also estimated to calculate the following molar ratios: H₂/CO₂, H₂/CO₂, and H₂/O₂. Formate biosynthesis was assumed to be performed by a NADPH-dependent formate dehydrogenase. The cumulative changes in the reaction Gibbs free energy (ΔrG′m) along the different pathways were determined assuming 1 mM as the standard metabolites concentration. The Calvin-Benson cycle was used as reference when evaluating a synthetic pathway's efficiency. A comparison between different synthetic carbon fixation pathways similar to those presented in FIG. 7-12 is presented in Table 8.

The pathway similar to FIG. 7 includes enzymatic conversion of phosphoenolpyruvate to oxaloacetate only.

The alternative pathway similar to FIG. 7 includes enzymatic conversion of phosphoenolpyruvate to pyruvate only.

In the pathway similar to FIG. 8, pyruvate produced by the enzymatic conversion of 2-phospho-D-glycerate to phosphoenolpyruvate followed by enzymatic conversion of phosphoenolpyruvate to pyruvate is enzymatically converted to acetyl-CoA.

In the pathway similar to FIG. 9, pyruvate created by the enzymatic conversion of 2-phospho-D-glycerate to phosphoenolpyruvate followed by enzymatic conversion of phosphoenolpyruvate to pyruvate is enzymatically converted to acetyl-CoA.

The pathway similar to FIG. 11 includes production of formate by the enzymatic conversion of 2-oxobutyrate and carbon dioxide.

The pathway similar to FIG. 12 includes production of formate by the enzymatic conversion of 2-oxobutyrate and carbon dioxide.

Overall, analysis based on reaction stoichiometry and mass balances reaction showed that the maximum theoretical yield for isopropanol could be increased by up to 50% compared to the Calvin-Benson cycle. Energy costs in synthetic pathways could be reduced by various means, such as, for example, the use of NADH-dependent instead of NADPH dependent activities and an acyl-CoA transferase instead of the malate thiokinase.

TABLE 8
Maximum theoretical yields calculated for different carbon fixing metabolic
pathways under H₂/CO₂ conditions (H₂ limitation) when maximizing isopropanol using
the C. necator stoichiometric model

| | | Y(ipOH/H₂) | | | | Cumulative |
|---|---|---|---|---|---|---|
| | Y(ipOH/CO₂) | [C-mmol/ | Y(H₂/CO₂) | Y(H₂/O) | Y(O₂/CO₂) | ΔrG^{m} |
| | [C-mmol/C-mmol] | mmol] | [mmol/mmol] | | | [kJ/mol] |
| Calvin-Benson Cycle | 1 | 0.196 | 5.100 | 4.857 | 5.100 | −125.6 |
| Pathway similar to | 1 | 0.261 | 3.833 | 9.200 | 3.833 | −141.2 |
| FIG. 7 (P1) | | | | | | |
| Alternative pathway | 1 | 0.275 | 3.633 | 11.474 | 3.633 | −100.7 |
| similar to FIG. 7 | | | | | | |
| Pathway similar to | 1 | 0.275 | 3.633 | 11.474 | 3.633 | −91 |
| FIG. 8 | | | | | | |
| Pathway similar to | 1 | 0.275 | 3.633 | 11.474 | 3.633 | −98.8 |
| FIG. 9 (P2) | | | | | | |
| Pathway similar to | 1 | 0.275 | 3.633 | 11.474 | 3.633 | −11.6 |
| FIG. 11 (P5) | | | | | | |
| Pathway similar to | 1 | 0.244 | 4.100 | 7.455 | 4.100 | −142 |
| FIG. 12 | | | | | | |

### Example 4: Materials and Methods for Strain Construction for Pathways P1, P2, P5 and P10 as Depicted in FIGS. 13, 14, 15 and 16

### Materials:

Media Recipes

First Conjugation Protocol:

Minimal plates for selection of first-crossover Cupriavidus necator.

| | g/L | For 500 ml of plates | |
|---|---|---|---|
| KH₂PO₄ | 1.15 | 0.575 g | |
| Na₂HPO₄ | 1.15 | 0.575 g | |
| NH₄Cl (0.1% w/v) | 1.00 | 0.5 g | |
| MgSO₄•7H₂O | 0.50 | 0.25 g | |
| CaCl₂•2H₂O | 0.062 | 0.031 g | Filter sterilize in ~243 ml |
| Trace metal solution | 1 ml/L | 0.5 ml | Filter sterilise |
| Agar | 15 g | 7.5 g in 250 ml | Autoclave |
| Fructose | 0.5% | 6.25 ml of 40% stock solution | Filter sterilise |
| Tetracycline | 25 μg/ml | 500 μL of 25 mg/ml stock solution | Filter sterilise |

Low salt LB (+sucrose+agar for plates) for selection of second crossover Cupriavidus necator, various levels of sucrose selection against SacB:

| | g/L | For 500 ml | |
|---|---|---|---|
| Tryptone | 10 | 5 g | |
| Yeast Extract | 5 | 2.5 g | |
| Agar (plates) | 15 | 7.5 g | Autoclave in 250 ml |
| For 15% sucrose | 150 g | 75 g | Filter sterilized in 250 ml |
| For 10% sucrose | 100 g | 50 g | Filter sterilized in 250 ml |
| For 5% sucrose, | 50 g | 25 g | Filter sterilized in 250 ml |
| 0.2% fructose | 2 g | 1 g | |

Second conjugation protocol: Plates for Cupriavidus necator first-crossover selection (LB —NaCl+gentamycin+tetracycline) and plates for second-crossover selection (LB —NaCl+gentamycin+10% sucrose):

| | g/L | For 500 ml | |
|---|---|---|---|
| Tryptone | 10 | 5 g | |
| Yeast Extract | 5 | 2.5 g | |
| Agar | 15 | 7.5 g | |
| For 10% sucrose | 100 g | 50 g | |
| For 2% gluconate | 20 g | 10 g | Autoclave in 250 ml |
| gentamycin | 5 μg/ml | 250 μl of 10 | Added after cooling |
| | | mg/ml stock | |
| For tetracycline | 5 μg/ml | 100 μl of 25 | Added after cooling |
| | | mg/ml stock | |

TSB (+agar for plates):

| | g/L | For 500 ml of plates | |
|---|---|---|---|
| Tryptone Soy Broth | 27.5 | 13.75 g | |
| without dextrose | | | |
| Agar | 15 | 7.5 g | |
| For 2% gluconate | 20 | 10 | Autoclave |

INV-2 with fructose and sodium formate:

| | Order | | Concentration |
|---|---|---|---|
| | to add | Component | in g/kg* |
| In water up to ~70% | 1 | Fructose | 9 |
| of final volume. | 2 | Sodium formate | 4.04 |
| pH to <5 using HCl | 3 | Urea | 2 |
| | 4 | K2HPO4 | 0.5325 |
| | 5 | NaH2HPO4•H2O | 0.3015 |
| In separate vessel: Dissolve | 1 | Citric acid | 0.0675 |
| in water in order given up | 2 | FeCl3•6H2O | 0.0285 |
| to ~10% of final volume. | 3 | CaCl2•2H2O | 0.0825 |
| | 4 | MgSO4•7H2O | 0.285 |
| In separate vessel: | | Trace elements** | 11.25 mL |
| Measure out trace element | | | |
| solution/dilute 10x | | | |

Add the metals and trace elements to the main volume.
pH to 6.5 using KOH. Filter sterilize.
*The amount of sodium formate in this recipe is equivalent to 2.7 g formic acid/kg, initial tests also tried using equivalent potassium formate, vs no formate. Further tests are to use the equivalent of 1.35 g formic acid/kg.
**Composition of trace elements solution:

| | |
|---|---|
| ZnSO4•7H2O | 250 mg/kg |
| MnCl2•4H2O | 12 mg/kg |
| H3BO3 | 330 mg/kg |
| CoCl2•6H2O | 330 mg/kg |
| NiSO4•6H2O | 300 mg/kg |
| Na2MoO4•2H2O | 50 mg/kg |
| CuSO4•5H2O | 50 mg/kg |

adjusted to pH = 3.5 using HCl 25% v/v.

For these embodiments, methods known in the art are then used to insert genes corresponding to the enzymes associated with the synthetic pathways shown in FIGS. 13, 14m, 15 and 16. Nonlimiting examples of nucleotide sequences and corresponding amino acid sequences of enzymes tetrahydrofolate ligase, malyl-CoA lyase, malate thiokinase, methenyl tetrahydrofolate cyclohydrolase, glycerate 2-kinase, hydroxypyruvate reductase, methylene-tetrahydrofolate dehydrogenase, serine-glyoxylate aminotransferase, phosphoenolpyruvate carboxylase and pyruvate formate-lyase inserted into the P1 pathway including are set forth in SEQ ID NOs:29 through 50, respectively.

Sequences obtained to construct strains for pathways P1, P2, P5 and P10 are set forth in Table 9. Details with respect to the strains and plasmids constructed are set forth in Tables 10 and 11.

TABLE 9
Sequences

| Accession number | | | | | | |
|---|---|---|---|---|---|---|
| or other sequence | EC | | | | | |
| identification | number | Source | construct | BDIGENE # | TUs | Pathways |
| SEQ ID NO: 51 | | homology for phaCAB | Hom1-pTac | BDIGENE0426 | TU1 | 1 A-D, 2A-D |
| | | region | | | | |
| KMT22112.1; | 6.3.4.3 | | RBS-fhs | BDIGENE0427 | TU1 | 1 A-D, 2A-D |
| SEQ ID NO: 52 | | | | | | |
| sp\|O58231.1; | 2.7.1.161 | Pyrococcus horikoshii | RBS-gck | BDIGENE0428 | TU1 | 1 A-D, 2A-D |
| SEQ ID NO: 53 | | OT3 | | | | |
| SEQ ID NO: 54 | | | pRbcL(M1) | BDIGENE0429 | TU2 | 1 A-D, 2A-D |
| AAA62654.1; | 6.2.1.9 | Methylobacterium | RBS-mtkA | BDIGENE0430 | TU2 | 1 A-D, 2A-D |
| SEQ ID NO: 55 | | extorquens AM1 | | | | |
| ACS39575.1; | | | RBS-mtkB | BDIGENE0431 | TU2 | 1 A-D, 2A-D |
| SEQ ID NO: 56 | | | | | | |
| ACS39577.1; | 4.1.3.24 | Methylobacterium | RBS-mcl-(BsmBl | BDIGENE0432 | TU2 | 1 A-D, 2A-D |
| SEQ ID NO: 57 | | extorquens AM1 | sites)-ter-Hom2 | | | |
| ACS39570.1; | 2.6.1.45 | Methylobacterium | Pnk-RBS-sga | BDIGENE0433 | TU3 | 1B 1D 2B 2D |
| SEQ ID NO: 58 | | extorquens AM1 | | | | |
| ACS39571.1; | 1.1.1.81 | Methylobacterium | RBS-hpr | BDIGENE0434 | TU3 | 1B 1D 2B 2D |
| SEQ ID NO: 59 | | extorquens AM1 | | | | |
| ACS39572.1; | 1.5.1.5 | Methylobacterium | RBS-mtdA | BDIGENE0435 | TU3 | 1B 1D 2B 2D |
| SEQ ID NO: 60 | | extorquens AM1 | | | | |
| ACS39573.1; | 3.5.4.9 | Methylobacterium | RBS-fch | BDIGENE0436 | TU3 | 1B 1D 2B 2D |
| SEQ ID NO: 61 | | extorquens AM1 | | | | |
| ACS39576.1; | 4.1.1.31 | Methylobacterium | pRbcl(M9)-RBS-ppc | BDIGENE0437 | TU4 | 1B 1D 2B 2D |
| SEQ ID NO: 62 | | extorquens AM1 | | | | |
| A0A0A6PUK1; | 2.3.1.54 | Clostridium butyricum | RBS-Pfl | BDIGENE0438 | TU4 | 1C 1D 2C |
| SEQ ID NO: 63 | | | | | | 2D |
| A0A0A6PUK1; | 2.3.1.54 | Clostridium butyricum | RBS-pfl | BDIGENE0439 | TU4 | 1C 1D 2C |
| SEQ ID NO: 64 | | | | | | 2D |
| | | homology for phaCAB | Hom1-pTac | BDIGENE0580 | P5 | P5, P5 op |
| SEQ ID NO: 65 | | region | | | TU1 | |
| U94348.2; | 6.2.1.1 | Pyrobaculum | RBS-acsA | BDIGENE0581 | P5 | P5, P5 op |
| SEQ ID NO: 66 | | aerophilum | | | TU1 | |
| EF566941.2; | 1.2.1.10 | Acinetobacter spp | RBS-aldH-Ter | BDIGENE0582 | P5 | P5, P5 op |
| SEQ ID NO: 67 | | strain HBS-2 | | | TUI | |
| SEQ ID NO: 68 | | | pRbcLM1 | BDIGENE0583 | P5 | P5, P5 op |
| | | | | | TU2, | |
| | | | | | TU2 | |
| | | | | | op | |
| D64136.1 ; | 4.1.2.43 | Methylomonas | RBS-rmpA | BDIGENE0584 | P5 | P5 |
| SEQ ID NO: 69 | | aminofaciens | | | TU2 | |
| GI: 648264618; | 5.3.1.27 | Methylococcus | RBS-rmpB | BDIGENE0585 | P5 | P5 |
| SEQ ID NO: 70 | | capsulatus | | | TU2 | |
| AAB03048; | 2.7.1.11 | Escherischia coli | Rbs-pfkA-Ter-Hom2 | BDIGENE0586 | P5 | P5 |
| SEQ ID NO: 71 | | | | | TU2 | |
| Genbank assembly; | 4.1.2.13 | Methylococcus | RBS-tkt | BDIGENE0587 | P5 | P5 operon |
| SEQ ID NO: 72 | | capsulatus | | | TU2 | |
| | | | | | OP | |
| GCA_000008325.1; | 2.2.1.1, | Methylococcus | fba-rmpA | BDIGENE0588- | P5 | P5 operon |
| SEQ ID NO: 73 | 4.1.2.43 | capsulatus | | 9 | TU2 | |
| | | | | | OP | |
| 3225737: 3239736; | 2.2.1.2, | Methylococcus | rmpB-tal | BDIGENE0590- | P5 | P5 operon |
| SEQ ID NO: 74 | 5.3.1.27 | capsulatus | | 1 | TU2 | |
| | | | | | OP | |
| AE017282.2; | 2.7.1.11 | Methylococcus | RBS-pfkA-Ter-Hom2 | BDIGENE0592 | P5 | P5 operon |
| SEQ ID NO: 75 | | capsulatus | | | TU2 | |
| | | | | | OP | |
| SEQ ID NO: 76 | | homology for phaCAB | Hom1-pTac | BDIGENE0593 | P10 | P10 A-D |
| | | region | | | TU1 | |
| Q96YK1; | 1.2.1.75 | Solfolobus tokodaii | RBS-mcr | BDIGENE0594 | P10 | P10 A-D |
| SEQ ID NO: 77 | | | | | TU1 | |
| AF159146.1; | 5.4.3.2 | Clostridium | RBS-KamA-Ter | BDIGENE0595 | P10 | P10 A-D |
| SEQ ID NO: 78 | | subterminale | | | TU1 | |
| AKA81705.1; | 2.6.1.18 | Pseudomonas | Pnk-RBS-BauA | BDIGENE0596 | P10 | P10.B P10D |
| SEQ ID NO: 79 | | fluorescens | | | TU3 | |
| NP_416958; | 1.1.1.40 | Escherischia coli | RBS-maeB-Ter | BDIGENE0597 | P10 | P10.B P10D |
| SEQ ID NO: 80 | | | | | TU3 | P2 |
| NP_415040.1; | 4.1.1.47 | Escherischia coli | pRbcL(M9)-RBS-gcl | BDIGENE0598 | P10 | P10.B P10D |
| SEQ ID NO: 81 | | | | | TU4 | |
| AAC76159; | 1.1.1.60 | Escherischia coli | RBS-gar | BDIGENE0599 | P10 | P10.B P10D |
| SEQ ID NO: 82 | | | | | TU4 | |
| AAB93855; | 2.7.1.31 | Escherischia coli | RBS-glxK | BDIGENE0600 | P10 | P10.B P10D |
| SEQ ID NO: 83 | | | | | TU4 | |
| SEQ ID NO: 84 | | | phaB2C2-LHA-term | BDIGENE0625 | | |
| SEQ ID NO: 85 | | | phaB2C2-RHA-term | BDIGENE0626 | | |
| SEQ ID NO: 86 | | | A0006-9-LHA-term | BDIGENE0627 | | |
| SEQ ID NO: 87 | | | A0006-9-RHA-term | BDIGENE0628 | | |
| P52489.1; | 2.7.1.40 | Saccharomyces | PNK-pyk | BDIGENE0791 | P10d, | P10C P10D |
| SEQ ID NO: 88 | | cerevisiae s288 | | | P2 | P2 |

TABLE 10
BDISC numbers for strains and plasmids

| Collection | | | |
|---|---|---|---|
| Number | Strain | Plasmid/genotype summary/pathway | Purpose |
| BDISC0086 | E. coli NEB5alpha | TcColE1oriTsacB (fdsGBACD) | Deletion fds operon |
| BDISC0087 | E. coli S17-1 | TcColE1oriTsacB (fdsGBACD) | Deletion fds operon |
| BDISC0112 | Cupriavidus necator | Wild Type | Wild Type |
| | H16 | | |
| BDISC0121 | E. coli S17-1 | p(TcColE1oriTsacB) | Control (EV) |
| BDISC0217 | E. coli NEB5alpha | TcColE1oriTsacB (ΔcbbLS-pl) | Deletion Rubisco (pl) |
| BDISC0218 | E. coli S17-1 | TcColE1oriTsacB (ΔcbbLS-pl) | Deletion Rubisco (pl) |
| BDISC0219 | E. coli NEB5alpha | TcColE1oriTsacB (ΔcbbLS-chr) | Deletion Rubisco (chr) |
| BDISC0220 | E. coli S17-1 | TcColE1oriTsacB (ΔcbbLS-chr) | Deletion Rubisco (chr) |
| BDISC0122 | E. coli S17-1 | p(TcColE1oriTsacB) ΔphaCAB | For control strain |
| BDISC0312 | Cupriavidus necator | ΔfdsGBACD | No use of formate |
| | H16 | | |
| BDISC0319 | Cupriavidus necator | ΔfdsGBACD, ΔcbbLS(pl) | Interim strain |
| | H16 | | |
| BDISC0334 | Cupriavidus necator | ΔfdsGBACD, ΔcbbLS(pl + chr) | Base strain |
| | H16 | | |
| BDISC0439 | E. coli NEB5alpha | pBBR1 Goldengate 1A | GG vector |
| BDISC0440 | E. coli NEB5alpha | pBBR1 Goldengate 1B | GG vector |
| BDISC0543 | E. coli NEB5alpha | TcColE1oriTsacB 2A | GG vector |
| BDISC0546 | E. coli NEB5alpha | Tc15aoriTsacB 2A | GG vector |
| BDISC0571 | E. coli NEB5alpha | Hom1-pTac | Gene order |
| BDISC0572 | E. coli NEB5alpha | RBS-acsA | Gene order |
| BDISC0573 | E. coli NEB5alpha | RBS-aldH-Ter | Gene order |
| BDISC0574 | E. coli NEB5alpha | pRbcLM1 | Gene order |
| BDISC0575 | E. coli NEB5alpha | RBS-rmpA | Gene order |
| BDISC0576 | E. coli NEB5alpha | RBS-rmpB | Gene order |
| BDISC0577 | E. coli NEB5alpha | Rbs-pfkA-Ter-Hom2 | Gene order |
| BDISC0578 | E. coli NEB5alpha | RBS-pfkA-Ter-Hom2 | Gene order |
| BDISC0579 | E. coli NEB5alpha | Hom1-pTac | Gene order |
| BDISC0580 | E. coli NEB5alpha | RBS-mcr | Gene order |
| BDISC0581 | E. coli NEB5alpha | RBS-KamA-Ter | Gene order |
| BDISC0582 | E. coli NEB5alpha | Pnk-RBS-BauA | Gene order |
| BDISC0583 | E. coli NEB5alpha | RBS-maeB-Ter | Gene order |
| BDISC0584 | E. coli NEB5alpha | pRbcL(M9)-RBS-gcl | Gene order |
| BDISC0585 | E. coli NEB5alpha | RBS-gar | Gene order |
| BDISC0586 | E. coli NEB5alpha | RBS-glxK | Gene order |
| BDISC0653 | E. coli NEB5alpha | P5 TU1 | Assembled TU |
| BDISC0654 | E. coli NEB5alpha | P5 TU2 | Assembled TU |
| BDISC0655 | E. coli NEB5alpha | P10 TU1 | Assembled TU |
| BDISC0656 | E. coli NEB5alpha | P10 TU3 | Assembled TU |
| BDISC0657 | E. coli NEB5alpha | P10 TU4 | Assembled TU |
| BDISC0658 | E. coli NEB5alpha | Tcp15aoriTsacB P5 | Assembled pathway |
| BDISC0659 | E. coli S17-1 | Tcp15aoriTsacB P5 | Insertion |
| BDISC0718 | E. coli NEB5alpha | Tcp15aoriTsacB P10 | Assembled pathway |
| BDISC0719 | E. coli S17-1 | Tcp15aoriTsacB P10 | Insertion |
| BDISC0757 | C. necator H16 | ΔfdsGBACD, ΔcbbLS, ΔphaCAB, P10 | P10 strain |
| BDISC0760 | E. coli NEB5alpha | RBS-tkt | Gene order |
| BDISC0761 | E. coli NEB5alpha | fba-rmpA | Gene order |
| BDISC0762 | E. coli NEB5alpha | rmpB-tal | Gene order |
| BDISC0783 | E. coli 5alpha | Tcp15aoriTsacB P5 operon | Assembled pathway |
| BDISC0784 | E. coli s17-1 | Tcp15aoriTsacB P5 operon | Insertion |
| BDISC0787 | C. necator H16 | ΔfdsGBACD, ΔcbbLS, ΔphaCAB, P1.1 | P1.A strain |
| BDISC0899 | E. coli NEB5a | pTc(p15aOriTSacB)-P10b | Assembled pathway |
| BDISC0900 | E. coli S17-1 | pTc(p15aOriTSacB)-P10b | Insertion |
| BDISC0928 | C. necator H16 | ΔfdsGBACD, ΔcbbLS, ΔphaCAB, P1.C | P1.C strain (prev. P2.1) |
| BDISC0930 | C. necator H16 | ΔfdsGBACD, ΔcbbLS, ΔphaCAB | Control strain |
| BDISC0932 | E. coli NEB5alpha | phaB2C2 LHA-term | synthetic level 0 part |
| BDISC0933 | E. coli NEB5alpha | term-phaB2C2 RHA | synthetic level 0 part |
| BDISC0935 | E. coli NEB5alpha | A0006-9 LHA-term | synthetic level 0 part |
| BDISC0937 | E. coli NEB5alpha | term-A0006-9 RHA | synthetic level 0 part |
| BDISC0978 | C. necator H16 | ΔfdsGBACD, ΔcbbLS | Base strain 2 |
| BDISC0989 | E. coli NEB5a | PNK-pyk | Synthetic gene |
| BDISC1016 | E. coli NEB5a | pTc(p15aOriTSacB)-p1.b | Assembled pathway |
| BDISC1017 | E. coli s17-1 | pTc(p15aOriTSacB)-p1.b | Insertion |
| BDISC1018 | E. coli NEB5a | pTc(p15aOriTSacB)-p2 | Assembled pathway |
| BDISC1019 | E. coli s17-1 | pTc(p15aOriTSacB)-p2 | Insertion |
| BDISC1020 | C. necator H16 | ΔfdsGBACD, ΔcbbLS, P5 (No PTAC) | P5 strain |
| BDISC1022 | C. necator H16 | ΔfdsGBACD, ΔcbbLS, P5 operon (No | P5 operon strain |
| | | PTAC) | |
| BDISC1025 | E. coli NEB5a | pTc(p15aOriTSacB)-P10.d- | Assembled pathway |
| BDISC1026 | E. coli s17-1 | pTc(p15aOriTSacB)-P10.d- | Insertion |
| BDISC1060 | C. necator H16 | ΔfdsGBACD, ΔcbbLS, ΔphaCAB, | P10C strain |
| | | P10C | |
| BDISC1062 | E. coli 5alpha | pTc(p15aOriTSacB)-P10.d+ | Assembled pathway |
| BDISC1063 | E. coli s17-1 | pTc(p15aOriTSacB)-P10.d+ | Insertion |
| BDISC1147 | E. coli 5alpha | pBBR1-1A-TU1 | Assembled TU (EF) |
| BDISC1148 | E. coli 5alpha | pBBR1-1B-TU2 | Assembled TU (EF) |
| BDISC1149 | E. coli 5alpha | pBBR1-1A-TU3 | Assembled TU (EF) |
| BDISC1150 | E. coli 5alpha | pBBR1-1B-TU4 | Assembled TU (EF) |
| BDI3C1151 | E. coli 5alpha | pTc-PIA | Assembled pathway |
| BDISC1152 | E. coli 5alpha | pTc-1C (was 2.1) | (EF) |
| | | | Assembled pathway |
| BDISC1162 | C. necator H16 | ΔfdsGBACD, ΔcbbLS, P1B | (EF) |
| | | | P1B strain |
| BDISC1164 | C. necator H16 | ΔfdsGBACD, ΔcbbLS, P1D | P1D strain |

TABLE 11
Strains and Vectors prepared for Strain construction.

| Strain | Genotype | BDISC | Formate |
|---|---|---|---|
| Δfds | Cupriavidus necator H16 ΔfdsGBACD | 0312 | N/A |
| Base | Cupriavidus necator H16 ΔfdsGBACD ΔcbbLS | 0334 | N/A |
| Base | Cupriavidus necator H16 ΔfdsGBACD ΔcbbLS | 0978 | N/A |
| 2 | | | |
| Control | Cupriavidus necator H16 ΔfdsGBACD ΔcbbLS | 0930 | N/A |
| | ΔphaCAB | | |
| P1A | Cupriavidus necator H16 ΔfdsGBACD ΔcbbLS | 0787 | Yes |
| | ΔphaCAB: pTac-fhs-gck pRbcl(M1)-mtkA-mtkB-mcl | | |
| P18 | Cupriavidus necator H16 ΔfdsGBACD ΔcbbLS | 1162 | Yes |
| | ΔphaCAB: pTac-fhs-gck pRbcl(M1)-mtkA-mtk8-mcl | | |
| | ΔA0006: PnK-sga-hpr pRbcl(M1)-mtdA-fch-ppc | | |
| P1C | Cupriavidus necator H16 ΔfdsGBACD ΔcbbLS | 0928 | Yes |
| | ΔphaCAB: pTac-fhs-gck pRbcl(M1)-mtkA-mtkB-mcl- | | |
| | pfl | | |
| P1D | Cupriavidus necator H16 ΔfdsGBACD ΔcbbLS | 1164 | Yes |
| | ΔphaCAB: pTac-fhs-gck pRbcl(M1)-mtkA-mtkB-mcl- | | |
| | pfl ΔA0006: PnK-sga-hpr pRbcl(M1)-mtdA-fch-ppc | | |
| P2A | Cupriavidus necator H16 ΔfdsGBACD ΔcbbLS | Conjugation | Yes |
| | ΔphaCAB: pTac-fhs-gck pRbcl(M1)-mtkA-mtk8-mcl | of | |
| | ΔphaC282: PnK-Pyk-Mae8 | equivalent | |
| P2B | Cupriavidus necator H16 ΔfdsGBACD ΔcbbLS | P1 strain + Yes | |
| | ΔphaCAB: pTac-fhs-gck pRbcl(M1)-mtkA-mtkB-mcl | BDISC1019 | |
| | ΔA0006: PnK-sga-hpr pRbcl(M1)-mtdA-fch-ppc | | |
| | ΔphaC2B2: PnK-Pyk-MaeB | | |
| P2D | Cupriavidus necator H16 ΔfdsGBACD ΔcbbLS | Yes | |
| | ΔphaCAB: pTac-fhs-gck pRbcl(M1)-mtkA-mtkB-mcl- | | |
| | pfl ΔA0006: PnK-sga-hpr pRbcl(M1)-mtdA-fch-ppc | | |
| | ΔphaC2B2: PnK-Pyk-Mae8 | | |
| P5 | Cupriavidus necator H16 ΔfdsGBACD ΔcbbLS | 1020 | Yes |
| | ΔphaCAB: acsA-AldH pRbcl(M1)-rmpA-rmp8-pfkA | | |
| P5-op | Cupriavidus necator H16 ΔfdsGBACD ΔcbbLS | 1022 | Yes |
| | ΔphaCAB: acsA-AldH pRbcl(M1)-tkt-fba-rmpA-rmpB- | | |
| | tal-pfkA | | |
| P10A | Cupriavidus necator H16 ΔfdsGBACD ΔcbbLS | 0757 | No |
| | ΔphaCAB: pTac-mcr-KamA pRbcl(M1)-mtkA-mtk8- | | |
| | mcl ΔphaC2B2: PnK-pyk ΔA0006: Pnk-BauA-mae8 | | |
| | pRbcL(M9)-gcl-gar-glxK | | |
| P10C | Cupriavidus necator H16 ΔfdsGBACD ΔcbbLS | 1060 | No |
| | ΔphaCAB: pTac-mcr-KamA pRbcl(M1)-mtkA-mtkB- | | |
| | mcl ΔphaC2B2: Pnk-pyk | | |
| P10D | Cupriavidus necator H16 ΔfdsGBACD ΔcbbLS | Conjugation | No |
| | ΔphaCAB: pTac-mcr-KamA pRbcl(M1)-mtkA-mtkB- | of BDISC1060 | |
| | mcl ΔphaC282: PnK-pyk ΔA0006: Pnk-BauA-maeB | with | |
| | pRbcL(M9)-gcl-gar-glxK | BDISC0900 | |

### Methods:

Deletion of Fds Operon

Homology arms were amplified from genomic DNA using Phu polymerase and used for Gibson assembly with p(TcColEloriTsacB) digested by Pvul. First crossover was confirmed with primer pairs in the vector and genome, confirming integration at the correct site. Second crossover was confirmed with primers either side of the site, outside the homology arms, to confirm the correct size and to sequence over the site from outside the region of recombination.

Deletion of cbbLS from Megaplasmid and Chromosome

Due to the matching operons, sequence similarity is very high between the megaplasmid and chromosome, especially downstream of the deletion site. Primers were designed to insert the homology arms into the p(TcColEloriTsacB) vector. The right homology arm (RHA) primers are all unselective due to the high degree of homology. The A left homology arm (LHA) Primers are unselective i.e. match the sequence for both chromosome and megaplasmid. B and C LHA forward Primers bind further upstream and vary in sequence between chromosome and megaplasmid, allowing selective amplification. Primer B will allow sequencing of the chromosomal cbbR gene.

Homology arms were amplified from genomic DNA with Option A primers, using Phu polymerase and used for Gibson assembly with p(TcColE1oriTsacB) digested by Pvul. This led to successful construction of the megaplasmid version of the deletion construct. However, no plasmids with chromosomal sequences in the homology arms were recovered. The chromosomal region was amplified specifically using B chromosomal Primers. This was then used as a template for the A Primers to amplify the shorter homology arms, resulting in successful construction of the chromosomal version of the deletion construct.

First crossover was confirmed with primer pairs in the vector and genome, confirming integration at the correct site. Second crossover is confirmed with primers either side of the site, outside the homology arms, to confirm the correct size and to sequence over the site. Sequencing was performed with B Primers and also confirmed integrity of the chromosomal cbbR gene.

Creation of Insertion Plasmids

The pathways are constructed of several genes, arranged into Transcriptional Units (TUs) of 2-3 genes with a promoter and terminator. The TUs are positioned between homology arms used to integrate in the genome.

The genes were ordered from Gen9 or DNA2.0, with promoters, terminators and homology arms appended as required, and with framing sequences to allow assembly using the Goldengate/Goldenbraid procedures (Table 9). The strategy for p5 operon TU2 required a large fragment containing the operon sequence, however this could not be synthesised. The genes were instead ordered in 3 parts of 1-2 genes each, retaining the native intergenic sequence of Methylococcus capsulatus between them by design of the overhangs. Typically, each TU was assembled first in pBBR1 Level 1 plasmids using Bsal, and then combined into Level 2 insertion vectors using Bbsl. Each assembly step was transformed into E. coli and checked by colony PCR and by sequencing over the joins. A number of genes/TUs were duplicated between pathways and could be reused.

Formate Testing

The base strain is ideally unable to metabolise formate, as well as unable to fix carbon by its native carbon fixation pathway. The deletion of both versions of Rubisco is fairly certain to remove the ability to fix carbon, as no other autotrophic carbon fixation pathways are known in these Cupriavidus necator. Carbon fixation cannot be tested in flasks as hydrogen cannot be supplied, so the confirmation of the loss of this ability will have to be confirmed in fermentation.

The deletion of the Fdh operon is initially proposed for disabling formate metabolism. The Fdh operon encodes the soluble formate dehydrogenase, and is likely to be sufficient to prevent growth on formate (Friedebold, J. O. R. G., & Bowien, B. (1993). Journal of bacteriology, 175(15), 4719-4728; Oh, J. I., & Bowien, B. (1998). Journal of Biological Chemistry, 273(41), 26349-26360). However, other formate dehydrogenases are known, all membrane-bound, and likely operons have been identified by homology (Pohlmann, A., Fricke, W. F., Reinecke, F., Kusian, B., Liesegang, H., Cramm, R., . . . & Strittmatter, A. (2006). Nature biotechnology, 24(10), 1257-1262). Membrane-bound formate dehydrogenases may not deplete formate biosynthesised inside the cell in a final strain, but could deplete formate in the media, which the formate-dependent pathways would need during initial optimisation work. Therefore, after deletion of Fdh, it is important to test the remaining formate dehydrogenase activity and see if it is low enough.

Formate dehydrogenase activity can be tested in flasks or in the fermenter. Growth on formate is difficult to determine in flasks as high levels of formate are toxic to the cells but low levels of formate lead to poor growth. The cells were grown on INV-2 based media containing fructose allowing growth, with formate at sub-toxic levels. OD samples and supernatant samples will be taken to see if there is a difference in growth rate and formate consumption between strains.

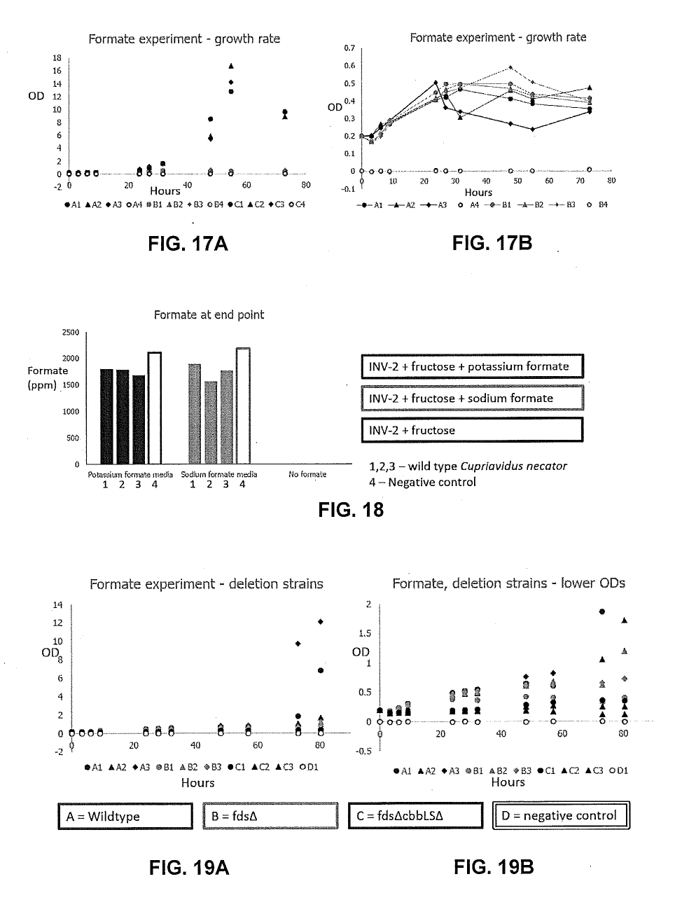

Consumption of formate by wild-type Cupriavidus necator was assessed. Wild type Cupriavidus necator was grown overnight in rich media before being washed in 0.85% NaCl and in INV-2+fructose media without formate. The cell suspension was inoculated into the INV-2+fructose media with potassium formate (A), INV-2+fructose media with sodium formate (B) or INV-2+fructose media without formate (C) to a calculated OD₆₀₀ of 0.2 and grown at 30° C. with shaking. Three flasks of each media were inoculated with a fourth flask left sterile as a negative control. OD was recorded over the experiment (FIG. 17A and FIG. 17B) and final levels of formate were tested to see if any had been consumed (FIG. 18). The cultures in INV-2 media containing no formate outgrew the cultures in INV-2 media containing formate so it is not clear from this result whether the strains are consuming the formate to contribute to growth, as the formate has a negative effect on growth.

The amount of fructose in the media was sufficient to fuel increased cell growth, as seen in the first formate tests with formate-free media (FIGS. 17A and 17B).

There was little difference between growth in INV-2 containing sodium formate vs potassium formate (FIG. 18).

The levels of formic acid dropped (by 13-28%) relative to the negative control, showing that the cells are consuming the formate. There is not much difference between formate consumption in INV-2 containing sodium formate vs potassium formate. No formate is detected in the controls.

Growth in media containing formate and consumption of formate by Cupriavidus necator base strains was also tested.

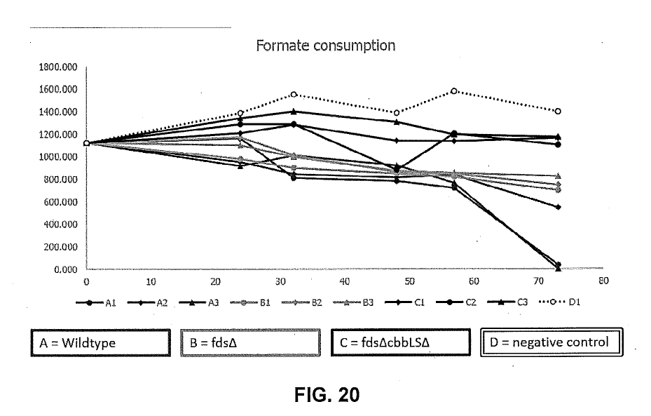

Wild type Cupriavidus necator, ΔfdsGBACD strain BDISC0312 and ΔfdsGBACD-ΔcbbLS(pl,chr) strain BDISC0334 were grown as above and inoculated into INV-2+fructose media with sodium formate to a calculated OD₆₀₀ of 0.2 and grown at 30° C. with shaking. 3 flasks were inoculated for each strain with a tenth flask left sterile as a negative control. As before, OD was recorded over the experiment (FIGS. 19A and 19B) and consumption of formate was monitored over the experiment (FIG. 20).

This test was done using INV-2 media containing half as much formate as the previous test and the same amount of fructose. Wild type Cupriavidus necator grew better in this media, reaching a higher OD, although still severely delayed compared to the wild type Cupriavidus necator in fructose-only INV-2 from the first tests. The ΔfdsGBACD strain, BDISC0312, grew a little slower than the wildtype to begin with but did not differ much until 73 h, when it did not increase in growth rate as wild type cells did. The ΔfdsGBACD-ΔcbbLS(pl,chr) strain, BDISC0334, grew a lot more slowly.

Formate levels seem to increase in the negative control (not inoculated, sterile media). The ΔfdsGBACD-ΔcbbLS(pl,chr) strain did not consume much formate, but also showed such a low OD this may be due to the small amount of cells to consume it. The wildtype and ΔfdsGBACD strain showed little difference up to the last timepoint, where the wildtype has consumed much of the formate.

The tests with wild type Cupriavidus necator and different formate media showed that formate at 2.7 g/L was enough to restrict growth of the cells in flask experiments. The second test done using lower levels of formate (1.35 g/L) achieved better growth. Results did not differ significantly between potassium and sodium formate media.

Wild type Cupriavidus necator showed a jump in growth rate towards the end of the experiment which may indicate that enough formate has been consumed to reduce its toxic effect on the cells. The ΔfdsGBACD strain, BDISC0312, did not differ much from the wild type until 73 h, when the growth rate did not show an increase. This suggests that formate consumption is reduced if not entirely inhibited. Formate consumption results agree with this, as formate drops rapidly towards the end of the experiment in the wildtype cultures. The drop in formate level seems to precede the increase in growth rate. This suggests that the ΔfdsGBACD cells do consume formate at a lower rate than the wildtype, but that formate consumption has not been abolished completely. A longer experiment, or a higher starting OD, may determine how different the rates of formate consumption are, or by using cells pre-grown in formate-containing media to allow pre-adaption.

The ΔfdsGBACD-ΔcbbLS(pl,chr) strain, BDISC0334, grew more slowly than the wild type or ΔfdsGBACD strain.

### OTHER EMBODIMENTS

The foregoing discussion discloses and describes merely exemplary embodiments of the disclosure. One skilled in the art will readily recognize from such discussion and from the accompanying drawings and claims, that various changes, modifications and variations can be made therein without departing from the spirit and scope of the disclosure as defined in the following claims.

1. A method of producing formate in a recombinant host, said method comprising:
enzymatically converting 2-methyl-isocitrate to pyruvate in said recombinant host using a protein having methylisocitrate lyase activity; and
enzymatically converting pyruvate to formate in said recombinant host using a protein having formate C-acetyltransferase activity.

2. The method of claim 1, further comprising:
enzymatically converting β-alanine to β-alanyl-CoA using a protein having CoA-transferase activity classified under EC 2.8.3.-; and
enzymatically converting β-alanyl-CoA to acrylol-CoA using a protein having acrylyl-CoA reductase activity; or
enzymatically converting 3-hydroxy-propanoate to 3-hydroxy-propanoyl-CoA using a protein having 3-hydroxypropionyl-CoA synthase activity and a protein having CoA-transferase activity; and
enzymatically converting 3-hydroxy-propanoyl-CoA to acrylol-CoA using a protein having β-alanyl-CoA ammonia-lyase activity.

3. The method of claim 2, wherein the protein having acrylyl-CoA reductase activity is classified under EC 1.3.1.84, the protein having 3-hydroxypropionyl-CoA synthase activity is classified under EC 6.2.1.36 or the protein having β-alanyl-CoA ammonia-lyase activity is classified under EC 4.3.1.6.

4. The method of claim 2, wherein said recombinant host overexpresses one or more genes encoding at least one protein having the activity of at least one enzyme selected from: a 2-methylisocitrate dehydratase, a methylisocitrate lyase, a succinate dehydrogenase (quinone), a fumarate reductase (quinol), a fumarate hydratase, a malate dehydrogenase, a 2-methylisocitrate dehydratase, a 2-methylcitrate synthase, an acrylyl-CoA reductase (NADPH), a β-alanyl-CoA:ammonia lyase, a glutamate dehydrogenase, a CoA-transferase, an alanine transaminase, a β-alanine pyruvate aminotransferase, a formate C-acetyltransferase, a malonyl-CoA reductase (malonate semialdehyde forming), an alanine-oxo-acid transaminase, and an acetyl-CoA carboxylase.

5-9. (canceled)

10. The method of claim 1, wherein the protein having methylisocitrate lyase activity is classified under EC 2.3.3.5.

11. A method of producing formate in a recombinant host, said method comprising:
enzymatically converting lactate to pyruvate in said recombinant host using a protein having L-lactate dehydrogenase activity and a protein having lactate-malate transhydrogenase activity; and
enzymatically converting pyruvate to formate in said recombinant host using a protein having formate C-acetyltransferase activity; or
enzymatically converting L-homoserine to 2-oxobutyrate in said recombinant host using a protein having threonine ammonia-lyase activity and a protein having cystathionine γ-lyase activity; and
enzymatically converting 2-oxobutyrate to formate in said recombinant host using a protein having formate C-acetyltransferase activity; or
enzymatically converting CO₂ to formate in said recombinant host using a protein having reductive NADP/NAPDH-dependent formate dehydrogenase activity.

12. The method of claim 11, wherein the protein having L-lactate dehydrogenase activity is classified under EC 1.1.1.27, the protein having lactate-malate transhydrogenase activity is classified under EC 1.1.99.7, the protein having threonine ammonia-lyase activity is classified under EC 4.3.1.19, the protein having cystathionine γ-lyase activity is classified under EC 4.4.1.1, the protein having formate C-acetyltransferase activity is classified under EC 2.3.1.54 or the protein having reductive NADP/NAPDH-dependent formate dehydrogenase activity is classified under EC 1.2.1.43 or EC 1.2.1.2.

13. (canceled)

14. The method of claim 11, wherein
said recombinant host overexpresses one or more genes encoding at least one protein having the activity of at least one enzyme selected from: an enoyl-CoA hydratase, a lactoyl-CoA dehydratase, a propionate CoA-transferase, a 3-hydroxypropionate dehydrogenase, a malonyl-CoA reductase (malonate semialdehyde-forming), an acetyl-CoA carboxylase, a formate C-acetyltransferase, a lactate-malate transhydrogenase, and a L-lactate dehydrogenase;
said recombinant host overexpresses one or more genes encoding at least one protein having the activity of at least one enzyme depicted in FIG. 6; or
said recombinant host overexpresses one or more genes encoding at least one protein having the activity of at least one enzyme selected from: a threonine ammonia-lyase, a cystathionine γ-lyase, a formate C-acetyltransferase, a 2-methylcitrate synthase, a 2-methylcitrate dehydratase, a 2-methylisocitrate dehydratase, a methylisocitrate lyase, a succinate dehydrogenase (quinone), a fumarate reductase (quinol), a fumarate hydratase, a malate dehydrogenase, a malate dehydrogenase (oxaloacetate-decarboxylating), an acetyl-CoA carboxylase, an aspartate kinase, an aspartate-semialdehyde dehydrogenase, a malate dehydrogenase, and a glutamate dehydrogenase.

15-22. (canceled)

23. A method of producing β-D-fructofuranose 6 phosphate in a recombinant host, said method comprising:
enzymatically converting formyl-CoA and NADH to formaldehyde and NAD⁺ in said recombinant host using a protein having acetaldehyde dehydrogenase activity;
enzymatically converting D-ribulose 5-phosphate and formaldehyde to hexulose 6-phosphate in said recombinant host using a protein having phosphoenolpyruvate carboxylase activity; and
enzymatically converting hexulose 6-phosphate to β-D-fructofuranose 6 phosphate in said recombinant host using a protein having 6-phospho-3-hexuloisomerase activity.

24. The method of claim 23, further comprising:
enzymatically converting formate, adenosine triphosphate, and succinyl-CoA to formyl-CoA, adenosine diphosphate, Pi, and succinate in said recombinant host using a protein having formyl-CoA transferase activity and a protein having acetate-CoA ligase activity.

25. The method of claim 24, wherein the protein having acetate-CoA ligase is classified under EC 6.2.1.1 or the protein having formyl-CoA transferase activity is classified under EC 2.8.3.16.

26. (canceled)

27. The method of claim 23, where the protein having acetaldehyde dehydrogenase activity is classified under EC 1.2.1.10, the protein having phosphoenolpyruvate carboxylase activity is classified under EC 4.1.1.31 or the protein having 6-phospho-3-hexuloisomerase activity is classified under EC 5.3.1.27.

28-29. (canceled)

30. The method of claim 1, wherein said recombinant host comprises an attenuation of one or more of the following genes: cbbL, cbbS, fdsG, fdsB, fdsA, fdsC, and fdsD or is a hydrogen-oxidizing microorganism.

31. (canceled)

32. The method of claim 30, wherein said hydrogen-oxidizing microorganism has an operable Calvin-Benson cycle.

33-39. (canceled)

40. A recombinant host comprising at least one exogenous nucleic acid encoding a methylisocitrate lyase and an anaplerotic enzyme.

41. The recombinant host of claim 40, wherein the anaplerotic enzyme is a pyruvate carboxylase, a phosphoenolpyruvate carboxylase, a malic enzyme, or an isocitrate dehydrogenase.

42. The recombinant host of claim 40, said recombinant host further comprising one or more of the following exogenous enzymes: 2-methylcitrate dehydratase, a methylisocitrate lyase, a succinate dehydrogenase (quinone), a fumarate reductase (quinol), a fumarate hydratase, a malate dehydrogenase, a 2-methylisocitrate dehydratase, a 2-methylcitrate synthase, an acrylyl-CoA reductase (NADPH), a β-alanyl-CoA:ammonia lyase, a glutamate dehydrogenase, a CoA-transferase, an alanine transaminase, a β-alanine pyruvate aminotransferase, a formate C-acetyltransferase, a malonyl-CoA reductase (malonate semialdehyde-forming), an acetyl-CoA carboxylase, an enoyl-CoA hydratase, a 3-hydroxypropionyl-CoA synthase, a lactoyl-CoA dehydratase, a propionate CoA-transferase, a L-lactate dehydrogenase, a lactate-malate transhydrogenase, a 3-hydroxypropionate dehydrogenase, a threonine ammonia-lyase, a cystathionine γ-lyase, a homoserine dehydrogenase, an aspartate-semialdehyde dehydrogenase, a malate dehydrogenase (oxaloacetate-decarboxylating), an aspartate kinase, a formate-tetrahydrofolate ligase, a methenyltetrahydrofolate cyclohydrolase, a glycine hydroxymethyltransferase, a serine-glyoxylate transaminase, a hydroxypyruvate reductase, a glycerate dehydrogenase, a glycerate 2-kinase, a phosphopyruvate hydratase, a phosphoenolpyruvate carboxylase, a malate-CoA ligase, a malyl-CoA lyase, a pyruvate kinase, a pyruvate carboxylase, a succinyl-CoA-L-malate CoA-transferase, a pyruvate synthase, a tartronate-semialdehyde synthase, an oxidoreductase with NAD(+) or NADP(+) as acceptor, a glycerate 3-kinase, a phosphoglycerate mutase (2,3-diphosphoglycerate-independent), a phosphoglycerate mutase (2,3-diphosphoglycerate-dependent), a pyruvate, phosphate dikinase, a pyruvate, water dikinase, a hydroxypyruvate isomerase, a 2-dehydro-3-deoxyglucarate aldolase, a 5-dehydro-4-deoxyglucarate dehydratase, a 2,5-dioxovalerate dehydrogenase, an acetate-CoA ligase, a formyl-CoA transferase, an aldehyde-alcohol dehydrogenase, a 6-phospho-3-hexuloisomerase, a 6-phosphofructokinase, a fructose-bisphosphate aldolase, a transketolase, a transaldolase, a ribulose phosphate 3-epimerase, a ribose-5-phosphate isomerase, a fructose-6-phosphate phosphoketolase, and a phosphate acetyltransferase.

43. The recombinant host of claim 40, wherein said recombinant host comprises an attenuation of one or more of the following genes: cbbL, cbbS, fdsG, fdsB, fdsA, fdsC, and fdsD;
said recombinant host overexpresses one or more genes encoding at least one protein having the activity of at least one enzyme depicted in FIGS. 3 to 12; or
said recombinant host overexpresses one or more genes encoding at least one protein having the activity of at least one enzyme selected from: a 2-methylcitrate dehydratase, a methylisocitrate lyase, a succinate dehydrogenase (quinone), a fumarate reductase (quinol), a fumarate hydratase, a malate dehydrogenase, a 2-methylisocitrate dehydratase, a 2-methylcitrate synthase, an acrylyl-CoA reductase (NADPH), a β-alanyl-CoA:ammonia lyase, a glutamate dehydrogenase, a CoA-transferase, an alanine transaminase, a β-alanine pyruvate aminotransferase, a formate C-acetyltransferase, a malonyl-CoA reductase (malonate semialdehyde-forming), an acetyl-CoA carboxylase, an enoyl-CoA hydratase, a 3-hydroxypropionyl-CoA synthase, a lactoyl-CoA dehydratase, a propionate CoA-transferase, a L-lactate dehydrogenase, a lactate-malate transhydrogenase, a 3-hydroxypropionate dehydrogenase, a threonine ammonia-lyase, a cystathionine γ-lyase, a homoserine dehydrogenase, an aspartate-semialdehyde dehydrogenase, a malate dehydrogenase (oxaloacetate-decarboxylating), an aspartate kinase, a formate-tetrahydrofolate ligase, a methenyltetrahydrofolate cyclohydrolase, a glycine hydroxymethyltransferase, a serine-glyoxylate transaminase, a hydroxypyruvate reductase, a glycerate dehydrogenase, a glycerate 2-kinase, a phosphopyruvate hydratase, a phosphoenolpyruvate carboxylase, a malate-CoA ligase, a malyl-CoA lyase, a pyruvate kinase, a pyruvate carboxylase, a succinyl-CoA-L-malate CoA-transferase, a pyruvate synthase, a tartronate-semialdehyde synthase, an oxidoreductase with NAD(+) or NADP(+) as acceptor, a glycerate 3-kinase, a phosphoglycerate mutase (2,3-diphosphoglycerate-independent), a phosphoglycerate mutase (2,3-diphosphoglycerate-dependent), a pyruvate, phosphate dikinase, a pyruvate, water dikinase, a hydroxypyruvate isomerase, a 2-dehydro-3-deoxyglucarate aldolase, a 5-dehydro-4-deoxyglucarate dehydratase, a 2,5-dioxovalerate dehydrogenase, an acetate-CoA ligase, a formyl-CoA transferase, an aldehyde-alcohol dehydrogenase, a 6-phospho-3-hexuloisomerase, a 6-phosphofructokinase, a fructose-bisphosphate aldolase, a transketolase, a transaldolase, a ribulose phosphate 3-epimerase, a ribose-5-phosphate isomerase, a fructose-6-phosphate phosphoketolase, and a phosphate acetyltransferase.

44-45. (canceled)

46. The recombinant host of claim 40, wherein said recombinant host is a hydrogen-oxidizing microorganism.

47. The recombinant host of claim 46, wherein said hydrogen-oxidizing microorganism has an operable Calvin-Benson cycle.

48. A method for more efficiently recycling reduced electron carriers or fixing carbon in a recombinant host comprising:
providing at least one microorganism capable of hydrogen oxidation, wherein the microorganism has an operable Calvin-Benson cycle;
attenuating the Calvin Benson cycle in said microorganism; and
utilizing the donated electrons or fixing carbon more efficiently than the microorganism having a Calvin Benson cycle.

49-51. (canceled)

52. The method of, claim 48 wherein the hydrogen-oxidizing microorganism with an operable Calvin-Benson cycle is selected from Cupriavidus necator, Hydrogenovibrio marinus, Rhodobacter capsulatus, Rhodobacter sphaeroides, Rhodospirillum rubrum, Thiobacillus ferrooxidans, and Xanthobacter flavus.
